SEQUENCE LISTING (1030 sequences)

SEQ ID NO: 1 - protein, 448 aa, synthetic construct
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, synthetic construct
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSS

SEQ ID NO: 3 - protein, 213 aa, synthetic construct
DIQMTQSPSSLSASVGDRVTITCSASSSVYYMYWFQQKPGKAPKLWIYSTSNLASGVPSRFSGSGSGTDYTLTISSMQPEDFATYYCQQRRNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 106 aa, synthetic construct
DIQMTQSPSSLSASVGDRVTITCSASSSVYYMYWFQQKPGKAPKLWIYSTSNLASGVPSRFSGSGSGTDYTLTISSMQPEDFATYYCQQRRNYPYTFGQGTKLEIK

SEQ ID NO: 5 - protein, 448 aa, synthetic construct; identical to SEQ ID NO: 1

SEQ ID NO: 6 - protein, 118 aa, synthetic construct; identical to SEQ ID NO: 2

SEQ ID NO: 7 - protein, 213 aa, synthetic construct; identical to SEQ ID NO: 3

SEQ ID NO: 8 - protein, 106 aa, synthetic construct; identical to SEQ ID NO: 4

SEQ ID NO: 9 - protein, 447 aa, synthetic construct
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 117 aa, synthetic construct
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSS

SEQ ID NO: 11 - protein, 219 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 13 - protein, 448 aa, synthetic construct
QVQLVQSGAEVKKPGASVKVSCKASGYTFSAYWIEWVRQAPGKGLEWVGEILPGSGNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 118 aa, synthetic construct
QVQLVQSGAEVKKPGASVKVSCKASGYTFSAYWIEWVRQAPGKGLEWVGEILPGSGNSRYNEKFKGRVTVTRDTSTNTAYMELSSLRSEDTAVYYCARSYDFAWFAYWGQGTLVTVSS

SEQ ID NO: 15 - protein, 220 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 113 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIK

SEQ ID NO: 17 - protein, 447 aa, synthetic construct
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 117 aa, synthetic construct
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSA

SEQ ID NO: 19 - protein, 213 aa, synthetic construct
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 106 aa, synthetic construct
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELK

SEQ ID NO: 21 - protein, 446 aa, synthetic construct
EVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 116 aa, synthetic construct
EVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSS

SEQ ID NO: 23 - protein, 219 aa, synthetic construct
DVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 112 aa, synthetic construct
DVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIK

SEQ ID NO: 25 - protein, 461 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 26 - protein, 122 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 27 - protein, 214 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 107 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 29 - protein, 448 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 118 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSA

SEQ ID NO: 31 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 33 - protein, 446 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 116 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 35 - protein, 214 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 107 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 37 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 39 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 41 - protein, 450 aa, synthetic construct
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 120 aa, synthetic construct
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSS

SEQ ID NO: 43 - protein, 214 aa, synthetic construct
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 107 aa, synthetic construct
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIK

SEQ ID NO: 45 - protein, 449 aa, synthetic construct
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 119 aa, synthetic construct
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSS

SEQ ID NO: 47 - protein, 214 aa, synthetic construct
DIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 107 aa, synthetic construct
DIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIK

SEQ ID NO: 49 - protein, 452 aa, synthetic construct
EVQLVESGGGLVQPGGSLRLSCAASGFTFNTYAMNWVRQAPGKGLEWVARIRSKSNNYATYYADSVKDRFTISRDDAKNSLYLQMNSLRAEDTAVYYCVRQWDYDVRAMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 122 aa, synthetic construct
EVQLVESGGGLVQPGGSLRLSCAASGFTFNTYAMNWVRQAPGKGLEWVARIRSKSNNYATYYADSVKDRFTISRDDAKNSLYLQMNSLRAEDTAVYYCVRQWDYDVRAMNYWGQGTLVTVSS

SEQ ID NO: 51 - protein, 214 aa, synthetic construct
DIQMTQSPSSLSASVGDRVTITCKASQDVDTAVAWYQQKPGKAPKLLIYWASTRLTGVPSRFSGSGSGTDFTLTISSLQPEDFATYYCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 107 aa, synthetic construct
DIQMTQSPSSLSASVGDRVTITCKASQDVDTAVAWYQQKPGKAPKLLIYWASTRLTGVPSRFSGSGSGTDFTLTISSLQPEDFATYYCQQYSSYPYTFGQGTKLEIK

SEQ ID NO: 53 - protein, 452 aa, synthetic construct
EVQLEESGGGLVKPGGSLRLSCAASGIDLSHYVVGWVRQAPGKGLEWVSIIYGSGRTYYANWAKGRFTISRDNSKNTLYLQMNSLRAEDTAVYYCARDASVSVYYWGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 122 aa, synthetic construct
EVQLEESGGGLVKPGGSLRLSCAASGIDLSHYVVGWVRQAPGKGLEWVSIIYGSGRTYYANWAKGRFTISRDNSKNTLYLQMNSLRAEDTAVYYCARDASVSVYYWGYFDLWGRGTLVTVSS

SEQ ID NO: 55 - protein, 217 aa, synthetic construct
DIQMTQSPSSLSASVGDRVTITCQASQSIGSELAWYQQKPGQAPKLLIYRASTLESGVPSRFSGSGSGTEFTFTISSLQPEDLATYYCQQGYTYSEIDNAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 110 aa, synthetic construct
DIQMTQSPSSLSASVGDRVTITCQASQSIGSELAWYQQKPGQAPKLLIYRASTLESGVPSRFSGSGSGTEFTFTISSLQPEDLATYYCQQGYTYSEIDNAFGQGTKLEIK

SEQ ID NO: 57 - protein, 451 aa, synthetic construct
EVQLEESGGGLVKPGGSLRLSCAASGIDLSSYGMGWVRQAPGKGLEWVSIISRNSVTYYATWAKGRFTISRDNSKNTLYLQMNSLRAEDTAVYYCARRATYSGALGYFDIWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 121 aa, synthetic construct
EVQLEESGGGLVKPGGSLRLSCAASGIDLSSYGMGWVRQAPGKGLEWVSIISRNSVTYYATWAKGRFTISRDNSKNTLYLQMNSLRAEDTAVYYCARRATYSGALGYFDIWGRGTLVTVSS

SEQ ID NO: 59 - protein, 217 aa, synthetic construct
GYDMTQSPSSVSASVGDRVTITCQASENIYSTLAWYQQKPGKAPKLLIYDAFDLASGVPSRFKGSGSGTEYTLTISSLQPEDFATYYCQQGYSGTNVDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 110 aa, synthetic construct
GYDMTQSPSSVSASVGDRVTITCQASENIYSTLAWYQQKPGKAPKLLIYDAFDLASGVPSRFKGSGSGTEYTLTISSLQPEDFATYYCQQGYSGTNVDNAFGGGTKVEIK

SEQ ID NO: 61 - protein, 452 aa, synthetic construct
QVQLQQWGAGLLKPSETLSLTCAVYGGSFSGYYWTWIRQPPGKGLEWIGEIDHSESTNYNPSLKSRVTISVDTSKNQFSLKLSSVTAADTAVYYCAGWFGELYHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 122 aa, synthetic construct
QVQLQQWGAGLLKPSETLSLTCAVYGGSFSGYYWTWIRQPPGKGLEWIGEIDHSESTNYNPSLKSRVTISVDTSKNQFSLKLSSVTAADTAVYYCAGWFGELYHYYYGMDVWGQGTTVTVSS

SEQ ID NO: 63 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 65 - protein, 452 aa, synthetic construct
EVQLVESGGGLVQPGGSLRLSCAASGFTFSSYLMTWVRQAPGKGLEWVANIKQDGSEKYYVDSVKGRFTISRDNAKNSLNLQMNSLRAEDTAVYYCTRDGYSSGRHYGMDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 122 aa, synthetic construct
EVQLVESGGGLVQPGGSLRLSCAASGFTFSSYLMTWVRQAPGKGLEWVANIKQDGSEKYYVDSVKGRFTISRDNAKNSLNLQMNSLRAEDTAVYYCTRDGYSSGRHYGMDVWGQGTTVIVSS

SEQ ID NO: 67 - protein, 214 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSNWPRTFGQGTKVEIK

SEQ ID NO: 69 - protein, 451 aa, synthetic construct
EVQLVESGGGLVQPGGSLRLSCAASGFTFSRHWMSWVRQAPGKGLEWVSEINPDSRTINYAPSVKGRFTISRDNAKNSLYLQMNSLRAEDTAVYYCARRVRIHYYGAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 121 aa, synthetic construct
EVQLVESGGGLVQPGGSLRLSCAASGFTFSRHWMSWVRQAPGKGLEWVSEINPDSRTINYAPSVKGRFTISRDNAKNSLYLQMNSLRAEDTAVYYCARRVRIHYYGAMDSWGQGTTVTVSS

SEQ ID NO: 71 - protein, 214 aa, synthetic construct
DIQLTQSQSFVSTSVGDRVTVTCRASQNVGSSLVWYQQKPGKSPKTLIYSASFLYSGVPSRFSGSGSGTEFTLTISSVQPEDFADYFCQQYNNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 107 aa, synthetic construct
DIQLTQSQSFVSTSVGDRVTVTCRASQNVGSSLVWYQQKPGKSPKTLIYSASFLYSGVPSRFSGSGSGTEFTLTISSVQPEDFADYFCQQYNNYPLTFGGGTKVEIK

SEQ ID NO: 73 - protein, 449 aa, synthetic construct
EVQLQQSGAELVKPGASVKLSCTTSGFNIIDTYIHWVNQKPDQGLEWIGRIDPADGNRKSDPKFQVKATITVDTSSNTAYLQLSSLTSGDTAVYYCARGYGNLNAMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 119 aa, synthetic construct
EVQLQQSGAELVKPGASVKLSCTTSGFNIIDTYIHWVNQKPDQGLEWIGRIDPADGNRKSDPKFQVKATITVDTSSNTAYLQLSSLTSGDTAVYYCARGYGNLNAMDSWGQGTSVTVSS

SEQ ID NO: 75 - protein, 214 aa, synthetic construct
DIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 107 aa, synthetic construct
DIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIK

SEQ ID NO: 77 - protein, 452 aa, synthetic construct
QVQLVQSGAEVKKPGATVKISCKVSGYTFTDYHMDWVQQAPGKGLEWMGDINPNNGGAIYNQKFKGRVTITADTSTDTAYMELSSLRSEDTAVYYCARSHYDYAGGAWFAYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 122 aa, synthetic construct
QVQLVQSGAEVKKPGATVKISCKVSGYTFTDYHMDWVQQAPGKGLEWMGDINPNNGGAIYNQKFKGRVTITADTSTDTAYMELSSLRSEDTAVYYCARSHYDYAGGAWFAYWGQGTLVTVSR

SEQ ID NO: 79 - protein, 220 aa, synthetic construct
EIVMTQSPGTLSLSPGERATLSCKSSQSLLFSGNQKNYLAWYQQKPGQAPRLLIYWASTRASGIPDRFSGSGSGTDFTLTISRLEPEDFAVYYCQQYYSFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 113 aa, synthetic construct
EIVMTQSPGTLSLSPGERATLSCKSSQSLLFSGNQKNYLAWYQQKPGQAPRLLIYWASTRASGIPDRFSGSGSGTDFTLTISRLEPEDFAVYYCQQYYSFPRTFGQGTKLEIK

SEQ ID NO: 81 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 117 aa, synthetic construct [sequence omitted]

SEQ ID NO: 83 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 113 aa, synthetic construct [sequence omitted]

SEQ ID NO: 85 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 117 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSS

SEQ ID NO: 87 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 107 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIK

SEQ ID NO: 89 - protein, 451 aa, synthetic construct
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 121 aa, synthetic construct [sequence omitted]

SEQ ID NO: 91 - protein, 214 aa, synthetic construct
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 107 aa, synthetic construct [sequence omitted]

SEQ ID NO: 93 - protein, 450 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 120 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSS

SEQ ID NO: 95 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 113 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIK

SEQ ID NO: 97 - protein, 451 aa, synthetic construct
EVQLLESGGGLVQPGGSLRLSCAASGFTFSDYDMSWVRQAPGKGLEWVSTIDLDSGSIYYADSVQGRFTISRDNSKNTLYLQMNSLRAEDTAVYYCAKDLHMGPEGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 121 aa, synthetic construct
EVQLLESGGGLVQPGGSLRLSCAASGFTFSDYDMSWVRQAPGKGLEWVSTIDLDSGSIYYADSVQGRFTISRDNSKNTLYLQMNSLRAEDTAVYYCAKDLHMGPEGPFDYWGQGTLVTVSS

SEQ ID NO: 99 - protein, 217 aa, synthetic construct
QSVLTQPPSASGTPGQRVTISCSGSSSNIGSNSVSWYQQLPGTAPKLLIYSDNHRPSGVPDRFSGSKSGTSASLAISGLRSEDEADYYCQGWDTSLSGHVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 110 aa, synthetic construct
QSVLTQPPSASGTPGQRVTISCSGSSSNIGSNSVSWYQQLPGTAPKLLIYSDNHRPSGVPDRFSGSKSGTSASLAISGLRSEDEADYYCQGWDTSLSGHVFGGGTKLTVL

SEQ ID NO: 101 - protein, 453 aa, synthetic construct
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 123 aa, synthetic construct
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 103 - protein, 218 aa, synthetic construct
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 111 aa, synthetic construct
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 105 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTNSAKFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTNSAKFDPWGQGTMVTVSS

SEQ ID NO: 107 - protein, 218 aa, synthetic construct
QSALTQPASVSGSPGQSITISCTGTSNDVGAYNYVSWYQQHPGKAPKLMISEVNKRPSGVSNRFSGSKSGNTASLTISGLQAEDEADYYCSSYTMGSTFMLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 111 aa, synthetic construct
QSALTQPASVSGSPGQSITISCTGTSNDVGAYNYVSWYQQHPGKAPKLMISEVNKRPSGVSNRFSGSKSGNTASLTISGLQAEDEADYYCSSYTMGSTFMLFGGGTKLTVL

SEQ ID NO: 109 - protein, 446 aa, synthetic construct
QVQLVESGGGVVQPGRSLRLSCAASGFSLTSYGVHWVRQAPGKGLEWVGVIWSGGSTDYADSVKGRFTISKDNSKNTVYLQMNSLRAEDTAVYYCARNSNNGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 116 aa, synthetic construct
QVQLVESGGGVVQPGRSLRLSCAASGFSLTSYGVHWVRQAPGKGLEWVGVIWSGGSTDYADSVKGRFTISKDNSKNTVYLQMNSLRAEDTAVYYCARNSNNGFAYWGQGTLVTVSS

SEQ ID NO: 111 - protein, 214 aa, synthetic construct
DIQLTQSPSSLSASVGDRVTITCRASQNIYSNLAWYQQKPGKAPKLLVYAAKNLASGVPSRFSGSGSGTDFTLTISSLQPEDFATYYCQHFYDTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 107 aa, synthetic construct
DIQLTQSPSSLSASVGDRVTITCRASQNIYSNLAWYQQKPGKAPKLLVYAAKNLASGVPSRFSGSGSGTDFTLTISSLQPEDFATYYCQHFYDTPWTFGQGTKVEIK

SEQ ID NO: 113 - protein, 449 aa, synthetic construct
QVQLQESGPELVKPGASVKMSCKASGYSFTAYNMHWVKQSHGKSLEWIGFIDPYSGIITYNQTFKGKATLTVDKSSSTAYMQLNSLTSEDSAVYYCARRGYYDGGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 119 aa, synthetic construct
QVQLQESGPELVKPGASVKMSCKASGYSFTAYNMHWVKQSHGKSLEWIGFIDPYSGIITYNQTFKGKATLTVDKSSSTAYMQLNSLTSEDSAVYYCARRGYYDGGFDYWGQGTTLTVSS

SEQ ID NO: 115 - protein, 214 aa, synthetic construct
DIQMTQTTSSLSASLGDRVTISCRASQDITNYLNWYQQKPDGTVKLLIYYTSRLHSGVPSRFSGSGSGTDYSLTISNLEQEDIATYFCQQDSKHPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 107 aa, synthetic construct
DIQMTQTTSSLSASLGDRVTISCRASQDITNYLNWYQQKPDGTVKLLIYYTSRLHSGVPSRFSGSGSGTDYSLTISNLEQEDIATYFCQQDSKHPRTFGGGTKLEIK

SEQ ID NO: 117 - protein, 452 aa, synthetic construct
QVQLQQPGTELVKPGASVKLSCKASGYTFTRYWINWVKQRPQGGLEWIGNIYPGSNITNYNEKFKNKATLTVDTSSNTAYMQLSSLTSDDSAVYYCAREGIYDGYFPLFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 122 aa, synthetic construct
QVQLQQPGTELVKPGASVKLSCKASGYTFTRYWINWVKQRPQGGLEWIGNIYPGSNITNYNEKFKNKATLTVDTSSNTAYMQLSSLTSDDSAVYYCAREGIYDGYFPLFPYWGQGTLVTVSA

SEQ ID NO: 119 - protein, 215 aa, synthetic construct
ENVLTQSPAIMAASPGEKVTMTCSASSSVSSGNFHWYQQKPGTSPKLWIYRTSNLASGVPARFSGSGSGTSYSLTISSMEAEDAATYYCQQWSGYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 108 aa, synthetic construct
ENVLTQSPAIMAASPGEKVTMTCSASSSVSSGNFHWYQQKPGTSPKLWIYRTSNLASGVPARFSGSGSGTSYSLTISSMEAEDAATYYCQQWSGYPWTFGGGTKLEIK

SEQ ID NO: 121 - protein, 453 aa, synthetic construct
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 123 aa, synthetic construct
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 123 - protein, 213 aa, synthetic construct
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 106 aa, synthetic construct
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 125 - protein, 446 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 116 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 127 - protein, 219 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 112 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 129 - protein, 446 aa, synthetic construct
QIQLVQSGPELKKPGETVKISCKASGYTFTKYGMNWVKQAPGKGLKWMGWINTYTEEPTYGDDFKGRFAFSLETSASTANLQINNLKSEDTATYFCARFGSAVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 116 aa, synthetic construct
QIQLVQSGPELKKPGETVKISCKASGYTFTKYGMNWVKQAPGKGLKWMGWINTYTEEPTYGDDFKGRFAFSLETSASTANLQINNLKSEDTATYFCARFGSAVDYWGQGTSVTVSS

SEQ ID NO: 131 - protein, 219 aa, synthetic construct
DIVMTQAAFSNPVTLGTSGSISCRSSKSLLHSNGITYLYWYLQKPGQSPQLLIYQMSNLASGVPDRFSSSGSGTDFTLRISRVEAEDVGVYYCAQNLELPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 112 aa, synthetic construct
DIVMTQAAFSNPVTLGTSGSISCRSSKSLLHSNGITYLYWYLQKPGQSPQLLIYQMSNLASGVPDRFSSSGSGTDFTLRISRVEAEDVGVYYCAQNLELPRTFGGGTKLEIK

SEQ ID NO: 133 - protein, 446 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 116 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 135 - protein, 219 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 112 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 137 - protein, 442 aa, synthetic construct
QVQLVQSGAEVKKPGASVKVSCKASGGTFSSYAISWVRQAPGQGLEWMGGIVPIFGTANYAQKFQGRVTITADESTSTAYMELSSLRSEDTAVYYCARDPFLHYWGQGTLVTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 112 aa, synthetic construct
QVQLVQSGAEVKKPGASVKVSCKASGGTFSSYAISWVRQAPGQGLEWMGGIVPIFGTANYAQKFQGRVTITADESTSTAYMELSSLRSEDTAVYYCARDPFLHYWGQGTLVT

SEQ ID NO: 139 - protein, 214 aa, synthetic construct
EIELTQSPGTLSLSPGERATLSCRASQSVSSSYLAWYQQKPGQAPRLLIYGASSRATGIPDRFSGSGSGTDFTLTISRLEPEDFAVYYCAQGELYPRQFGGGTKLDIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 107 aa, synthetic construct
EIELTQSPGTLSLSPGERATLSCRASQSVSSSYLAWYQQKPGQAPRLLIYGASSRATGIPDRFSGSGSGTDFTLTISRLEPEDFAVYYCAQGELYPRQFGGGTKLDI

SEQ ID NO: 141 - protein, 445 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 143 - protein, 216 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 145 - protein, 449 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 119 aa, synthetic construct [sequence omitted]

SEQ ID NO: 147 - protein, 213 aa, synthetic construct
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 106 aa, synthetic construct [sequence omitted]

SEQ ID NO: 149 - protein, 451 aa, synthetic construct
EVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 121 aa, synthetic construct [sequence omitted]

SEQ ID NO: 151 - protein, 214 aa, synthetic construct
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 107 aa, synthetic construct [sequence omitted]

SEQ ID NO: 153 - protein, 448 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 118 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSS

SEQ ID NO: 155 - protein, 214 aa, synthetic construct
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 107 aa, synthetic construct
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIK

SEQ ID NO: 157 - protein, 452 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 122 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 159 - protein, 215 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 108 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 161 - protein, 451 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 121 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 163 - protein, 215 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 165 - protein, 450 aa, synthetic construct
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 120 aa, synthetic construct
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSS

SEQ ID NO: 167 - protein, 215 aa, synthetic construct
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 108 aa, synthetic construct
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIK

SEQ ID NO: 169 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 171 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 173 - protein, 456 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 174 - protein, 117 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 175 - protein, 219 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 112 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 177 - protein, 455 aa, synthetic construct
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 125 aa, synthetic construct
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 179 - protein, 213 aa, synthetic construct
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 106 aa, synthetic construct
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 181 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 118 aa, synthetic construct [sequence omitted]

SEQ ID NO: 183 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 107 aa, synthetic construct [sequence omitted]

SEQ ID NO: 185 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 187 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 189 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 191 - protein, 217 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 193 - protein, 451 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 121 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSS

SEQ ID NO: 195 - protein, 215 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 197 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 199 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 201 - protein, 451 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 203 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 205 - protein, 449 aa, synthetic construct
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 119 aa, synthetic construct
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 207 - protein, 218 aa, synthetic construct
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 111 aa, synthetic construct
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 209 - protein, 445 aa, synthetic construct
QVQLVQSGAEVKKPGASVKVSCKASGYTFTSYWINWVRQAPGQGLEWMGNIFPSDSYSNYNKKFKDRVTMTRDTSTSTVYMELSSLRSEDTAVYYCARGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 115 aa, synthetic construct
QVQLVQSGAEVKKPGASVKVSCKASGYTFTSYWINWVRQAPGQGLEWMGNIFPSDSYSNYNKKFKDRVTMTRDTSTSTVYMELSSLRSEDTAVYYCARGSGFDYWGQGTLVTVSS

SEQ ID NO: 211 - protein, 214 aa, synthetic construct
EIVLTQSPATLSLSPGERATLSCRASQTIGTSIHWYQQKPGQAPRLLIYYASESISGIPARFSGSGSGTDFTLTISSLEPEDFAVYYCSQSFSWPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 107 aa, synthetic construct
EIVLTQSPATLSLSPGERATLSCRASQTIGTSIHWYQQKPGQAPRLLIYYASESISGIPARFSGSGSGTDFTLTISSLEPEDFAVYYCSQSFSWPFTFGQGTKLEIK

SEQ ID NO: 213 - protein, 446 aa, synthetic construct
QVQLQQSGPELVRPGTSVRISCKASGYTFTIYWLGWVKQRPGHGLEWIGNIFPGSAYINYNEKFKGKATLTADTSSSTAYMQLSSLTSEDSAVYFCAREGSNSGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 116 aa, synthetic construct
QVQLQQSGPELVRPGTSVRISCKASGYTFTIYWLGWVKQRPGHGLEWIGNIFPGSAYINYNEKFKGKATLTADTSSSTAYMQLSSLTSEDSAVYFCAREGSNSGYWGQGTTLTVSS

SEQ ID NO: 215 - protein, 220 aa, synthetic construct
DIVMTQSPSSLSVSAGEKVTMTCKSSQSLLNSGNQQNYLAWYQQKPGQPPKLLIYGASTRESGVPDRFTGSGSGTDFTLTINSVQAEDLAVYYCQSDHIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 113 aa, synthetic construct
DIVMTQSPSSLSVSAGEKVTMTCKSSQSLLNSGNQQNYLAWYQQKPGQPPKLLIYGASTRESGVPDRFTGSGSGTDFTLTINSVQAEDLAVYYCQSDHIYPYTFGGGTKLEIK

SEQ ID NO: 217 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 128 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 219 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 107 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 221 - protein, 449 aa, synthetic construct
QIQLVQSGPELKKPGETVKISCKASGYTFTHYSMHWVKQAPGKGLKWMGWINTYTGEPTYADDFKGRFAFSLETSASTAFLQINNLKNEDTATYFCATYYRYERDFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 119 aa, synthetic construct
QIQLVQSGPELKKPGETVKISCKASGYTFTHYSMHWVKQAPGKGLKWMGWINTYTGEPTYADDFKGRFAFSLETSASTAFLQINNLKNEDTATYFCATYYRYERDFDYWGQGTTLTVSS

SEQ ID NO: 223 - protein, 219 aa, synthetic construct
DVLMTQSPLSLPVSLGDQASISCRSSQSIVHSNGNTYLEWYLQKPGQSPKLLIYKVSNRFSGVPDRFSGSGSGTDFTLRISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 112 aa, synthetic construct
DVLMTQSPLSLPVSLGDQASISCRSSQSIVHSNGNTYLEWYLQKPGQSPKLLIYKVSNRFSGVPDRFSGSGSGTDFTLRISRVEAEDLGVYYCFQGSHVPYTFGGGTKLEIK

SEQ ID NO: 225 - protein, 450 aa, synthetic construct
QVQLVQSGAEVKKPGASVKVSCKASGYTFTGYYIHWVRQAPGQGLEWMGWIYPGNFNTKYNERFKGRVTMTTDTSTSTAYMELRSLRSDDTAVYYCAREDGSPYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 120 aa, synthetic construct
QVQLVQSGAEVKKPGASVKVSCKASGYTFTGYYIHWVRQAPGQGLEWMGWIYPGNFNTKYNERFKGRVTMTTDTSTSTAYMELRSLRSDDTAVYYCAREDGSPYYAMDYWGQGTSVTVSS

SEQ ID NO: 227 - protein, 219 aa, synthetic construct
DIVMTQTPLSLSVTPGQPASISCRSSQSLVHSNGNTFLYWYLQKPGQSPQLLIYRVSNRFSGVPDRFSGSGSGTDFTLKISRVEAEDVGVYYCFQATHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 112 aa, synthetic construct
DIVMTQTPLSLSVTPGQPASISCRSSQSLVHSNGNTFLYWYLQKPGQSPQLLIYRVSNRFSGVPDRFSGSGSGTDFTLKISRVEAEDVGVYYCFQATHVPWTFGGGTKVEIK

SEQ ID NO: 229 - protein, 448 aa, synthetic construct
QVQLVQSGAEVKKPGASVKVSCKASGYTFTYFYMNWVRQAPGQGLEWVGQINPNNGGTAYAQKFQGRVTMTRDTSTSTVYMELSSLRSEDTAVYYCARWVGTHYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 118 aa, synthetic construct
QVQLVQSGAEVKKPGASVKVSCKASGYTFTYFYMNWVRQAPGQGLEWVGQINPNNGGTAYAQKFQGRVTMTRDTSTSTVYMELSSLRSEDTAVYYCARWVGTHYFDYWGQGTTLTVSS

SEQ ID NO: 231 - protein, 218 aa, synthetic construct
EIVLTQSPATLSLSPGERATLSCRASQSVSSSSYTYIHWYQQKPGQAPRLLINFASNLESGIPARFSGSGSGTDFTLTISSLEPEDFAVYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 111 aa, synthetic construct
EIVLTQSPATLSLSPGERATLSCRASQSVSSSSYTYIHWYQQKPGQAPRLLINFASNLESGIPARFSGSGSGTDFTLTISSLEPEDFAVYYCQHSWEIPPTFGGGTKLEIK

SEQ ID NO: 233 - protein, 446 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 116 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 235 - protein, 214 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 107 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 237 - protein, 446 aa, synthetic construct
EVQLVQSGAEVKKPGASVKVSCKASGYTFTSYWMHWVRQAPGQRLEWIGEIAPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 116 aa, synthetic construct
EVQLVQSGAEVKKPGASVKVSCKASGYTFTSYWMHWVRQAPGQRLEWIGEIAPTNGRTNYIEKFKSRATLTVDKSASTAYMELSSLRSEDTAVYYCARGTRAYHYWGQGTMVTVSS

SEQ ID NO: 239 - protein, 214 aa, synthetic construct; identical to SEQ ID NO: 235

SEQ ID NO: 240 - protein, 107 aa, synthetic construct; identical to SEQ ID NO: 236

SEQ ID NO: 241 - protein, 446 aa, synthetic construct
EVQLVQSGAEVKKPGASVKVSCKASGYTFTSYWMHWVRQAPGQRLEWIGEINPANGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 116 aa, synthetic construct
EVQLVQSGAEVKKPGASVKVSCKASGYTFTSYWMHWVRQAPGQRLEWIGEINPANGRTNYIEKFKSRATLTVDKSASTAYMELSSLRSEDTAVYYCARGTRAYHYWGQGTMVTVSS

SEQ ID NO: 243 - protein, 214 aa, synthetic construct; identical to SEQ ID NO: 235

SEQ ID NO: 244 - protein, 107 aa, synthetic construct; identical to SEQ ID NO: 236

SEQ ID NO: 245 - protein, 446 aa, synthetic construct; identical to SEQ ID NO: 241

SEQ ID NO: 246 - protein, 116 aa, synthetic construct; identical to SEQ ID NO: 242

SEQ ID NO: 247 - protein, 214 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFAGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 107 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFAGTPLTFGQGTKVEIK

SEQ ID NO: 249 - protein, 446 aa, synthetic construct; identical to SEQ ID NO: 233

SEQ ID NO: 250 - protein, 116 aa, synthetic construct; identical to SEQ ID NO: 234

SEQ ID NO: 251 - protein, 214 aa, synthetic construct; identical to SEQ ID NO: 247

SEQ ID NO: 252 - protein, 107 aa, synthetic construct; identical to SEQ ID NO: 248

SEQ ID NO: 253 - protein, 446 aa, synthetic construct; identical to SEQ ID NO: 237

SEQ ID NO: 254 - protein, 116 aa, synthetic construct; identical to SEQ ID NO: 238

SEQ ID NO: 255 - protein, 214 aa, synthetic construct; identical to SEQ ID NO: 247

SEQ ID NO: 256 - protein, 107 aa, synthetic construct; identical to SEQ ID NO: 248

SEQ ID NO: 257 - protein, 450 aa, synthetic construct
QVQLQQSGPDLVKPGASVRISCKASGYTFAGHYVHWVKQRPGRGLEWIGWIFPGKVNTKYNEKFKGKATLTADKSSSTAYMQLSSLTSEDSAVYFCARVGYDYPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 120 aa, synthetic construct
QVQLQQSGPDLVKPGASVRISCKASGYTFAGHYVHWVKQRPGRGLEWIGWIFPGKVNTKYNEKFKGKATLTADKSSSTAYMQLSSLTSEDSAVYFCARVGYDYPYYFDYWGQGTTLTVSS

SEQ ID NO: 259 - protein, 214 aa, synthetic construct
DILLTQSPAILSVSPGDRVSFSCRASQSIGTSIHWYQQRTDGSPRLLIKYASESISGIPSRFSGSGSGTDFTLSINSVESEDVADYYCQQSSSWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 107 aa, synthetic construct
DILLTQSPAILSVSPGDRVSFSCRASQSIGTSIHWYQQRTDGSPRLLIKYASESISGIPSRFSGSGSGTDFTLSINSVESEDVADYYCQQSSSWPFTFGSGTKLEIK

SEQ ID NO: 261 - protein, 449 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 119 aa, synthetic construct [sequence omitted]

SEQ ID NO: 263 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 112 aa, synthetic construct [sequence omitted]

SEQ ID NO: 265 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 267 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 269 - protein, 455 aa, synthetic construct
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 125 aa, synthetic construct [sequence omitted]

SEQ ID NO: 271 - protein, 214 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 107 aa, synthetic construct [sequence omitted]

SEQ ID NO: 273 - protein, 460 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 130 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 275 - protein, 215 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 108 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 277 - protein, 443 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 113 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSS

SEQ ID NO: 279 - protein, 214 aa, synthetic construct
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 107 aa, synthetic construct
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIK

SEQ ID NO: 281 - protein, 451 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 121 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSS

SEQ ID NO: 283 - protein, 218 aa, synthetic construct
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 111 aa, synthetic construct
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELK

SEQ ID NO: 285 - protein, 458 aa, synthetic construct
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 286 - protein, 119 aa, synthetic construct
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSS

SEQ ID NO: 287 - protein, 212 aa, synthetic construct
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 105 aa, synthetic construct
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELK

SEQ ID NO: 289 - protein, 461 aa, synthetic construct
EVQLVESGGGLVQPGGSLRLSCVVSGFTFSYYDMHWVRQVTGKGLEWVSAIGTAGATYYPGSVKGRFTISRENAKNSLYLQMNSLRAGDTAVYYCARDRGYSGYDAYYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 290 - protein, 122 aa, synthetic construct
EVQLVESGGGLVQPGGSLRLSCVVSGFTFSYYDMHWVRQVTGKGLEWVSAIGTAGATYYPGSVKGRFTISRENAKNSLYLQMNSLRAGDTAVYYCARDRGYSGYDAYYFDFWGQGTLVTVSS

SEQ ID NO: 291 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 293 - protein, 454 aa, synthetic construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - protein, 124 aa, synthetic construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSS

SEQ ID NO: 295 - protein, 214 aa, synthetic construct; identical to SEQ ID NO: 291

SEQ ID NO: 296 - protein, 107 aa, synthetic construct; identical to SEQ ID NO: 292

SEQ ID NO: 297 - protein, 448 aa, synthetic construct
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 118 aa, synthetic construct
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSS

SEQ ID NO: 299 - protein, 218 aa, synthetic construct
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 300 - protein, 111 aa, synthetic construct
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIK

SEQ ID NO: 301 - protein, 443 aa, synthetic construct
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 113 aa, synthetic construct
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSS

SEQ ID NO: 303 - protein, 214 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 304 - protein, 107 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIK

SEQ ID NO: 305 - protein, 359 aa, synthetic construct
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 306 - protein, 118 aa, synthetic construct
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSS

SEQ ID NO: 307 - protein, 220 aa, synthetic construct
AEIVMTQTPSSKSAAVGDTVTIKCQASQSITSYLSWYQQKPGQPPKLLIYRASTLASGVPSRFKGSGSGTQFTLTISDLECADAATYYCQSNYGSYSTNYGVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 113 aa, synthetic construct
AEIVMTQTPSSKSAAVGDTVTIKCQASQSITSYLSWYQQKPGQPPKLLIYRASTLASGVPSRFKGSGSGTQFTLTISDLECADAATYYCQSNYGSYSTNYGVTFGGGTKVEIK

SEQ ID NO: 309 - protein, 217 aa, synthetic construct
DVVMTQTPASVSEPVGGTVTIKCQASQSIYSGLAWYQQKPGQPPKLLIYSASKLASGVPSRFKGSGSGTEYTLTISDLECADAATYYCQNYYYGISNGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 110 aa, synthetic construct
DVVMTQTPASVSEPVGGTVTIKCQASQSIYSGLAWYQQKPGQPPKLLIYSASKLASGVPSRFKGSGSGTEYTLTISDLECADAATYYCQNYYYGISNGWTFGGGTKVEIK

SEQ ID NO: 311 - protein, 446 aa, synthetic construct
QVQLVQSGAEVKKPGASVKVSCKASGYTFTSYHMHWVRQAPGQGLEWMGWINPYTGDTNYAQKFQGRVTMTRDTSISTAYMELSRLRSEDTAVYYCAREKVYMDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 116 aa, synthetic construct
QVQLVQSGAEVKKPGASVKVSCKASGYTFTSYHMHWVRQAPGQGLEWMGWINPYTGDTNYAQKFQGRVTMTRDTSISTAYMELSRLRSEDTAVYYCAREKVYMDIWGQGTLVTVSS

SEQ ID NO: 313 - protein, 215 aa, synthetic construct
DIELTQPPSVSVSPGQTASITCSGDSIPSKYAHWYQQKPGQAPVLVIYGKSHRPSGIPERFSGSNSGNTATLTISGTQAEDEADYYCAAWDLLGDGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - protein, 108 aa, synthetic construct
DIELTQPPSVSVSPGQTASITCSGDSIPSKYAHWYQQKPGQAPVLVIYGKSHRPSGIPERFSGSNSGNTATLTISGTQAEDEADYYCAAWDLLGDGWVFGGGTKLTVL

SEQ ID NO: 315 - protein, 451 aa, synthetic construct
EVQLVESGGGLVKPGGSLRLSCAASGFTFSDYGIHWVRQAPGKGLEWVGRIKSKTDGGITEYAAPVKGRFTISRDDSKNTLYLQMNSLKTEDTAVYYCARAIYYLEAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 121 aa, synthetic construct
EVQLVESGGGLVKPGGSLRLSCAASGFTFSDYGIHWVRQAPGKGLEWVGRIKSKTDGGITEYAAPVKGRFTISRDDSKNTLYLQMNSLKTEDTAVYYCARAIYYLEAFDVWGQGTLVTVSS

SEQ ID NO: 317 - protein, 215 aa, synthetic construct
DIELTQPPSVSVSPGQTASITCSGDSLGSYYVHWYQQKPGQAPVLVIYRNKQRPSGIPERFSGSNSGNTATLTISGTQAEDEADYYCQTYDWMYSSRVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 108 aa, synthetic construct
DIELTQPPSVSVSPGQTASITCSGDSLGSYYVHWYQQKPGQAPVLVIYRNKQRPSGIPERFSGSNSGNTATLTISGTQAEDEADYYCQTYDWMYSSRVFGGGTKLTVL

SEQ ID NO: 319 - protein, 456 aa, synthetic construct
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 320 - protein, 117 aa, synthetic construct
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSS

SEQ ID NO: 321 - protein, 219 aa, synthetic construct
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 112 aa, synthetic construct
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIK

SEQ ID NO: 323 - protein, 460 aa, synthetic construct
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 324 - protein, 121 aa, synthetic construct
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSS

SEQ ID NO: 325 - protein, 213 aa, synthetic construct
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 106 aa, synthetic construct
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELK

SEQ ID NO: 327 - protein, 450 aa, synthetic construct
QVQLVQSGSELKKPGASVKVSCKASGYTFTRYPMNWVRQAPGQGLEWMGWINTNTGNPTYAQGFTGRFVFSLDTSVSTAFLQISSLKAEDTAVYYCARERTNFYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 120 aa, synthetic construct
QVQLVQSGSELKKPGASVKVSCKASGYTFTRYPMNWVRQAPGQGLEWMGWINTNTGNPTYAQGFTGRFVFSLDTSVSTAFLQISSLKAEDTAVYYCARERTNFYDAFDIWGQGTMVTVSS

SEQ ID NO: 329 - protein, 214 aa, synthetic construct; identical to SEQ ID NO: 291

SEQ ID NO: 330 - protein, 107 aa, synthetic construct; identical to SEQ ID NO: 292

SEQ ID NO: 331 - protein, 450 aa, synthetic construct
QVQLVQSGSELKKPGASVKVSCKASGYTFNSYAMDWVRQAPGQGLEWMGWINTNTGNPTYAQAFTGRFVFSLDTSVSTAYLEISSLKAEDTAVYYCARERHGYFEAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 120 aa, synthetic construct
QVQLVQSGSELKKPGASVKVSCKASGYTFNSYAMDWVRQAPGQGLEWMGWINTNTGNPTYAQAFTGRFVFSLDTSVSTAYLEISSLKAEDTAVYYCARERHGYFEAFDIWGQGTTVTVSS

SEQ ID NO: 333 - protein, 214 aa, synthetic construct; identical to SEQ ID NO: 291

SEQ ID NO: 334 - protein, 107 aa, synthetic construct; identical to SEQ ID NO: 292

SEQ ID NO: 335 - protein, 450 aa, synthetic construct
EVQLVQSGSELKKPGASVKVSCKASGYTFTKYAMNWVRQVPGQGLEWMGWINTNTGNPTYAQGFTGRFVFSLDTSVRTAYLQISSLKAEDTAVYYCARKGGSYYDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 120 aa, synthetic construct
EVQLVQSGSELKKPGASVKVSCKASGYTFTKYAMNWVRQVPGQGLEWMGWINTNTGNPTYAQGFTGRFVFSLDTSVRTAYLQISSLKAEDTAVYYCARKGGSYYDWFDPWGQGTLVTVSS

SEQ ID NO: 337 - protein, 214 aa, synthetic construct; identical to SEQ ID NO: 291

SEQ ID NO: 338 - protein, 107 aa, synthetic construct; identical to SEQ ID NO: 292

SEQ ID NO: 339 - protein, 362 aa, synthetic construct
QVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 340 - protein, 121 aa, synthetic construct
QVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSS

SEQ ID NO: 341 - protein, 363 aa, synthetic construct
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 342 - protein, 122 aa, synthetic construct
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 343 - protein, 359 aa, synthetic construct
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 344 - protein, 118 aa, synthetic construct
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 345 - protein, 359 aa, synthetic construct
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 346 - protein, 118 aa, synthetic construct
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 347 - protein, 359 aa, synthetic construct
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 348 - protein, 118 aa, synthetic construct
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 349 - protein, 359 aa, synthetic construct
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 350 - protein, 118 aa, synthetic construct
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 351 - protein, 448 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 352 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 353 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 354 - protein, 107 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 355 - protein, 448 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 356 - protein, 119 aa, synthetic construct [sequence omitted]

SEQ ID NO: 357 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 358 - protein, 113 aa, synthetic construct [sequence omitted]

SEQ ID NO: 359 - protein, 447 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 360 - protein, 120 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 361 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 362 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 363 - protein, 450 aa, synthetic construct
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 364 - protein, 121 aa, synthetic construct
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSS

SEQ ID NO: 365 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 366 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 367 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 119 aa, synthetic construct; identical to SEQ ID NO: 352

SEQ ID NO: 369 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 107 aa, synthetic construct; identical to SEQ ID NO: 354

SEQ ID NO: 371 - protein, 241 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 372 - protein, 459 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 373 - protein, 120 aa, synthetic construct; identical to SEQ ID NO: 360

SEQ ID NO: 374 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 106 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 376 - protein, 450 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 120 aa, synthetic construct; identical to SEQ ID NO: 360

SEQ ID NO: 378 - protein, 213 aa, synthetic construct; identical to SEQ ID NO: 374

SEQ ID NO: 379 - protein, 106 aa, synthetic construct; identical to SEQ ID NO: 375

SEQ ID NO: 380 - protein, 447 aa, synthetic construct
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 117 aa, synthetic construct
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSS

SEQ ID NO: 382 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 383 - protein, 108 aa, synthetic construct; identical to SEQ ID NO: 366

SEQ ID NO: 384 - protein, 454 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 385 - protein, 125 aa, synthetic construct [sequence omitted]

SEQ ID NO: 386 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 387 - protein, 107 aa, synthetic construct [sequence omitted]

SEQ ID NO: 388 - protein, 459 aa, synthetic construct; identical to SEQ ID NO: 372

SEQ ID NO: 389 - protein, 120 aa, synthetic construct; identical to SEQ ID NO: 360

SEQ ID NO: 390 - protein, 213 aa, synthetic construct; identical to SEQ ID NO: 374

SEQ ID NO: 391 - protein, 106 aa, synthetic construct; identical to SEQ ID NO: 375

SEQ ID NO: 392 - protein, 459 aa, synthetic construct; identical to SEQ ID NO: 372

SEQ ID NO: 393 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 394 - protein, 213 aa, synthetic construct; identical to SEQ ID NO: 374

SEQ ID NO: 395 - protein, 106 aa, synthetic construct; identical to SEQ ID NO: 375

SEQ ID NO: 396 - protein, 462 aa, synthetic construct
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 397 - protein, 126 aa, synthetic construct
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSGGS

SEQ ID NO: 398 - protein, 216 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 399 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 400 - protein, 449 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 401 - protein, 120 aa, synthetic construct; identical to SEQ ID NO: 360

SEQ ID NO: 402 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 403 - protein, 106 aa, synthetic construct; identical to SEQ ID NO: 375

SEQ ID NO: 404 - protein, 450 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - protein, 120 aa, synthetic construct; identical to SEQ ID NO: 360

SEQ ID NO: 406 - protein, 213 aa, synthetic construct; identical to SEQ ID NO: 374

SEQ ID NO: 407 - protein, 106 aa, synthetic construct; identical to SEQ ID NO: 375

SEQ ID NO: 408 - protein, 448 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 410 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 411 - protein, 111 aa, synthetic construct; identical to SEQ ID NO: 362

SEQ ID NO: 412 - protein, 449 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 413 - protein, 119 aa, synthetic construct [sequence omitted]

SEQ ID NO: 414 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 415 - protein, 113 aa, synthetic construct [sequence omitted]

SEQ ID NO: 416 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 417 - protein, 119 aa, synthetic construct; identical to SEQ ID NO: 352

SEQ ID NO: 418 - protein, 214 aa, synthetic construct; identical to SEQ ID NO: 369

SEQ ID NO: 419 - protein, 107 aa, synthetic construct; identical to SEQ ID NO: 354

SEQ ID NO: 420 - protein, 451 aa, synthetic construct
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 421 - protein, 121 aa, synthetic construct; identical to SEQ ID NO: 364

SEQ ID NO: 422 - protein, 215 aa, synthetic construct; identical to SEQ ID NO: 382

SEQ ID NO: 423 - protein, 108 aa, synthetic construct; identical to SEQ ID NO: 366

SEQ ID NO: 424 - protein, 447 aa, synthetic construct
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - protein, 117 aa, synthetic construct; identical to SEQ ID NO: 381

SEQ ID NO: 426 - protein, 215 aa, synthetic construct; identical to SEQ ID NO: 382

SEQ ID NO: 427 - protein, 108 aa, synthetic construct; identical to SEQ ID NO: 366

SEQ ID NO: 428 - protein, 440 aa, synthetic construct
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 429 - protein, 115 aa, synthetic construct [sequence omitted]

SEQ ID NO: 430 - protein, 215 aa, synthetic construct
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 431 - protein, 108 aa, synthetic construct [sequence omitted]

SEQ ID NO: 432 - protein, 447 aa, synthetic construct
QIQLVQSGPELKKPGETVKISCKASGYTFTNYGMNWVRQAPGKGLKWVGWINTNTGEPTYADDFKGRFAFSLETSASTAYLQISNLKNEDMSTYFCAGSHDYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 433 - protein, 117 aa, synthetic construct
QIQLVQSGPELKKPGETVKISCKASGYTFTNYGMNWVRQAPGKGLKWVGWINTNTGEPTYADDFKGRFAFSLETSASTAYLQISNLKNEDMSTYFCAGSHDYSFAYWGQGTLVTVSA

SEQ ID NO: 434 - protein, 216 aa, synthetic construct
QAVVTQESALTTSPGETVTFTCRSSTGAVTTSNYANWVQEKPDHLFTGLIGGTNNRAPGVPARFSGSLIGDKAALTITGAQTEDEAIYFCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - protein, 109 aa, synthetic construct
QAVVTQESALTTSPGETVTFTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 436 - protein, 444 aa, synthetic construct
QVQLQQSGTELMKPGASVKISCKATGYTFSSYWIEWVKQRPGHGLEWIGEILPGSGSTNYNEKFKGKATFTVDTSSNTAYMQLSSLTSEDSAVYYCARIIRDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 437 - protein, 114 aa, synthetic construct
QVQLQQSGTELMKPGASVKISCKATGYTFSSYWIEWVKQRPGHGLEWIGEILPGSGSTNYNEKFKGKATFTVDTSSNTAYMQLSSLTSEDSAVYYCARIIRDFWGQGTTLTVSS

SEQ ID NO: 438 - protein, 214 aa, synthetic construct
DIQMTQSSSSFSVSLGDRVTITCKASEDIYNRLAWFQQKPGNAPRLLISGATSLETGVPSRFSGSRSGEDYTLIITSLQTEDVATYYCQQFWTTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 439 - protein, 107 aa, synthetic construct
DIQMTQSSSSFSVSLGDRVTITCKASEDIYNRLAWFQQKPGNAPRLLISGATSLETGVPSRFSGSRSGEDYTLIITSLQTEDVATYYCQQFWTTPPTFGGGTKLEIK

SEQ ID NO: 440 - protein, 443 aa, synthetic construct
QVQLQQSGAELMKPGASVKISCKATGYTFSRYWIEWVKQRPGHGLEWIGEILPGSGSTNYNEKFKGKATFTADTSSNTAYMQLTSLTSEDSAVYFCERRGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 113 aa, synthetic construct
QVQLQQSGAELMKPGASVKISCKATGYTFSRYWIEWVKQRPGHGLEWIGEILPGSGSTNYNEKFKGKATFTADTSSNTAYMQLTSLTSEDSAVYFCERRGAYWGQGTLVTVSA

SEQ ID NO: 442 - protein, 214 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYHCQQNWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - protein, 107 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYHCQQNWSTPPTFGGGTKLEIK

SEQ ID NO: 444 - protein, 452 aa, synthetic construct
QVQLQQPGAELVRPGASVKLSCKASGYTFTSYWMNWVKQRPGQGLEWIGMIDPSDSETHYSQMFKDKATLTVDQSSNTAYMHLSSLTSEDSAVYYCAREGYYFDGTRGIAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 445 - protein, 122 aa, synthetic construct
QVQLQQPGAELVRPGASVKLSCKASGYTFTSYWMNWVKQRPGQGLEWIGMIDPSDSETHYSQMFKDKATLTVDQSSNTAYMHLSSLTSEDSAVYYCAREGYYFDGTRGIAYWGQGTLVTVSV

SEQ ID NO: 446 - protein, 214 aa, synthetic construct
DIQMTQSTSSLSASLGDRVTISCRASQDISNYLNWYQQKPDGTVKPLIYYTSRLHSGVPSRFSGSGSGTDYSLTISNLDQEDIATYFCQQGNTLPFTFGSGTKLAIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 447 - protein, 107 aa, synthetic construct
DIQMTQSTSSLSASLGDRVTISCRASQDISNYLNWYQQKPDGTVKPLIYYTSRLHSGVPSRFSGSGSGTDYSLTISNLDQEDIATYFCQQGNTLPFTFGSGTKLAIE

SEQ ID NO: 448 - protein, 446 aa, synthetic construct
QVQLQQSGAEVMKPGASVKLSCKATGYTFTGYWIEWVKERPGHGLEWIGEILPGSGSTNYNEKFKGKATFTADPSSNTAYMQLSSLTTEDSAIYYCARDYGSFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 449 - protein, 116 aa, synthetic construct
QVQLQQSGAEVMKPGASVKLSCKATGYTFTGYWIEWVKERPGHGLEWIGEILPGSGSTNYNEKFKGKATFTADPSSNTAYMQLSSLTTEDSAIYYCARDYGSFGYWGQGTLVTVSA

SEQ ID NO: 450 - protein, 214 aa, synthetic construct
DIVMTQSQKFMSTSVGDRVSVTCKASQNVGTNVAWFQQKPGQSPKLLIYSASYRYSGVPDRFTGSGSGTDFTLTISNVQSEDLAEFFCQQYNTYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 107 aa, synthetic construct
DIVMTQSQKFMSTSVGDRVSVTCKASQNVGTNVAWFQQKPGQSPKLLIYSASYRYSGVPDRFTGSGSGTDFTLTISNVQSEDLAEFFCQQYNTYPLTFGAGTKLELK

SEQ ID NO: 452 - protein, 446 aa, synthetic construct
EVQLQQSGAELVRPGASVKLSCTASGFNIKDDYMHWMKQRPEQGLEWLGWIDPEIGATEYASKFQGKATMTADTSSNTAYLQLSSLTSEDTAVYYCVDDRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 453 - protein, 116 aa, synthetic construct
EVQLQQSGAELVRPGASVKLSCTASGFNIKDDYMHWMKQRPEQGLEWLGWIDPEIGATEYASKFQGKATMTADTSSNTAYLQLSSLTSEDTAVYYCVDDRRGMDYWGQGTSVTVSS

SEQ ID NO: 454 - protein, 219 aa, synthetic construct
DIVMTQDAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 455 - protein, 112 aa, synthetic construct
DIVMTQDAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 456 - protein, 449 aa, synthetic construct
QVSLKESGPGILQPSQTLSLTCSFSGFSLSTSGMAVGWIRQPSGRGLEWLANIWWDDSQHYNAALKSRLTISKDTSKNQVFLKIASVDTADTATYYCARSNWGRYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 457 - protein, 119 aa, synthetic construct
QVSLKESGPGILQPSQTLSLTCSFSGFSLSTSGMAVGWIRQPSGRGLEWLANIWWDDSQHYNAALKSRLTISKDTSKNQVFLKIASVDTADTATYYCARSNWGRYFDYWGQGTTLTVSS

SEQ ID NO: 458 - protein, 219 aa, synthetic construct
DIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCQQYYDYYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 459 - protein, 112 aa, synthetic construct
DIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCQQYYDYYTFGGGTKLEIK

SEQ ID NO: 460 - protein, 442 aa, synthetic construct
QVQLQQPGAELVKPGASVKLSCKASGYTFTTYYIYWVKQRPGQGLEWIGGINPRNGGTNFNEKFKTRATLTVDKSSSTAYMQLSSLTSEDSAVYYCTRTFYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 461 - protein, 112 aa, synthetic construct
QVQLQQPGAELVKPGASVKLSCKASGYTFTTYYIYWVKQRPGQGLEWIGGINPRNGGTNFNEKFKTRATLTVDKSSSTAYMQLSSLTSEDSAVYYCTRTFYWGQGTTLTVSS

SEQ ID NO: 462 - protein, 214 aa, synthetic construct
DIQMTQTTSSLSASLGDRVTISCSASQGIGNYLNWYQQKPDGTVKLLIYYTSSLNSGVPSRFSGSGSGTDYSLTISNLEPEDIATYFCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 463 - protein, 107 aa, synthetic construct
DIQMTQTTSSLSASLGDRVTISCSASQGIGNYLNWYQQKPDGTVKLLIYYTSSLNSGVPSRFSGSGSGTDYSLTISNLEPEDIATYFCQQYSKLPYTFGGGTKLEIK

SEQ ID NO: 464 - protein, 445 aa, synthetic construct
EVQLQQSGAELVRPGASVKLSCTASGFNIIVDYLHWVRQRPEQGLEWIGWIDPEIGSTEYASKFQGKATMTADTSSNTAYLQLSSLTSEDTAVYYCIIDGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 465 - protein, 115 aa, synthetic construct
EVQLQQSGAELVRPGASVKLSCTASGFNIIVDYLHWVRQRPEQGLEWIGWIDPEIGSTEYASKFQGKATMTADTSSNTAYLQLSSLTSEDTAVYYCIIDGTMDYWGQGTSVTVSS

SEQ ID NO: 466 - protein, 219 aa, synthetic construct
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - protein, 112 aa, synthetic construct
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLEYPFTFGSGTKLEIK

SEQ ID NO: 468 - protein, 447 aa, synthetic construct
EVQLQQSGAELVRPGASVKLSCTASGFNIKDDYIHWVKQRPEQGLEWIGWIDPENGDTKYASKFPGKATMTADTSSNTAYLQLSSLTSEDTAVYYCTASRTTALDYWGPGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - protein, 117 aa, synthetic construct
EVQLQQSGAELVRPGASVKLSCTASGFNIKDDYIHWVKQRPEQGLEWIGWIDPENGDTKYASKFPGKATMTADTSSNTAYLQLSSLTSEDTAVYYCTASRTTALDYWGPGTTLTVSS

SEQ ID NO: 470 - protein, 214 aa, synthetic construct
DIVMTQSQKFMSTSVGDRVSITCKASQSVRPAVAWYQQKPGQSPKALIYLASNRHTGVPDRFTGSGSGTDFTLTISNVQSEDLADYFCLQHWNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 471 - protein, 107 aa, synthetic construct
DIVMTQSQKFMSTSVGDRVSITCKASQSVRPAVAWYQQKPGQSPKALIYLASNRHTGVPDRFTGSGSGTDFTLTISNVQSEDLADYFCLQHWNYPYTFGGGTKLEIK

SEQ ID NO: 472 - protein, 5 aa, synthetic construct
SYWMH

SEQ ID NO: 473 - protein, 17 aa, synthetic construct
AIYPGNSETGYAQKFQG

SEQ ID NO: 474 - protein, 9 aa, synthetic construct
ENWDPGFAF

SEQ ID NO: 475 - protein, 10 aa, synthetic construct
SASSSVYYMY

SEQ ID NO: 476 - protein, 7 aa, synthetic construct
STSNLAS

SEQ ID NO: 477 - protein, 9 aa, synthetic construct
QQRRNYPYT

SEQ ID NO: 478 - protein, 5 aa, synthetic construct
SHFMH

SEQ ID NO: 479 - protein, 17 aa, synthetic construct
WIDPVTGGTKYAQNFQG

SEQ ID NO: 480 - protein, 11 aa, synthetic construct
EARADRGQFDK

SEQ ID NO: 481 - protein, 11 aa, synthetic construct
GGNNIGSKSVH

SEQ ID NO: 482 - protein, 7 aa, synthetic construct
YGSNRPS

SEQ ID NO: 483 - protein, 10 aa, synthetic construct
QVWDSSSDWV

SEQ ID NO: 484 - protein, 5 aa, synthetic construct
NYWMN

SEQ ID NO: 485 - protein, 19 aa, synthetic construct
EIRLKSNQYTTHYAESVKG

SEQ ID NO: 486 - protein, 6 aa, synthetic construct
HYYFDY

SEQ ID NO: 487 - protein, 16 aa, synthetic construct
RSSKSLLHSNGITYFF

SEQ ID NO: 488 - protein, 7 aa, synthetic construct
QMSNLAS

SEQ ID NO: 489 - protein, 9 aa, synthetic construct
AQNLELPPT

SEQ ID NO: 490 - protein, 5 aa, synthetic construct
AYWIE

SEQ ID NO: 491 - protein, 17 aa, synthetic construct
EILPGSGNSRYNEKFKG

SEQ ID NO: 492 - protein, 9 aa, synthetic construct
SYDFAWFAY

SEQ ID NO: 493 - protein, 17 aa, synthetic construct
KSSQSLLYSSNQKIYLA

SEQ ID NO: 494 - protein, 7 aa, synthetic construct
WASTRES

SEQ ID NO: 495 - protein, 9 aa, synthetic construct
QQYYRYPRT

SEQ ID NO: 496 - protein, 5 aa, synthetic construct
SYNMH

SEQ ID NO: 497 - protein, 17 aa, synthetic construct
YIYPGNGATNYNQKFQG

SEQ ID NO: 498 - protein, 8 aa, synthetic construct
GDSVPFAY

SEQ ID NO: 499 - protein, 10 aa, synthetic construct
SAHSSVSFMH

SEQ ID NO: 500 - protein, 7 aa, synthetic construct
STSSLAS

SEQ ID NO: 501 - protein, 9 aa, synthetic construct
QQRSSFPLT

SEQ ID NO: 502 - protein, 5 aa, synthetic construct
SYPMS

SEQ ID NO: 503 - protein, 17 aa, synthetic construct
YINNGGGNPYYPDTVKG

SEQ ID NO: 504 - protein, 7 aa, synthetic construct
QYYGFDY

SEQ ID NO: 505 - protein, 16 aa, synthetic construct
RSSQTIVHSNGKIYLE

SEQ ID NO: 506 - protein, 7 aa, synthetic construct
RVSKRFS

SEQ ID NO: 507 - protein, 9 aa, synthetic construct
FQGSHVPWT

SEQ ID NO: 508 - protein, 5 aa, synthetic construct
SFGMH

SEQ ID NO: 509 - protein, 17 aa, synthetic construct
YISSDSSAIYYADTVKG

SEQ ID NO: 510 - protein, 13 aa, synthetic construct
GRENIYYGSRLDY

SEQ ID NO: 511 - protein, 11 aa, synthetic construct
KASQNVDTNVA

SEQ ID NO: 512 - protein, 7 aa, synthetic construct
SASYRYS

SEQ ID NO: 513 - protein, 9 aa, synthetic construct
QQYNNYPFT

SEQ ID NO: 514 - protein, 5 aa, synthetic construct
NYDIN

SEQ ID NO: 515 - protein, 17 aa, synthetic construct
WIFPGDGSTQYNEKFKG

SEQ ID NO: 516 - protein, 9 aa, synthetic construct
QTTATWFAY

SEQ ID NO: 517 - protein, 11 aa, synthetic construct
RASQSISDYLH

SEQ ID NO: 518 - protein, 7 aa, synthetic construct
YASQSIS

SEQ ID NO: 519 - protein, 9 aa, synthetic construct
QNGHSFPLT

SEQ ID NO: 520 - protein, 6 aa, synthetic construct
SGYSWH

SEQ ID NO: 521 - protein, 16 aa, synthetic construct
YIHSSGSTNYNPSLKS

SEQ ID NO: 522 - protein, 7 aa, synthetic construct
YDDYFEY

SEQ ID NO: 523 - protein, 11 aa, synthetic construct
KASQNVGFNVA

SEQ ID NO: 524 - protein, 7 aa, synthetic construct
SASYRYS

SEQ ID NO: 525 - protein, 9 aa, synthetic construct
QQYNWYPFT

SEQ ID NO: 526 - protein, 5 aa, synthetic construct
NYVMH

SEQ ID NO: 527 - protein, 17 aa, synthetic construct
YINPYNDDVKYNEKFKG

SEQ ID NO: 528 - protein, 13 aa, synthetic construct
WGYYGSPLYYFDY

SEQ ID NO: 529 - protein, 10 aa, synthetic construct
RASSRLIYMH

SEQ ID NO: 530 - protein, 7 aa, synthetic construct
ATSNLAS

SEQ ID NO: 531 - protein, 9 aa, synthetic construct
QQWNSNPPT

SEQ ID NO: 532 - protein, 5 aa, synthetic construct
GYYMH

SEQ ID NO: 533 - protein, 17 aa, synthetic construct
RINPNNGVTLYNQKFKD

SEQ ID NO: 534 - protein, 11 aa, synthetic construct
STMITNYVMDY

SEQ ID NO: 535 - protein, 11 aa, synthetic construct
KASQSVSNDVA

SEQ ID NO: 536 - protein, 7 aa, synthetic construct
YTSSRYA

SEQ ID NO: 537 - protein, 9 aa, synthetic construct
QQDYNSPPT

SEQ ID NO: 538 - protein, 5 aa, synthetic construct
NFGMN

SEQ ID NO: 539 - protein, 17 aa, synthetic construct
WINTNTGEPRYAEEFKG

SEQ ID NO: 540 - protein, 10 aa, synthetic construct
DWDGAYFFDY

SEQ ID NO: 541 - protein, 11 aa, synthetic construct
KASQSVSNDVA

SEQ ID NO: 542 - protein, 7 aa, synthetic construct
FATNRYT

SEQ ID NO: 543 - protein, 9 aa, synthetic construct
QQDYSSPWT

SEQ ID NO: 544 - protein, 5 aa, synthetic construct
TYAMN

SEQ ID NO: 545 - protein, 19 aa, synthetic construct
RIRSKSNNYATYYADSVKD

SEQ ID NO: 546 - protein, 11 aa, synthetic construct
QWDYDVRAMNY

SEQ ID NO: 547 - protein, 11 aa, synthetic construct
KASQDVDTAVA

SEQ ID NO: 548 - protein, 7 aa, synthetic construct
WASTRLT

SEQ ID NO: 549 - protein, 9 aa, synthetic construct
QQYSSYPYT

SEQ ID NO: 550 - protein, 5 aa, synthetic construct
HYVVG

SEQ ID NO: 551 - protein, 16 aa, synthetic construct
IIYGSGRTYYANWAKG

SEQ ID NO: 552 - protein, 14 aa, synthetic construct
DASVSVYYWGYFDL

SEQ ID NO: 553 - protein, 11 aa, synthetic construct
QASQSIGSELA

SEQ ID NO: 554 - protein, 7 aa, synthetic construct
RASTLES

SEQ ID NO: 555 - protein, 12 aa, synthetic construct
QQGYTYSEIDNA

SEQ ID NO: 556 - protein, 5 aa, synthetic construct
SYGMG

SEQ ID NO: 557 - protein, 16 aa, synthetic construct
IISRNSVTYYATWAKG

SEQ ID NO: 558 - protein, 13 aa, synthetic construct
RATYSGALGYFDI

SEQ ID NO: 559 - protein, 11 aa, synthetic construct
QASENIYSTLA

SEQ ID NO: 560 - protein, 7 aa, synthetic construct
DAFDLAS

SEQ ID NO: 561 - protein, 12 aa, synthetic construct
QQGYSGTNVDNA

SEQ ID NO: 562 - protein, 5 aa, synthetic construct
GYYWT

SEQ ID NO: 563 - protein, 16 aa, synthetic construct
EIDHSESTNYNPSLKS

SEQ ID NO: 564 - protein, 14 aa, synthetic construct
WFGELYHYYYGMDV

SEQ ID NO: 565 - protein, 11 aa, synthetic construct
RASQSVSSYLA

SEQ ID NO: 566 - protein, 7 aa, synthetic construct
DASNRAT

SEQ ID NO: 567 - protein, 9 aa, synthetic construct
QQRSNWPLT

SEQ ID NO: 568 - protein, 5 aa, synthetic construct
SYLMT

SEQ ID NO: 569 - protein, 17 aa, synthetic construct
NIKQDGSEKYYVDSVKG

SEQ ID NO: 570 - protein, 13 aa, synthetic construct
DGYSSGRHYGMDV

SEQ ID NO: 571 - protein, 11 aa, synthetic construct
RASQSVSRYLA

SEQ ID NO: 572 - protein, 7 aa, synthetic construct
DASNRAT

SEQ ID NO: 573 - protein, 9 aa, synthetic construct
QQRSNWPRT

SEQ ID NO: 574 - protein, 5 aa, synthetic construct
RHWMS

SEQ ID NO: 575 - protein, 17 aa, synthetic construct
EINPDSRTINYAPSVKG

SEQ ID NO: 576 - protein, 12 aa, synthetic construct
RVRIHYYGAMDS

SEQ ID NO: 577 - protein, 11 aa, synthetic construct
RASQNVGSSLV

SEQ ID NO: 578 - protein, 7 aa, synthetic construct
SASFLYS

SEQ ID NO: 579 - protein, 9 aa, synthetic construct
QQYNNYPLT

SEQ ID NO: 580 - protein, 5 aa, synthetic construct
DTYIH

SEQ ID NO: 581 - protein, 17 aa, synthetic construct
RIDPADGNRKSDPKFQV

SEQ ID NO: 582 - protein, 10 aa, synthetic construct
GYGNLNAMDS

SEQ ID NO: 583 - protein, 11 aa, synthetic construct
HASQNINVWLN

SEQ ID NO: 584 - protein, 7 aa, synthetic construct
KASNLHT

SEQ ID NO: 585 - protein, 9 aa, synthetic construct
QQGQSYPLT

SEQ ID NO: 586 - protein, 5 aa, synthetic construct
DYHMD

SEQ ID NO: 587 - protein, 17 aa, synthetic construct
DINPNNGGAIYNQKFKG

SEQ ID NO: 588 - protein, 13 aa, synthetic construct
SHYDYAGGAWFAY

SEQ ID NO: 589 - protein, 17 aa, synthetic construct
KSSQSLLFSGNQKNYLA

SEQ ID NO: 590 - protein, 7 aa, synthetic construct
WASTRAS

SEQ ID NO: 591 - protein, 9 aa, synthetic construct
QQYYSFPRT

SEQ ID NO: 592 - protein, 5 aa, synthetic construct
GYYWS

SEQ ID NO: 593 - protein, 16 aa, synthetic construct
EINHSGSTNYNPSLKS

SEQ ID NO: 594 - protein, 9 aa, synthetic construct
DKWTWYFDL

SEQ ID NO: 595 - protein, 17 aa, synthetic construct
RSSQSVLYSSSNRNYLA

SEQ ID NO: 596 - protein, 7 aa, synthetic construct
WASTRES

SEQ ID NO: 597 - protein, 9 aa, synthetic construct
QQYYSTPRT

SEQ ID NO: 598 - protein, 5 aa, synthetic construct
SYAMS

SEQ ID NO: 599 - protein, 17 aa, synthetic construct
AINSQGKSTYYADSVKG

SEQ ID NO: 600 - protein, 8 aa, synthetic construct
WGDEGFDI

SEQ ID NO: 601 - protein, 11 aa, synthetic construct
RASQGISNWLA

SEQ ID NO: 602 - protein, 7 aa, synthetic construct
GASSLQS

SEQ ID NO: 603 - protein, 9 aa, synthetic construct
QQYSSFPTT

SEQ ID NO: 604 - protein, 5 aa, synthetic construct
GDWIH

SEQ ID NO: 605 - protein, 17 aa, synthetic construct
EISAAGGYTDYADSVKG

SEQ ID NO: 606 - protein, 12 aa, synthetic construct
ESRVSFEAAMDY

SEQ ID NO: 607 - protein, 11 aa, synthetic construct
RASQNIATDVA

SEQ ID NO: 608 - protein, 7 aa, synthetic construct
SASFLYS

SEQ ID NO: 609 - protein, 9 aa, synthetic construct
QQSEPEPYT

SEQ ID NO: 610 - protein, 5 aa, synthetic construct
SSYIS

SEQ ID NO: 611 - protein, 17 aa, synthetic construct
WIYAGTGSPSYNQKLQG

SEQ ID NO: 612 - protein, 11 aa, synthetic construct
HRDYYSNSLTY

SEQ ID NO: 613 - protein, 17 aa, synthetic construct
KSSQSVLNSGNQKNYLT

SEQ ID NO: 614 - protein, 7 aa, synthetic construct
WASTRES

SEQ ID NO: 615 - protein, 9 aa, synthetic construct
QSDYSYPYT

SEQ ID NO: 616 - protein, 5 aa, synthetic construct
DYDMS

SEQ ID NO: 617 - protein, 17 aa, synthetic construct
TIDLDSGSIYYADSVQG

SEQ ID NO: 618 - protein, 12 aa, synthetic construct
DLHMGPEGPFDY

SEQ ID NO: 619 - protein, 13 aa, synthetic construct
SGSSSNIGSNSVS

SEQ ID NO: 620 - protein, 7 aa, synthetic construct
SDNHRPS

SEQ ID NO: 621 - protein, 11 aa, synthetic construct
QGWDTSLSGHV

SEQ ID NO: 622 - protein, 5 aa, synthetic construct
SYWMH

SEQ ID NO: 623 - protein, 17 aa, synthetic construct
EIIPIFGHTNYNEKFKS

SEQ ID NO: 624 - protein, 14 aa, synthetic construct
GGYYYYPRQGFLDY

SEQ ID NO: 625 - protein, 15 aa, synthetic construct
KASQSVDYSGDSYMN

SEQ ID NO: 626 - protein, 7 aa, synthetic construct
AASDLES

SEQ ID NO: 627 - protein, 9 aa, synthetic construct
QQSHEDPFT

SEQ ID NO: 628 - protein, 5 aa, synthetic construct
SYAMS

SEQ ID NO: 629 - protein, 17 aa, synthetic construct
AISGSGGSTYYADSVKG

SEQ ID NO: 630 - protein, 8 aa, synthetic construct
TNSAKFDP

SEQ ID NO: 631 - protein, 14 aa, synthetic construct
TGTSNDVGAYNYVS

SEQ ID NO: 632 - protein, 7 aa, synthetic construct
EVNKRPS

SEQ ID NO: 633 - protein, 11 aa, synthetic construct
SSYTMGSTFML

SEQ ID NO: 634 - protein, 5 aa, synthetic construct
SYGVH

SEQ ID NO: 635 - protein, 16 aa, synthetic construct
VIWSGGSTDYADSVKG

SEQ ID NO: 636 - protein, 8 aa, synthetic construct
NSNNGFAY

SEQ ID NO: 637 - protein, 11 aa, synthetic construct
RASQNIYSNLA

SEQ ID NO: 638 - protein, 7 aa, synthetic construct
AAKNLAS

SEQ ID NO: 639 - protein, 9 aa, synthetic construct
QHFYDTPWT

SEQ ID NO: 640 - protein, 5 aa, synthetic construct
AYNMH

SEQ ID NO: 641 - protein, 17 aa, synthetic construct
FIDPYSGIITYNQTFKG

SEQ ID NO: 642 - protein, 10 aa, synthetic construct
RGYYDGGFDY

SEQ ID NO: 643 - protein, 11 aa, synthetic construct
RASQDITNYLN

SEQ ID NO: 644 - protein, 7 aa, synthetic construct
YTSRLHS

SEQ ID NO: 645 - protein, 9 aa, synthetic construct
QQDSKHPRT

SEQ ID NO: 646 - protein, 5 aa, synthetic construct
RYWIN

SEQ ID NO: 647 - protein, 17 aa, synthetic construct
NIYPGSNITNYNEKFKN

SEQ ID NO: 648 - protein, 13 aa, synthetic construct
EGIYDGYFPLFPY

SEQ ID NO: 649 - protein, 12 aa, synthetic construct
SASSSVSSGNFH

SEQ ID NO: 650 - protein, 7 aa, synthetic construct
RTSNLAS

SEQ ID NO: 651 - protein, 9 aa, synthetic construct
QQWSGYPWT

SEQ ID NO: 652 - protein, 7 aa, synthetic construct
SQSAAWN

SEQ ID NO: 653 - protein, 18 aa, synthetic construct
RIYYRSKWYNDYALSVKS

SEQ ID NO: 654 - protein, 11 aa, synthetic construct
GEGYGREGFAI

SEQ ID NO: 655 - protein, 11 aa, synthetic construct
RASQTISNTLA

SEQ ID NO: 656 - protein, 7 aa, synthetic construct
AASNLQS

SEQ ID NO: 657 - protein, 8 aa, synthetic construct
QQYLSWFT

SEQ ID NO: 658 - protein, 5 aa, synthetic construct
NYGMN

SEQ ID NO: 659 - protein, 17 aa, synthetic construct
WINTYTGEPTYGEDFKG

SEQ ID NO: 660 - protein, 7 aa, synthetic construct
FGNYVDY

SEQ ID NO: 661 - protein, 16 aa, synthetic construct
RSSKNLLHSNGITYLY

SEQ ID NO: 662 - protein, 7 aa, synthetic construct
QMSNLAS

SEQ ID NO: 663 - protein, 9 aa, synthetic construct
AQNLEIPRT

SEQ ID NO: 664 - protein, 5 aa, synthetic construct
KYGMN

SEQ ID NO: 665 - protein, 17 aa, synthetic construct
WINTYTEEPTYGDDFKG

SEQ ID NO: 666 - protein, 7 aa, synthetic construct
FGSAVDY

SEQ ID NO: 667 - protein, 16 aa, synthetic construct
RSSKSLLHSNGITYLY

SEQ ID NO: 668 - protein, 7 aa, synthetic construct
QMSNLAS

SEQ ID NO: 669 - protein, 9 aa, synthetic construct
AQNLELPRT

SEQ ID NO: 670 - protein, 5 aa, synthetic construct
NYGMN

SEQ ID NO: 671 - protein, 17 aa, synthetic construct
WINTYTGESTYADSFKG

SEQ ID NO: 672 - protein, 7 aa, synthetic construct
FAIKGDY

SEQ ID NO: 673 - protein, 16 aa, synthetic construct
RSTKSLLHSNGITYLY

SEQ ID NO: 674 - protein, 7 aa, synthetic construct
QMSNLAS

SEQ ID NO: 675 - protein, 9 aa, synthetic construct
AQNLEIPRT

SEQ ID NO: 676 - protein, 5 aa, synthetic construct
SYAIS

SEQ ID NO: 677 - protein, 17 aa, synthetic construct
GIVPIFGTANYAQKFQG

SEQ ID NO: 678 - protein, 6 aa, synthetic construct
DPFLHY

SEQ ID NO: 679 - protein, 12 aa, synthetic construct
RASQSVSSSYLA

SEQ ID NO: 680 - protein, 7 aa, synthetic construct
GASSRAT

SEQ ID NO: 681 - protein, 9 aa, synthetic construct
AQGELYPRQ

SEQ ID NO: 682 - protein, 5 aa, synthetic construct
SYAIS

SEQ ID NO: 683 - protein, 17 aa, synthetic construct
GIIPIFGTANYAQKFQG

SEQ ID NO: 684 - protein, 6 aa, synthetic construct
GLLWNY

SEQ ID NO: 685 - protein, 11 aa, synthetic construct
RASQSVSSNLA

SEQ ID NO: 686 - protein, 7 aa, synthetic construct
GASTTAS

SEQ ID NO: 687 - protein, 11 aa, synthetic construct
QQYNNWPPAYT

SEQ ID NO: 688 - protein, 5 aa, synthetic construct
SYVMS

SEQ ID NO: 689 - protein, 17 aa, synthetic construct
TISSGGSYTYYPDSVKG

SEQ ID NO: 690 - protein, 10 aa, synthetic construct
RGDSMITTDY

SEQ ID NO: 691 - protein, 11 aa, synthetic construct
KASQDVGTAVA

SEQ ID NO: 692 - protein, 7 aa, synthetic construct
WASTRHT

SEQ ID NO: 693 - protein, 8 aa, synthetic construct
QQYSSYRT

SEQ ID NO: 694 - protein, 5 aa, synthetic construct
DYAMS

SEQ ID NO: 695 - protein, 17 aa, synthetic construct
GINWQGGSTGYADSVKG

SEQ ID NO: 696 - protein, 12 aa, synthetic construct
ILGAGRGWYFDY

SEQ ID NO: 697 - protein, 11 aa, synthetic construct
SGDSLRSYYAS

SEQ ID NO: 698 - protein, 7 aa, synthetic construct
GANNRPS

SEQ ID NO: 699 - protein, 11 aa, synthetic construct
NSADSSGNHVV

SEQ ID NO: 700 - protein, 5 aa, synthetic construct
DTFIH

SEQ ID NO: 701 - protein, 17 aa, synthetic construct
RIDPANTNTKYDPKFQG

SEQ ID NO: 702 - protein, 9 aa, synthetic construct
GLYTYYFDY

SEQ ID NO: 703 - protein, 11 aa, synthetic construct
RASQSISNNLH

SEQ ID NO: 704 - protein, 7 aa, synthetic construct
FASQSIT

SEQ ID NO: 705 - protein, 9 aa, synthetic construct
QQGNSWPYT

SEQ ID NO: 706 - protein, 7 aa, synthetic construct
SGDYFWS

SEQ ID NO: 707 - protein, 16 aa, synthetic construct
HIHNSGTTYYNPSLKS

SEQ ID NO: 708 - protein, 12 aa, synthetic construct
DRGGDYYYGMDV

SEQ ID NO: 709 - protein, 12 aa, synthetic construct
RASQGISRSYLA

SEQ ID NO: 710 - protein, 7 aa, synthetic construct
GASSRAT

SEQ ID NO: 711 - protein, 9 aa, synthetic construct
QQFGSSPWT

SEQ ID NO: 712 - protein, 5 aa, synthetic construct
DYGMS

SEQ ID NO: 713 - protein, 17 aa, synthetic construct
GINWNGGSTGYADSVKG

SEQ ID NO: 714 - protein, 12 aa, synthetic construct
ILGAGRGWYFDL

SEQ ID NO: 715 - protein, 11 aa, synthetic construct
QGDSLRSYYAS

SEQ ID NO: 716 - protein, 7 aa, synthetic construct
GKNNRPS

SEQ ID NO: 717 - protein, 11 aa, synthetic construct
NSRDSSGNHVV

SEQ ID NO: 718 - protein, 5 aa, synthetic construct
RYVMS

SEQ ID NO: 719 - protein, 16 aa, synthetic construct
SISSGGRMYYPDTVKG

SEQ ID NO: 720 - protein, 12 aa, synthetic construct
GSIYDGYYVFPY

SEQ ID NO: 721 - protein, 12 aa, synthetic construct
SASSSVSSSYLY

SEQ ID NO: 722 - protein, 7 aa, synthetic construct
STSNLAS

SEQ ID NO: 723 - protein, 9 aa, synthetic construct
HQWSTYPPT

SEQ ID NO: 724 - protein, 5 aa, synthetic construct
DYNVN

SEQ ID NO: 725 - protein, 17 aa, synthetic construct
VINPKYGTTRYNQKFKG

SEQ ID NO: 726 - protein, 7 aa, synthetic construct
GLNAWDY

SEQ ID NO: 727 - protein, 11 aa, synthetic construct
GASENIYGALN

SEQ ID NO: 728 - protein, 7 aa, synthetic construct
GATNLED

SEQ ID NO: 729 - protein, 9 aa, synthetic construct
QNVLTTPYT

SEQ ID NO: 730 - protein, 5 aa, synthetic construct
GYFMN

SEQ ID NO: 731 - protein, 17 aa, synthetic construct
LINPYNGDSFYNQKFKG

SEQ ID NO: 732 - protein, 8 aa, synthetic construct
GLRRDFDY

SEQ ID NO: 733 - protein, 16 aa, synthetic construct
KSSQSLLDSDGKTYLN

SEQ ID NO: 734 - protein, 7 aa, synthetic construct
LVSELDS

SEQ ID NO: 735 - protein, 9 aa, synthetic construct
WQGTHFPRT

SEQ ID NO: 736 - protein, 7 aa, synthetic construct
SGGYYWS

SEQ ID NO: 737 - protein, 16 aa, synthetic construct
YIYYSGRTYNNPSLKS

SEQ ID NO: 738 - protein, 15 aa, synthetic construct
VATGRADYHFYAMDV

SEQ ID NO: 739 - protein, 11 aa, synthetic construct
SGDVLAKKSAR

SEQ ID NO: 740 - protein, 7 aa, synthetic construct
KDSERPS

SEQ ID NO: 741 - protein, 9 aa, synthetic construct
YSAADNNLV

SEQ ID NO: 742 - protein, 5 aa, synthetic construct
DSWIH

SEQ ID NO: 743 - protein, 17 aa, synthetic construct
WISPYGGSTYYADSVKG

SEQ ID NO: 744 - protein, 9 aa, synthetic construct
RHWPGGFDY

SEQ ID NO: 745 - protein, 11 aa, synthetic construct
RASQDVSTAVA

SEQ ID NO: 746 - protein, 7 aa, synthetic construct
SASFLYS

SEQ ID NO: 747 - protein, 9 aa, synthetic construct
QQYLYHPAT

SEQ ID NO: 748 - protein, 5 aa, synthetic construct
RYWMS

SEQ ID NO: 749 - protein, 17 aa, synthetic construct
NIKQDGSEKYYVDSVKG

SEQ ID NO: 750 - protein, 12 aa, synthetic construct
EGGWFGELAFDY

SEQ ID NO: 751 - protein, 12 aa, synthetic construct
RASQRVSSSYLA

SEQ ID NO: 752 - protein, 7 aa, synthetic construct
DASSRAT

SEQ ID NO: 753 - protein, 9 aa, synthetic construct
QQYGSLPWT

SEQ ID NO: 754 - protein, 5 aa, synthetic construct
SYIMM

SEQ ID NO: 755 - protein, 17 aa, synthetic construct
SIYPSGGITFYADTVKG

SEQ ID NO: 756 - protein, 11 aa, synthetic construct
IKLGTVTTVDY

SEQ ID NO: 757 - protein, 14 aa, synthetic construct
TGTSSDVGGYNYVS

SEQ ID NO: 758 - protein, 7 aa, synthetic construct
DVSNRPS

SEQ ID NO: 759 - protein, 10 aa, synthetic construct
SSYTSSSTRV

SEQ ID NO: 760 - protein, 5 aa, synthetic construct
SYAMS

SEQ ID NO: 761 - protein, 17 aa, synthetic construct
GISGSGGFTYYADSVKG

SEQ ID NO: 762 - protein, 12 aa, synthetic construct
PPRGYNYGPFDY

SEQ ID NO: 763 - protein, 11 aa, synthetic construct
GGNNIGSKSVH

SEQ ID NO: 764 - protein, 7 aa, synthetic construct
DDSDRPS

SEQ ID NO: 765 - protein, 11 aa, synthetic construct
QVWDSSSDHVV

SEQ ID NO: 766 - protein, 5 aa, synthetic construct
NYGMN

SEQ ID NO: 767 - protein, 17 aa, synthetic construct
WINTYTGEPTYTDDFKG

SEQ ID NO: 768 - protein, 12 aa, synthetic construct
GGFGSSYWYFDV

SEQ ID NO: 769 - protein, 11 aa, synthetic construct
KASQDVSIAVA

SEQ ID NO: 770 - protein, 7 aa, synthetic construct
SASYRYT

SEQ ID NO: 771 - protein, 9 aa, synthetic construct
QQHYITPLT

SEQ ID NO: 772 - protein, 5 aa, synthetic construct
TAGMQ

SEQ ID NO: 773 - protein, 17 aa, synthetic construct
WINTHSGVPKYAEDFKG

SEQ ID NO: 774 - protein, 12 aa, synthetic construct
SGFGSSYWYFDV

SEQ ID NO: 775 - protein, 11 aa, synthetic construct
KASQDVSTAVA

SEQ ID NO: 776 - protein, 7 aa, synthetic construct
SASYRYT

SEQ ID NO: 777 - protein, 9 aa, synthetic construct
QQHYITPLT

SEQ ID NO: 778 - protein, 5 aa, synthetic construct
SYGVH

SEQ ID NO: 779 - protein, 16 aa, synthetic construct
VIWTSGVTDYNSALMG

SEQ ID NO: 780 - protein, 11 aa, synthetic construct
DGDYDRYTMDY

SEQ ID NO: 781 - protein, 15 aa, synthetic construct
RASKSVSTSGYSYMH

SEQ ID NO: 782 - protein, 7 aa, synthetic construct
LASNLES

SEQ ID NO: 783 - protein, 9 aa, synthetic construct
QHSRELPYT

SEQ ID NO: 784 - protein, 5 aa, synthetic construct
SYWIN

SEQ ID NO: 785 - protein, 17 aa, synthetic construct
NIFPSDSYSNYNKKFKD

SEQ ID NO: 786 - protein, 6 aa, synthetic construct
GSGFDY

SEQ ID NO: 787 - protein, 11 aa, synthetic construct
RASQTIGTSIH

SEQ ID NO: 788 - protein, 7 aa, synthetic construct
YASESIS

SEQ ID NO: 789 - protein, 9 aa, synthetic construct
SQSFSWPFT

SEQ ID NO: 790 - protein, 5 aa, synthetic construct
IYWLG

SEQ ID NO: 791 - protein, 17 aa, synthetic construct
NIFPGSAYINYNEKFKG

SEQ ID NO: 792 - protein, 7 aa, synthetic construct
EGSNSGY

SEQ ID NO: 793 - protein, 17 aa, synthetic construct
KSSQSLLNSGNQQNYLA

SEQ ID NO: 794 - protein, 7 aa, synthetic construct
GASTRES

SEQ ID NO: 795 - protein, 9 aa, synthetic construct
QSDHIYPYT

SEQ ID NO: 796 - protein, 5 aa, synthetic construct
HYMMA

SEQ ID NO: 797 - protein, 17 aa, synthetic construct
RIGPSGGPTHYADSVKG

SEQ ID NO: 798 - protein, 19 aa, synthetic construct
YDSGYDYVAVAGPAEYFQH

SEQ ID NO: 799 - protein, 11 aa, synthetic construct
RASQSISTWLA

SEQ ID NO: 800 - protein, 7 aa, synthetic construct
KASNLHT

SEQ ID NO: 801 - protein, 9 aa, synthetic construct
QQYNSYSRT

SEQ ID NO: 802 - protein, 5 aa, synthetic construct
HYSMH

SEQ ID NO: 803 - protein, 17 aa, synthetic construct
WINTYTGEPTYADDFKG

SEQ ID NO: 804 - protein, 10 aa, synthetic construct
YYRYERDFDY

SEQ ID NO: 805 - protein, 16 aa, synthetic construct
RSSQSIVHSNGNTYLE

SEQ ID NO: 806 - protein, 7 aa, synthetic construct
KVSNRFS

SEQ ID NO: 807 - protein, 9 aa, synthetic construct
FQGSHVPYT

SEQ ID NO: 808 - protein, 5 aa, synthetic construct
GYYIH

SEQ ID NO: 809 - protein, 17 aa, synthetic construct
WIYPGNFNTKYNERFKG

SEQ ID NO: 810 - protein, 11 aa, synthetic construct
EDGSPYYAMDY

SEQ ID NO: 811 - protein, 16 aa, synthetic construct
RSSQSLVHSNGNTFLY

SEQ ID NO: 812 - protein, 7 aa, synthetic construct
RVSNRFS

SEQ ID NO: 813 - protein, 9 aa, synthetic construct
FQATHVPWT

SEQ ID NO: 814 - protein, 5 aa, synthetic construct
YFYMN

SEQ ID NO: 815 - protein, 17 aa, synthetic construct
QINPNNGGTAYAQKFQG

SEQ ID NO: 816 - protein, 9 aa, synthetic construct
WVGTHYFDY

SEQ ID NO: 817 - protein, 15 aa, synthetic construct
RASQSVSSSSYTYIH

SEQ ID NO: 818 - protein, 7 aa, synthetic construct
FASNLES

SEQ ID NO: 819 - protein, 9 aa, synthetic construct
QHSWEIPPT

SEQ ID NO: 820 - protein, 5 aa, synthetic construct
SYWMH

SEQ ID NO: 821 - protein, 17 aa, synthetic construct
EINPTNGRTNYIEKFKS

SEQ ID NO: 822 - protein, 7 aa, synthetic construct
GTRAYHY

SEQ ID NO: 823 - protein, 11 aa, synthetic construct
RASDNLYSNLA

SEQ ID NO: 824 - protein, 7 aa, synthetic construct
DATNLAD

SEQ ID NO: 825 - protein, 9 aa, synthetic construct
QHFWGTPLT

SEQ ID NO: 826 - protein, 5 aa, synthetic construct
SYWMH

SEQ ID NO: 827 - protein, 17 aa, synthetic construct
EIAPTNGRTNYIEKFKS

SEQ ID NO: 828 - protein, 7 aa, synthetic construct
GTRAYHY

SEQ ID NO: 829 - protein, 11 aa, synthetic construct
RASDNLYSNLA

SEQ ID NO: 830 - protein, 7 aa, synthetic construct
DATNLAD

SEQ ID NO: 831 - protein, 9 aa, synthetic construct
QHFWGTPLT

SEQ ID NO: 832 - protein, 5 aa, synthetic construct
SYWMH

SEQ ID NO: 833 - protein, 17 aa, synthetic construct
EINPANGRTNYIEKFKS

SEQ ID NO: 834 - protein, 7 aa, synthetic construct
GTRAYHY

SEQ ID NO: 835 - protein, 11 aa, synthetic construct
RASDNLYSNLA

SEQ ID NO: 836 - protein, 7 aa, synthetic construct
DATNLAD

SEQ ID NO: 837 - protein, 9 aa, synthetic construct
QHFWGTPLT

SEQ ID NO: 838 - protein, 5 aa, synthetic construct
SYWMH

SEQ ID NO: 839 - protein, 17 aa, synthetic construct
EINPANGRTNYIEKFKS

SEQ ID NO: 840 - protein, 7 aa, synthetic construct
GTRAYHY

SEQ ID NO: 841 - protein, 11 aa, synthetic construct
RASDNLYSNLA

SEQ ID NO: 842 - protein, 7 aa, synthetic construct
DATNLAD

SEQ ID NO: 843 - protein, 9 aa, synthetic construct
QHFAGTPLT

SEQ ID NO: 844 - protein, 5 aa, synthetic construct
SYWMH

SEQ ID NO: 845 - protein, 17 aa, synthetic construct
EINPTNGRTNYIEKFKS

SEQ ID NO: 846 - protein, 7 aa, synthetic construct
GTRAYHY

SEQ ID NO: 847 - protein, 11 aa, synthetic construct
RASDNLYSNLA

SEQ ID NO: 848 - protein, 7 aa, synthetic construct
DATNLAD

SEQ ID NO: 849 - protein, 9 aa, synthetic construct
QHFAGTPLT

SEQ ID NO: 850 - protein, 5 aa, synthetic construct
SYWMH

SEQ ID NO: 851 - protein, 17 aa, synthetic construct
EIAPTNGRTNYIEKFKS

SEQ ID NO: 852 - protein, 7 aa, synthetic construct
GTRAYHY

SEQ ID NO: 853 - protein, 11 aa, synthetic construct
RASDNLYSNLA

SEQ ID NO: 854 - protein, 7 aa, synthetic construct
DATNLAD

SEQ ID NO: 855 - protein, 9 aa, synthetic construct
QHFAGTPLT

SEQ ID NO: 856 - protein, 5 aa, synthetic construct
GHYVH

SEQ ID NO: 857 - protein, 17 aa, synthetic construct
WIFPGKVNTKYNEKFKG

SEQ ID NO: 858 - protein, 11 aa, synthetic construct
VGYDYPYYFDY

SEQ ID NO: 859 - protein, 11 aa, synthetic construct
RASQSIGTSIH

SEQ ID NO: 860 - protein, 7 aa, synthetic construct
YASESIS

SEQ ID NO: 861 - protein, 9 aa, synthetic construct
QQSSSWPFT

SEQ ID NO: 862 - protein, 6 aa, synthetic construct
SSNWWS

SEQ ID NO: 863 - protein, 16 aa, synthetic construct
EIYHSGSTNYNPSLKS

SEQ ID NO: 864 - protein, 10 aa, synthetic construct
WTGRTDAFDI

SEQ ID NO: 865 - protein, 16 aa, synthetic construct
RSSQSLLHSNGYNYLD

SEQ ID NO: 866 - protein, 7 aa, synthetic construct
LGSNRAS

SEQ ID NO: 867 - protein, 9 aa, synthetic construct
MQGTHWPLT

SEQ ID NO: 868 - protein, 5 aa, synthetic construct
SYGMH

SEQ ID NO: 869 - protein, 17 aa, synthetic construct
IIWFDGSSTYYADSVRG

SEQ ID NO: 870 - protein, 9 aa, synthetic construct
ELGRRYFDL

SEQ ID NO: 871 - protein, 11 aa, synthetic construct
RASQSVSSYLA

SEQ ID NO: 872 - protein, 7 aa, synthetic construct
DASKRAT

SEQ ID NO: 873 - protein, 10 aa, synthetic construct
QQRSKWPPWT

SEQ ID NO: 874 - protein, 5 aa, synthetic construct
SYAMN

SEQ ID NO: 875 - protein, 17 aa, synthetic construct
AISGSGGTTFYADSVKG

SEQ ID NO: 876 - protein, 16 aa, synthetic construct
DLGWSDSYYYYYGMDV

SEQ ID NO: 877 - protein, 11 aa, synthetic construct
RASQGIRNDLG

SEQ ID NO: 878 - protein, 7 aa, synthetic construct
AASRLHR

SEQ ID NO: 879 - protein, 9 aa, synthetic construct
LQHNSYPCS

SEQ ID NO: 880 - protein, 5 aa, synthetic construct
SYAIS

SEQ ID NO: 881 - protein, 17 aa, synthetic construct
GIIPIFGTANYAQKFQG

SEQ ID NO: 882 - protein, 21 aa, synthetic construct
APLRFLEWSTQDHYYYYYMDV

SEQ ID NO: 883 - protein, 11 aa, synthetic construct
QGDSLRSYYAT

SEQ ID NO: 884 - protein, 7 aa, synthetic construct
GENKRPS

SEQ ID NO: 885 - protein, 11 aa, synthetic construct
KSRDGSGQHLV

SEQ ID NO: 886 - protein, 5 aa, synthetic construct
TYTIH

SEQ ID NO: 887 - protein, 17 aa, synthetic construct
YINPRSGYTEYNQKFQD

SEQ ID NO: 888 - protein, 4 aa, synthetic construct
SYEF

SEQ ID NO: 889 - protein, 11 aa, synthetic construct
KASQNVGINVA

SEQ ID NO: 890 - protein, 7 aa, synthetic construct
SASYRYS

SEQ ID NO: 891 - protein, 9 aa, synthetic construct
HQYKTYPYT

SEQ ID NO: 892 - protein, 5 aa, synthetic construct
NYDMT

SEQ ID NO: 893 - protein, 17 aa, synthetic construct
SITSDGGSTYSRDSVKG

SEQ ID NO: 894 - protein, 12 aa, synthetic construct
DRGRYLPYYFDY

SEQ ID NO: 895 - protein, 16 aa, synthetic construct
RSSQSLEYSDGYSYLE

SEQ ID NO: 896 - protein, 7 aa, synthetic construct
EVSSRFS

SEQ ID NO: 897 - protein, 8 aa, synthetic construct
FQAIHDPT

SEQ ID NO: 898 - protein, 5 aa, synthetic construct
TYSVH

SEQ ID NO: 899 - protein, 16 aa, synthetic construct
RMWTAGDTSYNSAFTS

SEQ ID NO: 900 - protein, 11 aa, synthetic construct
SSYTSGYPFDS

SEQ ID NO: 901 - protein, 11 aa, synthetic construct
RASESVSKLMH

SEQ ID NO: 902 - protein, 7 aa, synthetic construct
LTSHLAS

SEQ ID NO: 903 - protein, 8 aa, synthetic construct
QQSRNDPT

SEQ ID NO: 904 - protein, 5 aa, synthetic construct
YYDMH

SEQ ID NO: 905 - protein, 16 aa, synthetic construct
AIGTAGATYYPGSVKG

SEQ ID NO: 906 - protein, 14 aa, synthetic construct
DRGYSGYDAYYFDF

SEQ ID NO: 907 - protein, 11 aa, synthetic construct
RASQSISSYLN

SEQ ID NO: 908 - protein, 7 aa, synthetic construct
AASSLQS

SEQ ID NO: 909 - protein, 9 aa, synthetic construct
QQSYSTPPT

SEQ ID NO: 910 - protein, 7 aa, synthetic construct
SSNYYWG

SEQ ID NO: 911 - protein, 16 aa, synthetic construct
NIYYRGYTYYNPSLKS

SEQ ID NO: 912 - protein, 14 aa, synthetic construct
EGSDYGDYVGAFDI

SEQ ID NO: 913 - protein, 11 aa, synthetic construct
RASQSISSYLN

SEQ ID NO: 914 - protein, 7 aa, synthetic construct
AASSLQS

SEQ ID NO: 915 - protein, 9 aa, synthetic construct
QQSYSTPPT

SEQ ID NO: 916 - protein, 5 aa, synthetic construct
DTYIH

SEQ ID NO: 917 - protein, 17 aa, synthetic construct
RIDPANGKANYDPKFQG

SEQ ID NO: 918 - protein, 9 aa, synthetic construct
GGGYYGMDY

SEQ ID NO: 919 - protein, 15 aa, synthetic construct
RASESVDSYGNSFMH

SEQ ID NO: 920 - protein, 7 aa, synthetic construct
LASNLES

SEQ ID NO: 921 - protein, 9 aa, synthetic construct
QQNNEDPLT

SEQ ID NO: 922 - protein, 5 aa, synthetic construct
RYWIE

SEQ ID NO: 923 - protein, 17 aa, synthetic construct
EILPGSGSTNYNEKFKG

SEQ ID NO: 924 - protein, 4 aa, synthetic construct
RGAY

SEQ ID NO: 925 - protein, 11 aa, synthetic construct
KASEDIYNRLA

SEQ ID NO: 926 - protein, 7 aa, synthetic construct
GATSLET

SEQ ID NO: 927 - protein, 9 aa, synthetic construct
QQQWSTPPT

SEQ ID NO: 928 - protein, 5 aa, synthetic construct
KPVMG

SEQ ID NO: 929 - protein, 16 aa, synthetic construct
AITSGTNTYYADSVKG

SEQ ID NO: 930 - protein, 10 aa, synthetic construct
DDYDVVEYPY

SEQ ID NO: 931 - protein, 6 aa, synthetic construct
SSYWMC

SEQ ID NO: 932 - protein, 18 aa, synthetic construct
CIYTGSSGSTSYASWAKG

SEQ ID NO: 933 - protein, 14 aa, synthetic construct
DYDYTAYAYGIMSL

SEQ ID NO: 934 - protein, 11 aa, synthetic construct
QASQSITSYLS

SEQ ID NO: 935 - protein, 7 aa, synthetic construct
RASTLAS

SEQ ID NO: 936 - protein, 14 aa, synthetic construct
QSNYGSYSTNYGVT

SEQ ID NO: 937 - protein, 6 aa, synthetic construct
SRYYMC

SEQ ID NO: 938 - protein, 17 aa, synthetic construct
CIYTGSGSTYYASWAKG

SEQ ID NO: 939 - protein, 7 aa, synthetic construct
EAGSFNL

SEQ ID NO: 940 - protein, 11 aa, synthetic construct
QASQSIYSGLA

SEQ ID NO: 941 - protein, 7 aa, synthetic construct
SASKLAS

SEQ ID NO: 942 - protein, 12 aa, synthetic construct
QNYYYGISNGWT

SEQ ID NO: 943 - protein, 5 aa, synthetic construct
SYHMH

SEQ ID NO: 944 - protein, 17 aa, synthetic construct
WINPYTGDTNYAQKFQG

SEQ ID NO: 945 - protein, 7 aa, synthetic construct
EKVYMDI

SEQ ID NO: 946 - protein, 11 aa, synthetic construct
SGDSIPSKYAH

SEQ ID NO: 947 - protein, 7 aa, synthetic construct
GKSHRPS

SEQ ID NO: 948 - protein, 11 aa, synthetic construct
AAWDLLGDGWV

SEQ ID NO: 949 - protein, 5 aa, synthetic construct
DYGIH

SEQ ID NO: 950 - protein, 19 aa, synthetic construct
RIKSKTDGGITEYAAPVKG

SEQ ID NO: 951 - protein, 10 aa, synthetic construct
AIYYLEAFDV

SEQ ID NO: 952 - protein, 11 aa, synthetic construct
SGDSLGSYYVH

SEQ ID NO: 953 - protein, 7 aa, synthetic construct
RNKQRPS

SEQ ID NO: 954 - protein, 11 aa, synthetic construct
QTYDWMYSSRV

SEQ ID NO: 955 - protein, 5 aa, synthetic construct
NYGMH

SEQ ID NO: 956 - protein, 16 aa, synthetic construct
YISSNSGTIYADAVKG

SEQ ID NO: 957 - protein, 9 aa, synthetic construct
GYFDGYYRF

SEQ ID NO: 958 - protein, 17 aa, synthetic construct
RSSQSLLSSGNQKNYLA

SEQ ID NO: 959 - protein, 7 aa, synthetic construct
HASTRQS

SEQ ID NO: 960 - protein, 8 aa, synthetic construct
LQHYSSPT

SEQ ID NO: 961 - protein, 5 aa, synthetic construct
NYDMA

SEQ ID NO: 962 - protein, 17 aa, synthetic construct
SISPGGGKTYYRDSVKG

SEQ ID NO: 963 - protein, 12 aa, synthetic construct
LGPAYSGEWFAY

SEQ ID NO: 964 - protein, 11 aa, synthetic construct
RASEGVNTRIH

SEQ ID NO: 965 - protein, 7 aa, synthetic construct
GASNLDS

SEQ ID NO: 966 - protein, 9 aa, synthetic construct
QQSWNVPHT

SEQ ID NO: 967 - protein, 5 aa, synthetic construct
RYPMN

SEQ ID NO: 968 - protein, 17 aa, synthetic construct
WINTNTGNPTYAQGFTG

SEQ ID NO: 969 - protein, 11 aa, synthetic construct
ERTNFYDAFDI

SEQ ID NO: 970 - protein, 11 aa, synthetic construct
RASQSISSYLN

SEQ ID NO: 971 - protein, 7 aa, synthetic construct
AASSLQS

SEQ ID NO: 972 - protein, 9 aa, synthetic construct
QQSYSTPPT

SEQ ID NO: 973 - protein, 5 aa, synthetic construct
SYAMD

SEQ ID NO: 974 - protein, 17 aa, synthetic construct
WINTNTGNPTYAQAFTG

SEQ ID NO: 975 - protein, 11 aa, synthetic construct
ERHGYFEAFDI

SEQ ID NO: 976 - protein, 11 aa, synthetic construct
RASQSISSYLN

SEQ ID NO: 977 - protein, 7 aa, synthetic construct
AASSLQS

SEQ ID NO: 978 - protein, 9 aa, synthetic construct
QQSYSTPPT

SEQ ID NO: 979 - protein, 5 aa, synthetic construct
KYAMN

SEQ ID NO: 980 - protein, 17 aa, synthetic construct
WINTNTGNPTYAQGFTG

SEQ ID NO: 981 - protein, 11 aa, synthetic construct
KGGSYYDWFDP

SEQ ID NO: 982 - protein, 11 aa, synthetic construct
RASQSISSYLN

SEQ ID NO: 983 - protein, 7 aa, synthetic construct
AASSLQS

SEQ ID NO: 984 - protein, 9 aa, synthetic construct
QQSYSTPPT

SEQ ID NO: 985 - protein, 5 aa, synthetic construct
AHIMG

SEQ ID NO: 986 - protein, 16 aa, synthetic construct
GISQGSITNYADSVKG

SEQ ID NO: 987 - protein, 13 aa, synthetic construct
ISYDYIKSVPFRY

SEQ ID NO: 988 - protein, 5 aa, synthetic construct
LWYMG

SEQ ID NO: 989 - protein, 16 aa, synthetic construct
SIGIGATTNYADSVKG

SEQ ID NO: 990 - protein, 14 aa, synthetic construct
FGWAYSGYHDDFLY

SEQ ID NO: 991 - protein, 5 aa, synthetic construct
QLDMG

SEQ ID NO: 992 - protein, 16 aa, synthetic construct
AINQGTTTYYADSVKG

SEQ ID NO: 993 - protein, 10 aa, synthetic construct
YLYDIWNHPY

SEQ ID NO: 994 - protein, 5 aa, synthetic construct
GKGMG

SEQ ID NO: 995 - protein, 16 aa, synthetic construct
AIGSGAITYYADSVKG

SEQ ID NO: 996 - protein, 10 aa, synthetic construct
YTTALDEYPY

SEQ ID NO: 997 - protein, 5 aa, synthetic construct
YYVMG

SEQ ID NO: 998 - protein, 16 aa, synthetic construct
SISYGASTYYADSVKG

SEQ ID NO: 999 - protein, 10 aa, synthetic construct
DFDSNYAHTY

SEQ ID NO: 1000 - protein, 5 aa, synthetic construct
IGYMG

SEQ ID NO: 1001 - protein, 16 aa, synthetic construct
SIASGTSTYYADSVKG

SEQ ID NO: 1002 - protein, 10 aa, synthetic construct
TQYIQDVHRY

SEQ ID NO: 1003 - protein, 5 aa, synthetic construct
SYGIS

SEQ ID NO: 1004 - protein, 17 aa, synthetic construct
WISAYNGYTNYAQKLQG

SEQ ID NO: 1005 - protein, 10 aa, synthetic construct
DLRGTNYFDY

SEQ ID NO: 1006 - protein, 11 aa, synthetic construct
RASQGISNWLA

SEQ ID NO: 1007 - protein, 7 aa, synthetic construct
AASSLLS

SEQ ID NO: 1008 - protein, 9 aa, synthetic construct
QQANSFPIT

SEQ ID NO: 1009 - protein, 5 aa, synthetic construct
SYWLH

SEQ ID NO: 1010 - protein, 17 aa, synthetic construct
MIDPSNSDTRFNPNFKD

SEQ ID NO: 1011 - protein, 10 aa, synthetic construct
YRSYVTPLDY

SEQ ID NO: 1012 - protein, 17 aa, synthetic construct
KSSQSLLYTSSQKNYLA

SEQ ID NO: 1013 - protein, 7 aa, synthetic construct
WASTRES

SEQ ID NO: 1014 - protein, 9 aa, synthetic construct
QQYYAYPWT

SEQ ID NO: 1015 - protein, 5 aa, synthetic construct
AYTMH

SEQ ID NO: 1016 - protein, 17 aa, synthetic construct
WIKPNNGLANYAQKFQG

SEQ ID NO: 1017 - protein, 9 aa, synthetic construct
SEITTEFDY

SEQ ID NO: 1018 - protein, 15 aa, synthetic construct
KSSESVDSYANSFLH

SEQ ID NO: 1019 - protein, 7 aa, synthetic construct
RASTRES

SEQ ID NO: 1020 - protein, 9 aa, synthetic construct
QQSKEDPLT

SEQ ID NO: 1021 - protein, 5 aa, synthetic construct
DYAMH

SEQ ID NO: 1022 - protein, 17 aa, synthetic construct
GITWNSYNIDYADSVKG

SEQ ID NO: 1023 - protein, 12 aa, synthetic construct
DDDYSNYVYFDY

SEQ ID NO: 1024 - protein, 11 aa, synthetic construct
RASQSISSYLN

SEQ ID NO: 1025 - protein, 7 aa, synthetic construct
AASSLQS

SEQ ID NO: 1026 - protein, 10 aa, synthetic construct
QQSYSTPPIT

SEQ ID NO: 1027 - protein, 454 aa, synthetic construct
QQQLEEYGGDLVQPEGSLTLTCKASGLDFSSSYWMCWVRQAPGKGLEWIACIYTGSSGSTSYASWAKGRFTISKTSSTTVTLQMTSLTAADTATYFCARDYDYTAYAYGIMSLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1028 - protein, 124 aa, synthetic construct
QQQLEEYGGDLVQPEGSLTLTCKASGLDFSSSYWMCWVRQAPGKGLEWIACIYTGSSGSTSYASWAKGRFTISKTSSTTVTLQMTSLTAADTATYFCARDYDYTAYAYGIMSLWGPGTLVTVSS

SEQ ID NO: 1029 - protein, 446 aa, synthetic construct
QEQLVESGGGLVQPEGSLTLTCTASGFSFSSRYYMCWVRQAPGKGLEWIGCIYTGSGSTYYASWAKGRVTISKTSSTTVTLQMTSLTAADTATYFCAREAGSFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1030 - protein, 116 aa, synthetic construct
QEQLVESGGGLVQPEGSLTLTCTASGFSFSSRYYMCWVRQAPGKGLEWIGCIYTGSGSTYYASWAKGRVTISKTSSTTVTLQMTSLTAADTATYFCAREAGSFNLWGPGTLVTVSS

(19) United States
(12) Patent Application Publication
(10) Pub. No.: US 2026/0022189 A1
(43) Pub. Date: Jan. 22, 2026

(54) DEGRADATION OF cMET USING A BISPECIFIC BINDING AGENT

(71) Applicant: EpiBiologics, Inc., San Mateo, CA (US)

(72) Inventors: Jonathan SITRIN, San Mateo, CA (US); Hai TRAN, San Mateo, CA (US); Lisa MARSHALL, San Mateo, CA (US); Kenneth NG, San Mateo, CA (US); Kimberly HOI, San Mateo, CA (US); Shyra GARDAI, San Mateo, CA (US); Katarina PANCE, San Francisco, CA (US); Josef GRAMESPACHER, Seaside, CA (US); Rami HANNOUSH, San Mateo, CA (US)

(21) Appl. No.: 19/200,494

(22) Filed: May 6, 2025

Related U.S. Application Data
(63) Continuation of application No. PCT/US2023/080417, filed on Nov. 17, 2023.
(60) Provisional application No. 63/384,371, filed on Nov. 18, 2022.
(60) Provisional application No. 63/479,515, filed on Jan. 11, 2023.

(51) Int. Cl. C07K 16/30; A61P 35/00; C07K 16/28

(52) U.S. Cl. CPC C07K 16/3092; A61P 35/00; C07K 16/2863; C07K 2317/31; C07K 2317/73; C07K 2317/76; C07K 2317/77

(57) ABSTRACT

The present disclosure provides methods of degrading a cMET protein on a target cell. The present disclosure further discloses binding agents that bind to a cMET protein and a degrading protein.

### CROSS-REFERENCE

This application is a continuation of International Application No. PCT/US2023/080417, filed Nov. 17, 2023, which claims the benefit of U.S. Provisional Application No. 63/384,371, filed Nov. 18, 2022, and U.S. Provisional Application No. 63/479,515, filed Jan. 11, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 16, 2025, is named 6563-705.301_SL.xml and is 974,000 bytes in size, and is incorporated by reference as if written herein in its entirety.

### BACKGROUND

Targeted protein degradation is a promising new therapeutic strategy compared to conventional inhibition-based therapeutics. Inhibitors rely on sustained, occupancy-driven pharmacology, necessitating high affinity binders capable of abrogating catalytic or binding functions. Inhibiting protein-protein interactions or scaffolding functions has been extremely challenging for standard binding-based small molecules. In contrast, protein degraders are catalytic and utilize event-driven pharmacology, alleviating the need for high affinity binders, and durably abrogate all protein functions at once. As such, degrader technologies such as proteolysis targeting chimeras (PROTACs) have had great success in targeting traditionally challenging proteins. A number of PROTACs are currently in clinical trials.

Most degrader technologies, including PROTACs, utilize an intracellular mechanism of action and have thus been largely limited to targeting proteins with cytoplasmic domains. However, recent approaches, such as LYTACs have been described for specifically degrading cell surface proteins. These utilize recycling glycan receptors such as the mannose-6-phosphate receptor (M6PR) or asialoglycoprotein receptor (ASGR) to target proteins for internalization and trafficking to the lysosome for degradation. These require complex glycans conjugated to antibodies or to small molecules to effect degradation of a membrane protein.

As a hybrid approach that is broadly applicable to many cell types, we recently described antibody-based PROTACs (AbTACs). AbTACs utilize a standard IgG bispecific antibody format to bring a cell surface E3 ligase (RNF43) into proximity of a membrane protein of interest (POI) to mediate its degradation through the lysosomal pathway. The traditional bispecific IgG scaffold on which the AbTAC is built possesses favorable pharmacokinetic properties relative to LYTACS and other small molecule based degraders. Furthermore, in contrast to other degradation modalities such as LYTACS and PROTACS, AbTACs are fully recombinant. However, there continues to exist a need for targeted protein degraders that efficiently and selectively induce the degradation of a target protein.

### SUMMARY

In one aspect, the present disclosure provides a method of degrading a target protein on a surface of a target cell, the method comprising: contacting a degrading protein and the target protein on the surface of the target cell with a binding agent, wherein the binding agent comprises: (i) a first binding domain that specifically binds to the degrading protein: (ii) a second binding domain that specifically binds to the target protein, wherein the target protein comprises cMET.

In some embodiments, the binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the binding agent is a multispecific antibody or a bispecific antibody. In some embodiments, the binding agent is a bispecific antibody.

In some embodiments, the degrading protein is CDH3, MUC1, CD276, TROP2, CD71, HER3, TNFRSF10B, ITGB6, PD-L1, EpCAM, TPBG, EGFR, MSTIR, EphA2, ADAM9, IGF1R, RNF43, RNF128, RNF130, or ZNRF3.

In some embodiments, the degrading protein is CDH3. In some embodiments, the first binding domain comprises a first binding domain variable heavy chain, and wherein the variable heavy chain of the first binding domain comprises at least 80% sequence identity to any one of SEQ ID NO: 106, 110, 114, 118 or 122. In some embodiments, the first binding domain variable heavy chain comprises at least 90% sequence identity to any one of SEQ ID NO: 106, 110, 114, 118 or 122. In some embodiments, the first binding domain variable heavy chain comprises any one of SEQ ID NO: 106, 110, 114, 118 or 122. In some embodiments, the first binding domain comprises a first binding domain variable light chain and wherein the first binding domain variable light chain comprises at least 80% sequence identity to any one of SEQ ID NO: 108, 112, 116, 120, or 124. In some embodiments, the first binding domain variable light chain comprises at least 90% sequence identity to any one of SEQ ID NO: 108, 112, 116, 120, or 124. In some embodiments, the first binding domain variable light chain comprises any one of SEQ ID NO: 108, 112, 116, 120, or 124. In some embodiments, the first binding domain binds to an epitope of the degrading protein on the target cell, wherein the epitope comprises at least 80% sequence identity to an epitope to which an antibody comprising any one of SEQ ID NO: 106, 110, 114, 118 or 122 and SEQ ID NO: any one of SEQ ID NO: 108, 112, 116, 120, or 124 binds. In some embodiments, the first binding domain binds to an epitope of the degrading protein on the target cell, wherein the epitope comprises at least 90% sequence identity to the epitope to which the antibody comprising any one of SEQ ID NO: 106, 110, 114, 118 or 122 and any one of SEQ ID NO: 108, 112, 116, 120, or 124 binds. In some embodiments, the first binding domain binds to an epitope of the degrading protein on the target cell that does not include any of the amino acids from the epitope to which an antibody comprising any one of SEQ ID NO: 106, 110, 114, 118 or 122 and any one of SEQ ID NO: 108, 112, 116, 120, or 124 binds.

In some embodiments, the degrading protein is MUC1. In some embodiments, the first binding domain comprises a first binding domain variable heavy chain, and wherein the first binding domain variable heavy chain comprises at least 80%, sequence identity to any one of SEQ ID NO: 6, 10, 14, 18, or 22. In some embodiments, the first binding domain variable heavy chain comprises at least 90% sequence identity to any one of SEQ ID NO: 6, 10, 14, 18, or 22. In some embodiments, the first binding domain variable heavy chain comprises any one of SEQ ID NO: 6, 10, 14, 18, or 22. In some embodiments, the first binding domain comprises a first binding domain variable light chain, and wherein the first binding domain variable light chain comprises at least 80% sequence identity to any one of SEQ ID NO: 8, 12, 16, 20, or 24. In some embodiments, the first binding domain variable light chain comprises at least 90% sequence identity to any one of SEQ ID NO: 8, 12, 16, 20, or 24. In some embodiments, the first binding domain variable light chain comprises any one of SEQ ID NO: 8, 12, 16, 20, or 24. In some embodiments, the first binding domain binds to an epitope of the degrading protein on the target cell, wherein the epitope comprises at least 80% sequence identity to an epitope to which an antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell, wherein the epitope comprises at least 90% sequence identity to the epitope to which the antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell that does not include any of the amino acids from the epitope to which the antibody comprising any one of SEQ ID NO: 6, 10, 14, 18, or 22 and any one of SEQ ID NO: 8, 12, 16, 20, or 24 binds.

In some embodiments, the degrading protein is CD276. In some embodiments, the first binding domain comprises a first binding domain variable heavy chain, and wherein the first binding domain variable heavy chain comprises at least 80%, sequence identity to any one of SEQ ID NO: 25, 26, 30, 34, or 38. In some embodiments, the first binding domain variable heavy chain comprises at least 90%, sequence identity to any one of SEQ ID NO: 26, 30, 34, or 38. In some embodiments, the first binding domain variable heavy chain comprises any one of SEQ ID NO: 26, 30, 34, or 38. In some embodiments, the first binding domain comprises a first binding domain variable light chain, and wherein the first binding domain variable light chain comprises at least 80% sequence identity to any one of SEQ ID NO: 28, 32, 36, or 40. In some embodiments, the first binding domain variable light chain comprises at least 90% sequence identity to any one of SEQ ID NO: 28, 32, 36, or 40. In some embodiments, the first binding domain variable light chain comprises any one of SEQ ID NO: 28, 32, 36, or 40. In some embodiments, the first binding domain binds to an epitope of the degrading protein on the target cell, wherein the epitope comprises at least 80% sequence identity to an epitope to which an antibody comprising any one of SEQ ID NO: 26, 30, 34, or 38 and any one of SEQ ID NO: 28, 32, 36, or 40 binds. In some embodiments, the first binding domain binds to an epitope e of the degrading protein on the target cell, wherein the epitope comprises at least 90% sequence identity to the epitope to which the antibody comprising any one of SEQ ID NO: 26, 30, 34, or 38 and any one of SEQ ID NO: 28, 32, 36, or 40 binds. In some embodiments, the first binding domain binds to an epitope of the degrading protein on the target cell that does not include any of the amino acids from the epitope to which the antibody comprising any one of SEQ ID NO: 26, 30, 34, or 38 and any one of SEQ ID NO: 28, 32, 36, or 40 binds.

In some embodiments, the degrading protein is TROP2. In some embodiments, the first binding domain comprises a first binding domain variable heavy chain, and wherein the first binding domain variable heavy chain comprises at least 80%, sequence identity to any one of SEQ ID NO: 198, 202, 206, 210, or 214. In some embodiments, the first binding domain variable heavy chain comprises at least 90%, sequence identity to any one of SEQ ID NO: 198, 202, 206, 210, or 214. In some embodiments, the first binding domain variable heavy chain comprises any one of SEQ ID NO: 198, 202, 206, 210, or 214. In some embodiments, the first binding domain comprises a first binding domain variable light chain, and wherein the first binding domain variable light chain comprises at least 80% sequence identity to any one of SEQ ID NO: 200, 204, 208, 212, or 216. In some embodiments, the first binding domain variable light chain comprises at least 90% sequence identity to any one of SEQ ID NO: 200, 204, 208, 212, or 216. In some embodiments, the first binding domain variable light chain comprises any one of SEQ ID NO: 200, 204, 208, 212, or 216. In some embodiments, the first binding domain binds to an epitope of the degrading protein on the target cell, wherein the epitope comprises at least 80% sequence identity to an epitope to which an antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell, wherein the epitope comprises at least 90% sequence identity to the epitope to which the antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell that does not include any of the amino acids from the epitope to which the antibody comprising any one of SEQ ID NO: 198, 202, 206, 210, or 214 and any one of SEQ ID NO: 200, 204, 208, 212, or 216 binds.

In some embodiments, wherein the degrading protein is selected from the group consisting of CD71. HER3. TNFRSF10B. ITGB6. PD-L1. EpCAM. TPBG. MSTIR. EphA2. ADAM9, IGF1R, and EGFR. In some embodiments, the degrading protein is RNF43, RNF128, RNF130, or ZNRF3. In some embodiments, the first binding domain comprises a first binding domain variable heavy chain, and wherein the first binding domain variable heavy chain comprises at least 80%, sequence identity to any one of variable heavy chain sequences listed in Table 1 or Table 4. In some embodiments, the first binding domain variable heavy chain comprises at least 90%, sequence identity to any one of variable heavy chain sequences listed in Table 1 or Table 4. In some embodiments, the first binding domain variable heavy chain comprises any one of variable heavy chain sequences listed in Table 1 or Table 4. In some embodiments, the first binding domain comprises a first binding domain variable light chain, and wherein the first binding domain variable light chain comprises at least 80% sequence identity to any one of variable light chain sequences listed in Table 1 or Table 4. In some embodiments, the first binding domain variable light chain comprises at least 90% sequence identity to any one of variable light chain sequences listed in Table 1 or Table 4. In some embodiments, the first binding domain variable light chain comprises any one of variable light chain sequences listed in Table 1 or Table 4. In some embodiments, the first binding domain binds to an epitope of the degrading protein on the target cell, wherein the epitope comprises at least 80% sequence identity to an epitope to which an antibody comprising any one of a variable heavy chain sequence or any one of variable light chain sequences listed in Table 1 or Table 4 binds. In some embodiments, the first binding domain binds to an epitope of the degrading protein on the target cell, wherein the epitope comprises at least 90% sequence identity to the epitope to which the antibody comprising any one of variable heavy chain sequence or any one of variable light chain sequences listed in Table 1 or Table 4 binds. In some embodiments, the first binding domain binds to an epitope of the degrading protein on the target cell that does not include any of the amino acids from the epitope to which the antibody comprising any one of variable heavy chain sequence or any one of variable light chain sequences listed in Table 1 or Table 4 binds.

In some embodiments, the second binding domain comprises a second binding domain variable heavy chain, and wherein the second binding domain variable heavy chain comprises at least 80%, sequence identity to any one of SEQ ID NO: 352, 356, 360, or 364. In some embodiments, the second binding domain variable heavy chain comprises at least 90%, sequence identity to any one of SEQ ID NO: 352, 356, 360, or 364. In some embodiments, the second binding domain variable heavy chain comprises any one of SEQ ID NO: 352, 356, 360, or 364. In some embodiments, the second binding domain comprises a second binding domain variable light chain, and wherein the second binding domain variable heavy chain comprises at least 80%, sequence identity to any one of SEQ ID NO: 354, 358, 362 or 366. In some embodiments, the second binding domain variable heavy chain comprises at least 90%, sequence identity to any one of SEQ ID NO: 354, 358, 362 or 366. In some embodiments, the second binding domain variable heavy chain comprises any one of SEQ ID NO: 354, 358, 362 or 366.

In some embodiments, the second binding domain binds to an epitope of the target protein on the target cell, wherein the epitope comprises at least 80% sequence identity to an epitope to which Amivantamab binds. In some embodiments, the second binding domain binds to an epitope of the target protein on the target cell, wherein the epitope comprises at least 90% sequence identity to the epitope to which Amivantamab binds. In some embodiments, the second binding domain binds to an epitope of the target protein on the target cell that does not include any of the amino acids from the epitope to which Amivantamab binds. In some embodiments, the second binding domain binds to an epitope of the target protein on the target cell, wherein the epitope comprises at least 80% sequence identity to an epitope to which Telisotuzumab binds. In some embodiments, the second binding domain binds to an epitope of the target protein on the target cell, wherein the epitope comprises at least 90% sequence identity to the epitope to which Telisotuzumab binds. In some embodiments, the second binding domain binds to an epitope of the target protein on the target cell that does not include any of the amino acids from the epitope to which Telisotuzumab binds. In some embodiments, the second binding domain binds to an epitope of the target protein on the target cell, wherein the epitope comprises at least 80% sequence identity to an epitope to which Onartuzumab binds. In some embodiments, the second binding domain binds to an epitope of the target protein on the target cell, wherein the epitope comprises at least 90% sequence identity to the epitope to which Onartuzumab binds. In some embodiments, the second binding domain binds to an epitope of the target protein on the target cell that does not include any of the amino acids from the epitope to which Onartuzumab binds. In some embodiments, the second binding domain binds to an epitope of the target protein on the target cell, wherein the epitope comprises at least 80% sequence identity to an epitope to which REGN5093s58 binds. In some embodiments, the second binding domain binds to an epitope of the target protein on the target cell, wherein the epitope comprises at least 90% sequence identity to the epitope to which REGN5093s58 binds. In some embodiments, the second binding domain binds to an epitope of the target protein on the target cell that does not include any of the amino acids from the epitope to which REGN5093s58 binds.

In some embodiments, following the contacting, cMET is internalized with the degrading protein into the target cell and cMET is degraded. In some embodiments, the degrading protein is recycled to the surface of the target cell after the cMET is internalized with the degrading protein into the target cell. In some embodiments, one or more of the degrading protein or the cMET are degraded after the cMET is internalized with the degrading protein into the target cell.

In some embodiments, the target cell is a cancer cell. In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, a gastric adenocarcinoma cell, non-small cell lung cancer cell, head and neck cancer cell, and cancers harboring cMET mutations including exon 14 deletions. In some embodiments, the cancer cell is gastric adenocarcinoma cell. In some embodiments, the cancer cell is non-small cell lung cancer cell. In some embodiments, the cancer cell comprises a mutation in a gene selected from a cMET exon 14 skipping mutation or a cMET duplication mutation. In some embodiments, the mutation comprises a cMET exon 14 skipping mutation. In some embodiments, the cancer cell comprises a cMET duplication mutation.

In some embodiments, expression of cMET in the cancer cell following the contacting with the binding agent is less than expression of cMET in a control cancer cell that is not contacted with the binding agent. In some embodiments, expression of cMET in the cancer cell following the contacting with the binding agent is at least 50% less than expression of cMET in a control cancer cell not contacted with the binding agent. In some embodiments, expression of cMET in the cancer cell following the contacting with the binding agent is at least 50% less than the expression of cMET in a control cancer cell contacted with a monospecific cMET binding agent.

In some embodiments, an amount of cMET on the surface of the cancer cell following the contacting with the binding agent is less than an amount of cMET on a surface of a control cancer cell not contacted with the binding agent. In some embodiments, the amount of cMET on the surface of the cancer cell following the contacting with the binding agent is at least 20% less than the amount of cMET on a surface of a control cancer cell not contacted with the binding agent. In some embodiments, an amount of cMET on the surface of the cancer cell following the contacting with the binding agent is at least 20% less than an amount of cMET on a surface of a control cancer cell contacted with a monospecific cMET binding agent. In some embodiments, an amount of cMET on the surface of the cancer cell is determined by staining the cancer cell with fluorescently labeled antibodies against cMET and measuring fluorescent intensity.

In some embodiments, an amount of cMET internalized for the cancer cell following the contacting with the binding agent is more than an amount of cMET internalized for a control cancer cell not contacted with the binding agent. In some embodiments, amount of cMET internalized for the cancer cell following the contacting with the binding agent is at least 20% more than the amount of cMET internalized for a control cancer cell not contacted with the binding agent. In some embodiments, an amount of cMET internalized for the cancer cell following the contacting with the binding agent is at least 20% more than an amount of cMET internalized for a control cancer cell contacted with a monospecific cMET binding agent. In some embodiments, the amount of cMET internalized is determined by the further step of labeling the binding agent with a fluorescent tag prior to the contacting with the degrading protein and the target protein and measuring the fluorescence of the fluorescent tag after the contacting with the degrading protein and the target protein, wherein the fluorescent tag selectively fluoresces under intracellular pH.

In some embodiments, an amount of cMET degraded in the cancer cell following the contacting with the binding agent is more than an amount of cMET degraded in a control cancer cell not contacted with the binding agent. In some embodiments, the amount of cMET degraded in the cancer cell following the contacting with the binding agent is at least 20% more than the amount of cMET degraded in a control cancer cell not contacted with the binding agent. In some embodiments, an amount of cMET degraded in the cancer cell following the contacting with the binding agent is at least 20% more than an amount of cMET degraded in a control cancer cell contacted with a monospecific cMET binding agent.

In some embodiments, an amount of cMET dimers on the cancer cell following the contacting with the binding agent is less than an amount of cMET dimers on a control cancer cell not contacted with the binding agent. In some embodiments, an amount of cMET dimers on the cancer cell following the contacting with the binding agent is less than an amount of cMET dimers on a control cancer cell contacted with a monospecific cMET binding agent. In some embodiments, an amount of cMET activation in the cancer cell following the contacting with the binding agent is within 50% of an amount of cMET activation in a control cancer cell not contacted with the binding agent.

In some embodiments, the monospecific cMET binding agent is Telisotuzumab. In some embodiments, the monospecific cMET binding agent is Onartuzumab. In some embodiments, the monospecific cMET binding agent is REGN5093s58.

In some embodiments, the method increases susceptibility of the cancer cell to cancer therapeutic agents or radiation therapy. In some embodiments, the cancer therapeutic agent is a cytotoxic agent. In some embodiments, the method reduces proliferation of the cancer cell. In some embodiments, the method induces death of the cancer cell. In some embodiments, the contacting is performed in vivo.

In one aspect, the present disclosure provides a method for treating cancer in a subject in need thereof, the method comprising: administering to the subject a binding agent, wherein the binding agent comprises: (i) a first binding domain that specifically binds to a degrading protein, wherein the degrading protein is expressed on a target cell: (ii) a second binding domain that specifically binds to the target protein, wherein the target protein comprises cMET.

In some embodiments, the binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the binding agent is a multispecific antibody or a bispecific antibody. In some embodiments, the binding agent is a bispecific antibody.

In some embodiments, the degrading protein is CDH3. MUC1. CD276. TROP2. CD71, HER3, TNFRSF10B, ITGB6, PD-L1, EpCAM, TPBG, EGFR, MSTIR, EphA2, ADAM9, IGF1R, RNF43, RNF128, RNF130, or ZNRF3.

In some embodiments, the degrading protein is CDH3. In some embodiments, the first binding domain comprises a first binding domain variable heavy chain, and wherein the variable heavy chain of the first binding domain comprises at least 80% sequence identity to any one of SEQ ID NO: 106, 110, 114, 118 or 122. In some embodiments, the first binding domain variable heavy chain comprises at least 90% sequence identity to any one of SEQ ID NO: 106, 110, 114, 118 or 122. In some embodiments, the first binding domain variable heavy chain comprises SEQ ID NO: any one of SEQ ID NO: 106, 110, 114, 118 or 122. In some embodiments, the first binding domain comprises a first binding domain variable light chain and wherein the first binding domain variable light chain comprises at least 80% sequence identity to any one of SEQ ID NO: 108, 112, 116, 120, or 124. In some embodiments, the first binding domain variable light chain comprises at least 90% sequence identity to any one of SEQ ID NO: 108, 112, 116, 120, or 124. In some embodiments, the first binding domain variable light chain comprises any one of SEQ ID NO: 108, 112, 116, 120, or 124. In some embodiments, the first binding domain binds to an epitope of the degrading protein on the target cell, wherein the epitope comprises at least 80% sequence identity to an epitope to which an antibody comprising any one of SEQ ID NO: 106, 110, 114, 118 or 122 and any one of SEQ ID NO: 108, 112, 116, 120, or 124 binds. In some embodiments, the first binding domain binds to an epitope of the degrading protein on the target cell, wherein the epitope comprises at least 90% sequence identity to the epitope to which the antibody comprising any one of SEQ ID NO: 106, 110, 114, 118 or 122 and any one of SEQ ID NO: 108, 112, 116, 120, or 124 binds. In some embodiments, the first binding domain binds to an epitope of the degrading protein on the target cell that does not include any of the amino acids from the epitope to which an antibody comprising any one of SEQ ID NO: 106, 110, 114, 118 or 122 and any one of SEQ ID NO: 108, 112, 116, 120, or 124 binds.

In some embodiments, wherein the degrading protein is MUC1. In some embodiments, the first binding domain comprises a first binding domain variable heavy chain, and wherein the first binding domain variable heavy chain comprises at least 80%, sequence identity to any one of SEQ ID NO: 6, 10, 14, 18, or 22. In some embodiments, the first binding domain variable heavy chain comprises at least 90% sequence identity to any one of SEQ ID NO: 6, 10, 14, 18, or 22. In some embodiments, the first binding domain variable heavy chain comprises any one of SEQ ID NO: 6, 10, 14, 18, or 22. In some embodiments, the first binding domain comprises a first binding domain variable light chain, and wherein the first binding domain variable light chain comprises at least 80% sequence identity to any one of SEQ ID NO: 8, 12, 16, 20, or 24. In some embodiments, the first binding domain variable light chain comprises at least 90% sequence identity to any one of SEQ ID NO: 8, 12, 16, 20, or 24. In some embodiments, the first binding domain variable light chain comprises any one of SEQ ID NO: 8, 12, 16, 20, or 24. In some embodiments, the first binding domain binds to an epitope of the degrading protein on the target cell, wherein the epitope comprises at least 80% sequence identity to an epitope to which an antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell, wherein the epitope comprises at least 90% sequence identity to the epitope to which the antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell that does not include any of the amino acids from the epitope to which the antibody comprising any one of SEQ ID NO: 6, 10, 14, 18, or 22 and any one of SEQ ID NO: 8, 12, 16, 20, or 24 binds.

In some embodiments, the degrading protein is CD276. In some embodiments, the first binding domain comprises a first binding domain variable heavy chain, and wherein the first binding domain variable heavy chain comprises at least 80%, sequence identity to any one of SEQ ID NO: 26, 30, 34, or 38. In some embodiments, the first binding domain variable heavy chain comprises at least 90%, sequence identity to any one of SEQ ID NO: 26, 30, 34, or 38. In some embodiments, the first binding domain variable heavy chain comprises any one of SEQ ID NO: 26, 30, 34, or 38. In some embodiments, the first binding domain comprises a first binding domain variable light chain, and wherein the first binding domain variable light chain comprises at least 80% sequence identity to any one of SEQ ID NO: 28, 32, 36, or 40. In some embodiments, the first binding domain variable light chain comprises at least 90% sequence identity to any one of SEQ ID NO: 28, 32, 36, or 40. In some embodiments, the first binding domain variable light chain comprises any one of SEQ ID NO: 28, 32, 36, or 40. In some embodiments, the first binding domain binds to an epitope of the degrading protein on the target cell, wherein the epitope comprises at least 80% sequence identity to an epitope to which an antibody comprising any one of SEQ ID NO: 26, 30, 34, or 38 and any one of SEQ ID NO: 28, 32, 36, or 40 binds. In some embodiments, the first binding domain binds to an epitope e of the degrading protein on the target cell, wherein the epitope comprises at least 90% sequence identity to the epitope to which the antibody comprising any one of SEQ ID NO: 26, 30, 34, or 38 and any one of SEQ ID NO: 28, 32, 36, or 40 binds. In some embodiments, the first binding domain binds to an epitope of the degrading protein on the target cell that does not include any of the amino acids from the epitope to which the antibody comprising any one of SEQ ID NO: 26, 30, 34, or 38 and any one of SEQ ID NO: 28, 32, 36, or 40 binds.

In some embodiments, the degrading protein is TROP2. In some embodiments, the first binding domain comprises a first binding domain variable heavy chain, and wherein the first binding domain variable heavy chain comprises at least 80%, sequence identity to any one of SEQ ID NO: 198, 202, 206, 210, or 214. In some embodiments, the first binding domain variable heavy chain comprises at least 90%, sequence identity to any one of SEQ ID NO: 198, 202, 206, 210, or 214. In some embodiments, the first binding domain variable heavy chain comprises any one of SEQ ID NO: 198, 202, 206, 210, or 214. In some embodiments, the first binding domain comprises a first binding domain variable light chain, and wherein the first binding domain variable light chain comprises at least 80% sequence identity to any one of SEQ ID NO: 200, 204, 208, 212, or 216. In some embodiments, the first binding domain variable light chain comprises at least 90% sequence identity to any one of SEQ ID NO: 200, 204, 208, 212, or 216. In some embodiments, the first binding domain variable light chain comprises any one of SEQ ID NO: 200, 204, 208, 212, or 216. In some embodiments, the first binding domain binds to an epitope of the degrading protein on the target cell, wherein the epitope comprises at least 80% sequence identity to an epitope to which an antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell, wherein the epitope comprises at least 90% sequence identity to the epitope to which the antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell that does not include any of the amino acids from the epitope to which the antibody comprising any one of SEQ ID NO: 198, 202, 206, 210, or 214 and any one of SEQ ID NO: 200, 204, 208, 212, or 216 binds.

In some embodiments, the degrading protein is selected from the group consisting of CD71, HER3, TNFRSF10B, ITGB6, PD-L1, EpCAM, TPBG, MST1R, EphA2, ADAM9, IGF1R, and EGFR. In some embodiments, the degrading protein is RNF43, RNF128, RNF130, or ZNRF3. In some embodiments, the first binding domain comprises a first binding domain variable heavy chain, and wherein the first binding domain variable heavy chain comprises at least 80%, sequence identity to any one of variable heavy chain sequences listed in Table 1 or Table 4. In some embodiments, the first binding domain variable heavy chain comprises at least 90%, sequence identity to any one of variable heavy chain sequences listed in Table 1 or Table 4. In some embodiments, the first binding domain variable heavy chain comprises any one of variable heavy chain sequences listed in Table 1 or Table 4. In some embodiments, the first binding domain comprises a first binding domain variable light chain, and wherein the first binding domain variable light chain comprises at least 80% sequence identity to any one of variable light chain sequences listed in Table 1 or Table 4. In some embodiments, the first binding domain variable light chain comprises at least 90% sequence identity to any one of variable light chain sequences listed in Table 1 or Table 4. In some embodiments, the first binding domain variable light chain comprises any one of variable light chain sequences listed in Table 1 or Table 4. In some embodiments, the first binding domain binds to an epitope of the degrading protein on the target cell, wherein the epitope comprises at least 80% sequence identity to an epitope to which an antibody comprising any one of variable heavy chain sequence or any one of variable light chain sequences listed in Table 1 or Table 4 binds. In some embodiments, the first binding domain binds to an epitope of the degrading protein on the target cell, wherein the epitope comprises at least 90% sequence identity to the epitope to which the antibody comprising any one of variable heavy chain sequence or any one of variable light chain sequences listed in Table 1 or Table 4 binds. In some embodiments, the first binding domain binds to an epitope of the degrading protein on the target cell that does not include any of the amino acids from the epitope to which the antibody comprising any one of variable heavy chain sequence or any one of variable light chain sequences listed in Table 1 or Table 4 binds.

In some embodiments, the second binding domain comprises a second binding domain variable heavy chain, and wherein the second binding domain variable heavy chain comprises at least 80%, sequence identity to any one of SEQ ID NO: 352, 356, 360, or 364. In some embodiments, the second binding domain variable heavy chain comprises at least 90%, sequence identity to any one of SEQ ID NO: 352, 356, 360, or 364. In some embodiments, the second binding domain variable heavy chain comprises any one of SEQ ID NO: 352, 356, 360, or 364. In some embodiments, the second binding domain comprises a second binding domain variable light chain, and wherein the second binding domain variable heavy chain comprises at least 80%, sequence identity to any one of SEQ ID NO: 354, 358, 362 or 366. In some embodiments, the second binding domain variable heavy chain comprises at least 90%, sequence identity to any one of SEQ ID NO: 354, 358, 362 or 366. In some embodiments, the second binding domain variable heavy chain comprises any one of SEQ ID NO: 354, 358, 362 or 366.

In some embodiments, the second binding domain binds to an epitope of the target protein on the target cell, wherein the epitope comprises at least 80% sequence identity to an epitope to which Amivantamab binds. In some embodiments, the second binding domain binds to an epitope of the target protein on the target cell, wherein the epitope comprises at least 90% sequence identity to the epitope to which Amivantamab binds. In some embodiments, the second binding domain binds to an epitope of the target protein on the target cell that does not include any of the amino acids from the epitope to which Amivantamab binds. In some embodiments, the second binding domain binds to an epitope of the target protein on the target cell, wherein the epitope comprises at least 80% sequence identity to an epitope to which Telisotuzumab binds. In some embodiments, the second binding domain binds to an epitope of the target protein on the target cell, wherein the epitope comprises at least 90% sequence identity to the epitope to which Telisotuzumab binds. In some embodiments, the second binding domain binds to an epitope of the target protein on the target cell that does not include any of the amino acids from the epitope to which Telisotuzumab binds. In some embodiments, the second binding domain binds to an epitope of the target protein on the target cell, wherein the epitope comprises at least 80% sequence identity to an epitope to which Onartuzumab binds. In some embodiments, the second binding domain binds to an epitope of the target protein on the target cell, wherein the epitope comprises at least 90% sequence identity to the epitope to which Onartuzumab binds. In some embodiments, the second binding domain binds to an epitope of the target protein on the target cell that does not include any of the amino acids from the epitope to which Onartuzumab binds. In some embodiments, the second binding domain binds to an epitope of the target protein on the target cell, wherein the epitope comprises at least 80% sequence identity to an epitope to which REGN5093s58 binds. In some embodiments, the second binding domain binds to an epitope of the target protein on the target cell, wherein the epitope comprises at least 90% sequence identity to the epitope to which REGN5093s58 binds. In some embodiments, the second binding domain binds to an epitope of the target protein on the target cell that does not include any of the amino acids from the epitope to which REGN5093s58 binds.

In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, a gastric adenocarcinoma cell, non-small cell lung cancer cell, head and neck cancer cell, and cancers harboring cMET mutations including exon 14 deletions. In some embodiments, the cancer cell is gastric adenocarcinoma cell. In some embodiments, the cancer cell is non-small cell lung cancer cell. In some embodiments, the cancer cell comprises a mutation in a gene selected from a cMET exon 14 skipping mutation or a cMET duplication mutation. In some embodiments, the mutation comprises a cMET exon 14 skipping mutation. In some embodiments, the cancer cell comprises a cMET duplication mutation.

In some embodiments, the method increases susceptibility of cancer cells to cancer therapeutic agents or radiation therapy. In some embodiments, the cancer therapeutic agent is a cytotoxic agent. In some embodiments, the method reduces proliferation of cancer cells. In some embodiments, the method induces death of cancer cells.

In one aspect, the present disclosure provides a binding agent comprising: (a) a first binding domain that specifically binds to a degrading protein or, wherein the degrading protein is CDH3, MUC1, CD276, or TROP2; and (b) a second binding domain that specifically binds to a target protein, wherein the target protein is cMET.

In some embodiments, the multispecific binding agent is a multispecific antibody, bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the binding agent is a multispecific antibody or a bispecific antibody. In some embodiments, the binding agent is a bispecific antibody.

In some embodiments, the degrading protein is CDH3. In some embodiments, the first binding domain comprises a first binding domain variable heavy chain, and wherein the variable heavy chain of the first binding domain comprises at least 80% sequence identity to any one of SEQ ID NO: 106, 110, 114, 118 or 122. In some embodiments, the first binding domain variable heavy chain comprises at least 90% sequence identity to any one of SEQ ID NO: 106, 110, 114, 118 or 122. In some embodiments, the first binding domain variable heavy chain comprises any one of SEQ ID NO: 106, 110, 114, 118 or 122. In some embodiments, the first binding domain comprises a first binding domain variable light chain and wherein the first binding domain variable light chain comprises at least 80% sequence identity to any one of SEQ ID NO: 108, 112, 116, 120, or 124. In some embodiments, the first binding domain variable light chain comprises at least 90% sequence identity to any one of SEQ ID NO: 108, 112, 116, 120, or 124. In some embodiments, the first binding domain variable light chain comprises any one of SEQ ID NO: 108, 112, 116, 120, or 124. In some embodiments, the first binding domain binds to an epitope of the degrading protein on the target cell, wherein the epitope comprises at least 80% sequence identity to an epitope to which an antibody comprising any one of SEQ ID NO: 106, 110, 114, 118 or 122 and any one of SEQ ID NO: 108, 112, 116, 120, or 124 binds. In some embodiments, the first binding domain binds to an epitope of the degrading protein on the target cell, wherein the epitope comprises at least 90% sequence identity to the epitope to which the antibody comprising any one of SEQ ID NO: 106, 110, 114, 118 or 122 and any one of SEQ ID NO: 108, 112, 116, 120, or 124 binds. In some embodiments, the first binding domain binds to an epitope of the degrading protein on the target cell that does not include any of the amino acids from the epitope to which an antibody comprising any one of SEQ ID NO: 106, 110, 114, 118 or 122 and any one of SEQ ID NO: 108, 112, 116, 120, or 124 binds.

In some embodiments, the degrading protein is MUC1. In some embodiments, the first binding domain comprises a first binding domain variable heavy chain, and wherein the first binding domain variable heavy chain comprises at least 80%, sequence identity to any one of SEQ ID NO: 6, 10, 14, 18, or 22. In some embodiments, the first binding domain variable heavy chain comprises at least 90% sequence identity to any one of SEQ ID NO: 6, 10, 14, 18, or 22. In some embodiments, the first binding domain variable heavy chain comprises any one of SEQ ID NO: 6, 10, 14, 18, or 22. In some embodiments, the first binding domain comprises a first binding domain variable light chain, and wherein the first binding domain variable light chain comprises at least 80% sequence identity to any one of SEQ ID NO: 8, 12, 16, 20, or 24. In some embodiments, the first binding domain variable light chain comprises at least 90% sequence identity to any one of SEQ ID NO: 8, 12, 16, 20, or 24. In some embodiments, the first binding domain variable light chain comprises any one of SEQ ID NO: 8, 12, 16, 20, or 24. In some embodiments, the first binding domain binds to an epitope of the degrading protein on the target cell, wherein the epitope comprises at least 80% sequence identity to an epitope to which an antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell, wherein the epitope comprises at least 90% sequence identity to the epitope to which the antibody comprising any one of SEQ ID NO: 6, 10, 14, 18, or 22 and any one of SEQ ID NO: 8, 12, 16, 20, or 24 binds. In some embodiments, the first binding domain binds to an epitope of the degrading protein on the target cell that does not include any of the amino acids from the epitope to which the antibody comprising any one of SEQ ID NO: 6, 10, 14, 18, or 22 and any one of SEQ ID NO: 8, 12, 16, 20, or 24 binds.

In some embodiments, the degrading protein is CD276. In some embodiments, the first binding domain comprises a first binding domain variable heavy chain, and wherein the first binding domain variable heavy chain comprises at least 80%, sequence identity to any one of SEQ ID NO: 26, 30, 34, or 38. In some embodiments, the first binding domain variable heavy chain comprises at least 90%, sequence identity to any one of SEQ ID NO: 26, 30, 34, or 38. In some embodiments, the first binding domain variable heavy chain comprises any one of SEQ ID NO: 26, 30, 34, or 38. In some embodiments, the first binding domain comprises a first binding domain variable light chain, and wherein the first binding domain variable light chain comprises at least 80% sequence identity to any one of SEQ ID NO: 28, 32, 36, or 40. In some embodiments, the first binding domain variable light chain comprises at least 90% sequence identity to any one of SEQ ID NO: 28, 32, 36, or 40. In some embodiments, the first binding domain variable light chain comprises any one of SEQ ID NO: 28, 32, 36, or 40. In some embodiments, the first binding domain binds to an epitope of the degrading protein on the target cell, wherein the epitope comprises at least 80% sequence identity to an epitope to which an antibody comprising any one of SEQ ID NO: 26, 30, 34, or 38 and any one of SEQ ID NO: 28, 32, 36, or 40 binds. In some embodiments, the first binding domain binds to an epitope e of the degrading protein on the target cell, wherein the epitope comprises at least 90% sequence identity to the epitope to which the antibody comprising any one of SEQ ID NO: 26, 30, 34, or 38 and any one of SEQ ID NO: 28, 32, 36, or 40 binds. In some embodiments, the first binding domain binds to an epitope of the degrading protein on the target cell that does not include any of the amino acids from the epitope to which the antibody comprising any one of SEQ ID NO: 26, 30, 34, or 38 and any one of SEQ ID NO: 28, 32, 36, or 40 binds. In some embodiments, the degrading protein is TROP2. In some embodiments, the first binding domain comprises a first binding domain variable heavy chain, and wherein the first binding domain variable heavy chain comprises at least 80%, sequence identity to any one of SEQ ID NO: 198, 202, 206, 210, or 214. In some embodiments, the first binding domain variable heavy chain comprises at least 90%, sequence identity to any one of SEQ ID NO: 198, 202, 206, 210, or 214. In some embodiments, the first binding domain variable heavy chain comprises any one of SEQ ID NO: 198, 202, 206, 210, or 214. In some embodiments, the first binding domain comprises a first binding domain variable light chain, and wherein the first binding domain variable light chain comprises at least 80% sequence identity to any one of SEQ ID NO: 200, 204, 208, 212, or 216. In some embodiments, the first binding domain variable light chain comprises at least 90% sequence identity to any one of SEQ ID NO: 200, 204, 208, 212, or 216. In some embodiments, the first binding domain variable light chain comprises any one of SEQ ID NO: 200, 204, 208, 212, or 216. In some embodiments, the first binding domain binds to an epitope of the degrading protein on the target cell, wherein the epitope comprises at least 80% sequence identity to an epitope to which an antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell, wherein the epitope comprises at least 90% sequence identity to the epitope to which the antibody comprising any one of SEQ ID NO: 198, 202, 206, 210, or 214 and any one of SEQ ID NO: 200, 204, 208, 212, or 216 binds. In some embodiments, the first binding domain binds to an epitope of the degrading protein on the target cell that does not include any of the amino acids from the epitope to which the antibody comprising any one of SEQ ID NO: 198, 202, 206, 210, or 214 and any one of SEQ ID NO: 200, 204, 208, 212, or 216 binds.

In some embodiments, the second binding domain comprises a second binding domain variable heavy chain, and wherein the second binding domain variable heavy chain comprises at least 80%, sequence identity to any one of SEQ ID NO: 352, 356, 360, or 364. In some embodiments, the second binding domain variable heavy chain comprises at least 90%, sequence identity to any one of SEQ ID NO: 352, 356, 360, or 364. In some embodiments, the second binding domain variable heavy chain comprises any one of SEQ ID NO: 352, 356, 360, or 364. In some embodiments, the second binding domain comprises a second binding domain variable light chain, and wherein the second binding domain variable heavy chain comprises at least 80%, sequence identity to any one of SEQ ID NO: 354, 358, 362 or 366. In some embodiments, the second binding domain variable heavy chain comprises at least 90%, sequence identity to any one of SEQ ID NO: 354, 358, 362 or 366. In some embodiments, the second binding domain variable heavy chain comprises any one of SEQ ID NO: 354, 358, 362 or 366.

In some embodiments, the second binding domain binds to an epitope of the target protein on the target cell, wherein the epitope comprises at least 80% sequence identity to an epitope to which Amivantamab binds. In some embodiments, the second binding domain binds to an epitope of the target protein on the target cell, wherein the epitope comprises at least 90% sequence identity to the epitope to which Amivantamab binds. In some embodiments, the second binding domain binds to an epitope of the target protein on the target cell that does not include any of the amino acids from the epitope to which Amivantamab binds. In some embodiments, the second binding domain binds to an epitope of the target protein on the target cell, wherein the epitope comprises at least 80% sequence identity to an epitope to which Telisotuzumab binds. In some embodiments, the second binding domain binds to an epitope of the target protein on the target cell, wherein the epitope comprises at least 90% sequence identity to the epitope to which Telisotuzumab binds. In some embodiments, the second binding domain binds to an epitope of the target protein on the target cell that does not include any of the amino acids from the epitope to which Telisotuzumab binds. In some embodiments, the second binding domain binds to an epitope of the target protein on the target cell, wherein the epitope comprises at least 80% sequence identity to an epitope to which Onartuzumab binds. In some embodiments, the second binding domain binds to an epitope of the target protein on the target cell, wherein the epitope comprises at least 90% sequence identity to the epitope to which Onartuzumab binds. In some embodiments, the second binding domain binds to an epitope of the target protein on the target cell that does not include any of the amino acids from the epitope to which Onartuzumab binds. In some embodiments, the second binding domain binds to an epitope of the target protein on the target cell, wherein the epitope comprises at least 80% sequence identity to an epitope to which REGN5093s58 binds. In some embodiments, the second binding domain binds to an epitope of the target protein on the target cell, wherein the epitope comprises at least 90% sequence identity to the epitope to which REGN5093s58 binds. In some embodiments, the second binding domain binds to an epitope of the target protein on the target cell that does not include any of the amino acids from the epitope to which REGN5093s58 binds.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “figure” and “FIG.” herein), of which:

FIG. 1 depicts a method of the present disclosure in which degradation of a target protein 112 (i.e., cMET) is mediated by binding of bifunctional binding agent 101.

FIGS. 2A-2D are charts depicting percentages of cMET cell surface removal in multiple cell types when treated with bispecific antibodies. FIG. 2A is a chart depicting the percentage of cMET cell surface removal in Hs746T cells treated with bispecific antibodies. FIG. 2B is a chart depicting the percentage of cMET cell surface removal in NCI-H1993 cells treated with bispecific antibodies. FIG. 2C is a chart depicting the percentage of cMET cell surface removal in NCI-H1975 cells treated with bispecific antibodies. FIG. 2D is a chart depicting the percentage of cMET cell surface removal in NCI-H596 cells treated with bispecific antibodies.

FIGS. 3A-3B are charts depicting percentage of cMET cell surface removal on target cells treated with various bispecific antibodies. FIG. 3A is a chart depicting percentage of cMET cell surface removal on NCI-H1975 target cells treated with various bispecific antibodies at 50 nM concentration. FIG. 3B is a chart depicting percentage of cMET cell surface removal on NCI-H1975 target cells treated with various bispecific antibodies at 50) nM concentration.

FIGS. 4A-4C are charts depicting cell surface removal of cMET. FIG. 4A is a chart depicting cell surface removal of cMET on NCI-H1975 target cells when treated with various bispecific antibodies where the antibody to the cMET target binds to different epitopes. FIG. 4B is a chart depicting cell surface removal of cMET on NCI-H596 target cells when treated with various bispecific antibodies where the antibody to the degrader binds to different epitopes. FIG. 4C is a chart depicting cell surface removal of cMET on Hs746T target cells when treated with various bispecific antibodies where the antibody to the degrader binds to different epitopes.

FIG. 5 is a chart depicting internalization of cMET on target cells when treated with various bispecific antibodies where the bispecific drove internalization above either single arm mAb targeting either the target or degrader.

FIGS. 6A-6C are charts depicting degradation of cMET on target cells when treated with various bispecific antibodies. FIG. 6A is a chart depicting internalization of cMET on NCI-H1975 target cells when treated with various bispecific antibodies. FIG. 6B is a chart depicting whole cell degradation of HS746t target cells when treated with various bispecific antibodies. FIG. 6C is a chart depicting whole cell degradation of NCI-H596 target cells when treated with various bispecific antibodies.

FIGS. 7A-7C depict the amount of cMET in target cells treated with various bispecific antibodies. FIG. 7A is an image of a Western blot depicting amount of cMET protein on target cells when treated with various bispecific antibodies. FIG. 7B is an image of a Western blot depicting amount of cMET protein on target cells when treated with various bispecific antibodies. FIG. 7C is a chart depicting whole cell degradation of cMET on target cells when treated with various bispecific antibodies.

FIGS. 8A-8E depict the amount of pERK and ERK in target cells treated with various bispecific antibodies. FIG. 8A is an image of a Western blot depicting amount of pERK and ERK protein in target cells when treated with various bispecific antibodies at different concentrations. FIG. 8B is an image of a Western blot depicting amount of pERK and ERK protein in target cells when treated with various bispecific antibodies at different concentrations. FIG. 8C is a chart depicting percentage of pERK to ERK in target cells when treated with various bispecific antibodies at different concentrations. FIG. 8D is a chart depicting percentage decrease in the amount of cMET and the ratio of pERK to ERK compared to PBS in target cells when treated with various bispecific antibodies at different concentrations. FIG. 8E is a chart depicting percentage decrease in the amount of cMET and the ratio of pERK to ERK in target cells when treated with various bispecific antibodies at different concentrations.

FIG. 9 is an image of a Western blot depicting amount of cMET protein on Hs746t target cells when treated with various bispecific antibodies.

FIGS. 10A-10D depict the amount of cMET dimerization on target cells treated with various bispecific antibodies. FIG. 10A is a graph showing cMET dimerization on target cells when treated with hepatocyte growth factor, the native ligand of cMET FIG. 10B is a graph showing cMET dimerization on target cells when treated with various bispecific antibodies comprising amivantamab cMET binding arms. FIG. 10C is a graph showing cMET dimerization on target cells when treated with various bispecific antibodies comprising onartuzumab cMET binding arms. FIG. 10D is a graph showing cMET dimerization on target cells when treated with various bispecific antibodies comprising telisotuzumab cMET binding arms.

### DETAILED DESCRIPTION

The present disclosure generally relates to binding agents, which bind to both a target protein and a degrading protein present on the surface of a target cell. In some embodiments, the present disclosure provides methods of degrading a target protein comprising contacting the target protein with a dual binding agent that simultaneously binds and a degrading protein, leading to cellular internalization of the target protein and subsequent degradation of the target protein.

### Definitions

Unless defined otherwise, all technical and scientific terms used herein have the same meaning as is commonly understood by one of skill in the art to which this disclosure belongs. All patents and publications referred to herein are incorporated by reference.

As used in the specification and claims, the singular form “a”, “an” and “the” includes plural references unless the context clearly dictates otherwise.

The terms “administer”, “administered”, “administers” and “administering” are defined as providing a composition to a subject via a route known in the art, including but not limited to intravenous, intraarterial, intrathecal, oral, parenteral, perineural, buccal, topical, transdermal, rectal, intramuscular, subcutaneous, intraosseous, transmucosal, intraperitoneal, or nerve root sheath routes of administration. In certain embodiments, oral routes of administering a composition can be used. The terms “administer”, “administered”, “administers” and “administering” a therapeutic protein should be understood to mean providing a therapeutic protein of the present disclosure or a prodrug of a therapeutic protein of the present disclosure to the individual in need.

The term “humanize” refers to replacement or substitution of certain amino acids in an antibody or nanobody derived from a non-human species, in particular in the framework regions and constant domains of the heavy and/or light chains, in order to avoid or minimize an immune response in humans.

As used herein, the terms “complementarity-determining region” or “CDR” within the context of antibodies or nanobodies refer to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have three CDR regions, each non-contiguous with the others (termed L1, L2, L3, H1, H2, H3) for the respective light (L) and heavy (H) chains. Nanobodies, in particular, generally comprise a single amino acid chain that can be considered to comprise four “framework sequences or regions” or FRs and three complementarity-determining regions” or CDRs. The nanobodies have three CDR regions, each non-contiguous with the others (termed CDR1, CDR2, CDR3). The delineation of the FR and CDR sequences is based on the IMGT unique numbering system for V-domains and V-like domains.

As used herein, the terms “nucleic acid molecule,” “polynucleotide.” “polynucleic acid.” and “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “nanobody” (Nb), as used herein, refers to the smallest antigen binding fragment or single variable domain (“VHH”) derived from naturally occurring heavy chain antibody and is known to the person skilled in the art. They are derived from heavy chain only antibodies, seen, for example, in camelid antibodies. The nanobodies hereof generally comprise a single amino acid chain that can be considered to comprise four “framework sequences” that make up the “scaffold” and three “complementarity-determining regions” or CDRs (as defined hereinbefore). It should be noted that the term “nanobody,” as used herein in its broadest sense, is not limited to a specific biological source or to a specific method of preparation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

As used herein, the terms “polypeptide,” “protein,” and “peptide” are used interchangeably herein, and refer to a polymeric form of amino acids of any length, which can include coded and non-coded amino acids, chemically or biochemically modified or derivatized amino acids, and polypeptides having modified peptide backbones.

The terms “subject,” “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a therapeutic protein described herein.

The term “specificity,” as used herein, refers to the ability of a protein binding domain, in particular, an immunoglobulin or an immunoglobulin fragment, such as a nanobody, to bind preferentially to one antigen versus a different antigen, and does not necessarily imply high affinity.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a therapeutic protein or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

A “degrading protein” or “degrader protein,” as that term is used herein, may encompasses a range of moieties including, but not limited to membrane associated internalizing protein, an internalizing receptor, a membrane associated degrading receptor, a degrading receptor, a surface moiety configured to internalize a binding agent, a surface moiety configured to degrade a binding agent, combinations thereof, or variants thereof.

An “internalizing protein,” as that term is used here, may encompass a range of moieties including, but not limited to membrane associated internalizing protein, an internalizing receptor, a surface moiety configured to internalize a binding agent, combinations thereof, or variants thereof.

Methods of Degrading cMET Proteins

Mesenchymal-epithelial transition factor (cMET) is a transmembrane protein that is a receptor for hepatocyte growth factor/scatter factor ligands. cMET is a receptor tyrosine kinase that is activated by binding of these specific ligands, including hepatocyte growth factor (HGF), and subsequent dimerization. Aberrant cMET function and/or expression is implicated in cancer, where it causes enhanced cell proliferation and drives tumor growth, invasion, metastasis, and angiogenesis.

Mutations that lead to cMET overexpression (known as upregulation or amplification) have been associated with a number of cancers, including colorectal cancer, non-small-cell lung carcinoma, breast cancer, renal cell carcinoma, and head and neck cancer. High expression of cMET is associated with poor prognosis in cancer patients and abnormal activation of cMET is associated with resistance to targeted therapies. Upregulation or overactivation of cMET can induce multiple signaling cascades that lead to motility, invasion, growth, and transformation. Therefore, the degradation of cMET in cancer is a promising treatment modality for cancer.

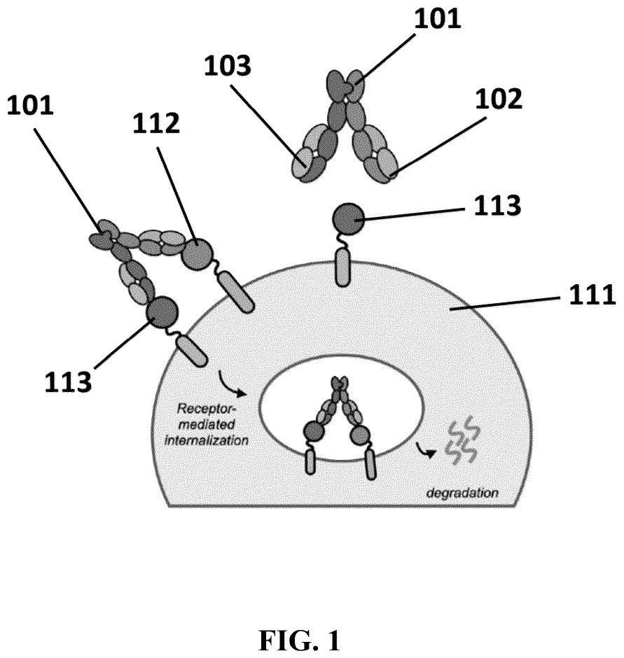

The present disclosure provides methods of degrading a cMET protein on a target cell as shown in FIG. 1. The method utilizes a binding agent 101 that binds specifically to both (i) an extracellular epitope on the cMET protein 112; and (2) an extracellular epitope on a membrane-associated internalizing protein 113 on a target cell 111, binding agent 101 comprises first binding domain 102 that selectively binds to the cMET protein 112 and second binding domain 103 that selectively binds to membrane-associated internalizing protein 113. Simultaneous binding of the binding agent 101 to the cMET protein 112 and the membrane-associated internalizing protein 113 leads to internalization of both the cMET protein 112 and the membrane-associated internalizing protein 113 into the target cell 111. Following internalization, the cMET protein 112 is degraded by the target cell 111 (e.g., via trafficking to the lysosome).

In some embodiments, the membrane-associated internalizing protein that is a cell-surface protein that internalizes upon binding of a binding agent (e.g., an antibody) to the protein. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1. STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, CD276, TPBG, MST1R, CDH3, EpCAM, TNFRSF10B, PD-L1, TROP2, EphA2, and CD71.

The present disclosure also provides methods of degrading an cMET protein on a target cell. The method utilizes a binding agent that binds specifically to both (1) an extracellular epitope on the cMET protein; and (2) an extracellular epitope on a membrane-associated degrading protein on a target cell. Binding agent comprises first binding domain that selectively binds to the cMET protein and second binding domain that selectively binds to membrane-associated degrading protein. Simultaneous binding of the binding agent to the cMET protein and the membrane-associated degrading protein leads to degradation of both the cMET protein and the membrane-associated degrading protein.

In some embodiments, the membrane-associated degrading protein is a cell-surface protein that degrades upon binding of a binding agent (e.g., an antibody) to the protein. In some embodiments, the membrane-associated degrading protein is RNF43, ZHFR3, RNF167, RNF128, and RNF130).

In one aspect, the present disclosure provides a method of degrading a cMET protein on a target cell, the method comprising:
contacting the cMET protein and a membrane-associated internalizing protein on the target cell with a binding agent, wherein the contacting of the cMET protein and the membrane-associated internalizing protein with the binding agent leads to internalization and degradation of the cMET protein; and
wherein the binding agent comprises: (a) a first binding domain that specifically binds to an extracellular epitope the membrane associated internalizing protein; and (b) a second binding domain that specifically binds to an extracellular epitope on the cMET protein:
wherein the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP. LYGE, CLDN18, LY6G6D, GPR56, CD276, TPBG, MST1R, CDH3, EpCAM, TNFRSF10B, PD-L1, TROP2, EphA2, and CD71.

In some embodiments, the binding agent comprises an antibody. In some embodiments, the binding agent comprises a multispecific antibody. In some embodiments, the binding agent comprises a bispecific antibody. In some embodiments, the binding agent comprises an IgG antibody. In some embodiments, the binding agent comprises a IgG antibody. In some embodiments, the binding agent comprises a knob and hole bispecific IgG. In some embodiments, the binding agent is not an antibody-drug conjugate (“ADC”). In some embodiments, the binding agent comprises a bispecific binding agent. In some embodiments, the binding agent comprises a bispecific antibody. In some embodiments, the binding agent comprises a bispecific diabody. In some embodiments, the binding agent comprises a bispecific Fab2. In some embodiments, the binding agent comprises a bispecific camelid antibody. In some embodiments, the binding agent comprises a bispecific peptibody scFv-Fc. In some embodiments, the binding agent comprises Fc-Fab. In some embodiments, the binding agent comprises a knob and hole bispecific Fc-Fab.

### Binding Agents

The binding agents of the present disclosure contain two binding domains: one specific for a degrading protein, and the other specific for a cMET protein. Binding agents of the disclosure include, without limitation, agents wherein the degrading protein binding domain and the cMET binding domain are each independently selected from an antibody (or half of an antibody), a nanobody, or a minibody, a Fab fragment, a single chain variable fragment (scFv), and a single domain antibody (sdAb), or a functional fragment thereof. These two binding domains can be the same type of molecule, or different. For example, binding agents of the disclosure include, without limitation, binding agents having an IgG that binds a degrading protein, and an scFv domain that binds cMET. The two binding domains of the binding agent can be connected through covalent bonds, non-covalent interactions, or a combination thereof.

The binding agent can generally take the form of a protein, glycoprotein, lipoprotein, phosphoprotein, and the like. Some binding agent of the disclosure take the form of antibodies or antibody derivatives. In some embodiments, the target protein binding domain is selected from the group consisting of a half antibody, a nanobody, or a minibody, a F(ab′) 2 fragment, a Fab fragment, a single chain variable fragment (scFv), and a single domain antibody (sdAb), or a functional fragment thereof. The two binding domains may together take the form of a bispecific antibody, a bispecific diabody, a bispecific camelid antibody or a bispecific peptibody, and the like. Antibody derivatives need not be derived from a specific wild type antibody. For example, one can employ known techniques such as phage display to generate and select for small proteins having a binding domain similar to an antibody complementarity-determining region (CDR). In some embodiments, the antigen-binding moiety includes an scFv. The binding domain can also be derived from a natural or synthetic ligand or receptor, whether soluble or membrane-bound, that specifically binds to the cMET protein.

Bispecific antibodies can be prepared by known methods. Embodiments of the disclosure include “knob-into-hole” bispecific antibodies, wherein the otherwise symmetric dimerization region of a bispecific binding agent is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies), and promotes the heterodimerization desired for a bispecific binding agent. See. e.g., Y. Xu et al., mAbs (2015) 7 (1): 231-42. In some embodiments, the bispecific binding agent has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V.

In some embodiments, the binding agent comprises an FcRn receptor recognition domain, to promote return of the binding agent to the extracellular space if the binding agent is internalized.

In another aspect, the present disclosure provides a binding agent comprising a antibody or antibody derivative, the binding agent comprising:
a) a first binding domain that specifically binds to an extracellular epitope of a cMET protein of a target cell; and
b) a second binding domain that specifically binds to an extracellular epitope of a membrane-associated internalizing protein on a target cell:
wherein the membrane associated internalizing protein is selected from CD205, CD166, SLC34A2, ITGB6, LRRC15, MUC16, SLC39A6, AXL, MMP14, CD40, CD228A, CD70, MUCSA, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71.

### Degrading Proteins

Methods and binding agents of the present disclosure may utilize membrane-associated degrading proteins to cause degradation of the cMET protein. The present disclosure may use the membrane-associated degrading proteins to cause ubiquitination upon binding of a binding agent to the membrane-associated degrading protein. By also binding to cMET at the first binding domain and binding to a membrane-associated degrading proteins using the second binding domain, the multifunctional binding agent can cause the cMET protein to be degraded with the membrane-associated degrading protein.

Membrane-associated degrading proteins for use in methods and bifunctional binding agents of the present disclosure can include a cell-surface protein that is degraded upon binding and/or internalization of a binding agent (e.g., an antibody) to the protein. Such membrane-associated degrading proteins can include cell-surface proteins that are targeted by antibody-drug conjugates, which can rely on degradation of the antibody-protein complex to ensure release of the conjugated drug. Examples of such membrane-associate degrading proteins useful for methods of the present disclosure can include, for example, TROP2. In some embodiments, the membrane-associated degrading protein is an E3 ligase. In some embodiments, the membrane-associated degrading protein is RNF43 (i.e., Ring Finger Protein 43).

Degrading proteins for use in methods and bifunctional binding agents of the present disclosure may include cell-surface protein that internalize upon binding of a binding agent (e.g., an antibody) to the protein. Such membrane-associate internalizing proteins include cell-surface proteins that are currently targeted by antibody-drug conjugates, which generally rely on internalization of the antibody-protein complex to ensure release of the conjugated drug. Examples of such membrane-associate internalizing proteins useful for methods of the present disclosure include, for example, CEACAM5 (i.e., CEA Cell Adhesion Molecule 5). CEACAM6 (i.e., CEA Cell Adhesion Molecule 6). HER3 (i.e., Receptor Tyrosine-Protein Kinase erbB-3), MUC1 (i.e., Mucin 1). CD205 (i.e., Lymphocyte Antigen 75). CD166 (i.e., Activated Leukocyte Cell Adhesion Molecule, also known as ALCAM), PRLR (i.e., Prolactin Receptor). SLC34A2 (i.e., Solute Carrier Family 34 Member 2). ITGB6 (i.e., Integrin Subunit Beta 6). LRRC15 (i.e., Leucine-Rich Repeat-Containing Protein 15). MUC16 (i.e., Mucin 16). SLC39A6 (i.e., Solute Carrier Family 39 Member 6). AXL (i.e., AXL Receptor Tyrosine Kinase). MMP14 (i.e., Matrix Metallopeptidase 14), CD40 (i.e., Cluster of Differentiation 40). CD228A (i.e., Melanotransferrin). CD70 (i.e., Cluster of Differentiation 70). MUC5A (i.e., Mucin 5A), CD44 (i.e., Homing Cell Adhesion Molecule). ITGB1 (i.e., Integrin beta-1). STn (e.g., Carbohydrate Antigen STn). KAAG1 (i.e., Kidney-Associated Antigen 1). DLK1 (i.e., Delta Likes Non-Canonical Notch Ligand 1). 5T4 (i.e., Oncofetal Antigen 5T4). SEZ6 (i.e., Seizure Related 6 Homolog), CD123 (i.e., Interleukin 3 Receptor). ADAM9 (i.e., A Disintegrin and A Metalloprotease 9). I-Ag7 (i.e., MHC Class II Molecule Ag7). ENPP3 (i.e., Ectonucleotide Pyrophosphatase/Phosphodiesterase 3), CD37 (i.e., Tetraspanin CD37). CD46 (i.e., CD46 Complement Regulatory Protein), CD56 (i.e., Neural Cell Adhesion Molecule), CD74 (i.e., Invariant Chain of MHC II). IGF1R (i.e., Insulin-like Growth Factor 1 Receptor). ROR1 (i.e., Receptor Tyrosine Kinase Like Orphan Receptor 1). CDH6 (i.e., Cadherin 6). ROR2 (i.e., Receptor Tyrosine Kinase Like Orphan Receptor 2). GPR20) (i.e., G Protein-Coupled Receptor 20). TM4SF1 (i.e., Transmembrane 4 L Size Family Member 1). B7-H4 (i.e., V-Set Domain Containing T Cell Activation Inhibitor 1). ALPP (i.e., Alkaline Phosphatase. Placental). LY6E (i.e., Lymphocyte Antigen 6 Family Member E). CLDN18 (i.e., Claudin 18). LY6G6D (i.e., Lymphocyte Antigen 6 Family Member G6D), GPR56 (i.e., Adhesion G Protein-Coupled Receptor G1). CDH3 (i.e. Chromodomain Helicase DNA Binding Protein 3). CD276 (i.e. Cluster of Differentiation 276). TROP2 (i.e. Trophoblast Cell-Surface Antigen 2). TNFRSF10B (i.e. TNF Receptor Superfamily Member 10). PD-L1 (i.e. Programmed death-ligand 1). EpCAM (i.e. Epithelial Cellular Adhesion Molecule). TPBG (i.e. Trophoblast Glycoprotein). EGFR (i.e. Epidermal Growth Factor Receptor). MST1R (i.e. Macrophage Stimulating 1 Receptor). EphA2 (i.e. Ephrin Receptor A2), and CD71 (Transferrin Receptor-1). It has been demonstrated that these proteins internalize into a cell upon binding of a binding agent (e.g., antibody) to an extracellular epitope of the protein.

In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, CD276, TPBG, MST1R, CDH3, EpCAM, TNFRSF10B, PD-L1, TROP2, EphA2, and CD71, In some embodiments, the membrane-associated internalizing protein is selected from CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71, In some embodiments, the membrane-associated internalizing protein is selected from CEACAM5, CEACAM6, HER3, MUC1, CD205, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71.

In some embodiments, the membrane-associated internalizing protein is selected from CEACAM5, CEACAM6, HER3, MUC1, CD205, CD166, PRLR, SLC34A2, ITGB6, LRRC15, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71.

In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, CD56, CD74, IGF1R, ROR1, CDH6, ROR2, GPR20, TM4SF1, B7-H4, ALPP, LY6E, CLDN18, LY6G6D, GPR56, and CD71. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, ROR2, GPR20, TM4SF1, B7-H4, ALPP, LY6E, CLDN18, LY6G6D, GPR56, and CD71. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ALPP, LY6E, CLDN18, LY6G6D, GPR56, and CD71.

In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, MMP14, CD40, CD228A, CD70, MUC5A, CD44, ITGB1, STn, KAAG1, DLK1, 5T4, SEZ6, CD123, ADAM9, I-Ag7, ENPP3, CD37, CD46, CD56, CD74, IGF1R, ROR1, CDH6, ROR2, GPR20, TM4SF1, and B7-H4.

In some embodiments, the membrane-associated internalizing protein is selected from CEACAM5. CEACAM6, HER3, MUC1. CD205. CD166, PRLR. SLC34A2. ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71, In some embodiments, the membrane-associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LYGE, CLDN18, LY6G6D, GPR56, and CD71.

In some embodiments, the membrane-associated internalizing protein is selected from SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LYGE, CLDN18, LY6G6D, GPR56, and CD71, In some embodiments, the membrane-associated internalizing protein is selected from CD205, CD166, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20), TM4SF1, B7-H4, ALPP, LY6E, CLDN18, LY6G6D, GPR56, and CD71.

In some embodiments, the membrane-associated internalizing protein is selected from SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71, In some embodiments, the membrane-associated internalizing protein is selected from CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from CD205, CD166, CD40, CD70, CD44, CD123, CD37, CD228, CD46, CD56, CD74, CDH6, and CD71, In some embodiments, the membrane-associated internalizing protein is selected from SLC34A2, ITGB6, LRRC15, MUC16, AXL/UFO, MMP14, MUC5A, ITGB1, STn, KAAG1, DLK1, 5T4, SEZ6, ADAM9, I-Ag7, ENPP3, IGF1R, ROR1, GPR20, TM4SF1, B7-H4, ALPP, LY6E, CLDN18, LY6G6D, GPR56, and CD71, In some embodiments, the membrane-associated internalizing protein is selected from CEACAM5, CEACAM6, HER3, MUC1, PRLR, SLC34A2, ITGB6, LRRC15, MUC16, SLC39A6, AXL, MMP14, MUC5A, ITGB1, STn, KAAG1, DLK1, 5T4, SEZ6, ADAM9, I-Ag7, ENPP3, IGF1R, ROR1, ROR2, GPR20, TM4SF1, B7-H4, ALPP, LY6E, CLDN18, LY6G6D, GPR56, and CD71.

In some embodiments, the membrane-associated internalizing protein is selected from SLC39A6, AXL, CD40, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71, In some embodiments, the membrane-associated internalizing protein is selected from SLC39A6, AXL, CD40, CD228, MUCSA, ITGB1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from SLC39A6. AXL. CD40. CD228. MUC5A. ITGB1. STn. KAAG1. DLK1. I-Ag7. ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from SLC39A6, AXL, CD40, CD228, MUC5A, ITGB1, STn, KAAG1, DLK1, 5T4, SEZ6, ADAM9, CD56, ROR1, GPR20, TM4SF1, B7-H4, ALPP, LY6E, CLDN18, LY6G6D, GPR56, and CD71, In some embodiments, the membrane-associated internalizing protein is selected from SLC39A6, AXL, CD40, CD228, MUC5A, ITGB1, STn, KAAG1, DLK1, 5T4, SEZ6, ADAM9, I-Ag7, ENPP3, CD46, TM4SF1, B7-H4, ALPP, LY6E, CLDN18, LY6G6D, GPR56, and CD71, In some embodiments, the membrane-associated internalizing protein is selected from SLC39A6, AXL/UFO, CD40, CD228, MUC5A, ITGB1, STn, KAAG1, DLK1, 5T4, SEZ6, ADAM9, I-Ag7, ENPP3, CD46, CD56, ROR1, GPR20, LY6E, CLDN18, LY6G6D, GPR56, and CD71, In some embodiments, the membrane-associated internalizing protein is selected from SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, and ALPP.

In some embodiments, the membrane-associated internalizing protein is selected from CD205, CD166, CD40, CD228, CD46, CD56, and CD71. In some embodiments, the membrane-associated internalizing protein is selected from SLC34A2, ITGB6, LRRC15, MUC16, SLC39A6, AXL, MUC5A, ITGB1, STn, KAAG1, DLK1, 5T4, SEZ6, ADAM9, I-Ag7, ENPP3, ROR1, GPR20, TM4SF1, B7-H4, ALPP, LY6E, CLDN18, LY6G6D, and GPR56.

In some embodiments, the membrane-associated internalizing protein is CDH3. In some embodiments, the membrane-associated internalizing protein is MUC1. In some embodiments, the membrane-associated internalizing protein is CD276. In some embodiments, the membrane-associated internalizing protein is TROP2. In some embodiments, the membrane-associated internalizing protein is CD71. In some embodiments, the membrane-associated internalizing protein is HER3. In some embodiments, the membrane-associated internalizing protein is TNFRSF10B. In some embodiments, the membrane-associated internalizing protein is ITGB6. In some embodiments, the membrane-associated internalizing protein is PD-L1. In some embodiments, the membrane-associated internalizing protein is EpCAM. In some embodiments, the membrane-associated internalizing protein is TPBG. In some embodiments, the membrane-associated internalizing protein is EGFR. In some embodiments, the membrane-associated internalizing protein is MST1R. In some embodiments, the membrane-associated internalizing protein is EphA2. In some embodiments, the membrane-associated internalizing protein is ADAM9. In some embodiments, the membrane-associated internalizing protein is IGF1R.

Degrading proteins for use in methods and bifunctional binding agents of the present disclosure may include cell-surface protein that degrade upon binding of a binding agent (e.g., an antibody) to the protein. Such membrane-associate degrading proteins include cell-surface proteins that are currently targeted by antibody-drug conjugates, which generally rely on degradation of the antibody-protein complex to ensure release of the conjugated drug. Examples of such membrane-associate degrading proteins useful for methods of the present disclosure include, for example, RNF43 (i.e. Ring Finger Protein 43), RNF128 (i.e. Ring Finger Protein 128), RNF130 (i.e. Ring Finger Protein 130), and ZNRF3 (i.e. Zinc and Ring Finger 3).

In some embodiments, the membrane-associated degrading protein is RNF43. In some embodiments, the membrane-associated degrading protein is RNF128. In some embodiments, the membrane-associated degrading protein is RNF130. In some embodiments, the membrane-associated degrading protein is ZNRF3.

### First Binding Region

In some embodiments, the first binding domain is derived from an antibody directed at a membrane associated internalizing protein. Such antibodies are known to those skilled in the art and can be incorporated into methods and binding agents of the present disclosure. For example, in some embodiments, the complementarity-determining regions (“CDR”) of known antibodies directed at the membrane associated internalizing protein of interest can be incorporated into binding agents and methods of the present disclosure using known techniques. Exemplary antibodies suitable for incorporation into the methods and binding agents of the present disclosure include those described below.

For example, antibodies targeting CEACAM5 are known in the art, including, for example the CC4 antibody disclosed in, for example, Zheng. Chaogu, et al., “A novel anti-CEACAM5 monoclonal antibody, CC4, suppresses colorectal tumor growth and enhances NK cells-mediated tumor immunity.” PloS one 6.6 (2011): e21146. Additional antibodies targeting CEACAM5 that are suitable for use in the present disclosure include, for example, the anti-CEACAM5 antibodies MN-14, MN-15, and MN-3, described, for example, in Blumenthal, Rosalyn D., Hans J. Hansen, and David M. Goldenberg. “Inhibition of adhesion, invasion, and metastasis by antibodies targeting CEACAM6 (NCA-90)) and CEACAM5 (Carcinoembryonic Antigen).” Cancer research 65.19 (2005): 8809-8817.

Antibodies targeting CEACAM6 are known in the art, including, for example, the anti-CEACAM6 antibodies sdAb, 2Ab, 4Ab described, for example in Wu. Shang-Jung, et al. “Migration and invasion of NSCLC suppressed by the downregulation of Src/focal adhesion kinase using single, double and tetra domain anti-CEACAM6 antibodies.” Translational oncology 14.7 (2021): 101057. Additional antibodies targeting CEACAM6 that are suitable for use in the present disclosure include, for example, the anti-CEACAM6 antibodies MN-3 and MN-15 as described, for example, in Blumenthal, Rosalyn D., Hans J. Hansen, and David M. Goldenberg. “Inhibition of adhesion, invasion, and metastasis by antibodies targeting CEACAM6 (NCA-90) and CEACAM5 (Carcinoembryonic Antigen).” Cancer research 65.19 (2005): 8809-8817.

Antibodies targeting HER3 (also known as ErbB-3) are known in the art, including, for example, the anti-HER3 antibody GSK2849330 described, for example, in Gan, Hui K., et al. “A phase I, first-in-human study of GSK2849330, an anti-HER3 monoclonal antibody, in HER3-expressing solid tumors.” The oncologist 26.10 (2021): e1844-e1853. Further anti-HER3 antibodies include, for example, Patritumab (U3-1287), which is described in, for example, Hashimoto, Yuuri, et al. “A Novel HER3-Targeting Antibody-Drug Conjugate, U3-1402, Exhibits Potent Therapeutic Efficacy through the Delivery of Cytotoxic Payload by Efficient Internalization Preclinical Evaluation of U3-1402, a HER3-Targeting ADC.” Clinical Cancer Research 25.23 (2019): 7151-7161.

Antibodies targeting MUC1 are known in the art, for example, including, the anti-MUC1 antibodies MY.1E12. KL6, 5E5, and TAB004 described in Bose, Mukulika, and Pinku Mukherjee. “Potential of anti-MUC1 antibodies as a targeted therapy for gastrointestinal cancers.” Vaccines 8.4 (2020): 659.

Antibodies targeting CD205 are known in the art, including, for example, the anti-CD205 antibody MEN1309/OBT076 described, for example, in Rieke, Damian T., and Ulrich Keller. “A CD205-directed antibody drug conjugate-lymphoma precision oncology or sophisticated chemotherapy?” Haematologica 105.11 (2020): 2504.

Antibodies targeting CD166 are known in the art, for example, the anti-CD166 antibody CX-2009 described in, for example, Boni, Valentina, et al. “Praluzatamab ravtansine, a CD166-targeting antibody-drug conjugate, in patients with advanced solid tumors: an open-label phase 1/2 trial of Praluzatamab ravtansine in patients with advanced tumors.” Clinical Cancer Research (2022).

Antibodies targeting PRLR are known in the art, for example, the anti-PRLR antibody ABBV-176 described in, for example, Anderson, Mark G., et al. “ABBV-176, a PRLR antibody drug conjugate with a potent DNA-damaging PBD cytotoxin and enhanced activity with PARP inhibition.” BMC cancer 21.1 (2021): 1-11.|). Additional antibodies targeting CEACAM6 that are suitable for use in the present disclosure include, for example, the anti-CEACAM6 antibody LFA102 described in Damiano, Jason S., et al. “Neutralization of Prolactin Receptor Function by Monoclonal Antibody LFA102, a Novel Potential Therapeutic for the Treatment of Breast Cancer Preclinical Development of Anti-PRLR Antibody LFA102.” Molecular cancer therapeutics 12.3 (2013): 295-305.

Antibodies targeting SCL34A2 are known in the art, for example the anti-NaPi2b antibody described in Lin, Kedan, et al. “Preclinical Development of an Anti-NaPi2b (SLC34A2) Antibody-Drug Conjugate as a Therapeutic for Non-Small Cell Lung and Ovarian CancersPreclinical Development of NaPi2b Antibody-Drug Conjugate.” Clinical Cancer Research 21.22 (2015): 5139-5150. Another antibody suitable for incorporation into binding agents of the present disclosure include the anti-SCL34A2 antibody MX35 described in Yin, Beatrice W T, et al. “Monoclonal antibody MX35 detects the membrane transporter NaPi2b (SLC34A2) in human carcinomas.” Cancer immunity 8.1 (2008).

Antibodies targeting ITGB6 are known in the art, including, for example the antibody SGN-B6A described in, for example, Patnaik, Amita, et al. “A phase I study of SGN-B6A, an antibody-drug conjugate targeting integrin beta-6, in patients with advanced solid tumors (SGN-B6A-001, Trial in Progress).” (2021). Another antibody suitable for incorporation into the present disclosure include the anti-ITGB6 antibodies TPS3144-TPS3144 described in Zheng. Xiaoxia, et al. “Silencing of ITGB6 inhibits the progression of cervical carcinoma via regulating JAK/STAT3 signaling pathway.” Annals of Translational Medicine 9.9 (2021).

Antibodies targeting LRRC15 are known in the art, including for example, the anti-LRCC15 antibody ABBV-085 described in, for example, Demetri, George D., et al. “First-in-Human Phase I Study of ABBV-085, an Antibody-Drug Conjugate Targeting LRRC15, in Sarcomas and Other Advanced Solid Tumors Phase I Study of ABBV-085, an LRRC15-Targeting ADC.” Clinical Cancer Research 27.13 (2021): 3556-3566; and Slemmons, Katherine K., et al. “LRRC15 antibody-drug conjugates show promise as osteosarcoma therapeutics in preclinical studies.” Pediatric blood & cancer 68.2 (2021): e28771]).

Antibodies targeting MUC16 are known in the art, including, for example, the anti-MUC16 antibody OC125 described in, for example, Rao, Thapi Dharma, et al. “Novel monoclonal antibodies against the proximal (carboxy-terminal) portions of MUC16.” Applied immunohistochemistry & molecular morphology: AIMM/official publication of the Society for Applied Immunohistochemistry 18.5 (2010): 462. Additional anti-MUC16 antibodies include, for example, those described in Aithal, Abhijit, et al. “MUC16 as a novel target for cancer therapy.” Expert opinion on therapeutic targets 22.8 (2018): 675-686; and Rao, Thapi Dharma, et al. “Antibodies against specific MUC16 glycosylation sites inhibit ovarian cancer growth.” ACS chemical biology 12.8 (2017): 2085-2096]).

Antibodies targeting SLC39A6 are known in the art, including, for example, the anti-SLC39A6 antibody described in Cui, Shen, et al., “SLC39A6: a potential target for diagnosis and therapy of esophageal carcinoma.” Journal of Translational Medicine 13 (2015): 321. Additional anti-SLC29A6 antibodies include, for example, those described in Sussman, Smith, et al. “SGN-LIVIA: A novel antibody-drug conjugate targeting LIV-1 for the treatment of metastatic breast cancer.” Mol Chancer Ther (2014) 13 (12): 2991-3000; and Wan and Wang “Role of SLC39A in the development and progression of liver cancer.” Oncology Letters 23.3. (2022): 77.

Antibodies targeting AXL are known in the art, including, for example, the AXL-specific antibody described in Vajkoczy, Knyazev, et al. “Dominant-negative inhibition of the Axl receptor tyrosine kinase suppresses brain tumor cell growth and invasion and prolongs survival.” Proceedings of the National Academy of Sciences 103.15 (2006): 5799-5804. An additional anti-AXL antibody includes, for example, the anti-AXL antibody 20G7-D9) described in Leconet, Chentouf, et al. “Therapeutic activity of anti-AXL antibody against triple-negative breast caser patient-derived xenografts and metastasis.” Clin Cancer Research 23.11 (2017): 2806-2816.

Antibodies targeting CD40 are known in the art, including, for example, are known in the art, including, for example, the anti-CD40) antibody described in Xu, Gao, et al. “Repulsive guidance molecule a blockade exerts the immunoregulatory function in DCs stimulated with ABP and LPS.” Human vaccines & immunotherapeutics 12.8 (2016): 2169-2180. Additional anti-CD40) antibodies include, for example, those described in Silvin, Chapuis, et al. “Elevated calprotectin and abnormal myeloid cell subsets discriminate severe from mild COVID-19.” Cell 182.6 (2020): 1401-1418; and in Ceglia, Zurawski, et al. “Anti-CD40) Antibody Fused to CD40 Ligand Is a Superagonist Platform for Adjuvant Intrinsic DC-Targeting Vaccines.” Frontiers in immunology 12:786144 (2021).

Antibodies targeting CD228 are known in the art, including, for example, the anti-MELTF antibody described in Sawaki, Kanda, et al. “Level of melanotransferrin in tissue and sera serves as a prognostic marker of gastric cancer.” Anticancer Research 39.11 (2019): 6125-6133. An additional anti-CD228 antibody includes, for example, that described in Singh, Eyford, et al. “Discovery of a Highly Conserved Peptide in the Iron Transporter Melanotransferrin that Traverses an Intact Blood Brain Barrier and Localizes in Neural Cells.” Frontiers in neuroscience 15:596976. (2021): 473.

Antibodies targeting MUC5A are known in the art, including, for example, the anti-MUC5A antibody MUC5: TR-3A described in Zuhdi Alimam, Piazza, et al. “Muc-5/5ac mucin messenger RNA and protein expression is a marker of goblet cell metaplasia in murine airways.” American journal of respiratory cell and molecular biology 22.3 (2000): 253-260. Additional anti-MUC5 antibodies include, for example, those described in Wang, Jin, et al. “Expression of survivin, MUC2 and MUC5 in colorectal cancer and their association with clinicopathological characteristics.” Oncology Letters 14.1 (2017): 1011-1016; and in Reis, David, et al. “Immunohistochemical study of MUC5AC expression in human gastric carcinomas using a novel monoclonal antibody.” International journal of cancer 74.1 (1997): 112-121.

Antibodies targeting ITGB1 are known in the art, including, for example, the anti-ITGB1 antibody described in Du, Yang, et al. “The circular RNA circSKA3 binds integrin β1 to induce invadopodium formation enhancing breast cancer invasion.” Molecular Therapy 28.5 (2020): 1287-1298. Additional anti-ITGB1 antibodies include, for example, those described in Kawahara, Niwa, et al. “Integrin β1 is an essential factor in vasculogenic mimicry of human cancer cells.” Cancer science 109.8 (2018): 2490-2496; and in Wang and Li. “Ropivacaine inhibits the proliferation and migration of colorectal cancer cells through ITGB1.” Bioengineered 12.1 (2021): 44-53.

Antibodies targeting STn are known in the art, including, for example, the anti-STn antibody described in Prendergast, da Silva, et al. “Novel anti-Sialyl-Tn monoclonal antibodies and antibody-drug conjugates demonstrate tumor specificity and anti-tumor activity.” mAbs 9, 4 (2017): 615-627. An additional anti-STn antibody includes, for example, that described in Eavarone, David A et al. “Humanized anti-Sialyl-Tn antibodies for the treatment of ovarian carcinoma.” PloS one 13, 7 (2018) e0201314.27.

Antibodies targeting KAAG1 are known in the art, including, for example, the anti-KAAG1 antibody anti-KAAG1 AB-3A described in US patent U.S. Pat. No. 9,393,302 B2.

Antibodies targeting DLK1 are known in the art, including, for example, the anti-DLK1 antibody anti-DLK1 SIP (EB3) described in Bujak, Ritz, et al. “A monoclonal antibody to human Dlk1 reveals differential expression in cancer and absence in healthy tissues.” Antibodies 4.2 (2015): 71-87. Additional anti-DLKL antibodies include, for example, those described in Takagi, Zhao, et al. “Delta-like 1 homolog (DLK1) as a possible therapeutic target and its application to radioimmunotherapy using 1251-labelled anti-DLK1 antibody in lung cancer models (HOT1801 and FIGHT004).” Lung Cancer 153 (2021): 134-142; and in Huang, Zhang, et al. “Up-regulation of DLK1 as an imprinted gene could contribute to human hepatocellular carcinoma.” Carcinogenesis 28.5 (2007): 1094-1103.

Antibodies targeting 5T4 are known in the art, including, for example, the anti-5T4 antibody anti-5T4 IgG1 described in Shapiro, Vaishampayan, et al. “First-in-human trial of an anti-5T4 antibody-monomethylauristatin conjugate, PF-06263507, in patients with advanced solid tumors.” Investigational New Drugs 35.3 (2017): 315-323. An additional anti-5T4 antibody includes, for example, that described in Owens, Sheard, et al. “Preclinical assessment of CAR T-cell therapy targeting the tumor antigen 5T4 in ovarian cancer.” Journal of Immunotherapy 41.3 (2018): 130-140.

Antibodies targeting SEZ6 are known in the art, including, for example, the anti-SEZ6 antibody described in Jiang, Chen, et al. “Correlation between human seizure-related gene 6 variants and idiopathic generalized epilepsy in a Southern Chinese Han population.” Neural Regeneration Research 7.2 (2012): 96-100. An additional anti-SEZ6 antibody includes, for example, that described in Kuhn, Koroniak, et al. “Secretome protein enrichment identifies physiological BACEI protease substrates in neurons.” The EMBO journal 31.14 (2012): 3157-3168.

Antibodies targeting ADAM9 are known in the art, including, for example, the anti-ADAM9 antibody described in Mazzocca, Coppari, et al. “A secreted form of ADAM9) promotes carcinoma invasion through tumor-stromal interactions.” Cancer research 65.11 (2005): 4728-4738. Additional anti-ADAM9 antibodies include, for example, those described in Zigrino, Mauch, et al. “Adam-9 expression and regulation in human skin melanoma and melanoma cell lines.” International journal of cancer 116.6 (2005): 853-859; and in Kim, Jeung, et al. “The Effect of Disintegrin-Metalloproteinase ADAM9 in Gastric Cancer Progression.” Molecular cancer therapeutics 13.12 (2014): 3074-3085.

Antibodies targeting I-Ag7 are known in the art, including, for example, the anti-I-Ag7 antibody described in Zhang, Crawford, et al. “Monoclonal antibody blocking the recognition of an insulin peptide-MHC complex modulates type 1 diabetes.” Proceedings of the National Academy of Sciences 111.7 (2014): 2656-2661. Additional antibodies targeting I-Ag7 include, for example, those described in Noorchashm, Hooman, et al. “I-Ag7-mediated antigen presentation by B lymphocytes is critical in overcoming a checkpoint in T cell tolerance to islet β cells of nonobese diabetic mice.” The Journal of Immunology 163.2 (1999): 743-750.; and in Gardiner, Richards, et al. “Conformation of MHC class II I-Ag7 is sensitive to the P° anchor amino acid in bound peptide.” International immunology 199 (2007): 1103-1113.

Antibodies targeting ENPP3 are known in the art, including, for example, the anti-ENPP3 antibody described in Boggavarapu, Lalitkumar, et al. “Compartmentalized gene expression profiling of receptive endometrium reveals progesterone regulated ENPP3 is differentially expressed and secreted in glycosylated form.” Scientific reports 6.1 (2016): 1-13. An additional anti-ENPP3 antibody includes, for example, that is described in Schiechl, Hermann, et al. “Basophils trigger fibroblast activation in cardiac allograft fibrosis development.” American Journal of Transplantation 16.9 (2016): 2574-2588.

Antibodies targeting CD46 are known in the art, including, for example, the anti-CD46 antibody anti-CD46 antibody YS5 described in Su, Liu, et al. “Targeting CD46 for both adenocarcinoma and neuroendocrine prostate cancer.” JCI insight 3.17 (2018) e121497. Additional anti-CD46 antibodies include, for example, those described in Carver-Ward, Hollanders, et al. “Progesterone does not potentiate the acrosome reaction in human spermatozoa: flow cytometric analysis using CD46 antibody.” Human reproduction 11.1 (1996): 121-126; and in Krey, Himmelreich, et al. “Function of bovine CD46 as a cellular receptor for bovine viral diarrhea virus is determined by complement control protein 1.” Journal of virology 80.8 (2006): 3912-3922.

Antibodies targeting CD56 are known in the art, including, for example, the anti-CD56 antibody described in Silvin, Chapuis, et al. “Elevated calprotectin and abnormal myeloid cell subsets discriminate severe from mild COVID-19.” Cell 182.6 (2020): 1401-1418. Additional anti-CD46 antibodies include, for example, those described in Zhan, Guo, et al. “Glioma stem-like cells evade interferon suppression through MBD3/NuRD complex-mediated STAT1 downregulation.” The Journal of experimental medicine 217, 5 (2020): e20191340; and in Feng, Wang et al. “Differential killing of CD56-expressing cells by drug-conjugated human antibodies targeting membrane-distal and membrane-proximal non-overlapping epitopes.” mAbs 8.4 (2016): 799-810.

Antibodies targeting ROR1 are known in the art, including, for example, the anti-ROR1 antibody anti-ROR1 4A5 described in Balakrishnan, Goodpaster, et al. “Analysis of ROR1 Protein Expression in Human Cancer and Normal Tissues.” Clinical Cancer Research 23.12 (2017): 3061-3071. Additional anti-ROR1 antibodies include, for example, those described in Baskar, Wiestner et al. “Targeting malignant B cells with an immunotoxin against ROR1,” mAbs. 4.3 (2012) 349-361; and in Zhang. Chen et al. “ROR1 is expressed in human breast cancer and associated with enhanced tumor-cell growth.” PloS one 7, 3 (2012): e31127.

Antibodies targeting GPR20 are known in the art, including, for example, the anti-GPR20 antibody described in Wheway, Schmidts, et al. “An siRNA-based functional genomics screen for the identification of regulators of ciliogenesis and ciliopathy genes.” Nature cell biology 17, 8 (2015): 1074-1087. An additional anti-GPR20) antibody includes, for example, that described in lida, Ahmed, et al. “Identification and Therapeutic Targeting of GPR20, Selectively Expressed in Gastrointestinal Stromal Tumors, with DS-6157a, a First-in-Class Antibody-Drug Conjugate.” Cancer Discovery 11.6 (2021): 1508-1523.

Antibodies targeting TM4SF1 are known in the art, including, for example, the anti-TM4SF1 antibody described in Zacharias, Frank, et al. “Regeneration of the lung alveolus by an evolutionarily conserved epithelial progenitor.” Nature 555,7695 (2018): 251-255. Additional antibodies targeting TM4SF1 include, for example, the anti-TM4SF1 antibody 8G4 described in Lin, Merley, et al. “TM4SF1: a new vascular therapeutic target in cancer.” Angiogenesis 17, 4 (2014): 897-907; and the anti-TM4SF1 antibody described in Wang, Sun, et al. “B7-H3 suppresses doxorubicin-induced senescence-like growth arrest in colorectal cancer through the AKT/TM4SF1/SIRT1 pathway” Cell death & disease 12, 5 (2021): 453.

Antibodies targeting B7-H4 are known in the art, including, for example, the anti-B7-H4 antibody described in Podojil, Glaser, et al. “Antibody targeting of B7-H4 enhances the immune response in urothelial carcinoma.” Oncoimmunology 9, 1 (2020): 1744897. Additional antibodies targeting B7-H4 include, for example, those described in Miao and Sun. “Development of a novel anti-B7-H4 antibody enhances anti-tumor immune response of human T cells.” Biomedicine & pharmacotherapy 141 (2021): 111913; and in Dangaj, Lanitis, et al. “Novel Recombinant Human B7-H4 Antibodies Overcome Tumoral Immune Escape to Potentiate T-Cell Antitumor Responses Overcoming B7-H4-Mediated T-Cell Inhibition.” Cancer research 73.15 (2013): 4820-4829.

Antibodies targeting ALPP are known in the art, including, for example, the anti-ALPP antibody anti-ALPP SP15 described in Zwolanek, Satue, et al. “Tracking mesenchymal stem cell contributions to regeneration in an immunocompetent cartilage regeneration model.” JCI insight 2.20 (2017) e87322. Additional antibodies targeting ALPP include, for example, those described in Chen, Chen, et al. “Placental alkaline phosphatase promotes Zika virus replication by stabilizing viral proteins through BIP.” MBio 11.5 (2020): e01716-20; and in Odörfer, Egerbacher, et al. “Hematopoietic bone marrow cells participate in endothelial, but not epithelial or mesenchymal cell renewal in adult rats.” Journal of cellular and molecular medicine 15.10 (2011): 2232-2244.

Antibodies targeting LY6E are known in the art, including, for example, the anti-LY6E antibody described in Mar, Rinkenberger, et al. “LY6E mediates an evolutionarily conserved enhancement of virus infection by targeting a late entry step.” Nature communications 9.1 (2018): 1-14. Additional antibodies targeting LY6E include, for example, the anti-LY6E antibody anti-LY6E MTS35 described in Langford, Outhwaite, et al. “Deletion of the Syncytin A receptor Ly6e impairs syncytiotrophoblast fusion and placental morphogenesis causing embryonic lethality in mice.” Scientific reports 8, 1 (2018): 3961; and the anti-LY6E antibody anti-LY6E 9B12 described in Dela Cruz Chuh, Josefa, et al. “Preclinical optimization of Ly6E-targeted ADCs for increased durability and efficacy of anti-tumor response.” MAbs 13.1 (2021).

Antibodies targeting CLDN18 are known in the art, including, for example, the anti-CLDN18 antibody described in Türeci, Mitnacht-Kraus, et al. “Characterization of zolbetuximab in pancreatic cancer models.” Oncoimmunology 8.1 (2019): e1523096. An additional anti-CLDN18 antibody includes, for example, that described in Matsusaka, Ushiku, et al. “Coupling CDH17 and CLDN18 markers for comprehensive membrane-targeted detection of human gastric cancer.” Oncotarget 7, 39 (2016): 64168-64181.

Antibodies targeting LY6G6D are known in the art, including, for example, the anti-LY6G6D antibody described in Sewda, Coppola, et al. “Cell-surface markers for colon adenoma and adenocarcinoma.” Oncotarget 7, 14 (2016): 17773-89. Additional anti-LY6G6D antibodies include, for example, the anti-LY6G6D antibody anti-LY6G6D clone 10C1 described in Corrales, Hipp, et al. “LY6G6D is a selectively expressed colorectal cancer antigen that can be used for targeting a therapeutic T-cell response by a T-cell engager.” Frontiers in immunology 13 (2022): 1008764; and the anti-LY6G6D antibody described in Wang, Sun, et al. “Novel Anti-LY6G6D/CD3 T Cell-Dependent Bispecific Antibody for the Treatment of Colorectal Cancer.” Molecular Cancer Therapeutics 21:6 (2022): 974-985.

Antibodies targeting GPR56 are known in the art, including, for example, the anti-GPR56 antibody anti-GPR56 10C7 described in Chatterjee, Zhang, et al. “Anti-GPR56 monoclonal antibody potentiates GPR56-mediated Src-Fak signaling to modulate cell adhesion.” Journal of Biological Chemistry 296 (2021) 100261. Additional anti-GPR56 antibodies include, for example, those described in Iguchi, Sakata, et al. “Orphan G protein-coupled receptor GPR56 regulates neural progenitor cell migration via a Gal2/13 and Rho pathway.” Journal of Biological Chemistry 283.21 (2008): 14469-14478; and in Chen, Yang, et al. “GPR56 is essential for testis development and male fertility in mice.” Developmental Dynamics 239.12 (2010): 3358-3367.

Antibodies targeting MMP14 are known in the art, including, for example, the anti-MMP14 antibody described in Zhang, Zhang, et al., “MMP-14 aggravates onset of severe preeclampsia by mediating soluble endoglin release.” European review for medical and pharmacological sciences 22, 5 (2018). 1209-1215. Additional anti-MMP14 antibodies includes, for example, those described in Fischer and Riedl “Inhibitory antibodies designed for matrix metalloproteinase modulation.” Molecules 24.12 (2019): 2265.

Antibodies targeting cMET are known in the art, including, for example, the anti-cMET antibody described in for example, Lee, D., et al., “Development of antibody-based c-Met inhibitors for targeted cancer therapy.” ImmunoTargets and therapy 4 (2015): 35-44. Additional anti-cMET antibodies include those described in, for example, Liu, L., et al., “LY2875358, a neutralizing and internalizing anti-MET bivalent antibody, inhibits HGF-dependent and HGF-independent MET activation and tumor growth.” Clinical Cancer Research 20.23 (2014): 6059-6070; and in Jin, H., “MetMAb, the one-armed 5D5 anti-c-Met antibody, inhibits orthotopic pancreatic tumor growth and improves survival.” Cancer Research 68, 11 (2008): 4360-8:

Antibodies targeting CD70 are known in the art, including, for example, the anti-CD70 antibody described in McEarchern, Oflazoglu, et al. “Engineered anti-CD70 antibody with multiple effector functions exhibits in vitro and in vivo antitumor activities.” Blood 109.3 (2007): 1185-1192. Additional anti-CD70 antibodies includes, for example, those described in Israel, Gulley, et al. “Anti-CD70 antibodies: a potential treatment for EBV+CD70-expressing lymphomas.” Molecular cancer therapeutics 4.12 (2005): 2037-2044.

Antibodies targeting CD44 are known in the art, including, for example, the anti-CD44 antibody described in Wang, Su, et al. “CD44 antibody-targeted liposomal nanoparticles for molecular imaging and therapy of hepatocellular carcinoma.” Biomaterials 33.20 (2012): 5107-5114. Additional anti-CD44 antibodies includes, for example, those described in Kania, Kehat-Stadler, and Kupfer. “CD44 antibodies inhibit osteoclast formation.” Journal of Bone and Mineral Research 12.8 (1997): 1155-1164; and in Kodama, Toda, et al. “Anti-CD44 antibody treatment lowers hyperglycemia and improves insulin resistance, adipose inflammation, and hepatic steatosis in diet-induced obese mice.” Diabetes 64.3 (2015): 867-875.

Antibodies targeting CD123 are known in the art, including, for example, the anti-CD123 antibody described in Lee, Yee, et al. “Efficacy of an Fc-modified anti-CD123 antibody (CSL362) combined with chemotherapy in xenograft models of acute myelogenous leukemia in immunodeficient mice.” haematologica 100.7 (2015): 914. An additional anti-CD123 antibody includes, for example, that described in Kovtun, Jones, et al. “A CD123-targeting antibody-drug conjugate, IMGN632, designed to eradicate AML while sparing normal bone marrow cells.” Blood advances 2.8 (2018): 848-858.

Antibodies targeting CD37 are known in the art, including, for example, the anti-CD37 antibodies described in Oostindie, van der Horst, et al. “DuoHexaBody-CD37®, a novel biparatopic CD37 antibody with enhanced Fc-mediated hexamerization as a potential therapy for B-cell malignancies.” Blood cancer journal 10.3 (2020): 1-13. Additional anti-CD37 antibodies includes, for example, those described in Deckert, Park, et al. “A novel anti-CD37 antibody-drug conjugate with multiple anti-tumor mechanisms for the treatment of B-cell malignancies.” Blood. The Journal of the American Society of Hematology 122.20 (2013): 3500-3510.

Antibodies targeting CD74 are known in the art, including, for example, the anti-CD74 antibody anti-CD74 LLI described in Stein, Mattes, et al. “CD74: a new candidate target for the immunotherapy of B-cell neoplasms.” Clinical Cancer Research 13.18 (2007): 5556s-5563s. An additional anti-CD74 antibody includes, for example, the anti-CD74 antibody anti-CD74 LN2 described in Burton, Ely, et al. “CD74 is expressed by multiple myeloma and is a promising target for therapy.” Clinical Cancer Research 10.19 (2004): 6606-6611.

Antibodies targeting IGF1R are known in the art, including, for example, the anti-IGF1R antibody described in Gong, Yao, et al. “High expression levels of total IGF-1R and sensitivity of NSCLC cells in vitro to an anti-IGF-1R antibody (R1507).” PloS one 4.10 (2009): e7273. An additional anti-IGF1R antibody includes, for example, the anti-IGF1R antibody described in Cao, Roth, et al. “Insulin-like growth factor I receptor and response to anti-IGF1R antibody therapy in osteosarcoma.” PloS one 9.8 (2014): e106249.

Antibodies targeting CDH6 are known in the art, including, for example, the anti-CDH6 antibody described in Bartolomé, Robles, et al. “CDH6-activated αIIbβ3 crosstalks with α2β1 to trigger cellular adhesion and invasion in metastatic ovarian and renal cancers.” Molecular Oncology 15.7 (2021): 1849-1865. An additional anti-CDH6 antibody includes, for example, the anti-CDH6 antibody described in Ji, Xu, et al. “miR-223-3p inhibits human osteosarcoma metastasis and progression by directly targeting CDH6.” Molecular Therapy 26.5 (2018): 1299-1312.

Antibodies targeting ROR2 are known in the art, including, for example, the anti-ROR2 antibody described in Morioka. Tanikawa, et al. “Orphan receptor tyrosine kinase ROR2 as a potential therapeutic target for osteosarcoma.” Cancer science 100.7 (2009): 1227-1233. Additional anti-ROR2 antibodies includes, for example, those described in Goydel, Weber, et al. “Affinity maturation, humanization, and co-crystallization of a rabbit anti-human ROR2 monoclonal antibody for therapeutic applications.” Journal of Biological Chemistry 295.18 (2020): 5995-6006.

Antibodies targeting CD71 are known in the art, including, for example, the anti-CD71 antibody anti-Tfr1 H68.4 described in Byrne, et al. “Ferristatin II promotes degradation of transferrin receptor-1 in vitro and in vivo.” PLOS One 8.7 (2013): e70199. Additional anti-CD71 antibodies include, for example, those described in Hamamichi, et al. “Novel method for screening functional antibody with comprehensive analysis of its immunoliposome.” Scientific reports 11.1 (2021): 1-13; and in Kono, et al. “Morphological definition of CD71 positive reticulocytes by various staining techniques and electron microscopy compared to reticulocytes detected by an automated hematology analyzer.” Clinica Chimica Acta 404.2 (2009): 105-110.

The antibodies described in the foregoing are merely exemplary and are not meant to limit in any way the scope of the present disclosure. Additional binding agents, including antibodies, suitable for incorporation into the methods and binding agents of the present disclosure will be evident to one of ordinary skill.

Although aspects of the present disclosure have been described with reference to the disclosed embodiments, one skilled in the art will readily appreciate that the specific examples disclosed are only illustrative of these aspects and in no way limit the present disclosure. Various modifications can be made without departing from the spirit of the present disclosure.

In some embodiments, the first binding domain comprises a heavy chain (HC) sequence, a variable heavy (VH) sequence, a light chain (LC) sequence, and a variable light (VL) sequence. In some embodiments, the first binding domain comprises an HC sequence and a VH sequence. The first binding domain comprising an HC sequence, a VH sequence, an LC sequence, and a VL sequence may comprises one or more sequences listed in Table 1 or 4. The first binding domain comprising an HC sequence, a VH sequence, an LC sequence, and a VL sequence may comprise at least 70% sequence identity to one or more sequences listed in Table 1 or 4. In some cases, the first binding domain comprising an HC sequence, a VH sequence, an LC sequence, and a VL sequence comprises at least 75% sequence identity to one or more sequences listed in Table 1 or 4. In some cases, the first binding domain comprising an HC sequence, a VH sequence, an LC sequence, and a VL sequence comprises at least 80% sequence identity to one or more sequences listed in Table 1 or 4. In some cases, the first binding domain comprising an HC sequence, a VH sequence, an LC sequence, and a VL sequence comprises at least 85% sequence identity to one or more sequences listed in Table 1 or 4. In some cases, the first binding domain comprising an HC sequence, a VH sequence, an LC sequence, and a VL sequence comprises at least 90% sequence identity to one or more sequences listed in Table 1 or 4. In some cases, the first binding domain comprising an HC sequence, a VH sequence, an LC sequence, and a VL sequence comprises at least 91% sequence identity to one or more sequences listed in Table 1 or 4. In some cases, the first binding domain comprising an HC sequence, a VH sequence, an LC sequence, and a VL sequence comprises at least 92% sequence identity to one or more sequences listed in Table 1 or 4. In some cases, the first binding domain comprising an HC sequence, a VH sequence, an LC sequence, and a VL sequence comprises at least 93% sequence identity to one or more sequences listed in Table 1 or 4. In some cases, the first binding domain comprising an HC sequence, a VH sequence, an LC sequence, and a VL sequence comprises at least 94% sequence identity to one or more sequences listed in Table 1 or 4. In some cases, the first binding domain comprising an HC sequence, a VH sequence, an LC sequence, and a VL sequence comprises at least 95% sequence identity to one or more sequences listed in Table 1 or 4. In some cases, the first binding domain comprising an HC sequence, a VH sequence, an LC sequence, and a VL sequence comprises at least 96% sequence identity to one or more sequences listed in Table 1 or 4. In some cases, the first binding domain comprising an HC sequence, a VH sequence, an LC sequence, and a VL sequence comprises at least 97% sequence identity to one or more sequences listed in Table 1 or 4. In some cases, the first binding domain comprising an HC sequence, a VH sequence, an LC sequence, and a VL sequence comprises at least 98% sequence identity to one or more sequences listed in Table 1 or 4. In some cases, the first binding domain comprising an HC sequence, a VH sequence, an LC sequence, and a VL sequence comprises at least 99% sequence identity to one or more sequences listed in Table 1 or 4. In some cases, the first binding domain comprising an HC sequence, a VH sequence, an LC sequence, and a VL sequence comprises at least 99.5% sequence identity to one or more sequences listed in Table 1 or 4. In some cases, the first binding domain comprising an HC sequence, a VH sequence, an LC sequence, and a VL sequence comprises at least 99.9% sequence identity to one or more sequences listed in Table 1 or 4.

In some embodiments, the first binding domain comprises an antibody comprising a heavy chain (HC) sequence, a variable heavy (VH) sequence, a light chain (LC) sequence, and a variable light (VL) sequence. In some embodiments, the first binding domain comprises an antibody comprising an HC sequence and a VH sequence. The first binding domain comprising an antibody comprising an HC sequence, a VH sequence, an LC sequence, and a VL sequence may comprise one or more sequences listed in Table 1 or 4. The first binding domain comprising an antibody comprising an HC sequence, a VH sequence, an LC sequence, and a VL sequence may comprise at least 70% sequence identity to one or more sequences listed in Table 1 or 4. In some cases, the first binding domain comprising an antibody comprising an HC sequence, a VH sequence, an LC sequence, and a VL sequence comprises at least 75% sequence identity to one or more sequences listed in Table 1 or 4. In some cases, the first binding domain comprising an antibody comprising an HC sequence, a VH sequence, an LC sequence, and a VL sequence comprises at least 80% sequence identity to one or more sequences listed in Table 1 or 4. In some cases, the first binding domain comprising an antibody comprising an HC sequence, a VH sequence, an LC sequence, and a VL sequence comprises at least 85% sequence identity to one or more sequences listed in Table 1 or 4. In some cases, the first binding domain comprising an antibody comprising an HC sequence, a VH sequence, an LC sequence, and a VL sequence comprises at least 90% sequence identity to one or more sequences listed in Table 1 or 4. In some cases, the first binding domain comprising an antibody comprising an HC sequence, a VH sequence, an LC sequence, and a VL sequence comprises at least 91% sequence identity to one or more sequences listed in Table 1 or 4. In some cases, the first binding domain comprising an antibody comprising an HC sequence, a VH sequence, an LC sequence, and a VL sequence comprises at least 92% sequence identity to one or more sequences listed in Table 1 or 4. In some cases, the first binding domain comprising an antibody comprising an HC sequence, a VH sequence, an LC sequence, and a VL sequence comprises at least 93% sequence identity to one or more sequences listed in Table 1 or 4. In some cases, the first binding domain comprising an antibody comprising an HC sequence, a VH sequence, an LC sequence, and a VL sequence comprises at least 94% sequence identity to one or more sequences listed in Table 1 or 4. In some cases, the first binding domain comprising an antibody comprising an HC sequence, a VH sequence, an LC sequence, and a VL sequence comprises at least 95% sequence identity to one or more sequences listed in Table 1 or 4. In some cases, the first binding domain comprising an antibody comprising an HC sequence, a VH sequence, an LC sequence, and a VL sequence comprises at least 96% sequence identity to one or more sequences listed in Table 1 or 4. In some cases, the first binding domain comprising an antibody comprising an HC sequence, a VH sequence, an LC sequence, and a VL sequence comprises at least 97% sequence identity to one or more sequences listed in Table 1 or 4. In some cases, the first binding domain comprising an antibody comprising an HC sequence, a VH sequence, an LC sequence, and a VL sequence comprises at least 98% sequence identity to one or more sequences listed in Table 1 or 4. In some cases, the first binding domain comprising an antibody comprising an HC sequence, a VH sequence, an LC sequence, and a VL sequence comprises at least 99% sequence identity to one or more sequences listed in Table 1 or 4. In some cases, the first binding domain comprising an antibody comprising an HC sequence, a VH sequence, an LC sequence, and a VL sequence comprises at least 99.5% sequence identity to one or more sequences listed in Table 1 or 4. In some cases, the first binding domain comprising an antibody comprising an HC sequence, a VH sequence, an LC sequence, and a VL sequence comprises at least 99.9% sequence identity to one or more sequences listed in Table 1 or 4.

In some embodiments, the first binding domain comprises sequences listed Table 1. In some embodiments, the first binding domain comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1.

In some cases, the first binding domain may bind the same epitope as any one of the antibodies listed in Table 1. The first binding domain may bind to an epitope that comprises about 70% sequence identity to the epitope to which any one of the antibodies listed in Table 1 binds. The first binding domain may bind to an epitope that comprises about 75% sequence identity to the epitope to which any one of the antibodies listed in Table 1 binds. The first binding domain may bind to an epitope that comprises about 80% sequence identity to the epitope to which any one of the antibodies listed in Table 1 binds. The first binding domain may bind to an epitope that comprises about 85% sequence identity to the epitope to which any one of the antibodies listed in Table 1 binds. The first binding domain may bind to an epitope that comprises about 90% sequence identity to the epitope to which any one of the antibodies listed in Table 1 binds. The first binding domain may bind to an epitope that comprises about 95% sequence identity to the epitope to which any one of the antibodies listed in Table 1 binds. The first binding domain may bind to an epitope that comprises about 99% sequence identity to the epitope to which any one of the antibodies listed in Table 1 binds.

The first binding domain may bind to an epitope that comprises about 70% sequence identity to the epitope to which any one of the antibodies listed in Table 1 binds with a similar affinity as any one of the antibodies listed in Table 1. The first binding domain may bind to an epitope that comprises about 75% sequence identity to the epitope to which any one of the antibodies listed in Table 1 binds with a similar affinity as any one of the antibodies listed in Table 1. The first binding domain may bind to an epitope that comprises about 80% sequence identity to the epitope to which any one of the antibodies listed in Table 1 binds with a similar affinity as any one of the antibodies listed in Table 1. The first binding domain may bind to an epitope that comprises about 85% sequence identity to the epitope to which any one of the antibodies listed in Table 1 binds with a similar affinity as any one of the antibodies listed in Table 1. The first binding domain may bind to an epitope that comprises about 90% sequence identity to the epitope to which any one of the antibodies listed in Table 1 binds with a similar affinity as any one of the antibodies listed in Table 1. The first binding domain may bind to an epitope that comprises about 95% sequence identity to the epitope to which any one of the antibodies listed in Table 1 binds with a similar affinity as any one of the antibodies listed in Table 1. The first binding domain may bind to an epitope that comprises about 99% sequence identity to the epitope to which any one of the antibodies listed in Table 1 binds with a similar affinity as any one of the antibodies listed in Table 1.

In some embodiments, the first binding domain may bind the same epitope as any one of the antibodies listed in Table 1 binds with a different affinity as compared to any one of the antibodies listed in Table 1. The first binding domain may bind to an epitope that comprises about 70% sequence identity to the epitope to which any one of the antibodies listed in Table 1 binds with a different affinity as compared to any one of the antibodies listed in Table 1. The first binding domain may bind to an epitope that comprises about 75% sequence identity to the epitope to which any one of the antibodies listed in Table 1 binds with a different affinity as compared to any one of the antibodies listed in Table 1. The first binding domain may bind to an epitope that comprises about 80% sequence identity to the epitope to which any one of the antibodies listed in Table 1 binds with a different affinity as compared to any one of the antibodies listed in Table 1. The first binding domain may bind to an epitope that comprises about 85% sequence identity to the epitope to which any one of the antibodies listed in Table 1 binds with a different affinity as compared to any one of the antibodies listed in Table 1. The first binding domain may bind to an epitope that comprises about 90% sequence identity to the epitope to which any one of the antibodies listed in Table 1 binds with a different affinity as compared to any one of the antibodies listed in Table 1. The first binding domain may bind to an epitope that comprises about 95% sequence identity to the epitope to which any one of the antibodies listed in Table 1 binds with a different affinity as compared to any one of the antibodies listed in Table 1. The first binding domain may bind to an epitope that comprises about 99% sequence identity to the epitope to which any one of the antibodies listed in Table 1 binds with a different affinity as compared to any one of the antibodies listed in Table 1.

The first binding domain may bind to an epitope that comprises a different epitope than the epitope to which any one of the antibodies listed in Table 1 binds. The first binding domain may bind to an epitope that comprises a different epitope than the epitope to which any one of the antibodies listed in Table 1 binds, wherein the epitopes do not bind to any of the same amino acids on the internalizing receptor protein. The first binding domain may bind to an epitope that comprises a different epitope than the epitope to which any one of the antibodies listed in Table 1 binds, wherein the epitopes bind to any one or more of the same amino acids on the internalizing receptor protein. The first binding domain may bind to an epitope that comprises a different epitope than the epitope to which any one of the antibodies listed in Table 1 binds, wherein the epitopes bind to any two or more of the same amino acids on the internalizing receptor protein. The first binding domain may bind to an epitope that comprises a different epitope than the epitope to which any one of the antibodies listed in Table 1 binds, wherein the epitopes bind to any three or more of the same amino acids on the internalizing receptor protein. The first binding domain may bind to an epitope that comprises a different epitope than the epitope to which any one of the antibodies listed in Table 1 binds, wherein the epitopes bind to any four or more of the same amino acids on the internalizing receptor protein. The first binding domain may bind to an epitope that comprises a different epitope than the epitope to which any one of the antibodies listed in Table 1 binds, wherein the epitopes bind to any five or more of the same amino acids on the internalizing receptor protein. The first binding domain may bind to an epitope that comprises a different epitope than the epitope to which any one of the antibodies listed in Table 1 binds, wherein the epitopes bind to any six or more of the same amino acids on the internalizing receptor protein. The first binding domain may bind to an epitope that comprises a different epitope than the epitope to which any one of the antibodies listed in Table 1 binds, wherein the epitopes bind to any seven or more of the same amino acids on the internalizing receptor protein. The first binding domain may bind to an epitope that comprises a different epitope than the epitope to which any one of the antibodies listed in Table 1 binds, wherein the epitopes bind to any eight or more of the same amino acids on the internalizing receptor protein. The first binding domain may bind to an epitope that comprises a different epitope than the epitope to which any one of the antibodies listed in Table 1 binds, wherein the epitopes bind to any nine or more of the same amino acids on the internalizing receptor protein. The first binding domain may bind to an epitope that comprises a different epitope than the epitope to which any one of the antibodies listed in Table 1 binds, wherein the epitopes bind to any ten or more of the same amino acids on the internalizing receptor protein.

In some embodiments, the antibodies targeting the degrader protein comprise sequences listed Table 1. In some embodiments, the antibodies targeting the degrader protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1.

In some cases, the antibodies targeting the degrader protein may bind the same epitope as any one of the antibodies listed in Table 1. The antibodies targeting the degrader protein may bind to an epitope that comprises about 70% sequence identity to the epitope to which any one of the antibodies listed in Table 1 binds. The antibodies targeting the degrader protein may bind to an epitope that comprises about 75% sequence identity to the epitope to which any one of the antibodies listed in Table 1 binds. The antibodies targeting the degrader protein may bind to an epitope that comprises about 80% sequence identity to the epitope to which any one of the antibodies listed in Table 1 binds. The antibodies targeting the degrader protein may bind to an epitope that comprises about 85% sequence identity to the epitope to which any one of the antibodies listed in Table 1 binds. The antibodies targeting the degrader protein may bind to an epitope that comprises about 90% sequence identity to the epitope to which any one of the antibodies listed in Table 1 binds. The antibodies targeting the degrader protein may bind to an epitope that comprises about 95% sequence identity to the epitope to which any one of the antibodies listed in Table 1 binds. The antibodies targeting the degrader protein may bind to an epitope that comprises about 99% sequence identity to the epitope to which any one of the antibodies listed in Table 1 binds.

The antibodies targeting the degrader protein may bind to an epitope that comprises a different epitope than the epitope to which any one of the antibodies listed in Table 1 binds. The antibodies targeting the degrader protein may bind to an epitope that comprises a different epitope than the epitope to which any one of the antibodies listed in Table 1 binds, wherein the epitopes do not bind to any of the same amino acids on the degrader protein. The antibodies targeting the degrader protein may bind to an epitope that comprises a different epitope than the epitope to which any one of the antibodies listed in Table 1 binds, wherein the epitopes bind to any one or more of the same amino acids on the degrader protein. The antibodies targeting the degrader protein may bind to an epitope that comprises a different epitope than the epitope to which any one of the antibodies listed in Table 1 binds, wherein the epitopes bind to any two or more of the same amino acids on the degrader protein. The antibodies targeting the degrader protein may bind to an epitope that comprises a different epitope than the epitope to which any one of the antibodies listed in Table 1 binds, wherein the epitopes bind to any three or more of the same amino acids on the degrader protein. The antibodies targeting the degrader protein may bind to an epitope that comprises a different epitope than the epitope to which any one of the antibodies listed in Table 1 binds, wherein the epitopes bind to any four or more of the same amino acids on the degrader protein. The antibodies targeting the degrader protein may bind to an epitope that comprises a different epitope than the epitope to which any one of the antibodies listed in Table 1 binds, wherein the epitopes bind to any five or more of the same amino acids on the degrader protein. The antibodies targeting the degrader protein may bind to an epitope that comprises a different epitope than the epitope to which any one of the antibodies listed in Table 1 binds, wherein the epitopes bind to any six or more of the same amino acids on the degrader protein. The antibodies targeting the degrader protein may bind to an epitope that comprises a different epitope than the epitope to which any one of the antibodies listed in Table 1 binds, wherein the epitopes bind to any seven or more of the same amino acids on the degrader protein. The antibodies targeting the degrader protein may bind to an epitope that comprises a different epitope than the epitope to which any one of the antibodies listed in Table 1 binds, wherein the epitopes bind to any eight or more of the same amino acids on the degrader protein. The antibodies targeting the degrader protein may bind to an epitope that comprises a different epitope than the epitope to which any one of the antibodies listed in Table 1 binds, wherein the epitopes bind to any nine or more of the same amino acids on the degrader protein. The antibodies targeting the degrader protein may bind to an epitope that comprises a different epitope than the epitope to which any one of the antibodies listed in Table 1 binds, wherein the epitopes bind to any ten or more of the same amino acids on the degrader protein.

TABLE 1
Exemplary antibody sequences targeting the internalizing receptor protein.

| Antibody | Arm 1 | SEQ ID | | SEQ ID | VH | SEQ ID | | SEQ ID | VL |
|---|---|---|---|---|---|---|---|---|---|
| IDs | Target | NO | HC sequence | NO | sequence | NO | LC sequence | NO | sequence |
| EPI1092 | CD71 | 1 | QVQLVQSGAEVKKPGASVKMSC | 2 | QVQLVQ | 3 | DIQMTQSPSSLS | 4 | DIQMTQ |
| EPI1091 | | | KASGYTFTSYWMHWVRQAPGQ | | SGAEVK | | ASVGDRVTITCS | | SPSSLSA |
| EPI1090 | | | GLEWIGAIYPGNSETGYAQKFQG | | KPGASV | | ASSSVYYMYWF | | SVGDRV |
| EPI1093 | | | RATLTADTSTSTAYMELSSLRSE | | KMSCKA | | QQKPGKAPKLW | | TITCSAS |
| EPI1177 | | | DTAVYYCTRENWDPGFAFWGQ | | SGYTFTS | | LYSTSNLASGVP | | SSVYYM |
| | | | GTLITVSSASTKGPSVFPLAPSSK | | YWMHW | | SRFSGSGSGTDY | | YWFQQ |
| | | | STSGGTAALGCLVKDYFPEPVTV | | VRQAPG | | TLTISSMQPEDF | | KPGKAP |
| | | | SWNSGALTSGVHTFPAVLQSSGL | | QGLEWI | | ATYYCQQRRNY | | KLWIYS |
| | | | YSLSSVVTVPSSSLGTQTYICNVN | | GAIYPG | | PYTFGQGTKLEI | | TSNLAS |
| | | | HKPSNTKVDKKVEPKSCDKTHT | | NSETGY | | KRTVAAPSVFIF | | GVPSRFS |
| | | | CPPCPAPELLGGPSVFLFPPKPKD | | AQKFQG | | PPSDEQLKSGTA | | GSGSGT |
| | | | TLMISRTPEVTCVVVDVSHEDPE | | RATLTA | | SVVCLLNNFYPR | | DYTLTIS |
| | | | VKFNWYVDGVEVHNAKTKPRE | | DTSTST | | EAKVQWKVDN | | SMQPED |
| | | | EQYNSTYRVVSVLTVLHQDWLN | | AYMELS | | ALQSGNSQESVT | | FATYYC |
| | | | GKEYKCKVSNKALPAPIEKTISK | | SLRSEDT | | EQDSKDSTYSLS | | QQRRNY |
| | | | AKGQPREPQVYTLPPSRDELTKN | | AVYYCT | | STLTLSKADYEK | | PYTFGQ |
| | | | QVSLWCLVKGFYPSDIAVEWES | | RENWDP | | HKVYACEVTHQ | | GTKLEIK |
| | | | NGQPENNYKTTPPVLDSDGSFFL | | GFAFWG | | GLSSPVTKSFNR | | |
| | | | YSKLTVDKSRWQQGNVFSCSVM | | QGTLITV | | GEC | | |
| | | | HEALHNHYTQKSLSLSPGK | | SS | | | | |
| EPI1237 | MUC1 | 5 | QVQLVQSGAEVKKPGASVKMSC | 6 | QVQLVQ | 7 | DIQMTQSPSSLS | 8 | DIQMTQ |
| EPI1118 | | | KASGYTFTSYWMHWVRQAPGQ | | SGAEVK | | ASVGDRVTITCS | | SPSSLSA |
| | | | GLEWIGAIYPGNSETGYAQKFQG | | KPGASV | | ASSSVYYMYWF | | SVGDRV |
| | | | RATLTADTSTSTAYMELSSLRSE | | KMSCKA | | QQKPGKAPKLW | | TITCSAS |
| | | | DTAVYYCTRENWDPGFAFWGQ | | SGYTFTS | | LYSTSNLASGVP | | SSVYYM |
| | | | GTLITVSSASTKGPSVFPLAPSSK | | YWMHW | | SRFSGSGSGTDY | | YWFQQ |
| | | | STSGGTAALGCLVKDYFPEPVTV | | VRQAPG | | TLTISSMQPEDF | | KPGKAP |
| | | | SWNSGALTSGVHTFPAVLQSSGL | | QGLEWI | | ATYYCQQRRNY | | KLWIYS |
| | | | YSLSSVVTVPSSSLGTQTYICNVN | | GAIYPG | | PYTFGQGTKLEI | | TSNLAS |
| | | | HKPSNTKVDKKVEPKSCDKTHT | | NSETGY | | KRTVAAPSVFIF | | GVPSRFS |
| | | | CPPCPAPELLGGPSVFLFPPKPKD | | AQKFQG | | PPSDEQLKSGTA | | GSGSGT |
| | | | TLMISRTPEVTCVVVDVSHEDPE | | RATLTA | | SVVCLLNNFYPR | | DYTLTIS |
| | | | VKFNWYVDGVEVHNAKTKPRE | | DTSTST | | EAKVQWKVDN | | SMQPED |
| | | | EQYNSTYRVVSVLTVLHQDWLN | | AYMELS | | ALQSGNSQESVT | | FATYYC |
| | | | GKEYKCKVSNKALPAPIEKTISK | | SLRSEDT | | EQDSKDSTYSLS | | QQRRNY |
| | | | AKGQPREPQVYTLPPSRDELTKN | | AVYYCT | | STLTLSKADYEK | | PYTFGQ |
| | | | QVSLWCLVKGFYPSDIAVEWES | | RENWDP | | HKVYACEVTHQ | | GTKLEIK |
| | | | NGQPENNYKTTPPVLDSDGSFFL | | GFAFWG | | GLSSPVTKSFNR | | |
| | | | YSKLTVDKSRWQQGNVFSCSVM | | QGTLITV | | GEC | | |
| | | | HEALHNHYTQKSLSLSPGK | | | | | | |
| EPI1238 | MUC1 | 9 | EVQLVESGGGLVQPGGSMRLSC | 10 | EVQLVE | 11 | DIVMTQSPLSNP | 12 | DIVMTQ |
| EPI1119 | | | VASGFPFSNYWMNWVRQAPGK | | SGGGLV | | VTPGEPASISCRS | | SPLSNPV |
| | | | GLEWVGEIRLKSNQYTTHYAES | | QPGGSM | | SKSLLHSNGITY | | TPGEPAS |
| | | | VKGRFTISRDDSKNSLYLQMNSL | | RLSCVA | | FFWYLQKPGQS | | ISCRSSK |
| | | | KTEDTAVYYCTRHYYFDYWGQ | | SGFPFSN | | PQLLIYQMSNLA | | SLLHSN |
| | | | GTLVTVSSASTKGPSVFPLAPSSK | | YWMNW | | SGVPDRFSGSGS | | GITYFF |
| | | | STSGGTAALGCLVKDYFPEPVTV | | VRQAPG | | GTDFTLRISRVE | | WYLQKP |
| | | | SWNSGALTSGVHTFPAVLQSSGL | | KGLEWV | | AEDVGVYYCAQ | | GQSPQL |
| | | | YSLSSVVTVPSSSLGTQTYICNVN | | GEIRLKS | | NLELPPTFGQGT | | LIYQMS |
| | | | HKPSNTKVDKKVEPKSCDKTHT | | NQYTTH | | KVEIKRTVAAPS | | NLASGV |
| | | | CPPCPAPELLGGPSVFLFPPKPKD | | YAESVK | | VFIFPPSDEQLKS | | PDRFSGS |
| | | | TLMISRTPEVTCVVVDVSHEDPE | | GRFTISR | | GTASVVCLLNN | | GSGTDF |
| | | | VKFNWYVDGVEVHNAKTKPRE | | DDSKNS | | FYPREAKVQWK | | TLRISRV |
| | | | EQYNSTYRVVSVLTVLHQDWLN | | LYLQMN | | VDNALQSGNSQ | | EAEDVG |
| | | | GKEYKCKVSNKALPAPIEKTISK | | SLKTED | | ESVTEQDSKDST | | VYYCAQ |
| | | | AKGQPREPQVYTLPPSRDELTKN | | TAVYYC | | YSLSSTLTLSKA | | NLELPPT |
| | | | QVSLWCLVKGFYPSDIAVEWES | | TRHYYF | | DYEKHKVYACE | | FGQGTK |
| | | | NGQPENNYKTTPPVLDSDGSFFL | | DYWGQ | | VTHQGLSSPVTK | | VEIK |
| | | | YSKLTVDKSRWQQGNVFSCSVM | | GTLVTV | | SFNRGEC | | |
| | | | HEALHNHYTQKSLSLSPGK | | SS | | | | |
| EPI1240 | MUC1 | 13 | QVQLVQSGAEVKKPGASVKVSC | 14 | QVQLVQ | 15 | DIQMTQSPSSLS | 16 | DIQMTQ |
| EPI221 | | | KASGYTFSAYWIEWVRQAPGKG | | SGAEVK | | ASVGDRVTITCK | | SPSSLSA |
| EPI1121 | | | LEWVGEILPGSGNSRYNEKFKGR | | KPGASV | | SSQSLLYSSNQK | | SVGDRV |
| | | | VTVTRDTSTNTAYMELSSLRSED | | KVSCKA | | IYLAWYQQKPG | | TITCKSS |
| | | | TAVYYCARSYDFAWFAYWGQG | | SGYTFS | | KAPKLLIYWAST | | QSLLYSS |
| | | | TLVTVSSASTKGPSVFPLAPSSKS | | AYWIEW | | RESGVPSRFSGS | | NQKIYL |
| | | | TSGGTAALGCLVKDYFPEPVTVS | | VRQAPG | | GSGTDFTFTISSL | | AWYQQ |
| | | | WNSGALTSGVHTFPAVLQSSGL | | KGLEWV | | QPEDIATYYCQQ | | KPGKAP |
| | | | YSLSSVVTVPSSSLGTQTYICNVN | | GEILPGS | | YYRYPRTFGQG | | KLLIYW |
| | | | HKPSNTKVDKKVEPKSCDKTHT | | GNSRYN | | TKVEIKRTVAAP | | ASTRES |
| | | | CPPCPAPELLGGPSVFLFPPKPKD | | EKFKGR | | SVFIFPPSDEQLK | | GVPSRFS |
| | | | TLMISRTPEVTCVVVDVSHEDPE | | VTVTRD | | SGTASVVCLLN | | GSGSGT |
| | | | VKFNWYVDGVEVHNAKTKPRE | | TSTNTA | | NFYPREAKVQW | | DFTFTIS |
| | | | EQYNSTYRVVSVLTVLHQDWLN | | YMELSS | | KVDNALQSGNS | | SLQPEDI |
| | | | GKEYKCKVSNKALPAPIEKTISK | | LRSEDT | | QESVTEQDSKDS | | ATYYCQ |
| | | | AKGQPREPQVYTLPPSRDELTKN | | AVYYCA | | TYSLSSTLTLSK | | QYYRYP |
| | | | QVSLWCLVKGFYPSDIAVEWES | | RSYDFA | | ADYEKHKVYAC | | RTFGQG |
| | | | NGQPENNYKTTPPVLDSDGSFFL | | WFAYW | | EVTHQGLSSPVT | | TKVEIK |
| | | | YSKLTVDKSRWQQGNVFSCSVM | | GQGTLV | | KSFNRGEC | | |
| | | | HEALHNHYTQKSLSLSPGK | | TVSS | | | | |
| EPI1241 | MUC1 | 17 | QAQLVQSGAEVVKPGASVKMSC | 18 | QAQLVQ | 19 | EIVLTQSPATMS | 20 | EIVLTQS |
| EPI1122 | | | KASGYTFTSYNMHWVKQTPGQ | | SGAEVV | | ASPGERVTITCS | | PATMSA |
| | | | GLEWIGYIYPGNGATNYNQKFQ | | KPGASV | | AHSSVSFMHWF | | SPGERV |
| | | | GKATLTADPSSSTAYMQISSLTSE | | KMSCKA | | QQKPGTSPKLWI | | TITCSAH |
| | | | DSAVYFCARGDSVPFAYWGQGT | | SGYTFTS | | YSTSSLASGVPA | | SSVSFM |
| | | | LVTVSAASTKGPSVFPLAPSSKST | | YNMHW | | RFGGSGSGTSYS | | HWFQQ |
| | | | SGGTAALGCLVKDYFPEPVTVS | | VKQTPG | | LTISSMEAEDAA | | KPGTSP |
| | | | WNSGALTSGVHTFPAVLQSSGL | | QGLEWI | | TYYCQQRSSFPL | | KLWIYS |
| | | | YSLSSVVTVPSSSLGTQTYICNVN | | GYIYPG | | TFGAGTKLELK | | TSSLASG |
| | | | HKPSNTKVDKKVEPKSCDKTHT | | NGATNY | | RTVAAPSVFIFPP | | VPARFG |
| | | | CPPCPAPELLGGPSVFLFPPKPKD | | NQKFQG | | SDEQLKSGTASV | | GSGSGT |
| | | | TLMISRTPEVTCVVVDVSHEDPE | | KATLTA | | VCLLNNFYPRE | | SYSLTIS |
| | | | VKFNWYVDGVEVHNAKTKPRE | | DPSSSTA | | AKVQWKVDNA | | SMEAED |
| | | | EQYNSTYRVVSVLTVLHQDWLN | | YMQISS | | LQSGNSQESVTE | | AATYYC |
| | | | GKEYKCKVSNKALPAPIEKTISK | | LTSEDS | | QDSKDSTYSLSS | | QQRSSFP |
| | | | AKGQPREPQVYTLPPSRDELTKN | | AVYFCA | | TLTLSKADYEK | | LTFGAG |
| | | | QVSLWCLVKGFYPSDIAVEWES | | RGDSVP | | HKVYACEVTHQ | | TKLELK |
| | | | NGQPENNYKTTPPVLDSDGSFFL | | FAYWG | | GLSSPVTKSFNR | | |
| | | | YSKLTVDKSRWQQGNVFSCSVM | | QGTLVT | | GEC | | |
| | | | HEALHNHYTQKSLSLSPGK | | VSA | | | | |
| EPI1242 | MUC1 | 21 | EVKLVESGGGLVAPGGSLKLSC | 22 | EVKLVE | 23 | DVLMTQTPLSLP | 24 | DVLMTQ |
| EPI2212 | | | AASGFTFSSYPMSWVRQTPEKRL | | SGGGLV | | VVSLGDQASISCR | | TPLSLPV |
| EPI1123 | | | EWVAYINNGGGNPYYPDTVKGR | | APGGSL | | SSQTIVHSNGKI | | SLGDQA |
| | | | FTISRDNAKNTLYLQMSSLKSED | | KLSCAA | | YLEWYLQKPGQ | | SISCRSS |
| | | | TAIYYCIRQYYGFDYWGQGTTL | | SGFTFSS | | SPKLLIYRVSKR | | QTIVHS |
| | | | TVSSASTKGPSVFPLAPSSKSTSG | | YPMSW | | FSGVPDRFSGSG | | NGKIYL |
| | | | GTAALGCLVKDYFPEPVTVSWN | | VRQTPE | | SGTDFTLKISRV | | EWYLQK |
| | | | SGALTSGVHTFPAVLQSSGLYSL | | KRLEWV | | EAEDLGVYYCF | | PGQSPK |
| | | | SSVVTVPSSSLGTQTYICNVNHK | | AYINNG | | QGSHVPWTFGG | | LLIYRVS |
| | | | PSNTKVDKKVEPKSCDKTHTCPP | | GGNPYY | | GTKLEIKRTVAA | | KRFSGV |
| | | | CPAPELLGGPSVFLFPPKPKDTL | | PDTVKG | | PSVFIFPPSDEQL | | PDRFSGS |
| | | | MISRTPEVTCVVVDVSHEDPEVK | | RFTISRD | | KSGTASVVCLL | | GSGTDF |
| | | | FNWYVDGVEVHNAKTKPREEQ | | NAKNTL | | NNFYPREAKVQ | | TLKISRV |
| | | | YNSTYRVVSVLTVLHQDWLNGK | | YLQMSS | | WKVDNALQSG | | EAEDLG |
| | | | EYKCKVSNKALPAPIEKTISKAK | | LKSEDT | | NSQESVTEQDSK | | VYYCFQ |
| | | | GQPREPQVYTLPPSRDELTKNQV | | AIYYCIR | | DSTYSLSSTLTL | | GSHVPW |
| | | | SLWCLVKGFYPSDIAVEWESNG | | QYYGFD | | SKADYEKHKVY | | TFGGGT |
| | | | QPENNYKTTPPVLDSDGSFFLYS | | YWGQG | | ACEVTHQGLSSP | | KLEIK |
| | | | KLTVDKSRWQQGNVFSCSVMHE | | TTLTVSS | | VTKSFNRGEC | | |
| | | | ALHNHYTQKSLSLSPGK | | | | | | |
| EPI1243 | CD276 | 25 | EVQLVESGGGLVQPGGSLRLSCA | 26 | EVQLVE | 27 | DIQLTQSPSFLSA | 28 | DIQLTQS |
| EPI1135 | | | ASGFTFSSFGMHWVRQAPGKGL | | SGGGLV | | SVGDRVTITCKA | | PSFLSAS |
| | | | EWVAYISSDSSAIYYADTVKGRF | | QPGGSL | | SQNVDTNVAW | | VGDRVT |
| | | | TISRDNAKNSLYLQMNSLRDEDT | | RLSCAA | | YQQKPGKAPKA | | ITCKASQ |
| | | | AVYYCGRGRENIYYGSRLDYWG | | SGFTFSS | | LIYSASYRYSGV | | NVDTNV |
| | | | QGTTVTVSSASTKGPSVFPLAPSS | | FGMHW | | PSRFSGSGSGTD | | AWYQQ |
| | | | KSTSGGTAALGCLVKDYFPEPVT | | VRQAPG | | FTLTISSLQPEDF | | KPGKAP |
| | | | VSWNSGALTSGVHTFPAVLQSS | | KGLEWV | | ATYYCQQYNNY | | KALIYS |
| | | | GLYSLSSVVTVPSSSLGTQTYICN | | AYISSDS | | PFTFGQGTKLEI | | ASYRYS |
| | | | VNHKPSNTKVDKRVEPKSCDKT | | SAIYYA | | KRTVAAPSVFIF | | GVPSRFS |
| | | | HTCPPCPAPELLGGPSVFLFPPKP | | DTVKGR | | PPSDEQLKSGTA | | GSGSGT |
| | | | KDTLMISRTPEVTCVVVDVSHED | | FTISRDN | | SVVCLLNNFYPR | | DFTLTIS |
| | | | PEVKFNWYVDGVEVHNAKTKP | | AKNSLY | | EAKVQWKVDN | | SLQPEDF |
| | | | REEQYNSTYRVVSVLTVLHQDW | | LQMNSL | | ALQSGNSQESVT | | ATYYCQ |
| | | | LNGKEYKCKVSNKALPAPIEKTI | | RDEDTA | | EQDSKDSTYSLS | | QYNNYP |
| | | | SKAKGQPREPQVYTLPPSRDELT | | VYYCGR | | STLTLSKADYEK | | FTFGQG |
| | | | KNQVSLWCLVKGFYPSDIAVEW | | GRENIY | | HKVYACEVTHQ | | TKLEIK |
| | | | ESNGQPENNYKTTPPVLDSDGSF | | YGSRLD | | GLSSPVTKSFNR | | |
| | | | FLYSKLTVDKSRWQQGNVFSCS | | YWGQG | | GEC | | |
| | | | VMHEALHNHYTQKSLSLSPGKG | | TTVTVS | | | | |
| | | | GSHHHHHH | | S | | | | |
| EPI1244 | CD276 | 29 | QVQLQQSGAELVKPGASVKLSC | 30 | QVQLQQ | 31 | DIVMTQSPATLS | 32 | DIVMTQ |
| EPI1136 | | | KASGYTFTNYDINWVRQRPEQG | | SGAELV | | VTPGDRVSLSCR | | SPATLSV |
| | | | LEWIGWIFPGDGSTQYNEKFKGK | | KPGASV | | ASQSISDYLHW | | TPGDRV |
| | | | ATLTTDTSSSTAYMQLSRLTSED | | KLSCKA | | YQQKSHESPRLL | | SLSCRAS |
| | | | SAVYFCARQTTATWFAYWGQG | | SGYTFT | | IKYASQSISGIPS | | QSISDYL |
| | | | TLVTVSAASTKGPSVFPLAPSSKS | | NYDINW | | RFSGSGSGSDFT | | HWYQQ |
| | | | TSGGTAALGCLVKDYFPEPVTVS | | VRQRPE | | LSINSVEPEDVG | | KSHESP |
| | | | WNSGALTSGVHTFPAVLQSSGL | | QGLEWI | | VYYCQNGHSFP | | RLLIKY |
| | | | YSLSSVVTVPSSSLGTQTYICNVN | | GWIFPG | | LTFGAGTKLELK | | ASQSISG |
| | | | HKPSNTKVDKKVEPKSCDKTHT | | DGSTQY | | RTVAAPSVFIFPP | | IPSRFSG |
| | | | CPPCPAPELLGGPSVFLFPPKPKD | | NEKFKG | | SDEQLKSGTASV | | SGSGSD |
| | | | TLMISRTPEVTCVVVDVSHEDPE | | KATLTT | | VCLLNNFYPRE | | FTLSINS |
| | | | VKFNWYVDGVEVHNAKTKPRE | | DTSSSTA | | AKVQWKVDNA | | VEPEDV |
| | | | EQYNSTYRVVSVLTVLHQDWLN | | YMQLSR | | LQSGNSQESVTE | | GVYYCQ |
| | | | GKEYKCKVSNKALPAPIEKTISK | | LTSEDS | | QDSKDSTYSLSS | | NGHSFP |
| | | | AKGQPREPQVYTLPPSRDELTKN | | AVYFCA | | TLTLSKADYEK | | LTFGAG |
| | | | QVSLWCLVKGFYPSDIAVEWES | | RQTTAT | | HKVYACEVTHQ | | TKLELK |
| | | | NGQPENNYKTTPPVLDSDGSFFL | | WFAYW | | GLSSPVTKSFNR | | |
| | | | YSKLTVDKSRWQQGNVFSCSVM | | GQGTLV | | GEC | | |
| | | | HEALHNHYTQKSLSLSPGK | | TVSA | | | | |
| EPI1245 | CD276 | 33 | EVQLQESGPGLVKPSETLSLTCA | 34 | EVQLQE | 35 | DIQMTQSPSSLS | 36 | DIQMTQ |
| EPI2213 | | | VTGYSITSGYSWHWIRQFPGNGL | | SGPGLV | | ASVGDRVTITCK | | SPSSLSA |
| EPI1137 | | | EWMGYIHSSGSTNYNPSLKSRISI | | KPSETLS | | ASQNVGFNVAW | | SVGDRV |
| | | | SRDTSKNQFFLKLSSVTAADTAV | | LTCAVT | | YQQKPGKSPKA | | TITCKAS |
| | | | YYCAGYDDYFEYWGQGTTVTV | | GYSITSG | | LIYSASYRYSGV | | QNVGFN |
| | | | SSASTKGPSVFPLAPSSKSTSGGT | | YSWHWI | | PSRFSGSGSGTD | | VAWYQ |
| | | | AALGCLVKDYFPEPVTVSWNSG | | RQFPGN | | FTLTISSLQPEDF | | QKPGKS |
| | | | ALTSGVHTFPAVLQSSGLYSLSS | | GLEWM | | AEYFCQQYNWY | | PKALIYS |
| | | | VVTVPSSSLGTQTYICNVNHKPS | | GYIHSSG | | PFTFGQGTKLEI | | ASYRYS |
| | | | NTKVDKKVEPKSCDKTHTCPPCP | | STNYNP | | KRTVAAPSVFIF | | GVPSRFS |
| | | | APELLGGPSVFLFPPKPKDTLMIS | | SLKSRISI | | PPSDEQLKSGTA | | GSGSGT |
| | | | RTPEVTCVVVDVSHEDPEVKFN | | SRDTSK | | SVVCLLNNFYPR | | DFTLTIS |
| | | | WYVDGVEVHNAKTKPREEQYN | | NQFFLK | | EAKVQWKVDN | | SLQPEDF |
| | | | STYRVVSVLTVLHQDWLNGKEY | | LSSVTA | | ALQSGNSQESVT | | AEYFCQ |
| | | | KCKVSNKALPAPIEKTISKAKGQ | | ADTAVY | | EQDSKDSTYSLS | | QYNWY |
| | | | PREPQVYTLPPSRDELTKNQVSL | | YCAGYD | | STLTLSKADYEK | | PFTFGQ |
| | | | WCLVKGFYPSDIAVEWESNGQP | | DYFEYW | | HKVYACEVTHQ | | GTKLEIK |
| | | | ENNYKTTPPVLDSDGSFFLYSKL | | GQGTTV | | GLSSPVTKSFNR | | |
| | | | TVDKSRWQQGNVFSCSVMHEAL | | TVSS | | GEC | | |
| | | | HNHYTQKSLSLSPGK | | | | | | |
| EPI1246 | CD276 | 37 | QVQLVQSGAEVKKPGSSVKVSC | 38 | QVQLVQ | 39 | EIVLTQSPATLSL | 40 | EIVLTQS |
| EPI2214 | | | KASGYTFTNYVMHWVRQAPGQ | | SGAEVK | | SPGERATLSCRA | | PATLSLS |
| EPI1138 | | | GLEWMGYINPYNDDVKYNEKF | | KPGSSV | | SSRLIYMHWYQ | | PGERAT |
| | | | KGRVTITADESTSTAYMELSSLR | | KVSCKA | | QKPGQAPRPLIY | | LSCRASS |
| | | | SEDTAVYYCARWGYYGSPLYYF | | SGYTFT | | ATSNLASGIPAR | | RLIYMH |
| | | | DYWGQGTLVTVSSASTKGPSVF | | NYVMH | | FSGSGSGTDFTL | | WYQQK |
| | | | PLAPSSKSTSGGTAALGCLVKDY | | WVRQAP | | TISSLEPEDFAV | | PGQAPR |
| | | | FPEPVTVSWNSGALTSGVHTFPA | | GQGLEW | | YYCQQWNSNPP | | PLIYATS |
| | | | VLQSSGLYSLSSVVTVPSSSLGTQ | | MGYINP | | TFGQGTKVEIKR | | NLASGIP |
| | | | TYICNVNHKPSNTKVDKKVEPKS | | YNDDVK | | TVAAPSVFIFPPS | | ARFSGS |
| | | | CDKTHTCPPCPAPELLGGPSVFLF | | YNEKFK | | DEQLKSGTASV | | GSGTDF |
| | | | PPKPKDTLMISRTPEVTCVVVDV | | GRVTIT | | VCLLNNFYPRE | | TLTISSL |
| | | | SHEDPEVKFNWYVDGVEVHNA | | ADESTS | | AKVQWKVDNA | | EPEDFA |
| | | | KTKPREEQYNSTYRVVSVLTVL | | TAYMEL | | LQSGNSQESVTE | | VYYCQQ |
| | | | HQDWLNGKEYKCKVSNKALPA | | SSLRSED | | QDSKDSTYSLSS | | WNSNPP |
| | | | PIEKTISKAKGQPREPQVYTLPPS | | TAVYYC | | TLTLSKADYEK | | TFGQGT |
| | | | RDELTKNQVSLWCLVKGFYPSDI | | ARWGY | | HKVYACEVTHQ | | KVEIK |
| | | | AVEWESNGQPENNYKTTPPVLD | | YGSPLY | | GLSSPVTKSFNR | | |
| | | | SDGSFFLYSKLTVDKSRWQQGN | | YFDYW | | GEC | | |
| | | | VFSCSVMHEALHNHYTQKSLSLS | | GQGTLV | | | | |
| | | | PGK | | TVSS | | | | |
| EPI1247 | TPBG | 41 | EVQLQQSGPDLVKPGASVKISCK | 42 | EVQLQQ | 43 | SIVMTQTPTSLL | 44 | SIVMTQ |
| | | | ASGYSFTGYYMHWVKQSPGKG | | SGPDLV | | VSAGDRVTITCK | | TPTSLLV |
| | | | LEWIGRINPNNGVTLYNQKFKD | | KPGASV | | ASQSVSNDVAW | | SAGDRV |
| | | | KATLTVDKSSTTAYMELRSLTSE | | KISCKAS | | YQQKPGQSPKL | | TITCKAS |
| | | | DSAVYYCARSTMITNYVMDYW | | GYSFTG | | LISYTSSRYAGV | | QSVSND |
| | | | GQGTSVTVSSASTKGPSVFPLAP | | YYMHW | | PDRFSGSGYGTD | | VAWYQ |
| | | | SSKSTSGGTAALGCLVKDYFPEP | | VKQSPG | | FTLTISSVQAED | | QKPGQS |
| | | | VTVSWNSGALTSGVHTFPAVLQ | | KGLEWI | | AAVYFCQQDYN | | PKLLISY |
| | | | SSGLYSLSSVVTVPSSSLGTQTYI | | GRINPN | | SPPTFGGGTKLE | | TSSRYA |
| | | | CNVNHKPSNTKVDKKVEPKSCD | | NGVTLY | | IKRTVAAPSVFIF | | GVPDRF |
| | | | KTHTCPPCPAPELLGGPSVFLFPP | | NQKFKD | | PPSDEQLKSGTA | | SGSGYG |
| | | | KPKDTLMISRTPEVTCVVVDVSH | | KATLTV | | SVVCLLNNFYPR | | TDFTLTI |
| | | | EDPEVKFNWYVDGVEVHNAKT | | DKSSTT | | EAKVQWKVDN | | SSVQAE |
| | | | KPREEQYNSTYRVVSVLTVLHQ | | AYMELR | | ALQSGNSQESVT | | DAAVYF |
| | | | DWLNGKEYKCKVSNKALPAPIE | | SLTSEDS | | EQDSKDSTYSLS | | CQQDYN |
| | | | KTISKAKGQPREPQVYTLPPSRD | | AVYYCA | | STLTLSKADYEK | | SPPTFGG |
| | | | ELTKNQVSLWCLVKGFYPSDIAV | | RSTMIT | | HKVYACEVTHQ | | GTKLEIK |
| | | | EWESNGQPENNYKTTPPVLDSD | | NYVMD | | GLSSPVTKSFNR | | |
| | | | GSFFLYSKLTVDKSRWQQGNVF | | YWGQG | | GEC | | |
| | | | SCSVMHEALHNHYTQKSLSLSPG | | TSVTVSS | | | | |
| | | | K | | | | | | |
| EPI1248 | TPBG | 45 | EVQLVESGGGLVQPGGSLRLSCA | 46 | EVQLVE | 47 | DIQMTQSPSSLS | 48 | DIQMTQ |
| EPI1522 | | | ASGYTFTNFGMNWVRQAPGKG | | SGGGLV | | ASVGDRVTITCK | | SPSSLSA |
| | | | LEWVAWINTNTGEPRYAEEFKG | | QPGGSL | | ASQSVSNDVAW | | SVGDRV |
| | | | RFTISRDNAKNSLYLQMNSLRAE | | RLSCAA | | YQQKPGKAPKL | | TITCKAS |
| | | | DTAVYYCARDWDGAYFFDYWG | | SGYTFT | | LIYFATNRYTGV | | QSVSND |
| | | | QGTLVTVSSASTKGPSVFPLAPSS | | NFGMN | | PSRFSGSGYGTD | | VAWYQ |
| | | | KSTSGGTAALGCLVKDYFPEPVT | | WVRQAP | | FTLTISSLQPEDF | | QKPGKA |
| | | | VSWNSGALTSGVHTFPAVLQSS | | GKGLEW | | ATYYCQQDYSS | | PKLLIYF |
| | | | GLYSLSSVVTVPSSSLGTQTYICN | | VAWINT | | PWTFGQGTKVEI | | ATNRYT |
| | | | VNHKPSNTKVDKKVEPKSCDKT | | NTGEPR | | KRTVAAPSVFIF | | GVPSRFS |
| | | | HTCPPCPAPELLGGPSVFLFPPKP | | YAEEFK | | PPSDEQLKSGTA | | GSGYGT |
| | | | KDTLMISRTPEVTCVVVDVSHED | | GRFTISR | | SVVCLLNNFYPR | | DFTLTIS |
| | | | PEVKFNWYVDGVEVHNAKTKP | | DNAKNS | | EAKVQWKVDN | | SLQPEDF |
| | | | REEQYNSTYRVVSVLTVLHQDW | | LYLQMN | | ALQSGNSQESVT | | ATYYCQ |
| | | | LNGKEYKCKVSNKALPAPIEKTI | | SLRAED | | EQDSKDSTYSLS | | QDYSSP |
| | | | SKAKGQPREPQVYTLPPSRDELT | | TAVYYC | | STLTLSKADYEK | | WTFGQG |
| | | | KNQVSLWCLVKGFYPSDIAVEW | | ARDWD | | HKVYACEVTHQ | | TKVEIK |
| | | | ESNGQPENNYKTTPPVLDSDGSF | | GAYFFD | | GLSSPVTKSFNR | | |
| | | | FLYSKLTVDKSRWQQGNVFSCS | | YWGQG | | GEC | | |
| | | | VMHEALHNHYTQKSLSLSPGK | | TLVTVS | | | | |
| | | | | | S | | | | |
| EPI1249 | TPBG | 49 | EVQLVESGGGLVQPGGSLRLSCA | 50 | EVQLVE | 51 | DIQMTQSPSSLS | 52 | DIQMTQ |
| EPI1523 | | | ASGFTFNTYAMNWVRQAPGKG | | SGGGLV | | ASVGDRVTITCK | | SPSSLSA |
| | | | LEWVARIRSKSNNYATYYADSV | | QPGGSL | | ASQDVDTAVA | | SVGDRV |
| | | | KDRFTISRDDAKNSLYLQMNSLR | | RLSCAA | | WYQQKPGKAPK | | TITCKAS |
| | | | AEDTAVYYCVRQWDYDVRAMN | | SGFTFNT | | LLIYWASTRLTG | | QDVDTA |
| | | | YWGQGTLVTVSSASTKGPSVFPL | | YAMNW | | VPSRFSGSGSGT | | VAWYQ |
| | | | APSSKSTSGGTAALGCLVKDYFP | | VRQAPG | | DFTLTISSLQPED | | QKPGKA |
| | | | EPVTVSWNSGALTSGVHTFPAVL | | KGLEWV | | FATYYCQQYSS | | PKLLIY |
| | | | QSSGLYSLSSVVTVPSSSLGTQTY | | ARIRSKS | | YPYTFGQGTKL | | WASTRL |
| | | | ICNVNHKPSNTKVDKKVEPKSC | | NNYATY | | EIKRTVAAPSVFI | | TGVPSR |
| | | | DKTHTCPPCPAPELLGGPSVFLFP | | YADSVK | | FPPSDEQLKSGT | | FSGSGS |
| | | | PKPKDTLMISRTPEVTCVVVDVS | | DRFTISR | | ASVVCLLNNFY | | GTDFTL |
| | | | HEDPEVKFNWYVDGVEVHNAK | | DDAKNS | | PREAKVQWKVD | | TISSLQP |
| | | | TKPREEQYNSTYRVVSVLTVLH | | LYLQMN | | NALQSGNSQES | | EDFATY |
| | | | QDWLNGKEYKCKVSNKALPAPI | | SLRAED | | VTEQDSKDSTYS | | YCQQYS |
| | | | EKTISKAKGQPREPQVYTLPPSR | | TAVYYC | | LSSTLTLSKADY | | SYPYTF |
| | | | DELTKNQVSLWCLVKGFYPSDIA | | VRQWD | | EKHKVYACEVT | | GQGTKL |
| | | | VEWESNGQPENNYKTTPPVLDS | | YDVRA | | HQGLSSPVTKSF | | EIK |
| | | | DGSFFLYSKLTVDKSRWQQGNV | | MNYWG | | NRGEC | | |
| | | | FSCSVMHEALHNHYTQKSLSLSP | | QGTLVT | | | | |
| | | | GK | | VSS | | | | |
| EPI1250 | TPBG | 53 | EVQLEESGGGLVKPGGSLRLSCA | 54 | EVQLEE | 55 | DIQMTQSPSSLS | 56 | DIQMTQ |
| EPI1524 | | | ASGIDLSHYVVGWVRQAPGKGL | | SGGGLV | | ASVGDRVTITCQ | | SPSSLSA |
| | | | EWVSIIYGSGRTYYANWAKGRF | | KPGGSL | | ASQSIGSELAWY | | SVGDRV |
| | | | TISRDNSKNTLYLQMNSLRAEDT | | RLSCAA | | QQKPGQAPKLLI | | TITCQAS |
| | | | AVYYCARDASVSVYYWGYFDL | | SGIDLSH | | YRASTLESGVPS | | QSIGSEL |
| | | | WGRGTLVTVSSASTKGPSVFPLA | | YVVGW | | RFSGSGSGTEFT | | AWYQQ |
| | | | PSSKSTSGGTAALGCLVKDYFPE | | VRQAPG | | FTISSLQPEDLAT | | KPGQAP |
| | | | PVTVSWNSGALTSGVHTFPAVL | | KGLEWV | | YYCQQGYTYSEI | | KLLIYR |
| | | | QSSGLYSLSSVVTVPSSSLGTQTY | | SIIYGSG | | DNAFGQGTKLEI | | ASTLES |
| | | | ICNVNHKPSNTKVDKKVEPKSC | | RTYYAN | | KRTVAAPSVFIF | | GVPSRFS |
| | | | DKTHTCPPCPAPELLGGPSVFLFP | | WAKGRF | | PPSDEQLKSGTA | | GSGSGT |
| | | | PKPKDTLMISRTPEVTCVVVDVS | | TISRDNS | | SVVCLLNNFYPR | | EFTFTIS |
| | | | HEDPEVKFNWYVDGVEVHNAK | | KNTLYL | | EAKVQWKVDN | | SLQPED |
| | | | TKPREEQYNSTYRVVSVLTVLH | | QMNSLR | | ALQSGNSQESVT | | LATYYC |
| | | | QDWLNGKEYKCKVSNKALPAPI | | AEDTAV | | EQDSKDSTYSLS | | QQGYTY |
| | | | EKTISKAKGQPREPQVYTLPPSR | | YYCARD | | STLTLSKADYEK | | SEIDNAF |
| | | | DELTKNQVSLWCLVKGFYPSDIA | | ASVSVY | | HKVYACEVTHQ | | GQGTKL |
| | | | VEWESNGQPENNYKTTPPVLDS | | YWGYF | | GLSSPVTKSFNR | | EIK |
| | | | DGSFFLYSKLTVDKSRWQQGNV | | DLWGR | | GEC | | |
| | | | FSCSVMHEALHNHYTQKSLSLSP | | GTLVTV | | | | |
| | | | GK | | SS | | | | |
| EPI1251 | TPBG | 57 | EVQLEESGGGLVKPGGSLRLSCA | 58 | EVQLEE | 59 | GYDMTQSPSSV | 60 | GYDMT |
| EPI1525 | | | ASGIDLSSYGMGWVRQAPGKGL | | SGGGLV | | SASVGDRVTITC | | QSPSSVS |
| | | | EWVSIISRNSVTYYATWAKGRFT | | KPGGSL | | QASENIYSTLAW | | ASVGDR |
| | | | ISRDNSKNTLYLQMNSLRAEDTA | | RLSCAA | | YQQKPGKAPKL | | VTITCQ |
| | | | VYYCARRATYSGALGYFDIWGR | | SGIDLSS | | LIYDAFDLASGV | | ASENIYS |
| | | | GTLVTVSSASTKGPSVFPLAPSSK | | YGMGW | | PSRFKGSGSGTE | | TLAWYQ |
| | | | STSGGTAALGCLVKDYFPEPVTV | | VRQAPG | | YTLTISSLQPEDF | | QKPGKA |
| | | | SWNSGALTSGVHTFPAVLQSSGL | | KGLEWV | | ATYYCQQGYSG | | PKLLIYD |
| | | | YSLSSVVTVPSSSLGTQTYICNVN | | SIISRNS | | TNVDNAFGGGT | | AFDLAS |
| | | | HKPSNTKVDKKVEPKSCDKTHT | | VTYYAT | | KVEIKRTVAAPS | | GVPSRF |
| | | | CPPCPAPELLGGPSVFLFPPKPKD | | WAKGRF | | VFIFPPSDEQLKS | | KGSGSG |
| | | | TLMISRTPEVTCVVVDVSHEDPE | | TISRDNS | | GTASVVCLLNN | | TEYTLTI |
| | | | VKFNWYVDGVEVHNAKTKPRE | | KNTLYL | | FYPREAKVQWK | | SSLQPED |
| | | | EQYNSTYRVVSVLTVLHQDWLN | | QMNSLR | | VDNALQSGNSQ | | FATYYC |
| | | | GKEYKCKVSNKALPAPIEKTISK | | AEDTAV | | ESVTEQDSKDST | | QQGYSG |
| | | | AKGQPREPQVYTLPPSRDELTKN | | YYCARR | | YSLSSTLTLSKA | | TNVDNA |
| | | | QVSLWCLVKGFYPSDIAVEWES | | ATYSGA | | DYEKHKVYACE | | FGGGTK |
| | | | NGQPENNYKTTPPVLDSDGSFFL | | LGYFDI | | VTHQGLSSPVTK | | VEIK |
| | | | YSKLTVDKSRWQQGNVFSCSVM | | WGRGTL | | SFNRGEC | | |
| | | | HEALHNHYTQKSLSLSPGK | | VTVSS | | | | |
| EPI1252 | TPBG | 61 | QVQLQQWGAGLLKPSETLSLTC | 62 | QVQLQQ | 63 | EIVLTQSPATLSL | 64 | EIVLTQS |
| EPI1526 | | | AVYGGSFSGYYWTWIRQPPGKG | | WGAGLL | | SPGERATLSCRA | | PATLSLS |
| | | | LEWIGEIDHSESTNYNPSLKSRVT | | KPSETLS | | SQSVSSYLAWY | | PGERAT |
| | | | ISVDTSKNQFSLKLSSVTAADTA | | LTCAVY | | QQKPGQAPRLLI | | LSCRAS |
| | | | VYYCAGWFGELYHYYYGMDV | | GGSFSG | | YDASNRATGIPA | | QSVSSY |
| | | | WGQGTTVTVSSASTKGPSVFPLA | | YYWTWI | | RFSGSGSGTDFT | | LAWYQ |
| | | | PSSKSTSGGTAALGCLVKDYFPE | | RQPPGK | | LTISSLEPEDFAV | | QKPGQA |
| | | | PVTVSWNSGALTSGVHTFPAVL | | GLEWIG | | YYCQQRSNWPL | | PRLLIYD |
| | | | QSSGLYSLSSVVTVPSSSLGTQTY | | EIDHSES | | TFGGGTKVEIKR | | ASNRAT |
| | | | ICNVNHKPSNTKVDKKVEPKSC | | TNYNPS | | TVAAPSVFIFPPS | | GIPARFS |
| | | | DKTHTCPPCPAPELLGGPSVFLFP | | LKSRVTI | | DEQLKSGTASV | | GSGSGT |
| | | | PKPKDTLMISRTPEVTCVVVDVS | | SVDTSK | | VCLLNNFYPRE | | DFTLTIS |
| | | | HEDPEVKFNWYVDGVEVHNAK | | NQFSLK | | AKVQWKVDNA | | SLEPEDF |
| | | | TKPREEQYNSTYRVVSVLTVLH | | LSSVTA | | LQSGNSQESVTE | | AVYYCQ |
| | | | QDWLNGKEYKCKVSNKALPAPI | | ADTAVY | | QDSKDSTYSLSS | | QRSNWP |
| | | | EKTISKAKGQPREPQVYTLPPSR | | YCAGWF | | TLTLSKADYEK | | LTFGGG |
| | | | DELTKNQVSLWCLVKGFYPSDIA | | GELYHY | | HKVYACEVTHQ | | TKVEIK |
| | | | VEWESNGQPENNYKTTPPVLDS | | YYGMD | | GLSSPVTKSFNR | | |
| | | | DGSFFLYSKLTVDKSRWQQGNV | | VWGQG | | GEC | | |
| | | | FSCSVMHEALHNHYTQKSLSLSP | | TTVTVS | | | | |
| | | | GK | | S | | | | |
| EPI1253 | MST1R | 65 | EVQLVESGGGLVQPGGSLRLSCA | 66 | EVQLVE | 67 | EIVLTQSPATLSL | 68 | EIVLTQS |
| EPI1139 | | | ASGFTFSSYLMTWVRQAPGKGL | | SGGGLV | | SPGERATLSCRA | | PATLSLS |
| | | | EWVANIKQDGSEKYYVDSVKGR | | QPGGSL | | SQSVSRYLAWY | | PGERAT |
| | | | FTISRDNAKNSLNLQMNSLRAED | | RLSCAA | | QQKPGQAPRLLI | | LSCRAS |
| | | | TAVYYCTRDGYSSGRHYGMDV | | SGFTFSS | | YDASNRATGIPA | | QSVSRY |
| | | | WGQGTTVIVSSASTKGPSVFPLA | | YLMTW | | RFSGSGSGTDFT | | LAWYQ |
| | | | PSSKSTSGGTAALGCLVKDYFPE | | VRQAPG | | LTISSLEPEDFAV | | QKPGQA |
| | | | PVTVSWNSGALTSGVHTFPAVL | | KGLEWV | | YYCQQRSNWPR | | PRLLIYD |
| | | | QSSGLYSLSSVVTVPSSSLGTQTY | | ANIKQD | | TFGQGTKVEIKR | | ASNRAT |
| | | | ICNVNHKPSNTKVDKKVEPKSC | | GSEKYY | | TVAAPSVFIFPPS | | GIPARFS |
| | | | DKTHTCPPCPAPELLGGPSVFLFP | | VDSVKG | | DEQLKSGTASV | | GSGSGT |
| | | | PKPKDTLMISRTPEVTCVVVDVS | | RFTISRD | | VCLLNNFYPRE | | DFTLTIS |
| | | | HEDPEVKFNWYVDGVEVHNAK | | NAKNSL | | AKVQWKVDNA | | SLEPEDF |
| | | | TKPREEQYNSTYRVVSVLTVLH | | NLQMNS | | LQSGNSQESVTE | | AVYYCQ |
| | | | QDWLNGKEYKCKVSNKALPAPI | | LRAEDT | | QDSKDSTYSLSS | | QRSNWP |
| | | | EKTISKAKGQPREPQVYTLPPSR | | AVYYCT | | TLTLSKADYEK | | RTFGQG |
| | | | DELTKNQVSLWCLVKGFYPSDIA | | RDGYSS | | HKVYACEVTHQ | | TKVEIK |
| | | | VEWESNGQPENNYKTTPPVLDS | | GRHYG | | GLSSPVTKSFNR | | |
| | | | DGSFFLYSKLTVDKSRWQQGNV | | MDVWG | | GEC | | |
| | | | FSCSVMHEALHNHYTQKSLSLSP | | QGTTVI | | | | |
| | | | IGK | | VSS | | | | |
| EPI1254 | MST1R | 69 | EVQLVESGGGLVQPGGSLRLSCA | 70 | EVQLVE | 71 | DIQLTQSQSFVS | 72 | DIQLTQS |
| EPI1140 | | | ASGFTFSRHWMSWVRQAPGKGL | | SGGGLV | | TSVGDRVTVTC | | QSFVSTS |
| | | | EWVSEINPDSRTINYAPSVKGRF | | QPGGSL | | RASQNVGSSLV | | VGDRVT |
| | | | TISRDNAKNSLYLQMNSLRAEDT | | RLSCAA | | WYQQKPGKSPK | | VTCRAS |
| | | | AVYYCARRVRIHYYGAMDSWG | | SGFTFSR | | TLIYSASFLYSG | | QNVGSS |
| | | | QGTTVTVSSASTKGPSVFPLAPSS | | HWMSW | | VPSRFSGSGSGT | | LVWYQ |
| | | | KSTSGGTAALGCLVKDYFPEPVT | | VRQAPG | | EFTLTISSVQPED | | QKPGKS |
| | | | VSWNSGALTSGVHTFPAVLQSS | | KGLEWV | | FADYFCQQYNN | | PKTLIYS |
| | | | GLYSLSSVVTVPSSSLGTQTYICN | | SEINPDS | | YPLTFGGGTKV | | ASFLYS |
| | | | VNHKPSNTKVDKKVEPKSCDKT | | RTINYAP | | EIKRTVAAPSVFI | | GVPSRFS |
| | | | HTCPPCPAPELLGGPSVFLFPPKP | | SVKGRF | | FPPSDEQLKSGT | | GSGSGT |
| | | | KDTLMISRTPEVTCVVVDVSHED | | TISRDNA | | ASVVCLLNNFY | | EFTLTIS |
| | | | PEVKFNWYVDGVEVHNAKTKP | | KNSLYL | | PREAKVQWKVD | | SVQPED |
| | | | REEQYNSTYRVVSVLTVLHQDW | | QMNSLR | | NALQSGNSQES | | FADYFC |
| | | | LNGKEYKCKVSNKALPAPIEKTI | | AEDTAV | | VTEQDSKDSTYS | | QQYNNY |
| | | | SKAKGQPREPQVYTLPPSRDELT | | YYCARR | | LSSTLTLSKADY | | PLTFGG |
| | | | KNQVSLWCLVKGFYPSDIAVEW | | VRIHYY | | EKHKVYACEVT | | GTKVEI |
| | | | ESNGQPENNYKTTPPVLDSDGSF | | GAMDS | | HQGLSSPVTKSF | | K |
| | | | FLYSKLTVDKSRWQQGNVFSCS | | WGQGTT | | NRGEC | | |
| | | | VMHEALHNHYTQKSLSLSPGK | | VTVSS | | | | |
| EPI1255 | MST1R | 73 | EVQLQQSGAELVKPGASVKLSC | 74 | EVQLQQ | 75 | DIQMNQSPSSLS | 76 | DIQMNQ |
| EPI1141 | | | TTSGFNIIDTYIHWVNQKPDQGL | | SGAELV | | ASLGDTITITCH | | SPSSLSA |
| | | | EWIGRIDPADGNRKSDPKFQVKA | | KPGASV | | ASQNINVWLNW | | SLGDTIT |
| | | | TITVDTSSNTAYLQLSSLTSGDTA | | KLSCTTS | | YQQKPGNIPKLL | | ITCHASQ |
| | | | VYYCARGYGNLNAMDSWGQGT | | GFNIIDT | | IYKASNLHTGVP | | NINVWL |
| | | | SVTVSSASTKGPSVFPLAPSSKST | | YIHWVN | | SRFSGSGSGTGF | | NWYQQ |
| | | | SGGTAALGCLVKDYFPEPVTVS | | QKPDQG | | TLTISSLQPEDIA | | KPGNIPK |
| | | | WNSGALTSGVHTFPAVLQSSGL | | LEWIGRI | | TYYCQQGQSYP | | LLIYKAS |
| | | | YSLSSVVTVPSSSLGTQTYICNVN | | DPADGN | | LTFGGGTKLEIK | | NLHTGV |
| | | | HKPSNTKVDKKVEPKSCDKTHT | | RKSDPK | | RTVAAPSVFIFPP | | PSRFSGS |
| | | | CPPCPAPELLGGPSVFLFPPKPKD | | FQVKAT | | SDEQLKSGTASV | | GSGTGF |
| | | | TLMISRTPEVTCVVVDVSHEDPE | | ITVDTSS | | VCLLNNFYPRE | | TLTISSL |
| | | | VKFNWYVDGVEVHNAKTKPRE | | NTAYLQ | | AKVQWKVDNA | | QPEDIAT |
| | | | EQYNSTYRVVSVLTVLHQDWLN | | LSSLTSG | | LQSGNSQESVTE | | YYCQQG |
| | | | GKEYKCKVSNKALPAPIEKTISK | | DTAVYY | | QDSKDSTYSLSS | | QSYPLTF |
| | | | AKGQPREPQVYTLPPSRDELTKN | | CARGYG | | TLTLSKADYEK | | GGGTKL |
| | | | QVSLWCLVKGFYPSDIAVEWES | | NLNAM | | HKVYACEVTHQ | | EIK |
| | | | NGQPENNYKTTPPVLDSDGSFFL | | DSWGQ | | GLSSPVTKSFNR | | |
| | | | YSKLTVDKSRWQQGNVFSCSVM | | GTSVTV | | GEC | | |
| | | | HEALHNHYTQKSLSLSPGK | | SS | | | | |
| EPI1256 | MST1R | 77 | QVQLVQSGAEVKKPGATVKISC | 78 | QVQLVQ | 79 | EIVMTQSPGTLS | 80 | EIVMTQ |
| EPI1142 | | | KVSGYTFTDYHMDWVQQAPGK | | SGAEVK | | LSPGERATLSCK | | SPGTLSL |
| | | | GLEWMGDINPNNGGAIYNQKFK | | KPGATV | | SSQSLLFSGNQK | | SPGERA |
| | | | GRVTITADTSTDTAYMELSSLRS | | KISCKVS | | NYLAWYQQKP | | TLSCKSS |
| | | | EDTAVYYCARSHYDYAGGAWF | | GYTFTD | | GQAPRLLIYWA | | QSLLFSG |
| | | | AYWGQGTLVTVSRASTKGPSVF | | YHMDW | | STRASGIPDRFS | | NQKNYL |
| | | | PLAPSSKSTSGGTAALGCLVKDY | | VQQAPG | | GSGSGTDFTLTI | | AWYQQ |
| | | | FPEPVTVSWNSGALTSGVHTFPA | | KGLEW | | SRLEPEDFAVYY | | KPGQAP |
| | | | VLQSSGLYSLSSVVTVPSSSLGTQ | | MGDINP | | CQQYYSFPRTFG | | RLLIYW |
| | | | TYICNVNHKPSNTKVDKKVEPKS | | NNGGAI | | QGTKLEIKRTVA | | ASTRAS |
| | | | CDKTHTCPPCPAPELLGGPSVFLF | | YNQKFK | | APSVFIFPPSDEQ | | GIPDRFS |
| | | | PPKPKDTLMISRTPEVTCVVVDV | | GRVTIT | | LKSGTASVVCLL | | GSGSGT |
| | | | SHEDPEVKFNWYVDGVEVHNA | | ADTSTD | | NNFYPREAKVQ | | DFTLTIS |
| | | | KTKPREEQYNSTYRVVSVLTVL | | TAYMEL | | WKVDNALQSG | | RLEPEDF |
| | | | HQDWLNGKEYKCKVSNKALPA | | SSLRSED | | NSQESVTEQDSK | | AVYYCQ |
| | | | PIEKTISKAKGQPREPQVYTLPPS | | TAVYYC | | DSTYSLSSTLTL | | QYYSFP |
| | | | RDELTKNQVSLWCLVKGFYPSDI | | ARSHYD | | SKADYEKHKVY | | RTFGQG |
| | | | AVEWESNGQPENNYKTTPPVLD | | YAGGA | | ACEVTHQGLSSP | | TKLEIK |
| | | | SDGSFFLYSKLTVDKSRWQQGN | | WFAYW | | VTKSFNRGEC | | |
| | | | VFSCSVMHEALHNHYTQKSLSLS | | GQGTLV | | | | |
| | | | PGK | | TVSR | | | | |
| EPI1257 | HER3 | 81 | QVQLQQWGAGLLKPSETLSLTC | 82 | QVQLQQ | 83 | DIEMTQSPDSLA | 84 | DIEMTQ |
| | | | AVYGGSFSGYYWSWIRQPPGKG | | WGAGLL | | VSLGERATINCR | | SPDSLA |
| | | | LEWIGEINHSGSTNYNPSLKSRV | | KPSETLS | | SSQSVLYSSSNR | | VSLGER |
| | | | TISVETSKNQFSLKLSSVTAADTA | | LTCAVY | | NYLAWYQQNP | | ATINCRS |
| | | | VYYCARDKWTWYFDLWGRGTL | | GGSFSG | | GQPPKLLIYWAS | | SQSVLY |
| | | | VTVSSASTKGPSVFPLAPSSKSTS | | YYWSWI | | TRESGVPDRFSG | | SSSNRN |
| | | | GGTAALGCLVKDYFPEPVTVSW | | RQPPGK | | SGSGTDFTLTISS | | YLAWY |
| | | | NSGALTSGVHTFPAVLQSSGLYS | | GLEWIG | | LQAEDVAVYYC | | QQNPGQ |
| | | | LSSVVTVPSSSLGTQTYICNVNH | | EINHSGS | | QQYYSTPRTFG | | PPKLLIY |
| | | | KPSNTKVDKKVEPKSCDKTHTC | | TNYNPS | | QGTKVEIKRTV | | WASTRE |
| | | | PPCPAPELLGGPSVFLFPPKPKDT | | LKSRVTI | | AAPSVFIFPPSDE | | SGVPDR |
| | | | LMISRTPEVTCVVVDVSHEDPEV | | SVETSK | | QLKSGTASVVC | | FSGSGS |
| | | | KFNWYVDGVEVHNAKTKPREE | | NQFSLK | | LLNNFYPREAK | | GTDFTL |
| | | | QYNSTYRVVSVLTVLHQDWLNG | | LSSVTA | | VQWKVDNALQ | | TISSLQA |
| | | | KEYKCKVSNKALPAPIEKTISKA | | ADTAVY | | SGNSQESVTEQD | | EDVAVY |
| | | | KGQPREPQVYTLPPSRDELTKNQ | | YCARDK | | SKDSTYSLSSTL | | YCQQYY |
| | | | VSLWCLVKGFYPSDIAVEWESN | | WTWYF | | TLSKADYEKHK | | STPRTFG |
| | | | GQPENNYKTTPPVLDSDGSFFLY | | DLWGR | | VYACEVTHQGL | | QGTKVE |
| | | | SKLTVDKSRWQQGNVFSCSVMH | | GTLVTV | | SSPVTKSFNRGE | | IK |
| | | | EALHNHYTQKSLSLSPGK | | SS | | C | | |
| EPI1258 | HER3 | 85 | EVQLLESGGGLVQPGGSLRLSCA | 86 | EVQLLE | 87 | DIQMTQSPSSLS | 88 | DIQMTQ |
| EPI1168 | | | ASGFTFSSYAMSWVRQAPGKGL | | SGGGLV | | ASVGDRVTITCR | | SPSSLSA |
| | | | EWVSAINSQGKSTYYADSVKGR | | QPGGSL | | ASQGISNWLAW | | SVGDRV |
| | | | FTISRDNSKNTLYLQMNSLRAED | | RLSCAA | | YQQKPGKAPKL | | TITCRAS |
| | | | TAVYYCARWGDEGFDIWGQGT | | SGFTFSS | | LIYGASSLQSGV | | QGISNW |
| | | | LVTVSSASTKGPSVFPLAPSSKST | | YAMSW | | PSRFSGSGSGTD | | LAWYQ |
| | | | SGGTAALGCLVKDYFPEPVTVS | | VRQAPG | | FTLTISSLQPEDF | | QKPGKA |
| | | | WNSGALTSGVHTFPAVLQSSGL | | KGLEWV | | ATYYCQQYSSFP | | PKLLIYG |
| | | | YSLSSVVTVPSSSLGTQTYICNVN | | SAINSQG | | TTFGQGTKVEIK | | ASSLQS |
| | | | HKPSNTKVDKRVEPKSCDKTHT | | KSTYYA | | RTVAAPSVFIFPP | | GVPSRFS |
| | | | CPPCPAPELLGGPSVFLFPPKPKD | | DSVKGR | | SDEQLKSGTASV | | GSGSGT |
| | | | TLMISRTPEVTCVVVDVSHEDPE | | FTISRDN | | VCLLNNFYPRE | | DFTLTIS |
| | | | VKFNWYVDGVEVHNAKTKPRE | | SKNTLY | | AKVQWKVDNA | | SLQPEDF |
| | | | EQYNSTYRVVSVLTVLHQDWLN | | LQMNSL | | LQSGNSQESVTE | | ATYYCQ |
| | | | GKEYKCKVSNKALPAPIEKTISK | | RAEDTA | | QDSKDSTYSLSS | | QYSSFPT |
| | | | AKGQPREPQVYTLPPSRDELTKN | | VYYCAR | | TLTLSKADYEK | | TFGQGT |
| | | | QVSLWCLVKGFYPSDIAVEWES | | WGDEGF | | HKVYACEVTHQ | | KVEIK |
| | | | NGQPENNYKTTPPVLDSDGSFFL | | DIWGQG | | GLSSPVTKSFNR | | |
| | | | YSKLTVDKSRWQQGNVFSCSVM | | TLVTVS | | GEC | | |
| | | | HEALHNHYTQKSLSLSPGK | | S | | | | |
| EPI1259 | HER3 | 89 | EVQLVESGGGLVQPGGSLRLSCA | 90 | EVQLVE | 91 | DIQMTQSPSSLS | 92 | DIQMTQ |
| EPI1167 | | | ASGFTLSGDWIHWVRQAPGKGL | | SGGGLV | | ASVGDRVTITCR | | SPSSLSA |
| | | | EWVGEISAAGGYTDYADSVKGR | | QPGGSL | | ASQNIATDVAW | | SVGDRV |
| | | | FTISADTSKNTAYLQMNSLRAED | | RLSCAA | | YQQKPGKAPKL | | TITCRAS |
| | | | TAVYYCARESRVSFEAAMDYW | | SGFTLSG | | LIYSASFLYSGV | | QNIATD |
| | | | GQGTLVTVSSASTKGPSVFPLAP | | DWIHWV | | PSRFSGSGSGTD | | VAWYQ |
| | | | SSKSTSGGTAALGCLVKDYFPEP | | RQAPGK | | FTLTISSLQPEDF | | QKPGKA |
| | | | VTVSWNSGALTSGVHTFPAVLQ | | GLEWVG | | ATYYCQQSEPEP | | PKLLIYS |
| | | | SSGLYSLSSVVTVPSSSLGTQTYI | | EISAAG | | YTFGQGTKVEIK | | ASFLYS |
| | | | CNVNHKPSNTKVDKKVEPKSCD | | GYTDYA | | RTVAAPSVFIFPP | | GVPSRFS |
| | | | KTHTCPPCPAPELLGGPSVFLFPP | | DSVKGR | | SDEQLKSGTASV | | GSGSGT |
| | | | KPKDTLMISRTPEVTCVVVDVSH | | FTISADT | | VCLLNNFYPRE | | DFTLTIS |
| | | | EDPEVKFNWYVDGVEVHNAKT | | SKNTAY | | AKVQWKVDNA | | SLQPEDF |
| | | | KPREEQYNSTYRVVSVLTVLHQ | | LQMNSL | | LQSGNSQESVTE | | ATYYCQ |
| | | | DWLNGKEYKCKVSNKALPAPIE | | RAEDTA | | QDSKDSTYSLSS | | QSEPEPY |
| | | | KTISKAKGQPREPQVYTLPPSRD | | VYYCAR | | TLTLSKADYEK | | TFGQGT |
| | | | ELTKNQVSLWCLVKGFYPSDIAV | | ESRVSFE | | HKVYACEVTHQ | | KVEIK |
| | | | EWESNGQPENNYKTTPPVLDSD | | AAMDY | | GLSSPVTKSFNR | | |
| | | | GSFFLYSKLTVDKSRWQQGNVF | | WGQGTL | | GEC | | |
| | | | SCSVMHEALHNHYTQKSLSLSPG | | VTVSS | | | | |
| | | | K | | | | | | |
| EPI1260 | HER3 | 93 | QVQLVQSGAEVKKPGASVKVSC | 94 | QVQLVQ | 95 | DIVMTQSPDSLA | 96 | DIVMTQ |
| | | | KASGYTFRSSYISWVRQAPGQGL | | SGAEVK | | VSLGERATINCK | | SPDSLA |
| | | | EWMGWIYAGTGSPSYNQKLQG | | KPGASV | | SSQSVLNSGNQ | | VVSLGER |
| | | | RVTMTTDTSTSTAYMELRSLRSD | | KVSCKA | | KNYLTWYQQKP | | ATINCKS |
| | | | DTAVYYCARHRDYYSNSLTYW | | SGYTFR | | GQPPKLLIYWAS | | SQSVLN |
| | | | GQGTLVTVSSASTKGPSVFPLAP | | SSYISW | | TRESGVPDRFSG | | SGNQKN |
| | | | SSKSTSGGTAALGCLVKDYFPEP | | VRQAPG | | SGSGTDFTLTISS | | YLTWYQ |
| | | | VTVSWNSGALTSGVHTFPAVLQ | | QGLEW | | LQAEDVAVYYC | | QKPGQP |
| | | | SSGLYSLSSVVTVPSSSLGTQTYI | | MGWIYA | | QSDYSYPYTFG | | PKLLIY |
| | | | CNVNHKPSNTKVDKKVEPKSCD | | GTGSPS | | QGTKLEIKRTVA | | WASTRE |
| | | | KTHTCPPCPAPELLGGPSVFLFPP | | YNQKLQ | | APSVFIFPPSDEQ | | SGVPDR |
| | | | KPKDTLMISRTPEVTCVVVDVSH | | GRVTMT | | LKSGTASVVCLL | | FSGSGS |
| | | | EDPEVKFNWYVDGVEVHNAKT | | TDTSTST | | NNFYPREAKVQ | | GTDFTL |
| | | | KPREEQYNSTYRVVSVLTVLHQ | | AYMELR | | WKVDNALQSG | | TISSLQA |
| | | | DWLNGKEYKCKVSNKALPAPIE | | SLRSDD | | NSQESVTEQDSK | | EDVAVY |
| | | | KTISKAKGQPREPQVYTLPPSRD | | TAVYYC | | DSTYSLSSTLTL | | YCQSDY |
| | | | ELTKNQVSLWCLVKGFYPSDIAV | | ARHRDY | | SKADYEKHKVY | | SYPYTF |
| | | | EWESNGQPENNYKTTPPVLDSD | | YSNSLT | | ACEVTHQGLSSP | | GQGTKL |
| | | | GSFFLYSKLTVDKSRWQQGNVF | | YWGQG | | VTKSFNRGEC | | EIK |
| | | | SCSVMHEALHNHYTQKSLSLSPG | | TLVTVS | | | | |
| | | | K | | S | | | | |
| EPI1261 | HER3 | 97 | EVQLLESGGGLVQPGGSLRLSCA | 98 | EVQLLE | 99 | QSVLTQPPSASG | 100 | QSVLTQ |
| | | | ASGFTFSDYDMSWVRQAPGKGL | | SGGGLV | | TPGQRVTISCSG | | PPSASGT |
| | | | EWVSTIDLDSGSIYYADSVQGRF | | QPGGSL | | SSSNIGSNSVSW | | PGQRVTI |
| | | | TISRDNSKNTLYLQMNSLRAEDT | | RLSCAA | | YQQLPGTAPKL | | SCSGSSS |
| | | | AVYYCAKDLHMGPEGPFDYWG | | SGFTFSD | | LIYSDNHRPSGV | | NIGSNSV |
| | | | QGTLVTVSSASTKGPSVFPLAPSS | | YDMSW | | PDRFSGSKSGTS | | SWYQQL |
| | | | KSTSGGTAALGCLVKDYFPEPVT | | VRQAPG | | ASLAISGLRSED | | PGTAPK |
| | | | VSWNSGALTSGVHTFPAVLQSS | | KGLEWV | | EADYYCQGWD | | LLIYSDN |
| | | | GLYSLSSVVTVPSSSLGTQTYICN | | STIDLDS | | TSLSGHVFGGGT | | HRPSGV |
| | | | VNHKPSNTKVDKKVEPKSCDKT | | GSIYYA | | KLTVLRTVAAPS | | PDRFSGS |
| | | | HTCPPCPAPELLGGPSVFLFPPKP | | DSVQGR | | VFIFPPSDEQLKS | | KSGTSA |
| | | | KDTLMISRTPEVTCVVVDVSHED | | FTISRDN | | GTASVVCLLNN | | SLAISGL |
| | | | PEVKFNWYVDGVEVHNAKTKP | | SKNTLY | | FYPREAKVQWK | | RSEDEA |
| | | | REEQYNSTYRVVSVLTVLHQDW | | LQMNSL | | VDNALQSGNSQ | | DYYCQG |
| | | | LNGKEYKCKVSNKALPAPIEKTI | | RAEDTA | | ESVTEQDSKDST | | WDTSLS |
| | | | SKAKGQPREPQVYTLPPSRDELT | | VYYCAK | | YSLSSTLTLSKA | | GHVFGG |
| | | | KNQVSLWCLVKGFYPSDIAVEW | | DLHMGP | | DYEKHKVYACE | | GTKLTV |
| | | | ESNGQPENNYKTTPPVLDSDGSF | | EGPFDY | | VTHQGLSSPVTK | | L |
| | | | FLYSKLTVDKSRWQQGNVFSCS | | WGQGTL | | SFNRGEC | | |
| | | | VMHEALHNHYTQKSLSLSPGK | | VTVSS | | | | |
| EPI1262 | ADAM9 | 101 | EVQLVESGGGLVKPGGSLRLSCA | 102 | EVQLVE | 103 | DIVMTQSPDSLA | 104 | DIVMTQ |
| | | | ASGFTFSSYWMHWVRQAPGKG | | SGGGLV | | VSLGERATISCK | | SPDSLA |
| | | | LEWVGEIIPIFGHTNYNEKFKSRF | | KPGGSL | | ASQSVDYSGDS | | VSLGER |
| | | | TISLDNSKNTLYLQMGSLRAEDT | | RLSCAA | | YMNWYQQKPG | | ATISCKA |
| | | | AVYYCARGGYYYYPRQGFLDY | | SGFTFSS | | QPPKLLIYAASD | | SQSVDY |
| | | | WGQGTTVTVSSASTKGPSVFPLA | | YWMHW | | LESGIPARFSGS | | SGDSYM |
| | | | PSSKSTSGGTAALGCLVKDYFPE | | VRQAPG | | GSGTDFTLTISSL | | NWYQQ |
| | | | PVTVSWNSGALTSGVHTFPAVL | | KGLEWV | | EPEDFATYYCQ | | KPGQPP |
| | | | QSSGLYSLSSVVTVPSSSLGTQTY | | GEIIPIFG | | QSHEDPFTFGQG | | KLLIYA |
| | | | ICNVNHKPSNTKVDKKVEPKSC | | HTNYNE | | TKLEIKRTVAAP | | ASDLES |
| | | | DKTHTCPPCPAPELLGGPSVFLFP | | KFKSRF | | SVFIFPPSDEQLK | | GIPARFS |
| | | | PKPKDTLMISRTPEVTCVVVDVS | | TISLDNS | | SGTASVVCLLN | | GSGSGT |
| | | | HEDPEVKFNWYVDGVEVHNAK | | KNTLYL | | NFYPREAKVQW | | DFTLTIS |
| | | | TKPREEQYNSTYRVVSVLTVLH | | QMGSLR | | KVDNALQSGNS | | SLEPEDF |
| | | | QDWLNGKEYKCKVSNKALPAPI | | AEDTAV | | QESVTEQDSKDS | | ATYYCQ |
| | | | EKTISKAKGQPREPQVYTLPPSR | | YYCARG | | TYSLSSTLTLSK | | QSHEDP |
| | | | DELTKNQVSLWCLVKGFYPSDIA | | GYYYYP | | ADYEKHKVYAC | | FTFGQG |
| | | | VEWESNGQPENNYKTTPPVLDS | | RQGFLD | | EVTHQGLSSPVT | | TKLEIK |
| | | | DGSFFLYSKLTVDKSRWQQGNV | | YWGQG | | KSFNRGEC | | |
| | | | FSCSVMHEALHNHYTQKSLSLSP | | TTVTVS | | | | |
| | | | GK | | S | | | | |
| EPI1263 | CDH3 | 105 | EVQLLESGGGLVQPGGSLRLSCA | 106 | EVQLLE | 107 | QSALTQPASVSG | 108 | QSALTQ |
| EPI2215 | | | ASGFTFSSYAMSWVRQAPGKGL | | SGGGLV | | SPGQSITISCTGT | | PASVSG |
| EPI1606 | | | EWVSAISGSGGSTYYADSVKGRF | | QPGGSL | | SNDVGAYNYVS | | SPGQSIT |
| | | | TISRDNSKNTLYLQMNSLRAEDT | | RLSCAA | | WYQQHPGKAPK | | ISCTGTS |
| | | | AVYYCAKTNSAKFDPWGQGTM | | SGFTFSS | | LMISEVNKRPSG | | NDVGAY |
| | | | VTVSSASTKGPSVFPLAPSSKSTS | | YAMSW | | VSNRFSGSKSGN | | NYVSW |
| | | | GGTAALGCLVKDYFPEPVTVSW | | VRQAPG | | TASLTISGLQAE | | YQQHPG |
| | | | NSGALTSGVHTFPAVLQSSGLYS | | KGLEWV | | DEADYYCSSYT | | KAPKLM |
| | | | LSSVVTVPSSSLGTQTYICNVNH | | SAISGSG | | MGSTFMLFGGG | | ISEVNKR |
| | | | KPSNTKVDKKVEPKSCDKTHTC | | GSTYYA | | TKLTVLRTVAA | | PSGVSN |
| | | | PPCPAPELLGGPSVFLFPPKPKDT | | DSVKGR | | PSVFIFPPSDEQL | | RFSGSKS |
| | | | LMISRTPEVTCVVVDVSHEDPEV | | FTISRDN | | KSGTASVVCLL | | GNTASL |
| | | | KFNWYVDGVEVHNAKTKPREE | | SKNTLY | | NNFYPREAKVQ | | TISGLQA |
| | | | QYNSTYRVVSVLTVLHQDWLNG | | LQMNSL | | WKVDNALQSG | | EDEADY |
| | | | KEYKCKVSNKALPAPIEKTISKA | | RAEDTA | | NSQESVTEQDSK | | YCSSYT |
| | | | KGQPREPQVYTLPPSRDELTKNQ | | VYYCAK | | DSTYSLSSTLTL | | MGSTFM |
| | | | VSLWCLVKGFYPSDIAVEWESN | | TNSAKF | | SKADYEKHKVY | | LFGGGT |
| | | | GQPENNYKTTPPVLDSDGSFFLY | | DPWGQ | | ACEVTHQGLSSP | | KLTVL |
| | | | SKLTVDKSRWQQGNVFSCSVMH | | GTMVTV | | VTKSFNRGEC | | |
| | | | EALHNHYTQKSLSLSPGK | | SS | | | | |
| EPI1264 | CDH3 | 109 | QVQLVESGGGVVQPGRSLRLSC | 110 | QVQLVE | 111 | DIQLTQSPSSLSA | 112 | DIQLTQS |
| EPI2216 | | | AASGFSLTSYGVHWVRQAPGKG | | SGGGVV | | SVGDRVTITCRA | | PSSLSAS |
| EPI1607 | | | LEWVGVIWSGGSTDYADSVKGR | | QPGRSL | | SQNIYSNLAWY | | VGDRVT |
| | | | FTISKDNSKNTVYLQMNSLRAED | | RLSCAA | | QQKPGKAPKLL | | ITCRASQ |
| | | | TAVYYCARNSNNGFAYWGQGT | | SGFSLTS | | VYAAKNLASGV | | NIYSNL |
| | | | LVTVSSASTKGPSVFPLAPSSKST | | YGVHW | | PSRFSGSGSGTD | | AWYQQ |
| | | | SGGTAALGCLVKDYFPEPVTVS | | VRQAPG | | FTLTISSLQPEDF | | KPGKAP |
| | | | WNSGALTSGVHTFPAVLQSSGL | | KGLEWV | | ATYYCQHFYDT | | KLLVYA |
| | | | YSLSSVVTVPSSSLGTQTYICNVN | | GVIWSG | | PWTFGQGTKVEI | | AKNLAS |
| | | | HKPSNTKVDKKVEPKSCDKTHT | | GSTDYA | | KRTVAAPSVFIF | | GVPSRFS |
| | | | CPPCPAPELLGGPSVFLFPPKPKD | | DSVKGR | | PPSDEQLKSGTA | | GSGSGT |
| | | | TLMISRTPEVTCVVVDVSHEDPE | | FTISKDN | | SVVCLLNNFYPR | | DFTLTIS |
| | | | VKFNWYVDGVEVHNAKTKPRE | | SKNTVY | | EAKVQWKVDN | | SLQPEDF |
| | | | EQYNSTYRVVSVLTVLHQDWLN | | LQMNSL | | ALQSGNSQESVT | | ATYYCQ |
| | | | GKEYKCKVSNKALPAPIEKTISK | | RAEDTA | | EQDSKDSTYSLS | | HFYDTP |
| | | | AKGQPREPQVYTLPPSRDELTKN | | VYYCAR | | STLTLSKADYEK | | WTFGQG |
| | | | QVSLWCLVKGFYPSDIAVEWES | | NSNNGF | | HKVYACEVTHQ | | TKVEIK |
| | | | NGQPENNYKTTPPVLDSDGSFFL | | AYWGQ | | GLSSPVTKSFNR | | |
| | | | YSKLTVDKSRWQQGNVFSCSVM | | GTLVTV | | GEC | | |
| | | | HEALHNHYTQKSLSLSPGK | | SS | | | | |
| EPI1265 | CDH3 | 113 | QVQLQESGPELVKPGASVKMSC | 114 | QVQLQE | 115 | DIQMTQTTSSLS | 116 | DIQMTQ |
| EPI2217 | | | KASGYSFTAYNMHWVKQSHGK | | SGPELV | | ASLGDRVTISCR | | TTSSLSA |
| EPI1734 | | | SLEWIGFIDPYSGIITYNQTFKGK | | KPGASV | | ASQDITNYLNW | | SLGDRV |
| | | | ATLTVDKSSSTAYMQLNSLTSED | | KMSCKA | | YQQKPDGTVKL | | TISCRAS |
| | | | SAVYYCARRGYYDGGFDYWGQ | | SGYSFT | | LIYYTSRLHSGV | | QDITNY |
| | | | GTTLTVSSASTKGPSVFPLAPSSK | | AYNMH | | PSRFSGSGSGTD | | LNWYQ |
| | | | STSGGTAALGCLVKDYFPEPVTV | | WVKQS | | YSLTISNLEQEDI | | QKPDGT |
| | | | SWNSGALTSGVHTFPAVLQSSGL | | HGKSLE | | ATYFCQQDSKH | | VKLLIY |
| | | | YSLSSVVTVPSSSLGTQTYICNVN | | WIGFIDP | | PRTFGGGTKLEI | | YTSRLH |
| | | | HKPSNTKVDKKVEPKSCDKTHT | | YSGIITY | | KRTVAAPSVFIF | | SGVPSRF |
| | | | CPPCPAPELLGGPSVFLFPPKPKD | | NQTFKG | | PPSDEQLKSGTA | | SGSGSG |
| | | | TLMISRTPEVTCVVVDVSHEDPE | | KATLTV | | SVVCLLNNFYPR | | TDYSLTI |
| | | | VKFNWYVDGVEVHNAKTKPRE | | DKSSST | | EAKVQWKVDN | | SNLEQE |
| | | | EQYNSTYRVVSVLTVLHQDWLN | | AYMQL | | ALQSGNSQESVT | | DIATYFC |
| | | | GKEYKCKVSNKALPAPIEKTISK | | NSLTSE | | EQDSKDSTYSLS | | QQDSKH |
| | | | AKGQPREPQVYTLPPSRDELTKN | | DSAVYY | | STLTLSKADYEK | | PRTFGG |
| | | | QVSLWCLVKGFYPSDIAVEWES | | CARRGY | | HKVYACEVTHQ | | GTKLEIK |
| | | | NGQPENNYKTTPPVLDSDGSFFL | | YDGGFD | | GLSSPVTKSFNR | | |
| | | | YSKLTVDKSRWQQGNVFSCSVM | | YWGQG | | GEC | | |
| | | | HEALHNHYTQKSLSLSPGK | | TTLTVSS | | | | |
| EPI1266 | CDH3 | 117 | QVQLQQPGTELVKPGASVKLSC | 118 | QVQLQQ | 119 | ENVLTQSPAIMA | 120 | ENVLTQ |
| EPI1735 | | | KASGYTFTRYWINWVKQRPQGG | | PGTELV | | ASPGEKVTMTC | | SPAIMA |
| | | | LEWIGNIYPGSNITNYNEKFKNK | | KPGASV | | SASSSVSSGNFH | | ASPGEK |
| | | | ATLTVDTSSNTAYMQLSSLTSDD | | KLSCKA | | WYQQKPGTSPK | | VTMTCS |
| | | | SAVYYCAREGIYDGYFPLFPYW | | SGYTFT | | LWIYRTSNLASG | | ASSSVSS |
| | | | GQGTLVTVSAASTKGPSVFPLAP | | RYWINW | | VPARFSGSGSGT | | GNFHW |
| | | | SSKSTSGGTAALGCLVKDYFPEP | | VKQRPQ | | SYSLTISSMEAE | | YQQKPG |
| | | | VTVSWNSGALTSGVHTFPAVLQ | | GGLEWI | | DAATYYCQQWS | | TSPKLWI |
| | | | SSGLYSLSSVVTVPSSSLGTQTYI | | GNIYPGS | | GYPWTFGGGTK | | YRTSNL |
| | | | CNVNHKPSNTKVDKKVEPKSCD | | NITNYN | | LEIKRTVAAPSV | | ASGVPA |
| | | | KTHTCPPCPAPELLGGPSVFLFPP | | EKFKNK | | FIFPPSDEQLKSG | | RFSGSGS |
| | | | KPKDTLMISRTPEVTCVVVDVSH | | ATLTVD | | TASVVCLLNNF | | GTSYSL |
| | | | EDPEVKFNWYVDGVEVHNAKT | | TSSNTA | | YPREAKVQWKV | | TISSMEA |
| | | | KPREEQYNSTYRVVSVLTVLHQ | | YMQLSS | | DNALQSGNSQE | | EDAATY |
| | | | DWLNGKEYKCKVSNKALPAPIE | | LTSDDS | | SVTEQDSKDSTY | | YCQQWS |
| | | | KTISKAKGQPREPQVYTLPPSRD | | AVYYCA | | SLSSTLTLSKAD | | GYPWTF |
| | | | ELTKNQVSLWCLVKGFYPSDIAV | | REGIYD | | YEKHKVYACEV | | GGGTKL |
| | | | EWESNGQPENNYKTTPPVLDSD | | GYFPLFP | | THQGLSSPVTKS | | EIK |
| | | | GSFFLYSKLTVDKSRWQQGNVF | | YWGQG | | FNRGEC | | |
| | | | SCSVMHEALHNHYTQKSLSLSPG | | TLVTVS | | | | |
| | | | K | | A | | | | |
| EPI1267 | CDH3 | 121 | QVQLQQSGPGLVKPSQTLSLTCA | 122 | QVQLQQ | 123 | DIQMTQSPSSLS | 124 | DIQMTQ |
| EPI1736 | | | ISGDSVSSQSAAWNWIRQSPSRG | | SGPGLV | | ASVGDRVTITCR | | SPSSLSA |
| | | | LEWLGRIYYRSKWYNDYALSVK | | KPSQTLS | | ASQTISNTLAWY | | SVGDRV |
| | | | SRITINPDTSKNQFSLQLNSVTPE | | LTCAISG | | QQKPGKAPKLLI | | TITCRAS |
| | | | DTAVYYCARGEGYGREGFAIWG | | DSVSSQ | | YAASNLQSGVP | | QTISNTL |
| | | | QGTLVTVSSASTKGPSVFPLAPSS | | SAAWN | | SRFSGSGSGTDF | | AWYQQ |
| | | | KSTSGGTAALGCLVKDYFPEPVT | | WIRQSPS | | TLTISSLQPEDFA | | KPGKAP |
| | | | VSWNSGALTSGVHTFPAVLQSS | | RGLEWL | | TYYCQQYLSWF | | KLLIYA |
| | | | GLYSLSSVVTVPSSSLGTQTYICN | | GRIYYR | | TFGQGTKVEIKR | | ASNLQS |
| | | | VNHKPSNTKVDKKVEPKSCDKT | | SKWYN | | TVAAPSVFIFPPS | | GVPSRFS |
| | | | HTCPPCPAPELLGGPSVFLFPPKP | | DYALSV | | DEQLKSGTASV | | GSGSGT |
| | | | KDTLMISRTPEVTCVVVDVSHED | | KSRITIN | | VCLLNNFYPRE | | DFTLTIS |
| | | | PEVKFNWYVDGVEVHNAKTKP | | PDTSKN | | AKVQWKVDNA | | SLQPEDF |
| | | | REEQYNSTYRVVSVLTVLHQDW | | QFSLQL | | LQSGNSQESVTE | | ATYYCQ |
| | | | LNGKEYKCKVSNKALPAPIEKTI | | NSVTPE | | QDSKDSTYSLSS | | QYLSWF |
| | | | SKAKGQPREPQVYTLPPSRDELT | | DTAVYY | | TLTLSKADYEK | | TFGQGT |
| | | | KNQVSLWCLVKGFYPSDIAVEW | | CARGEG | | HKVYACEVTHQ | | KVEIK |
| | | | ESNGQPENNYKTTPPVLDSDGSF | | YGREGF | | GLSSPVTKSFNR | | |
| | | | FLYSKLTVDKSRWQQGNVFSCS | | AIWGQG | | GEC | | |
| | | | VMHEALHNHYTQKSLSLSPGK | | TLVTVS | | | | |
| | | | | | S | | | | |
| EPI1268 | EpCAM | 125 | QVQLVQSGPEVKKPGASVKVSC | 126 | QVQLVQ | 127 | DIVMTQSPLSLP | 128 | DIVMTQ |
| EPI1147 | | | KASGYTFTNYGMNWVRQAPGQ | | SGPEVK | | VTPGEPASISCRS | | SPLSLPV |
| | | | GLEWMGWINTYTGEPTYGEDFK | | KPGASV | | SKNLLHSNGITY | | TPGEPAS |
| | | | GRFAFSLDTSASTAYMELSSLRS | | KVSCKA | | LYWYLQKPGQS | | ISCRSSK |
| | | | EDTAVYFCARFGNYVDYWGQG | | SGYTFT | | PQLLIYQMSNLA | | NLLHSN |
| | | | SLVTVSSASTKGPSVFPLAPSSKS | | NYGMN | | SGVPDRFSSSGS | | GITYLY |
| | | | TSGGTAALGCLVKDYFPEPVTVS | | WVRQAP | | GTDFTLKISRVE | | WYLQKP |
| | | | WNSGALTSGVHTFPAVLQSSGL | | GQGLEW | | AEDVGVYYCAQ | | GQSPQL |
| | | | YSLSSVVTVPSSSLGTQTYICNVN | | MGWINT | | NLEIPRTFGQGT | | LIYQMS |
| | | | HKPSNTKVDKKVEPKSCDKTHT | | YTGEPT | | KVEIKRTVAAPS | | NLASGV |
| | | | CPPCPAPELLGGPSVFLFPPKPKD | | YGEDFK | | VFIFPPSDEQLKS | | PDRFSSS |
| | | | TLMISRTPEVTCVVVDVSHEDPE | | GRFAFS | | GTASVVCLLNN | | GSGTDF |
| | | | VKFNWYVDGVEVHNAKTKPRE | | LDTSAS | | FYPREAKVQWK | | TLKISRV |
| | | | EQYNSTYRVVSVLTVLHQDWLN | | TAYMEL | | VDNALQSGNSQ | | EAEDVG |
| | | | GKEYKCKVSNKALPAPIEKTISK | | SSLRSED | | ESVTEQDSKDST | | VYYCAQ |
| | | | AKGQPREPQVYTLPPSRDELTKN | | TAVYFC | | YSLSSTLTLSKA | | NLEIPRT |
| | | | QVSLWCLVKGFYPSDIAVEWES | | ARFGNY | | DYEKHKVYACE | | FGQGTK |
| | | | NGQPENNYKTTPPVLDSDGSFFL | | VDYWG | | VTHQGLSSPVTK | | VEIK |
| | | | YSKLTVDKSRWQQGNVFSCSVM | | QGSLVT | | SFNRGEC | | |
| | | | HEALHNHYTQKSLSLSPGK | | VSS | | | | |
| EPI1269 | EpCAM | 129 | QIQLVQSGPELKKPGETVKISCK | 130 | QIQLVQ | 131 | DIVMTQAAFSNP | 132 | DIVMTQ |
| EPI1148 | | | ASGYTFTKYGMNWVKQAPGKG | | SGPELK | | VTLGTSGSISCR | | AAFSNP |
| | | | LKWMGWINTYTEEPTYGDDFKG | | KPGETV | | SSKSLLHSNGIT | | VTLGTS |
| | | | RFAFSLETSASTANLQINNLKSED | | KISCKAS | | YLYWYLQKPGQ | | GSISCRS |
| | | | TATYFCARFGSAVDYWGQGTSV | | GYTFTK | | SPQLLIYQMSNL | | SKSLLHS |
| | | | TVSSASTKGPSVFPLAPSSKSTSG | | YGMNW | | ASGVPDRFSSSG | | NGITYL |
| | | | GTAALGCLVKDYFPEPVTVSWN | | VKQAPG | | SGTDFTLRISRV | | YWYLQ |
| | | | SGALTSGVHTFPAVLQSSGLYSL | | KGLKW | | EAEDVGVYYCA | | KPGQSP |
| | | | SSVVTVPSSSLGTQTYICNVNHK | | MGWINT | | QNLELPRTFGGG | | QLLIYQ |
| | | | PSNTKVDKKVEPKSCDKTHTCPP | | YTEEPT | | TKLEIKRTVAAP | | MSNLAS |
| | | | CPAPELLGGPSVFLFPPKPKDTL | | YGDDFK | | SVFIFPPSDEQLK | | GVPDRF |
| | | | MISRTPEVTCVVVDVSHEDPEVK | | GRFAFS | | SGTASVVCLLN | | SSSGSGT |
| | | | FNWYVDGVEVHNAKTKPREEQ | | LETSAST | | NFYPREAKVQW | | DFTLRIS |
| | | | YNSTYRVVSVLTVLHQDWLNGK | | ANLQIN | | KVDNALQSGNS | | RVEAED |
| | | | EYKCKVSNKALPAPIEKTISKAK | | NLKSED | | QESVTEQDSKDS | | VGVYYC |
| | | | GQPREPQVYTLPPSRDELTKNQV | | TATYFC | | TYSLSSTLTLSK | | AQNLEL |
| | | | SLWCLVKGFYPSDIAVEWESNG | | ARFGSA | | ADYEKHKVYAC | | PRTFGG |
| | | | QPENNYKTTPPVLDSDGSFFLYS | | VDYWG | | EVTHQGLSSPVT | | GTKLEIK |
| | | | KLTVDKSRWQQGNVFSCSVMHE | | QGTSVT | | KSFNRGEC | | |
| | | | ALHNHYTQKSLSLSPGK | | VSS | | | | |
| EPI1270 | EpCAM | 133 | EVQLVQSGPGLVQPGGSVRISCA | 134 | EVQLVQ | 135 | DIQMTQSPSSLS | 136 | DIQMTQ |
| EPI1149 | | | ASGYTFTNYGMNWVKQAPGKG | | SGPGLV | | ASVGDRVTITCR | | SPSSLSA |
| | | | LEWMGWINTYTGESTYADSFKG | | QPGGSV | | STKSLLHSNGIT | | SVGDRV |
| | | | RFTFSLDTSASAAYLQINSLRAED | | RISCAAS | | YLYWYQQKPG | | TITCRST |
| | | | TAVYYCARFAIKGDYWGQGTLL | | GYTFTN | | KAPKLLIYQMS | | KSLLHS |
| | | | TVSSASTKGPSVFPLAPSSKSTSG | | YGMNW | | NLASGVPSRFSS | | NGITYL |
| | | | GTAALGCLVKDYFPEPVTVSWN | | VKQAPG | | SGSGTDFTLTISS | | YWYQQ |
| | | | SGALTSGVHTFPAVLQSSGLYSL | | KGLEW | | LQPEDFATYYC | | KPGKAP |
| | | | SSVVTVPSSSLGTQTYICNVNHK | | MGWINT | | AQNLEIPRTFGQ | | KLLIYQ |
| | | | PSNTKVDKKVEPKSCDKTHTCPP | | YTGEST | | GTKVELKRTVA | | MSNLAS |
| | | | CPAPELLGGPSVFLFPPKPKDTL | | YADSFK | | APSVFIFPPSDEQ | | GVPSRFS |
| | | | MISRTPEVTCVVVDVSHEDPEVK | | GRFTFSL | | LKSGTASVVCLL | | SSGSGT |
| | | | FNWYVDGVEVHNAKTKPREEQ | | DTSASA | | NNFYPREAKVQ | | DFTLTIS |
| | | | YNSTYRVVSVLTVLHQDWLNGK | | AYLQIN | | WKVDNALQSG | | SLQPEDF |
| | | | EYKCKVSNKALPAPIEKTISKAK | | SLRAED | | NSQESVTEQDSK | | ATYYCA |
| | | | GQPREPQVYTLPPSRDELTKNQV | | TAVYYC | | DSTYSLSSTLTL | | QNLEIPR |
| | | | SLWCLVKGFYPSDIAVEWESNG | | ARFAIK | | SKADYEKHKVY | | TFGQGT |
| | | | QPENNYKTTPPVLDSDGSFFLYS | | GDYWG | | ACEVTHQGLSSP | | KVELK |
| | | | KLTVDKSRWQQGNVFSCSVMHE | | QGTLLT | | VTKSFNRGEC | | |
| | | | ALHNHYTQKSLSLSPGK | | VSS | | | | |
| EPI1271 | EpCAM | 137 | QVQLVQSGAEVKKPGASVKVSC | 138 | QVQLVQ | 139 | EIELTQSPGTLSL | 140 | EIELTQS |
| | | | KASGGTFSSYAISWVRQAPGQGL | | SGAEVK | | SPGERATLSCRA | | PGTLSLS |
| | | | EWMGGIVPIFGTANYAQKFQGR | | KPGASV | | SQSVSSSYLAW | | PGERAT |
| | | | VTITADESTSTAYMELSSLRSEDT | | KVSCKA | | YQQKPGQAPRL | | LSCRAS |
| | | | AVYYCARDPFLHYWGQGTLVT | | SGGTFSS | | LIYGASSRATGIP | | QSVSSS |
| | | | ASTKGPSVFPLAPSSKSTSGGTA | | YAISWV | | DRFSGSGSGTDF | | YLAWY |
| | | | ALGCLVKDYFPEPVTVSWNSGA | | RQAPGQ | | TLTISRLEPEDFA | | QQKPGQ |
| | | | LTSGVHTFPAVLQSSGLYSLSSV | | GLEWM | | VYYCAQGELYP | | APRLLIY |
| | | | VTVPSSSLGTQTYICNVNHKPSN | | GGIVPIF | | RQFGGGTKLDIR | | GASSRA |
| | | | TKVDKKVEPKSCDKTHTCPPCPA | | GTANYA | | TVAAPSVFIFPPS | | TGIPDRF |
| | | | PELLGGPSVFLFPPKPKDTLMISR | | QKFQGR | | DEQLKSGTASV | | SGSGSG |
| | | | TPEVTCVVVDVSHEDPEVKFNW | | VTITADE | | VCLLNNFYPRE | | TDFTLTI |
| | | | YVDGVEVHNAKTKPREEQYNST | | STSTAY | | AKVQWKVDNA | | SRLEPED |
| | | | YRVVSVLTVLHQDWLNGKEYK | | MELSSL | | LQSGNSQESVTE | | FAVYYC |
| | | | CKVSNKALPAPIEKTISKAKGQP | | RSEDTA | | QDSKDSTYSLSS | | AQGELY |
| | | | REPQVYTLPPSRDELTKNQVSLW | | VYYCAR | | TLTLSKADYEK | | PRQFGG |
| | | | CLVKGFYPSDIAVEWESNGQPEN | | DPFLHY | | HKVYACEVTHQ | | GTKLDI |
| | | | NYKTTPPVLDSDGSFFLYSKLTV | | WGQGTL | | GLSSPVTKSFNR | | |
| | | | DKSRWQQGNVFSCSVMHEALH | | VT | | GEC | | |
| | | | NHYTQKSLSLSPGK | | | | | | |
| EPI1272 | EpCAM | 141 | QVQLVQSGAEVKKPGSSVKVSC | 142 | QVQLVQ | 143 | EIVMTQSPATLS | 144 | EIVMTQ |
| EPI1150 | | | KASGGTFSSYAISWVRQAPGQGL | | SGAEVK | | VSPGERATLSCR | | SPATLSV |
| | | | EWMGGIIPIFGTANYAQKFQGRV | | KPGSSV | | ASQSVSSNLAW | | SPGERA |
| | | | TITADESTSTAYMELSSLRSEDTA | | KVSCKA | | YQQKPGQAPRLI | | TLSCRA |
| | | | VYYCARGLLWNYWGQGTLVTV | | SGGTFSS | | IYGASTTASGIP | | SQSVSS |
| | | | SSASTKGPSVFPLAPSSKSTSGGT | | YAISWV | | ARFSASGSGTDF | | NLAWY |
| | | | AALGCLVKDYFPEPVTVSWNSG | | RQAPGQ | | TLTISSLQSEDFA | | QQKPGQ |
| | | | ALTSGVHTFPAVLQSSGLYSLSS | | GLEWM | | VYYCQQYNNW | | APRLIIY |
| | | | VVTVPSSSLGTQTYICNVNHKPS | | GGIIPIFG | | PPAYTFGQGTKL | | GASTTA |
| | | | NTKVDKKVEPKSCDKTHTCPPCP | | TANYAQ | | EIKRTVAAPSVFI | | SGIPARF |
| | | | APELLGGPSVFLFPPKPKDTLMIS | | KFQGRV | | FPPSDEQLKSGT | | SASGSG |
| | | | RTPEVTCVVVDVSHEDPEVKFN | | TITADES | | ASVVCLLNNFY | | TDFTLTI |
| | | | WYVDGVEVHNAKTKPREEQYN | | TSTAYM | | PREAKVQWKVD | | SSLQSED |
| | | | STYRVVSVLTVLHQDWLNGKEY | | ELSSLRS | | NALQSGNSQES | | FAVYYC |
| | | | KCKVSNKALPAPIEKTISKAKGQ | | EDTAVY | | VTEQDSKDSTYS | | QQYNN |
| | | | PREPQVYTLPPSRDELTKNQVSL | | YCARGL | | LSSTLTLSKADY | | WPPAYT |
| | | | WCLVKGFYPSDIAVEWESNGQP | | LWNYW | | EKHKVYACEVT | | FGQGTK |
| | | | ENNYKTTPPVLDSDGSFFLYSKL | | GQGTLV | | HQGLSSPVTKSF | | LEIK |
| | | | TVDKSRWQQGNVFSCSVMHEAL | | TVSS | | NRGEC | | |
| | | | HNHYTQKSLSLSPGK | | | | | | |
| EPI1273 | TNFR | 145 | EVQLVESGGGLVQPGGSLRLSCA | 146 | EVQLVE | 147 | DIQMTQSPSSLS | 148 | DIQMTQ |
| EPI1151 | SF10B | | ASGFTFSSYVMSWVRQAPGKGL | | SGGGLV | | ASVGDRVTITCK | | SPSSLSA |
| | | | EWVATISSGGSYTYYPDSVKGRF | | QPGGSL | | ASQDVGTAVA | | SVGDRV |
| | | | TISRDNAKNTLYLQMNSLRAEDT | | RLSCAA | | WYQQKPGKAPK | | TITCKAS |
| | | | AVYYCARRGDSMITTDYWGQG | | SGFTFSS | | LLIYWASTRHTG | | QDVGTA |
| | | | TLVTVSSASTKGPSVFPLAPSSKS | | YVMSW | | VPSRFSGSGSGT | | VAWYQ |
| | | | TSGGTAALGCLVKDYFPEPVTVS | | VRQAPG | | DFTLTISSLQPED | | QKPGKA |
| | | | WNSGALTSGVHTFPAVLQSSGL | | KGLEWV | | FATYYCQQYSS | | PKLLIY |
| | | | YSLSSVVTVPSSSLGTQTYICNVN | | ATISSGG | | YRTFGQGTKVEI | | WASTRH |
| | | | HKPSNTKVDKKVEPKSCDKTHT | | SYTYYP | | KRTVAAPSVFIF | | TGVPSR |
| | | | CPPCPAPELLGGPSVFLFPPKPKD | | DSVKGR | | PPSDEQLKSGTA | | FSGSGS |
| | | | TLMISRTPEVTCVVVDVSHEDPE | | FTISRDN | | SVVCLLNNFYPR | | GTDFTL |
| | | | VKFNWYVDGVEVHNAKTKPRE | | AKNTLY | | EAKVQWKVDN | | TISSLQP |
| | | | EQYNSTYRVVSVLTVLHQDWLN | | LQMNSL | | ALQSGNSQESVT | | EDFATY |
| | | | GKEYKCKVSNKALPAPIEKTISK | | RAEDTA | | EQDSKDSTYSLS | | YCQQYS |
| | | | AKGQPREPQVYTLPPSRDELTKN | | VYYCAR | | STLTLSKADYEK | | SYRTFG |
| | | | QVSLWCLVKGFYPSDIAVEWES | | RGDSMI | | HKVYACEVTHQ | | QGTKVE |
| | | | NGQPENNYKTTPPVLDSDGSFFL | | TTDYWG | | GLSSPVTKSFNR | | IK |
| | | | YSKLTVDKSRWQQGNVFSCSVM | | QGTLVT | | GEC | | |
| | | | HEALHNHYTQKSLSLSPGK | | VSS | | | | |
| EPI1274 | TNFR | 149 | EVQLVQSGGGVERPGGSLRLSC | 150 | EVQLVQ | 151 | SELTQDPAVSVA | 152 | SELTQD |
| EPI1152 | SF10B | | AASGFTFDDYAMSWVRQAPGK | | SGGGVE | | LGQTVRITCSGD | | PAVSVA |
| | | | GLEWVSGINWQGGSTGYADSVK | | RPGGSL | | SLRSYYASWYQ | | LGQTVR |
| | | | GRVTISRDNAKNSLYLQMNSLR | | RLSCAA | | QKPGQAPVLVIY | | ITCSGDS |
| | | | AEDTAVYYCAKILGAGRGWYFD | | SGFTFD | | GANNRPSGIPDR | | LRSYYA |
| | | | YWGKGTTVTVSSASTKGPSVFPL | | DYAMS | | FSGSSSGNTASL | | SWYQQ |
| | | | APSSKSTSGGTAALGCLVKDYFP | | WVRQAP | | TITGAQAEDEAD | | KPGQAP |
| | | | EPVTVSWNSGALTSGVHTFPAVL | | GKGLEW | | YYCNSADSSGN | | VLVIYG |
| | | | QSSGLYSLSSVVTVPSSSLGTQTY | | VSGINW | | HVVFGGGTKLT | | ANNRPS |
| | | | ICNVNHKPSNTKVDKKVEPKSC | | QGGSTG | | VLRTVAAPSVFI | | GIPDRFS |
| | | | DKTHTCPPCPAPELLGGPSVFLFP | | YADSVK | | FPPSDEQLKSGT | | GSSSGN |
| | | | PKPKDTLMISRTPEVTCVVVDVS | | GRVTISR | | ASVVCLLNNFY | | TASLTIT |
| | | | HEDPEVKFNWYVDGVEVHNAK | | DNAKNS | | PREAKVQWKVD | | GAQAED |
| | | | TKPREEQYNSTYRVVSVLTVLH | | LYLQMN | | NALQSGNSQES | | EADYYC |
| | | | QDWLNGKEYKCKVSNKALPAPI | | SLRAED | | VTEQDSKDSTYS | | NSADSS |
| | | | EKTISKAKGQPREPQVYTLPPSR | | TAVYYC | | LSSTLTLSKADY | | GNHVVF |
| | | | DELTKNQVSLWCLVKGFYPSDIA | | AKILGA | | EKHKVYACEVT | | GGGTKL |
| | | | VEWESNGQPENNYKTTPPVLDS | | GRGWYF | | HQGLSSPVTKSF | | TVL |
| | | | DGSFFLYSKLTVDKSRWQQGNV | | DYWGK | | NRGEC | | |
| | | | FSCSVMHEALHNHYTQKSLSLSP | | GTTVTV | | | | |
| | | | GK | | SS | | | | |
| EPI1275 | TNFR | 153 | EVQLQQSGAEVVKPGASVKLSC | 154 | EVQLQQ | 155 | EIVMTQSPATLS | 156 | EIVMTQ |
| EPI1153 | SF10B | | KASGFNIKDTFIHWVKQAPGQGL | | SGAEVV | | VSPGERATLSCR | | SPATLSV |
| | | | EWIGRIDPANTNTKYDPKFQGKA | | KPGASV | | ASQSISNNLHW | | SPGERA |
| | | | TITTDTSSNTAYMELSSLRSEDTA | | KLSCKA | | YQQKPGQAPRL | | TLSCRA |
| | | | VYYCVRGLYTYYFDYWGQGTL | | SGFNIKD | | LIKFASQSITGIP | | SQSISNN |
| | | | VTVSSASTKGPSVFPLAPSSKSTS | | TFIHWV | | ARFSGSGSGTEF | | LHWYQ |
| | | | GGTAALGCLVKDYFPEPVTVSW | | KQAPGQ | | TLTISSLQSEDFA | | QKPGQA |
| | | | NSGALTSGVHTFPAVLQSSGLYS | | GLEWIG | | VYYCQQGNSWP | | PRLLIKF |
| | | | LSSVVTVPSSSLGTQTYICNVNH | | RIDPANT | | YTFGQGTKLEIK | | ASQSITG |
| | | | KPSNTKVDKKVEPKSCDKTHTC | | NTKYDP | | RTVAAPSVFIFPP | | IPARFSG |
| | | | PPCPAPELLGGPSVFLFPPKPKDT | | KFQGKA | | SDEQLKSGTASV | | SGSGTEF |
| | | | LMISRTPEVTCVVVDVSHEDPEV | | TITTDTS | | VCLLNNFYPRE | | TLTISSL |
| | | | KFNWYVDGVEVHNAKTKPREE | | SNTAYM | | AKVQWKVDNA | | QSEDFA |
| | | | QYNSTYRVVSVLTVLHQDWLNG | | ELSSLRS | | LQSGNSQESVTE | | VYYCQQ |
| | | | KEYKCKVSNKALPAPIEKTISKA | | EDTAVY | | QDSKDSTYSLSS | | GNSWPY |
| | | | KGQPREPQVYTLPPSRDELTKNQ | | YCVRGL | | TLTLSKADYEK | | TFGQGT |
| | | | VSLWCLVKGFYPSDIAVEWESN | | YTYYFD | | HKVYACEVTHQ | | KLEIK |
| | | | GQPENNYKTTPPVLDSDGSFFLY | | YWGQG | | GLSSPVTKSFNR | | |
| | | | SKLTVDKSRWQQGNVFSCSVMH | | TLVTVS | | GEC | | |
| | | | EALHNHYTQKSLSLSPGK | | S | | | | |
| EPI1276 | TNFR | 157 | QVQLQESGPGLVKPSQTLSLTCT | 158 | QVQLQE | 159 | EIVLTQSPGTLSL | 160 | EIVLTQS |
| EPI1154 | SF10B | | VSGGSISSGDYFWSWIRQLPGKG | | SGPGLV | | SPGERATLSCRA | | PGTLSLS |
| | | | LEWIGHIHNSGTTYYNPSLKSRV | | KPSQTLS | | SQGISRSYLAWY | | PGERAT |
| | | | TISVDTSKKQFSLRLSSVTAADT | | LTCTVS | | QQKPGQAPSLLI | | LSCRAS |
| | | | AVYYCARDRGGDYYYGMDVW | | GGSISSG | | YGASSRATGIPD | | QGISRSY |
| | | | GQGTTVTVSSASTKGPSVFPLAP | | DYFWS | | RFSGSGSGTDFT | | LAWYQ |
| | | | SSKSTSGGTAALGCLVKDYFPEP | | WIRQLP | | LTISRLEPEDFA | | QKPGQA |
| | | | VTVSWNSGALTSGVHTFPAVLQ | | GKGLEW | | VYYCQQFGSSP | | PSLLIYG |
| | | | SSGLYSLSSVVTVPSSSLGTQTYI | | IGHIHNS | | WTFGQGTKVEI | | ASSRAT |
| | | | CNVNHKPSNTKVDKKVEPKSCD | | GTTYYN | | KRTVAAPSVFIF | | GIPDRFS |
| | | | KTHTCPPCPAPELLGGPSVFLFPP | | PSLKSR | | PPSDEQLKSGTA | | GSGSGT |
| | | | KPKDTLMISRTPEVTCVVVDVSH | | VTISVDT | | SVVCLLNNFYPR | | DFTLTIS |
| | | | EDPEVKFNWYVDGVEVHNAKT | | SKKQFS | | EAKVQWKVDN | | RLEPEDF |
| | | | KPREEQYNSTYRVVSVLTVLHQ | | LRLSSVT | | ALQSGNSQESVT | | AVYYCQ |
| | | | DWLNGKEYKCKVSNKALPAPIE | | AADTAV | | EQDSKDSTYSLS | | QFGSSP |
| | | | KTISKAKGQPREPQVYTLPPSRD | | YYCARD | | STLTLSKADYEK | | WTFGQG |
| | | | ELTKNQVSLWCLVKGFYPSDIAV | | RGGDYY | | HKVYACEVTHQ | | TKVEIK |
| | | | EWESNGQPENNYKTTPPVLDSD | | YGMDV | | GLSSPVTKSFNR | | |
| | | | GSFFLYSKLTVDKSRWQQGNVF | | WGQGTT | | GEC | | |
| | | | SCSVMHEALHNHYTQKSLSLSPG | | VTVSS | | | | |
| | | | K | | | | | | |
| EPI1277 | TNFR | 161 | EVQLVQSGGGVERPGGSLRLSC | 162 | EVQLVQ | 163 | SSELTQDPAVSV | 164 | SSELTQ |
| EPI1155 | SF10B | | AASGFTFDDYGMSWVRQAPGK | | SGGGVE | | ALGQTVRITCQG | | DPAVSV |
| | | | GLEWVSGINWNGGSTGYADSVK | | RPGGSL | | DSLRSYYASWY | | ALGQTV |
| | | | GRVTISRDNAKNSLYLQMNSLR | | RLSCAA | | QQKPGQAPVLVI | | RITCQG |
| | | | AEDTAVYYCAKILGAGRGWYFD | | SGFTFD | | YGKNNRPSGIPD | | DSLRSY |
| | | | LWGKGTTVTVSSASTKGPSVFPL | | DYGMS | | RFSGSSSGNTAS | | YASWY |
| | | | APSSKSTSGGTAALGCLVKDYFP | | WVRQAP | | LTITGAQAEDEA | | QQKPGQ |
| | | | EPVTVSWNSGALTSGVHTFPAVL | | GKGLEW | | DYYCNSRDSSG | | APVLVI |
| | | | QSSGLYSLSSVVTVPSSSLGTQTY | | VSGINW | | NHVVFGGGTKL | | YGKNNR |
| | | | ICNVNHKPSNTKVDKKVEPKSC | | NGGSTG | | TVLRTVAAPSVF | | PSGIPDR |
| | | | DKTHTCPPCPAPELLGGPSVFLFP | | YADSVK | | IFPPSDEQLKSGT | | FSGSSSG |
| | | | PKPKDTLMISRTPEVTCVVVDVS | | GRVTISR | | ASVVCLLNNFY | | NTASLTI |
| | | | HEDPEVKFNWYVDGVEVHNAK | | DNAKNS | | PREAKVQWKVD | | TGAQAE |
| | | | TKPREEQYNSTYRVVSVLTVLH | | LYLQMN | | NALQSGNSQES | | DEADYY |
| | | | QDWLNGKEYKCKVSNKALPAPI | | SLRAED | | VTEQDSKDSTYS | | CNSRDS |
| | | | EKTISKAKGQPREPQVYTLPPSR | | TAVYYC | | LSSTLTLSKADY | | SGNHVV |
| | | | DELTKNQVSLWCLVKGFYPSDIA | | AKILGA | | EKHKVYACEVT | | FGGGTK |
| | | | VEWESNGQPENNYKTTPPVLDS | | GRGWYF | | HQGLSSPVTKSF | | LTVL |
| | | | DGSFFLYSKLTVDKSRWQQGNV | | DL WGK | | NRGEC | | |
| | | | FSCSVMHEALHNHYTQKSLSLSP | | GTTVTV | | | | |
| | | | GK | | SS | | | | |
| EPI1278 | ITGB6 | 165 | EVQLVESGGGLVQPGGSLRLSCA | 166 | EVQLVE | 167 | EIVLTQSPATLSL | 168 | EIVLTQS |
| EPI1164 | | | ASGFTFSRYVMSWVRQAPGKGL | | SGGGLV | | SPGERATLSCSA | | PATLSLS |
| | | | EWVASISSGGRMYYPDTVKGRF | | QPGGSL | | SSSVSSSYLYWY | | PGERAT |
| | | | TISRDNAKNSLYLQMNSLRAEDT | | RLSCAA | | QQKPGQAPRLLI | | LSCSASS |
| | | | AVYYCARGSIYDGYYVFPYWGQ | | SGFTFSR | | YSTSNLASGIPA | | SVSSSYL |
| | | | GTLVTVSSASTKGPSVFPLAPSSK | | YVMSW | | RFSGSGSGTDFT | | YWYQQ |
| | | | STSGGTAALGCLVKDYFPEPVTV | | VRQAPG | | LTISSLEPEDFAV | | KPGQAP |
| | | | SWNSGALTSGVHTFPAVLQSSGL | | KGLEWV | | YYCHQWSTYPP | | RLLIYST |
| | | | YSLSSVVTVPSSSLGTQTYICNVN | | ASISSGG | | TFGGGTKVEIKR | | SNLASGI |
| | | | HKPSNTKVDKKVEPKSCDKTHT | | RMYYPD | | TVAAPSVFIFPPS | | PARFSGS |
| | | | CPPCPAPELLGGPSVFLFPPKPKD | | TVKGRF | | DEQLKSGTASV | | GSGTDF |
| | | | TLMISRTPEVTCVVVDVSHEDPE | | TISRDNA | | VCLLNNFYPRE | | TLTISSL |
| | | | VKFNWYVDGVEVHNAKTKPRE | | KNSLYL | | AKVQWKVDNA | | EPEDFA |
| | | | EQYNSTYRVVSVLTVLHQDWLN | | QMNSLR | | LQSGNSQESVTE | | VYYCHQ |
| | | | GKEYKCKVSNKALPAPIEKTISK | | AEDTAV | | QDSKDSTYSLSS | | WSTYPP |
| | | | AKGQPREPQVYTLPPSRDELTKN | | YYCARG | | TLTLSKADYEK | | TFGGGT |
| | | | QVSLWCLVKGFYPSDIAVEWES | | SIYDGY | | HKVYACEVTHQ | | KVEIK |
| | | | NGQPENNYKTTPPVLDSDGSFFL | | YVFPYW | | GLSSPVTKSFNR | | |
| | | | YSKLTVDKSRWQQGNVFSCSVM | | GQGTLV | | GEC | | |
| | | | HEALHNHYTQKSLSLSPGK | | TVSS | | | | |
| EPI1279 | ITGB6 | 169 | QFQLVQSGAEVKKPGASVKVSC | 170 | QFQLVQ | 171 | DIQMTQSPSSLS | 172 | DIQMTQ |
| EPI1407 | | | KASGYSFTDYNVNWVRQAPGQ | | SGAEVK | | ASVGDRVTITCG | | SPSSLSA |
| | | | GLEWIGVINPKYGTTRYNQKFK | | KPGASV | | ASENIYGALNW | | SVGDRV |
| | | | GRATLTVDKSTSTAYMELSSLRS | | KVSCKA | | YQQKPGKAPKL | | TITCGAS |
| | | | EDTAVYYCTRGLNAWDYWGQG | | SGYSFT | | LIYGATNLEDGV | | ENIYGA |
| | | | TLVTVSSASTKGPSVFPLAPSSKS | | DYNVN | | PSRFSGSGSGRD | | LNWYQ |
| | | | TSGGTAALGCLVKDYFPEPVTVS | | WVRQAP | | YTFTISSLQPEDI | | QKPGKA |
| | | | WNSGALTSGVHTFPAVLQSSGL | | GQGLEW | | ATYYCQNVLTT | | PKLLIYG |
| | | | YSLSSVVTVPSSSLGTQTYICNVN | | IGVINPK | | PYTFGQGTKLEI | | ATNLED |
| | | | HKPSNTKVDKKVEPKSCDKTHT | | YGTTRY | | KRTVAAPSVFIF | | GVPSRFS |
| | | | CPPCPAPELLGGPSVFLFPPKPKD | | NQKFKG | | PPSDEQLKSGTA | | GSGSGR |
| | | | TLMISRTPEVTCVVVDVSHEDPE | | RATLTV | | SVVCLLNNFYPR | | DYTFTIS |
| | | | VKFNWYVDGVEVHNAKTKPRE | | DKSTST | | EAKVQWKVDN | | SLQPEDI |
| | | | EQYNSTYRVVSVLTVLHQDWLN | | AYMELS | | ALQSGNSQESVT | | ATYYCQ |
| | | | GKEYKCKVSNKALPAPIEKTISK | | SLRSEDT | | EQDSKDSTYSLS | | NVLTTP |
| | | | AKGQPREPQVYTLPPSRDELTKN | | AVYYCT | | STLTLSKADYEK | | YTFGQG |
| | | | QVSLWCLVKGFYPSDIAVEWES | | RGLNA | | HKVYACEVTHQ | | TKLEIK |
| | | | NGQPENNYKTTPPVLDSDGSFFL | | WDYWG | | GLSSPVTKSFNR | | |
| | | | YSKLTVDKSRWQQGNVFSCSVM | | QGTLVT | | GEC | | |
| | | | HEALHNHYTQKSLSLSPGK | | VSS | | | | |
| EPI1280 | ITGB6 | 173 | QVQLVQSGAEVKKPGASVKVSC | 174 | QVQLVQ | 175 | DVVMTQSPLSLP | 176 | DVVMT |
| EPI1165 | | | KASGYSFSGYFMNWVRQAPGQ | | SGAEVK | | VTLGQPASISCK | | QSPLSLP |
| | | | GLEWMGLINPYNGDSFYNQKFK | | KPGASV | | SSQSLLDSDGKT | | VTLGQP |
| | | | GRVTMTRQTSTSTVYMELSSLRS | | KVSCKA | | YLNWLFQRPGQ | | ASISCKS |
| | | | EDTAVYYCVRGLRRDFDYWGQ | | SGYSFS | | SPRRLIYLVSEL | | SQSLLDS |
| | | | GTLVTVSSASTKGPSVFPLAPSSK | | GYFMN | | DSGVPDRFSGSG | | DGKTYL |
| | | | STSGGTAALGCLVKDYFPEPVTV | | WVRQAP | | SGTDFTLKISRV | | NWLFQR |
| | | | SWNSGALTSGVHTFPAVLQSSGL | | GQGLEW | | EAEDVGVYYC | | PGQSPR |
| | | | YSLSSVVTVPSSSLGTQTYICNVN | | MGLINP | | WQGTHFPRTFG | | RLIYLVS |
| | | | HKPSNTKVDKKVEPKSCDKTHT | | YNGDSF | | GGTKLEIKRTVA | | ELDSGV |
| | | | CPPCPAPELLGGPSVFLFPPKPKD | | YNQKFK | | APSVFIFPPSDEQ | | PDRFSGS |
| | | | TLMISRTPEVTCVVVDVSHEDPE | | GRVTMT | | LKSGTASVVCLL | | GSGTDF |
| | | | VKFNWYVDGVEVHNAKTKPRE | | RQTSTST | | NNFYPREAKVQ | | TLKISRV |
| | | | EQYNSTYRVVSVLTVLHQDWLN | | VYMELS | | WKVDNALQSG | | EAEDVG |
| | | | GKEYKCKVSNKALPAPIEKTISK | | SLRSEDT | | NSQESVTEQDSK | | VYYCW |
| | | | AKGQPREPQVYTLPPSRDELTKN | | AVYYCV | | DSTYSLSSTLTL | | QGTHFP |
| | | | QVSLWCLVKGFYPSDIAVEWES | | RGLRRD | | SKADYEKHKVY | | RTFGGG |
| | | | NGQPENNYKTTPPVLDSDGSFFL | | FDYWG | | ACEVTHQGLSSP | | TKLEIK |
| | | | YSKLTVDKSRWQQGNVFSCSVM | | QGTLVT | | VTKSFNRGEC | | |
| | | | HEALHNHYTQKSLSLSPGKGGS | | VSS | | | | |
| | | | HHHHHH | | | | | | |
| EPI1281 | ITGB6 | 177 | QVQLQESGPGLVKPSQTLSLTCT | 178 | QVQLQE | 179 | SYELTQPSSVSV | 180 | SYELTQ |
| EPI1608 | | | VSGGSISSGGYYWSWIRQHPGK | | SGPGLV | | SPGQTARITCSG | | PSSVSVS |
| | | | GLEWIGYIYYSGRTYNNPSLKSR | | KPSQTLS | | DVLAKKSARWF | | PGQTARI |
| | | | VTISVDTSKNQFSLKLSSVTAAD | | LTCTVS | | HQKPGQAPVLVI | | TCSGDV |
| | | | TAVYYCARVATGRADYHFYAM | | GGSISSG | | YKDSERPSGIPE | | LAKKSA |
| | | | DVWGQGTTVTVSSASTKGPSVF | | GYYWS | | RFSGSSSGTTVT | | RWFHQK |
| | | | PLAPSSKSTSGGTAALGCLVKDY | | WIRQHP | | LTISGAQVEDEA | | PGQAPV |
| | | | FPEPVTVSWNSGALTSGVHTFPA | | GKGLEW | | AYYCYSAADNN | | LVIYKD |
| | | | VLQSSGLYSLSSVVTVPSSSLGTQ | | IGYIYYS | | LVFGGGTKLTV | | SERPSGI |
| | | | TYICNVNHKPSNTKVDKKVEPKS | | GRTYNN | | LRTVAAPSVFIF | | PERFSGS |
| | | | CDKTHTCPPCPAPELLGGPSVFLF | | PSLKSR | | PPSDEQLKSGTA | | SSGTTV |
| | | | PPKPKDTLMISRTPEVTCVVVDV | | VTISVDT | | SVVCLLNNFYPR | | TLTISGA |
| | | | SHEDPEVKFNWYVDGVEVHNA | | SKNQFS | | EAKVQWKVDN | | QVEDEA |
| | | | KTKPREEQYNSTYRVVSVLTVL | | LKLSSV | | ALQSGNSQESVT | | AYYCYS |
| | | | HQDWLNGKEYKCKVSNKALPA | | TAADTA | | EQDSKDSTYSLS | | AADNNL |
| | | | PIEKTISKAKGQPREPQVYTLPPS | | VYYCAR | | STLTLSKADYEK | | VFGGGT |
| | | | RDELTKNQVSLWCLVKGFYPSDI | | VATGRA | | HKVYACEVTHQ | | KLTVL |
| | | | AVEWESNGQPENNYKTTPPVLD | | DYHFYA | | GLSSPVTKSFNR | | |
| | | | SDGSFFLYSKLTVDKSRWQQGN | | MDVWG | | GEC | | |
| | | | VFSCSVMHEALHNHYTQKSLSLS | | QGTTVT | | | | |
| | | | PGK | | VSS | | | | |
| EPI1282 | PD-L1 | 181 | EVQLVESGGGLVQPGGSLRLSCA | 182 | EVQLVE | 183 | DIQMTQSPSSLS | 184 | DIQMTQ |
| | | | ASGFTFSDSWIHWVRQAPGKGL | | SGGGLV | | ASVGDRVTITCR | | SPSSLSA |
| | | | EWVAWISPYGGSTYYADSVKGR | | QPGGSL | | ASQDVSTAVAW | | SVGDRV |
| | | | FTISADTSKNTAYLQMNSLRAED | | RLSCAA | | YQQKPGKAPKL | | TITCRAS |
| | | | TAVYYCARRHWPGGFDYWGQG | | SGFTFSD | | LIYSASFLYSGV | | QDVSTA |
| | | | TLVTVSSASTKGPSVFPLAPSSKS | | SWIHWV | | PSRFSGSGSGTD | | VAWYQ |
| | | | TSGGTAALGCLVKDYFPEPVTVS | | RQAPGK | | FTLTISSLQPEDF | | QKPGKA |
| | | | WNSGALTSGVHTFPAVLQSSGL | | GLEWVA | | ATYYCQQYLYH | | PKLLIYS |
| | | | YSLSSVVTVPSSSLGTQTYICNVN | | WISPYG | | PATFGQGTKVEI | | ASFLYS |
| | | | HKPSNTKVDKKVEPKSCDKTHT | | GSTYYA | | KRTVAAPSVFIF | | GVPSRFS |
| | | | CPPCPAPELLGGPSVFLFPPKPKD | | DSVKGR | | PPSDEQLKSGTA | | GSGSGT |
| | | | TLMISRTPEVTCVVVDVSHEDPE | | FTISADT | | SVVCLLNNFYPR | | DFTLTIS |
| | | | VKFNWYVDGVEVHNAKTKPRE | | SKNTAY | | EAKVQWKVDN | | SLQPEDF |
| | | | EQYNSTYRVVSVLTVLHQDWLN | | LQMNSL | | ALQSGNSQESVT | | ATYYCQ |
| | | | GKEYKCKVSNKALPAPIEKTISK | | RAEDTA | | EQDSKDSTYSLS | | QYLYHP |
| | | | AKGQPREPQVYTLPPSRDELTKN | | VYYCAR | | STLTLSKADYEK | | ATFGQG |
| | | | QVSLWCLVKGFYPSDIAVEWES | | RHWPGG | | HKVYACEVTHQ | | TKVEIK |
| | | | NGQPENNYKTTPPVLDSDGSFFL | | FDYWG | | GLSSPVTKSFNR | | |
| | | | YSKLTVDKSRWQQGNVFSCSVM | | QGTLVT | | GEC | | |
| | | | HEALHNHYTQKSLSLSPGK | | VSS | | | | |
| EPI1283 | PD-L1 | 184 | EVQLVESGGGLVQPGGSLRLSCA | 186 | EVQLVE | 187 | EIVLTQSPGTLSL | 188 | EIVLTQS |
| | | | ASGFTFSRYWMSWVRQAPGKGL | | SGGGLV | | SPGERATLSCRA | | PGTLSLS |
| | | | EWVANIKQDGSEKYYVDSVKGR | | QPGGSL | | SQRVSSSYLAW | | PGERAT |
| | | | FTISRDNAKNSLYLQMNSLRAED | | RLSCAA | | YQQKPGQAPRL | | LSCRAS |
| | | | TAVYYCAREGGWFGELAFDYW | | SGFTFSR | | LIYDASSRATGIP | | QRVSSS |
| | | | GQGTLVTVSSASTKGPSVFPLAP | | YWMSW | | DRFSGSGSGTDF | | YLAWY |
| | | | SSKSTSGGTAALGCLVKDYFPEP | | VRQAPG | | TLTISRLEPEDFA | | QQKPGQ |
| | | | VTVSWNSGALTSGVHTFPAVLQ | | KGLEWV | | VYYCQQYGSLP | | APRLLIY |
| | | | SSGLYSLSSVVTVPSSSLGTQTYI | | ANIKQD | | WTFGQGTKVEI | | DASSRA |
| | | | CNVNHKPSNTKVDKKVEPKSCD | | GSEKYY | | KRTVAAPSVFIF | | TGIPDRF |
| | | | KTHTCPPCPAPELLGGPSVFLFPP | | VDSVKG | | PPSDEQLKSGTA | | SGSGSG |
| | | | KPKDTLMISRTPEVTCVVVDVSH | | RFTISRD | | SVVCLLNNFYPR | | TDFTLTI |
| | | | EDPEVKFNWYVDGVEVHNAKT | | NAKNSL | | EAKVQWKVDN | | SRLEPED |
| | | | KPREEQYNSTYRVVSVLTVLHQ | | YLQMNS | | ALQSGNSQESVT | | FAVYYC |
| | | | DWLNGKEYKCKVSNKALPAPIE | | LRAEDT | | EQDSKDSTYSLS | | QQYGSL |
| | | | KTISKAKGQPREPQVYTLPPSRD | | AVYYCA | | STLTLSKADYEK | | PWTFGQ |
| | | | ELTKNQVSLWCLVKGFYPSDIAV | | REGGWF | | HKVYACEVTHQ | | GTKVEI |
| | | | EWESNGQPENNYKTTPPVLDSD | | GELAFD | | GLSSPVTKSFNR | | K |
| | | | GSFFLYSKLTVDKSRWQQGNVF | | YWGQG | | GEC | | |
| | | | SCSVMHEALHNHYTQKSLSLSPG | | TLVTVS | | | | |
| | | | K | | S | | | | |
| EPI1284 | PD-L1 | 189 | EVQLLESGGGLVQPGGSLRLSCA | 190 | EVQLLE | 191 | QSALTQPASVSG | 192 | QSALTQ |
| EPI1730 | | | ASGFTFSSYIMMWVRQAPGKGL | | SGGGLV | | SPGQSITISCTGT | | PASVSG |
| | | | EWVSSIYPSGGITFYADTVKGRF | | QPGGSL | | SSDVGGYNYVS | | SPGQSIT |
| | | | TISRDNSKNTLYLQMNSLRAEDT | | RLSCAA | | WYQQHPGKAPK | | ISCTGTS |
| | | | AVYYCARIKLGTVTTVDYWGQG | | SGFTFSS | | LMIYDVSNRPSG | | SDVGGY |
| | | | TLVTVSSASTKGPSVFPLAPSSKS | | YIMMW | | VSNRFSGSKSGN | | NYVSW |
| | | | TSGGTAALGCLVKDYFPEPVTVS | | VRQAPG | | TASLTISGLQAE | | YQQHPG |
| | | | WNSGALTSGVHTFPAVLQSSGL | | KGLEWV | | DEADYYCSSYT | | KAPKLM |
| | | | YSLSSVVTVPSSSLGTQTYICNVN | | SSIYPSG | | SSSTRVFGTGTK | | TYDVSN |
| | | | HKPSNTKVDKKVEPKSCDKTHT | | GITFYA | | VTVLRTVAAPS | | RPSGVS |
| | | | CPPCPAPELLGGPSVFLFPPKPKD | | DTVKGR | | VFIFPPSDEQLKS | | NRFSGS |
| | | | TLMISRTPEVTCVVVDVSHEDPE | | FTISRDN | | GTASVVCLLNN | | KSGNTA |
| | | | VKFNWYVDGVEVHNAKTKPRE | | SKNTLY | | FYPREAKVQWK | | SLTISGL |
| | | | EQYNSTYRVVSVLTVLHQDWLN | | LQMNSL | | VDNALQSGNSQ | | QAEDEA |
| | | | GKEYKCKVSNKALPAPIEKTISK | | RAEDTA | | ESVTEQDSKDST | | DYYCSS |
| | | | AKGQPREPQVYTLPPSRDELTKN | | VYYCAR | | YSLSSTLTLSKA | | YTSSSTR |
| | | | QVSLWCLVKGFYPSDIAVEWES | | IKLGTVT | | DYEKHKVYACE | | VFGTGT |
| | | | NGQPENNYKTTPPVLDSDGSFFL | | TVDYW | | VTHQGLSSPVTK | | KVTVL |
| | | | YSKLTVDKSRWQQGNVFSCSVM | | GQGTLV | | SFNRGEC | | |
| | | | HEALHNHYTQKSLSLSPGK | | TVSS | | | | |
| EPI1285 | PD-L1 | 193 | EVQLLESGGGLVQPGGSLRLSCA | 194 | EVQLLE | 195 | SYVLTQPPSVSV | 196 | SYVLTQ |
| EPI1731 | | | ASGFTFSSYAMSWVRQAPGKGL | | SGGGLV | | APGQTARITCGG | | PPSVSV |
| | | | EWVSGISGSGGFTYYADSVKGRF | | QPGGSL | | NNIGSKSVHWY | | APGQTA |
| | | | TISRDNSKNTLYLQMNSLRAEDT | | RLSCAA | | QQKPGQAPVLV | | RITCGG |
| | | | AVYYCAKPPRGYNYGPFDYWG | | SGFTFSS | | VYDDSDRPSGIP | | NNIGSKS |
| | | | QGTLVTVSSASTKGPSVFPLAPSS | | YAMSW | | ERFSGSNSGNTA | | NHWYQ |
| | | | KSTSGGTAALGCLVKDYFPEPVT | | VRQAPG | | TLTISRVEAGDE | | QKPGQA |
| | | | VSWNSGALTSGVHTFPAVLQSS | | KGLEWV | | ADYYCQVWDSS | | PVLVVY |
| | | | GLYSLSSVVTVPSSSLGTQTYICN | | SGISGSG | | SDHVVFGGGTK | | DDSDRP |
| | | | VNHKPSNTKVDKKVEPKSCDKT | | GFTYYA | | LTVLRTVAAPSV | | SGIPERF |
| | | | HTCPPCPAPELLGGPSVFLFPPKP | | DSVKGR | | FIFPPSDEQLKSG | | SGSNSG |
| | | | KDTLMISRTPEVTCVVVDVSHED | | FTISRDN | | TASVVCLLNNF | | NTATLTI |
| | | | PEVKFNWYVDGVEVHNAKTKP | | SKNTLY | | YPREAKVQWKV | | SRVEAG |
| | | | REEQYNSTYRVVSVLTVLHQDW | | LQMNSL | | DNALQSGNSQE | | DEADYY |
| | | | LNGKEYKCKVSNKALPAPIEKTI | | RAEDTA | | SVTEQDSKDSTY | | CQVWDS |
| | | | SKAKGQPREPQVYTLPPSRDELT | | VYYCAK | | SLSSTLTLSKAD | | SSDHVV |
| | | | KNQVSLWCLVKGFYPSDIAVEW | | PPRGYN | | YEKHKVYACEV | | FGGGTK |
| | | | ESNGQPENNYKTTPPVLDSDGSF | | YGPFDY | | THQGLSSPVTKS | | LTVL |
| | | | FLYSKLTVDKSRWQQGNVFSCS | | WGQGTL | | FNRGEC | | |
| | | | VMHEALHNHYTQKSLSLSPGK | | VTVSS | | | | |
| EPI1296 | TROP2 | 197 | QVQLQQSGSELKKPGASVKVSC | 198 | QVQLQQ | 199 | DIQLTQSPSSLSA | 200 | DIQLTQS |
| EPI2218 | | | KASGYTFTNYGMNWVKQAPGQ | | SGSELK | | SVGDRVSITCKA | | PSSLSAS |
| EPI1166 | | | GLKWMGWINTYTGEPTYTDDFK | | KPGASV | | SQDVSIAVAWY | | VGDRVS |
| | | | GRFAFSLDTSVSTAYLQISSLKAD | | KVSCKA | | QQKPGKAPKLLI | | ITCKASQ |
| | | | DTAVYFCARGGFGSSYWYFDV | | SGYTFT | | YSASYRYTGVP | | DVSIAV |
| | | | WGQGSLVTVSSASTKGPSVFPLA | | NYGMN | | DRFSGSGSGTDF | | AWYQQ |
| | | | PSSKSTSGGTAALGCLVKDYFPE | | WVKQA | | TLTISSLQPEDFA | | KPGKAP |
| | | | PVTVSWNSGALTSGVHTFPAVL | | PGQGLK | | VYYCQQHYITPL | | KLLIYSA |
| | | | QSSGLYSLSSVVTVPSSSLGTQTY | | WMGWI | | TFGAGTKVEIKR | | SYRYTG |
| | | | ICNVNHKPSNTKVDKKVEPKSC | | NTYTGE | | TVAAPSVFIFPPS | | VPDRFS |
| | | | DKTHTCPPCPAPELLGGPSVFLFP | | PTYTDD | | DEQLKSGTASV | | GSGSGT |
| | | | PKPKDTLMISRTPEVTCVVVDVS | | FKGRFA | | VCLLNNFYPRE | | DFTLTIS |
| | | | HEDPEVKFNWYVDGVEVHNAK | | FSLDTSV | | AKVQWKVDNA | | SLQPEDF |
| | | | TKPREEQYNSTYRVVSVLTVLH | | STAYLQI | | LQSGNSQESVTE | | AVYYCQ |
| | | | QDWLNGKEYKCKVSNKALPAPI | | SSLKAD | | QDSKDSTYSLSS | | QHYITPL |
| | | | EKTISKAKGQPREPQVYTLPPSR | | DTAVYF | | TLTLSKADYEK | | TFGAGT |
| | | | DELTKNQVSLWCLVKGFYPSDIA | | CARGGF | | HKVYACEVTHQ | | KVEIK |
| | | | VEWESNGQPENNYKTTPPVLDS | | GSSYWY | | GLSSPVTKSFNR | | |
| | | | DGSFFLYSKLTVDKSRWQQGNV | | FDVWG | | GEC | | |
| | | | FSCSVMHEALHNHYTQKSLSLSP | | QGSLVT | | | | |
| | | | GK | | VSS | | | | |
| EPI1297 | TROP2 | 201 | QVQLVQSGAEVKKPGASVKVSC | 202 | QVQLVQ | 203 | DIQMTQSPSSLS | 204 | DIQMTQ |
| EPI2219 | | | KASGYTFTTAGMQWVRQAPGQ | | SGAEVK | | ASVGDRVTITCK | | SPSSLSA |
| EPI1737 | | | GLEWMGWINTHSGVPKYAEDFK | | KPGASV | | ASQDVSTAVAW | | SVGDRV |
| | | | GRVTISADTSTSTAYLQLSSLKSE | | KVSCKA | | YQQKPGKAPKL | | TITCKAS |
| | | | DTAVYYCARSGFGSSYWYFDV | | SGYTFT | | LIYSASYRYTGV | | QDVSTA |
| | | | WGQGTLVTVSSASTKGPSVFPLA | | TAGMQ | | PSRFSGSGSGTD | | VAWYQ |
| | | | PSSKSTSGGTAALGCLVKDYFPE | | WVRQAP | | FTLTISSLQPEDF | | QKPGKA |
| | | | PVTVSWNSGALTSGVHTFPAVL | | GQGLEW | | AVYYCQQHYIT | | PKLLIYS |
| | | | QSSGLYSLSSVVTVPSSSLGTQTY | | MGWINT | | PLTFGQGTKLEI | | ASYRYT |
| | | | ICNVNHKPSNTKVDKKVEPKSC | | HSGVPK | | KRTVAAPSVFIF | | GVPSRFS |
| | | | DKTHTCPPCPAPELLGGPSVFLFP | | YAEDFK | | PPSDEQLKSGTA | | GSGSGT |
| | | | PKPKDTLMISRTPEVTCVVVDVS | | GRVTISA | | SVVCLLNNFYPR | | DFTLTIS |
| | | | HEDPEVKFNWYVDGVEVHNAK | | DTSTST | | EAKVQWKVDN | | SLQPEDF |
| | | | TKPREEQYNSTYRVVSVLTVLH | | AYLQLS | | ALQSGNSQESVT | | AVYYCQ |
| | | | QDWLNGKEYKCKVSNKALPAPI | | SLKSED | | EQDSKDSTYSLS | | QHYITPL |
| | | | EKTISKAKGQPREPQVYTLPPSR | | TAVYYC | | STLTLSKADYEK | | TFGQGT |
| | | | DELTKNQVSLWCLVKGFYPSDIA | | ARSGFG | | HKVYACEVTHQ | | KLEIK |
| | | | VEWESNGQPENNYKTTPPVLDS | | SSYWYF | | GLSSPVTKSFNR | | |
| | | | DGSFFLYSKLTVDKSRWQQGNV | | DVWGQ | | GEC | | |
| | | | FSCSVMHEALHNHYTQKSLSLSP | | GTLVTV | | | | |
| | | | GK | | SS | | | | |
| EPI1298 | TROP2 | 205 | QVQLQESGPGLVKPSETLSLTCT | 206 | QVQLQE | 207 | DIVMTQSPDSLA | 208 | DIVMTQ |
| EPI1738 | | | VSGGSISSYGVHWIRQPPGKGLE | | SGPGLV | | VSLGERATINCR | | SPDSLA |
| | | | WIGVIWTSGVTDYNSALMGRVT | | KPSETLS | | ASKSVSTSGYSY | | VSLGER |
| | | | ISVDTSKNQFSLKLSSVTAADTA | | LTCTVS | | MHWYQQKPGQ | | ATINCR |
| | | | VYYCARDGDYDRYTMDYWGQ | | GGSISSY | | PPKLLIYLASNL | | ASKSVS |
| | | | GTLVTVSSASTKGPSVFPLAPSSK | | GVHWIR | | ESGVPDRFSGSG | | TSGYSY |
| | | | STSGGTAALGCLVKDYFPEPVTV | | QPPGKG | | SGTDFTLTISSLQ | | MHWYQ |
| | | | SWNSGALTSGVHTFPAVLQSSGL | | LEWIGVI | | AEDVAVYYCQH | | QKPGQP |
| | | | YSLSSVVTVPSSSLGTQTYICNVN | | WTSGVT | | SRELPYTFGQGT | | PKLLIYL |
| | | | HKPSNTKVDKKVEPKSCDKTHT | | DYNSAL | | KLEIKRTVAAPS | | ASNLES |
| | | | CPPCPAPELLGGPSVFLFPPKPKD | | MGRVTI | | VFIFPPSDEQLKS | | GVPDRF |
| | | | TLMISRTPEVTCVVVDVSHEDPE | | SVDTSK | | GTASVVCLLNN | | SGSGSG |
| | | | VKFNWYVDGVEVHNAKTKPRE | | NQFSLK | | FYPREAKVQWK | | TDFTLTI |
| | | | EQYNSTYRVVSVLTVLHQDWLN | | LSSVTA | | VDNALQSGNSQ | | SSLQAE |
| | | | GKEYKCKVSNKALPAPIEKTISK | | ADTAVY | | ESVTEQDSKDST | | DVAVYY |
| | | | AKGQPREPQVYTLPPSRDELTKN | | YCARDG | | YSLSSTLTLSKA | | CQHSRE |
| | | | QVSLWCLVKGFYPSDIAVEWES | | DYDRYT | | DYEKHKVYACE | | LPYTFG |
| | | | NGQPENNYKTTPPVLDSDGSFFL | | MDYWG | | VTHQGLSSPVTK | | QGTKLEI |
| | | | YSKLTVDKSRWQQGNVFSCSVM | | QGTLVT | | SFNRGEC | | K |
| | | | HEALHNHYTQKSLSLSPGK | | VSS | | | | |
| EPI1299 | TROP2 | 209 | QVQLVQSGAEVKKPGASVKVSC | 210 | QVQLVQ | 211 | EIVLTQSPATLSL | 212 | EIVLTQS |
| EPI1739 | | | KASGYTFTSYWINWVRQAPGQG | | SGAEVK | | SPGERATLSCRA | | PATLSLS |
| | | | LEWMGNIFPSDSYSNYNKKFKD | | KPGASV | | SQTIGTSIHWYQ | | PGERAT |
| | | | RVTMTRDTSTSTVYMELSSLRSE | | KVSCKA | | QKPGQAPRLLIY | | LSCRAS |
| | | | DTAVYYCARGSGFDYWGQGTL | | SGYTFTS | | YASESISGIPARF | | QTIGTSI |
| | | | VTVSSASTKGPSVFPLAPSSKSTS | | YWINWV | | SGSGSGTDFTLT | | HWYQQ |
| | | | GGTAALGCLVKDYFPEPVTVSW | | RQAPGQ | | ISSLEPEDFAVY | | KPGQAP |
| | | | NSGALTSGVHTFPAVLQSSGLYS | | GLEWM | | YCSQSFSWPFTF | | RLLIYY |
| | | | LSSVVTVPSSSLGTQTYICNVNH | | GNIFPSD | | GQGTKLEIKRTV | | ASESISG |
| | | | KPSNTKVDKKVEPKSCDKTHTC | | SYSNYN | | AAPSVFIFPPSDE | | IPARFSG |
| | | | PPCPAPELLGGPSVFLFPPKPKDT | | KKFKDR | | QLKSGTASVVC | | SGSGTD |
| | | | LMISRTPEVTCVVVDVSHEDPEV | | VTMTRD | | LLNNFYPREAK | | FTLTISS |
| | | | KFNWYVDGVEVHNAKTKPREE | | TSTSTV | | NQWKVDNALQ | | LEPEDF |
| | | | QYNSTYRVVSVLTVLHQDWLNG | | YMELSS | | SGNSQESVTEQD | | AVYYCS |
| | | | KEYKCKVSNKALPAPIEKTISKA | | LRSEDT | | SKDSTYSLSSTL | | QSFSWP |
| | | | KGQPREPQVYTLPPSRDELTKNQ | | AVYYCA | | TLSKADYEKHK | | FTFGQG |
| | | | VSLWCLVKGFYPSDIAVEWESN | | RGSGFD | | VYACEVTHQGL | | TKLEIK |
| | | | GQPENNYKTTPPVLDSDGSFFLY | | YWGQG | | SSPVTKSFNRGE | | |
| | | | SKLTVDKSRWQQGNVFSCSVMH | | TLVTVS | | C | | |
| | | | EALHNHYTQKSLSLSPGK | | S | | | | |
| EPI1300 | TROP2 | 213 | QVQLQQSGPELVRPGTSVRISCK | 214 | QVQLQQ | 215 | DIVMTQSPSSLS | 216 | DIVMTQ |
| EPI1740 | | | ASGYTFTIYWLGWVKQRPGHGL | | SGPELV | | VSAGEKVTMTC | | SPSSLSV |
| | | | EWIGNIFPGSAYINYNEKFKGKA | | RPGTSV | | KSSQSLLNSGNQ | | SAGEKV |
| | | | TLTADTSSSTAYMQLSSLTSEDS | | RISCKAS | | QNYLAWYQQK | | TMTCKS |
| | | | AVYFCAREGSNSGYWGQGTTLT | | GYTFTIY | | PGQPPKLLIYGA | | SQSLLNS |
| | | | VSSASTKGPSVFPLAPSSKSTSGG | | WLGWV | | STRESGVPDRFT | | GNQQNY |
| | | | TAALGCLVKDYFPEPVTVSWNS | | KQRPGH | | GSGSGTDFTLTI | | LAWYQ |
| | | | GALTSGVHTFPAVLQSSGLYSLS | | GLEWIG | | NSVQAEDLAVY | | QKPGQP |
| | | | SVVTVPSSSLGTQTYICNVNHKP | | NIFPGSA | | YCQSDHIYPYTF | | PKLLIYG |
| | | | SNTKVDKKVEPKSCDKTHTCPPC | | YINYNE | | GGGTKLEIKRTV | | ASTRES |
| | | | PAPELLGGPSVFLFPPKPKDTLMI | | KFKGKA | | AAPSVFIFPPSDE | | GVPDRF |
| | | | SRTPEVTCVVVDVSHEDPEVKFN | | TLTADT | | QLKSGTASVVC | | TGSGSG |
| | | | WYVDGVEVHNAKTKPREEQYN | | SSSTAY | | LLNNFYPREAK | | TDFTLTI |
| | | | STYRVVSVLTVLHQDWLNGKEY | | MQLSSL | | VQWKVDNALQ | | NSVQAE |
| | | | KCKVSNKALPAPIEKTISKAKGQ | | TSEDSA | | SGNSQESVTEQD | | DLAVYY |
| | | | PREPQVYTLPPSRDELTKNQVSL | | VYFCAR | | SKDSTYSLSSTL | | CQSDHI |
| | | | WCLVKGFYPSDIAVEWESNGQP | | EGSNSG | | TLSKADYEKHK | | YPYTFG |
| | | | ENNYKTTPPVLDSDGSFFLYSKL | | YWGQG | | VYACEVTHQGL | | GGTKLEI |
| | | | TVDKSRWQQGNVFSCSVMHEAL | | TTLTVSS | | SSPVTKSFNRGE | | K |
| | | | HNHYTQKSLSLSPGK | | | | C | | |
| EPI1301 | EphA2 | 217 | EVQLLESGGGLVQPGGSLRLSCA | 218 | EVQLLE | 219 | DIQMTQSPSSLS | 220 | DIQMTQ |
| EPI1732 | | | ASGFTFSHYMMAWVRQAPGKG | | SGGGLV | | ASVGDRVTITCR | | SPSSLSA |
| | | | LEWVSRIGPSGGPTHYADSVKGR | | QPGGSL | | ASQSISTWLAW | | SVGDRV |
| | | | FTISRDNSKNTLYLQMNSLRAED | | RLSCAA | | YQQKPGKAPKL | | TITCRAS |
| | | | TAVYYCAGYDSGYDYVAVAGP | | SGFTFSH | | LIYKASNLHTGV | | QSISTWL |
| | | | AEYFQHWGQGTLVTVSSASTKG | | YMMAW | | PSRFSGSGSGTE | | AWYQQ |
| | | | PSVFPLAPSSKSTSGGTAALGCL | | VRQAPG | | FSLTISGLQPDDF | | KPGKAP |
| | | | VKDYFPEPVTVSWNSGALTSGV | | KGLEWV | | ATYYCQQYNSY | | KLLIYK |
| | | | HTFPAVLQSSGLYSLSSVVTVPSS | | SRIGPSG | | SRTFGQGTKVEI | | ASNLHT |
| | | | SLGTQTYICNVNHKPSNTKVDK | | GPTHYA | | KRTVAAPSVFIF | | GVPSRFS |
| | | | KVEPKSCDKTHTCPPCPAPELLG | | DSVKGR | | PPSDEQLKSGTA | | GSGSGT |
| | | | GPSVFLFPPKPKDTLMISRTPEVT | | FTISRDN | | SVVCLLNNFYPR | | EFSLTIS |
| | | | CVVVDVSHEDPEVKFNWYVDG | | SKNTLY | | EAKVQWKVDN | | GLQPDD |
| | | | VEVHNAKTKPREEQYNSTYRVV | | LQMNSL | | ALQSGNSQESVT | | FATYYC |
| | | | SVLTVLHQDWLNGKEYKCKVSN | | RAEDTA | | EQDSKDSTYSLS | | QQYNSY |
| | | | KALPAPIEKTISKAKGQPREPQV | | VYYCAG | | STLTLSKADYEK | | SRTFGQ |
| | | | YTLPPSRDELTKNQVSLWCLVK | | YDSGYD | | HKVYACEVTHQ | | GTKVEI |
| | | | GFYPSDIAVEWESNGQPENNYKT | | YVAVAG | | GLSSPVTKSFNR | | K |
| | | | TPPVLDSDGSFFLYSKLTVDKSR | | PAEYFQ | | GEC | | |
| | | | WQQGNVFSCSVMHEALHNHYT | | HWGQG | | | | |
| | | | QKSLSLSPGK | | TLVTVS | | | | |
| | | | | | S | | | | |
| EPI1302 | EphA2 | 221 | QIQLVQSGPELKKPGETVKISCK | 222 | QIQLVQ | 223 | DVLMTQSPLSLP | 224 | DVLMTQ |
| EPI1733 | | | ASGYTFTHYSMHWVKQAPGKG | | SGPELK | | VSLGDQASISCR | | SPLSLPV |
| | | | LKWMGWINTYTGEPTYADDFK | | KPGETV | | SSQSIVHSNGNT | | SLGDQA |
| | | | GRFAFSLETSASTAFLQINNLKNE | | KISCKAS | | YLEWYLQKPGQ | | SISCRSS |
| | | | DTATYFCATYYRYERDFDYWGQ | | GYTFTH | | SPKLLIYKVSNR | | QSIVHSN |
| | | | GTTLTVSSASTKGPSVFPLAPSSK | | YSMHW | | FSGVPDRFSGSG | | GNTYLE |
| | | | STSGGTAALGCLVKDYFPEPVTV | | VKQAPG | | SGTDFTLRISRV | | WYLQKP |
| | | | SWNSGALTSGVHTFPAVLQSSGL | | KGLKW | | EAEDLGVYYCF | | GQSPKL |
| | | | YSLSSVVTVPSSSLGTQTYICNVN | | MGWINT | | QGSHVPYTFGG | | LIYKVS |
| | | | HKPSNTKVDKKVEPKSCDKTHT | | YTGEPT | | GTKLEIKRTVAA | | NRFSGV |
| | | | CPPCPAPELLGGPSVFLFPPKPKD | | YADDFK | | PSVFIFPPSDEQL | | PDRFSGS |
| | | | TLMISRTPEVTCVVVDVSHEDPE | | GRFAFS | | KSGTASVVCLL | | GSGTDF |
| | | | VKFNWYVDGVEVHNAKTKPRE | | LETSAST | | NNFYPREAKVQ | | TLRISRV |
| | | | EQYNSTYRVVSVLTVLHQDWLN | | AFLQIN | | WKVDNALQSG | | EAEDLG |
| | | | GKEYKCKVSNKALPAPIEKTISK | | NLKNED | | NSQESVTEQDSK | | VYYCFQ |
| | | | AKGQPREPQVYTLPPSRDELTKN | | TATYFC | | DSTYSLSSTLTL | | GSHVPY |
| | | | QVSLWCLVKGFYPSDIAVEWES | | ATYYRY | | SKADYEKHKVY | | TFGGGT |
| | | | NGQPENNYKTTPPVLDSDGSFFL | | ERDFDY | | ACEVTHQGLSSP | | KLEIK |
| | | | YSKLTVDKSRWQQGNVFSCSVM | | WGQGTT | | VTKSFNRGEC | | |
| | | | HEALHNHYTQKSLSLSPGK | | LTVSS | | | | |
| EPI1303 | EphA2 | 225 | QVQLVQSGAEVKKPGASVKVSC | 226 | QVQLVQ | 227 | DIVMTQTPLSLS | 228 | DIVMTQ |
| EPI1609 | | | KASGYTFTGYYIHWVRQAPGQG | | SGAEVK | | VTPGQPASISCR | | TPLSLSV |
| | | | LEWMGWIYPGNFNTKYNERFKG | | KPGASV | | SSQSLVHSNGNT | | TPGQPA |
| | | | RVTMTTDTSTSTAYMELRSLRSD | | KVSCKA | | FLYWYLQKPGQ | | SISCRSS |
| | | | DTAVYYCAREDGSPYYAMDYW | | SGYTFT | | SPQLLIYRVSNR | | QSLVHS |
| | | | GQGTSVTVSSASTKGPSVFPLAP | | GYYIHW | | FSGVPDRFSGSG | | NGNTFL |
| | | | SSKSTSGGTAALGCLVKDYFPEP | | VRQAPG | | SGTDFTLKISRV | | YWYLQ |
| | | | VTVSWNSGALTSGVHTFPAVLQ | | QGLEW | | EAEDVGVYYCF | | KPGQSP |
| | | | SSGLYSLSSVVTVPSSSLGTQTYI | | MGWIYP | | QATHVPWTFGG | | QLLIYR |
| | | | CNVNHKPSNTKVDKKVEPKSCD | | GNFNTK | | GTKVEIKRTVA | | VSNRFS |
| | | | KTHTCPPCPAPELLGGPSVFLFPP | | YNERFK | | APSVFIFPPSDEQ | | GVPDRF |
| | | | KPKDTLMISRTPEVTCVVVDVSH | | GRVTMT | | LKSGTASVVCLL | | SGSGSG |
| | | | EDPEVKFNWYVDGVEVHNAKT | | TDTSTST | | NNFYPREAKVQ | | TDFTLKI |
| | | | KPREEQYNSTYRVVSVLTVLHQ | | AYMELR | | WKVDNALQSG | | SRVEAE |
| | | | DWLNGKEYKCKVSNKALPAPIE | | SLRSDD | | NSQESVTEQDSK | | DVGVYY |
| | | | KTISKAKGQPREPQVYTLPPSRD | | TAVYYC | | DSTYSLSSTLTL | | CFQATH |
| | | | ELTKNQVSLWCLVKGFYPSDIAV | | AREDGS | | SKADYEKHKVY | | VPWTFG |
| | | | EWESNGQPENNYKTTPPVLDSD | | PYYAMD | | ACEVTHQGLSSP | | GGTKVE |
| | | | GSFFLYSKLTVDKSRWQQGNVF | | YWGQG | | VTKSFNRGEC | | IK |
| | | | SCSVMHEALHNHYTQKSLSLSPG | | TSVTVSS | | | | |
| | | | K | | | | | | |
| EPI1304 | EphA2 | 229 | QVQLVQSGAEVKKPGASVKVSC | 230 | QVQLVQ | 231 | EIVLTQSPATLSL | 232 | EIVLTQS |
| EPI1552 | | | KASGYTFTYFYMNWVRQAPGQ | | SGAEVK | | SPGERATLSCRA | | PATLSLS |
| | | | GLEWVGQINPNNGGTAYAQKFQ | | KPGASV | | SQSVSSSSYTYI | | PGERAT |
| | | | GRVTMTRDTSTSTVYMELSSLRS | | KVSCKA | | HWYQQKPGQAP | | LSCRAS |
| | | | EDTAVYYCARWVGTHYFDYWG | | SGYTFT | | RLLINFASNLES | | QSVSSSS |
| | | | QGTTLTVSSASTKGPSVFPLAPSS | | YFYMN | | GIPARFSGSGSG | | YTYIHW |
| | | | KSTSGGTAALGCLVKDYFPEPVT | | WVRQAP | | TDFTLTISSLEPE | | YQQKPG |
| | | | VSWNSGALTSGVHTFPAVLQSS | | GQGLEW | | DFAVYYCQHSW | | QAPRLLI |
| | | | GLYSLSSVVTVPSSSLGTQTYICN | | VGQINP | | EIPPTFGGGTKL | | NFASNL |
| | | | VNHKPSNTKVDKKVEPKSCDKT | | NNGGTA | | EIKRTVAAPSVFI | | ESGIPAR |
| | | | HTCPPCPAPELLGGPSVFLFPPKP | | YAQKFQ | | FPPSDEQLKSGT | | FSGSGS |
| | | | KDTLMISRTPEVTCVVVDVSHED | | GRVTMT | | ASVVCLLNNFY | | GTDFTL |
| | | | PEVKFNWYVDGVEVHNAKTKP | | RDTSTST | | PREAKVQWKVD | | TISSLEP |
| | | | REEQYNSTYRVVSVLTVLHQDW | | VYMELS | | NALQSGNSQES | | EDFAVY |
| | | | LNGKEYKCKVSNKALPAPIEKTI | | SLRSEDT | | VTEQDSKDSTYS | | YCQHSW |
| | | | SKAKGQPREPQVYTLPPSRDELT | | AVYYCA | | LSSTLTLSKADY | | EIPPTFG |
| | | | KNQVSLWCLVKGFYPSDIAVEW | | RWVGT | | EKHKVYACEVT | | GGTKLEI |
| | | | ESNGQPENNYKTTPPVLDSDGSF | | HYFDY | | HQGLSSPVTKSF | | K |
| | | | FLYSKLTVDKSRWQQGNVFSCS | | WGQGTT | | NRGEC | | |
| | | | VMHEALHNHYTQKSLSLSPGK | | LTVSS | | | | |
| EPI1323 | CD71 | 233 | EVQLVQSGAEVKKPGASVKVSC | 234 | EVQLVQ | 235 | DIQMTQSPSSLS | 236 | DIQMTQ |
| | | | KASGYTFTSYWMHWVRQAPGQ | | SGAEVK | | ASVGDRVTITCR | | SPSSLSA |
| | | | RLEWIGEINPTNGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFW | | PKLLVY |
| | | | LSSVVTVPSSSLGTQTYICNVNH | | GEINPTN | | GTPLTFGQGTK | | DATNLA |
| | | | KPSNTKVDKKVEPKSCDKTHTC | | GRTNYI | | VEIKRTVAAPSV | | DGVPSR |
| | | | PPCPAPELLGGPSVFLFPPKPKDT | | EKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFW |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | GQGTKV |
| | | | GQPENNYKTTPPVLDSDGSFFLY | | HYWGQ | | THQGLSSPVTKS | | EIK |
| | | | SKLTVDKSRWQQGNVFSCSVMH | | GTMVTV | | FNRGEC | | |
| | | | EALHNHYTQKSLSLSPGK | | SS | | | | |
| EPI1324 | CD71 | 237 | EVQLVQSGAEVKKPGASVKVSC | 238 | EVQLVQ | 239 | DIQMTQSPSSLS | 240 | DIQMTQ |
| | | | KASGYTFTSYWMHWVRQAPGQ | | SGAEVK | | ASVGDRVTITCR | | SPSSLSA |
| | | | RLEWIGEIAPTNGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFW | | PKLLVY |
| | | | LSSVVTVPSSSLGTQTYICNVNH | | GEIAPTN | | GTPLTFGQGTK | | DATNLA |
| | | | KPSNTKVDKKVEPKSCDKTHTC | | GRTNYI | | VEIKRTVAAPSV | | DGVPSR |
| | | | PPCPAPELLGGPSVFLFPPKPKDT | | EKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFW |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | GQGTKV |
| | | | GQPENNYKTTPPVLDSDGSFFLY | | HYWGQ | | THQGLSSPVTKS | | EIK |
| | | | SKLTVDKSRWQQGNVFSCSVMH | | GTMVTV | | FNRGEC | | |
| | | | EALHNHYTQKSLSLSPGK | | SS | | | | |
| EPI1325 | CD71 | 241 | EVQLVQSGAEVKKPGASVKVSC | 242 | EVQLVQ | 243 | DIQMTQSPSSLS | 244 | DIQMTQ |
| | | | KASGYTFTSYWMHWVRQAPGQ | | SGAEVK | | ASVGDRVTITCR | | SPSSLSA |
| | | | RLEWIGEINPANGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFW | | PKLLVY |
| | | | LSSVVTVPSSSLGTQTYICNVNH | | GEINPA | | GTPLTFGQGTK | | DATNLA |
| | | | KPSNTKVDKKVEPKSCDKTHTC | | NGRTNY | | VEIKRTVAAPSV | | DGVPSR |
| | | | PPCPAPELLGGPSVFLFPPKPKDT | | IEKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFW |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | GQGTKV |
| | | | GQPENNYKTTPPVLDSDGSFFLY | | HYWGQ | | THQGLSSPVTKS | | EIK |
| | | | SKLTVDKSRWQQGNVFSCSVMH | | GTMVTV | | FNRGEC | | |
| | | | EALHNHYTQKSLSLSPGK | | SS | | | | |
| EPI1326 | CD71 | 245 | EVQLVQSGAEVKKPGASVKVSC | 246 | EVQLVQ | 247 | DIQMTQSPSSLS | 248 | DIQMTQ |
| EPI1180 | | | KASGYTFTSYWMHWVRQAPGQ | | SGAEVK | | ASVGDRVTITCR | | SPSSLSA |
| EPI1181 | | | RLEWIGEINPANGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFA | | PKLLVY |
| | | | LSSVVTVPSSSLGTQTYICNVNH | | GEINPA | | GTPLTFGQGTK | | DATNLA |
| | | | KPSNTKVDKKVEPKSCDKTHTC | | NGRTNY | | VEIKRTVAAPSV | | DGVPSR |
| | | | PPCPAPELLGGPSVFLFPPKPKDT | | IEKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFA |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | |
| | | | GQPENNYKTTPPVLDSDGSFFLY | | HYWGQ | | THQGLSSPVTKS | | GQGTKV |
| | | | SKLTVDKSRWQQGNVFSCSVMH | | GTMVTV | | FNRGEC | | EIK |
| | | | EALHNHYTQKSLSLSPGK | | SS | | | | |
| EPI1327 | CD71 | 249 | EVQLVQSGAEVKKPGASVKVSC | 250 | EVQLVQ | 251 | DIQMTQSPSSLS | 252 | DIQMTQ |
| EPI1178 | | | KASGYTFTSYWMHWVRQAPGQ | | SGAEVK | | ASVGDRVTITCR | | SPSSLSA |
| EPI1182 | | | RLEWIGEINPTNGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFA | | PKLLVY |
| | | | LSSVVTVPSSSLGTQTYICNVNH | | GEINPTN | | GTPLTFGQGTK | | DATNLA |
| | | | KPSNTKVDKKVEPKSCDKTHTC | | GRTNYI | | VEIKRTVAAPSV | | DGVPSR |
| | | | PPCPAPELLGGPSVFLFPPKPKDT | | EKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFA |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | GQGTKV |
| | | | GQPENNYKTTPPVLDSDGSFFLY | | HYWGQ | | THQGLSSPVTKS | | EIK |
| | | | SKLTVDKSRWQQGNVFSCSVMH | | GTMVTV | | FNRGEC | | |
| | | | EALHNHYTQKSLSLSPGK | | SS | | | | |
| EPI1328 | CD71 | 253 | EVQLVQSGAEVKKPGASVKVSC | 254 | EVOLVQ | 255 | DIQMTQSPSSLS | 256 | DIQMTQ |
| EPI1179 | | | KASGYTFTSYWMHWVRQAPGQ | | SGAEVK | | ASVGDRVTITCR | | SPSSLSA |
| EPI1183 | | | RLEWIGEIAPTNGRTNYIEKFKSR | | KPGASV | | ASDNLYSNLAW | | SVGDRV |
| | | | ATLTVDKSASTAYMELSSLRSED | | KVSCKA | | YQQKPGKSPKL | | TITCRAS |
| | | | TAVYYCARGTRAYHYWGQGTM | | SGYTFTS | | LVYDATNLADG | | DNLYSN |
| | | | VTVSSASTKGPSVFPLAPSSKSTS | | YWMHW | | VPSRFSGSGSGT | | LAWYQ |
| | | | GGTAALGCLVKDYFPEPVTVSW | | VRQAPG | | DYTLTISSLQPE | | QKPGKS |
| | | | NSGALTSGVHTFPAVLQSSGLYS | | QRLEWI | | DFATYYCQHFA | | PKLLVY |
| | | | LSSVVTVPSSSLGTQTYICNVNH | | GEIAPTN | | GTPLTFGQGTK | | DATNLA |
| | | | KPSNTKVDKKVEPKSCDKTHTC | | GRTNYI | | VEIKRTVAAPSV | | DGVPSR |
| | | | PPCPAPELLGGPSVFLFPPKPKDT | | EKFKSR | | FIFPPSDEQLKSG | | FSGSGS |
| | | | LMISRTPEVTCVVVDVSHEDPEV | | ATLTVD | | TASVVCLLNNF | | GTDYTL |
| | | | KFNWYVDGVEVHNAKTKPREE | | KSASTA | | YPREAKVQWKV | | TISSLQP |
| | | | QYNSTYRVVSVLTVLHQDWLNG | | YMELSS | | DNALQSGNSQE | | EDFATY |
| | | | KEYKCKVSNKALPAPIEKTISKA | | LRSEDT | | SVTEQDSKDSTY | | YCQHFA |
| | | | KGQPREPQVYTLPPSRDELTKNQ | | AVYYCA | | SLSSTLTLSKAD | | GTPLTF |
| | | | VSLWCLVKGFYPSDIAVEWESN | | RGTRAY | | YEKHKVYACEV | | GQGTKV |
| | | | GQPENNYKTTPPVLDSDGSFFLY | | HYWGQ | | THQGLSSPVTKS | | EIK |
| | | | SKLTVDKSRWQQGNVFSCSVMH | | GTMVTV | | FNRGEC | | |
| | | | EALHNHYTQKSLSLSPGK | | SS | | | | |
| EPI1329 | CD71 | 257 | QVQLQQSGPDLVKPGASVRISCK | 258 | QVQLQQ | 259 | DILLTOSPAILSV | 260 | DILLTOS |
| EPI1184 | | | ASGYTFAGHYVHWVKQRPGRG | | SGPDLV | | SPGDRVSFSCRA | | PAILSVS |
| | | | LEWIGWIFPGKVNTKYNEKFKG | | KPGASV | | SQSIGTSIHWYQ | | PGDRVS |
| | | | KATLTADKSSSTAYMQLSSLTSE | | RISCKAS | | QRTDGSPRLLIK | | FSCRAS |
| | | | DSAVYFCARVGYDYPYYFDYW | | GYTFAG | | YASESISGIPSRF | | QSIGTSI |
| | | | GQGTTLTVSSASTKGPSVFPLAPS | | HYVHW | | SGSGSGTDFTLSI | | HWYQQ |
| | | | SKSTSGGTAALGCLVKDYFPEPV | | VKQRPG | | NSVESEDVADY | | RTDGSP |
| | | | TVSWNSGALTSGVHTFPAVLQSS | | RGLEWI | | YCQQSSSWPFTF | | RLLIKY |
| | | | GLYSLSSVVTVPSSSLGTQTYICN | | GWIFPG | | GSGTKLEIKRTV | | ASESISG |
| | | | VNHKPSNTKVDKKVEPKSCDKT | | KVNTKY | | AAPSVFIFPPSDE | | IPSRFSG |
| | | | HTCPPCPAPELLGGPSVFLFPPKP | | NEKFKG | | QLKSGTASVVC | | SGSGTD |
| | | | KDTLMISRTPEVTCVVVDVSHED | | KATLTA | | LLNNFYPREAK | | FTLSINS |
| | | | PEVKFNWYVDGVEVHNAKTKP | | DKSSST | | VQWKVDNALQ | | VESEDV |
| | | | REEQYNSTYRVVSVLTVLHQDW | | AYMQLS | | SGNSQESVTEQD | | ADYYCQ |
| | | | LNGKEYKCKVSNKALPAPIEKTI | | SLTSEDS | | SKDSTYSLSSTL | | QSSSWP |
| | | | SKAKGQPREPQVYTLPPSRDELT | | AVYFCA | | TLSKADYEKHK | | FTFGSGT |
| | | | KNQVSLWCLVKGFYPSDIAVEW | | RVGYDY | | VYACEVTHQGL | | KLEIK |
| | | | ESNGQPENNYKTTPPVLDSDGSF | | PYYFDY | | SSPVTKSFNRGE | | |
| | | | FLYSKLTVDKSRWQQGNVFSCS | | WGQGTT | | C | | |
| | | | VMHEALHNHYTQKSLSLSPGK | | LTVSS | | | | |
| EPI1621 | IGF1R | 261 | QVQLQESGPGLVKPSGTLSLTCA | 262 | QVQLQE | 263 | DVVMTQSPLSLP | 264 | DVVMT |
| EPI1741 | | | VSGGSISSSNWWSWVRQPPGKG | | SGPGLV | | VTPGEPASISCRS | | QSPLSLP |
| | | | LEWIGEIYHSGSTNYNPSLKSRV | | KPSGTLS | | SQSLLHSNGYN | | VTPGEP |
| | | | TISVDKSKNQFSLKLSSVTAADT | | LTCAVS | | YLDWYLQKPGQ | | ASISCRS |
| | | | AVYYCARWTGRTDAFDIWGQG | | GGSISSS | | SPQLLIYLGSNR | | SQSLLHS |
| | | | TMVTVSSASTKGPSVFPLAPSSK | | NWWSW | | ASGVPDRFSGSG | | NGYNYL |
| | | | STSGGTAALGCLVKDYFPEPVTV | | VRQPPG | | SGTDFTLKISRV | | DWYLQ |
| | | | SWNSGALTSGVHTFPAVLQSSGL | | KGLEWI | | EAEDVGVYYCM | | KPGQSP |
| | | | YSLSSVVTVPSSSLGTQTYICNVN | | GEIYHS | | QGTHWPLTFGQ | | QLLIYLG |
| | | | HKPSNTKVDKKVEPKSCDKTHT | | GSTNYN | | GTKVEIKRTVA | | SNRASG |
| | | | CPPCPAPELLGGPSVFLFPPKPKD | | PSLKSR | | APSVFIFPPSDEQ | | VPDRFS |
| | | | TLMISRTPEVTCVVVDVSHEDPE | | VTISVD | | LKSGTASVVCLL | | GSGSGT |
| | | | VKFNWYVDGVEVHNAKTKPRE | | KSKNQF | | NNFYPREAKVQ | | DFTLKIS |
| | | | EQYNSTYRVVSVLTVLHQDWLN | | SLKLSSV | | WKVDNALQSG | | RVEAED |
| | | | GKEYKCKVSNKALPAPIEKTISK | | TAADTA | | NSQESVTEQDSK | | VGVYYC |
| | | | AKGQPREPQVYTLPPSRDELTKN | | VYYCAR | | DSTYSLSSTLTL | | MQGTH |
| | | | QVSLWCLVKGFYPSDIAVEWES | | WTGRTD | | SKADYEKHKVY | | WPLTFG |
| | | | NGQPENNYKTTPPVLDSDGSFFL | | AFDIWG | | ACEVTHQGLSSP | | QGTKVE |
| | | | YSKLTVDKSRWQQGNVFSCSVM | | QGTMVT | | VTKSFNRGEC | | IK |
| | | | HEALHNHYTQKSLSLSPGK | | VSS | | | | |
| EPI1622 | IGF1R | 265 | QVELVESGGGVVQPGRSQRLSC | 266 | QVELVE | 267 | EIVLTQSPATLSL | 268 | EIVLTQS |
| EPI1742 | | | AASGFTFSSYGMHWVRQAPGKG | | SGGGVV | | SPGERATLSCRA | | PATLSLS |
| | | | LEWVAIIWFDGSSTYYADSVRGR | | QPGRSQ | | SQSVSSYLAWY | | PGERAT |
| | | | FTISRDNSKNTLYLQMNSLRAED | | RLSCAA | | QQKPGQAPRLLI | | LSCRAS |
| | | | TAVYFCARELGRRYFDLWGRGT | | SGFTFSS | | YDASKRATGIPA | | QSVSSY |
| | | | LVSVSSASTKGPSVFPLAPSSKST | | YGMHW | | RFSGSGSGTDFT | | LAWYQ |
| | | | SGGTAALGCLVKDYFPEPVTVS | | VRQAPG | | LTISSLEPEDFAV | | QKPGQA |
| | | | WNSGALTSGVHTFPAVLQSSGL | | KGLEWV | | YYCQQRSKWPP | | PRLLIYD |
| | | | YSLSSVVTVPSSSLGTQTYICNVN | | AIIWFDG | | WTFGQGTKVES | | ASKRAT |
| | | | HKPSNTKVDKKVEPKSCDKTHT | | SSTYYA | | KRTVAAPSVFIF | | GIPARFS |
| | | | CPPCPAPELLGGPSVFLFPPKPKD | | DSVRGR | | PPSDEQLKSGTA | | GSGSGT |
| | | | TLMISRTPEVTCVVVDVSHEDPE | | FTISRDN | | SVVCLLNNFYPR | | DFTLTIS |
| | | | VKFNWYVDGVEVHNAKTKPRE | | SKNTLY | | EAKVQWKVDN | | SLEPEDF |
| | | | EQYNSTYRVVSVLTVLHQDWLN | | LQMNSL | | ALQSGNSQESVT | | AVYYCQ |
| | | | GKEYKCKVSNKALPAPIEKTISK | | RAEDTA | | EQDSKDSTYSLS | | QRSKWP |
| | | | AKGQPREPQVYTLPPSRDELTKN | | VYFCAR | | STLTLSKADYEK | | PWTFGQ |
| | | | QVSLWCLVKGFYPSDIAVEWES | | ELGRRY | | HKVYACEVTHQ | | GTKVES |
| | | | NGQPENNYKTTPPVLDSDGSFFL | | FDLWGR | | GLSSPVTKSFNR | | K |
| | | | YSKLTVDKSRWQQGNVFSCSVM | | GTLVSV | | GEC | | |
| | | | HEALHNHYTQKSLSLSPGK | | SS | | | | |
| EPI1623 | IGF1R | 269 | EVQLLESGGGLVQPGGSLRLSCT | 270 | EVQLLE | 271 | DIQMTQFPSSLS | 272 | DIQMTQ |
| EPI1743 | | | ASGFTFSSYAMNWVRQAPGKGL | | SGGGLV | | ASVGDRVTITCR | | FPSSLSA |
| | | | EWVSAISGSGGTTFYADSVKGRF | | QPGGSL | | ASQGIRNDLGW | | SVGDRV |
| | | | TISRDNSRTTLYLQMNSLRAEDT | | RLSCTA | | YQQKPGKAPKR | | TITCRAS |
| | | | AVYYCAKDLGWSDSYYYYYGM | | SGFTFSS | | LIYAASRLHRGV | | QGIRND |
| | | | DVWGQGTTVTVSSASTKGPSVF | | YAMNW | | PSRFSGSGSGTE | | LGWYQ |
| | | | PLAPSSKSTSGGTAALGCLVKDY | | VRQAPG | | FTLTISSLQPEDF | | QKPGKA |
| | | | FPEPVTVSWNSGALTSGVHTFPA | | KGLEWV | | ATYYCLQHNSY | | PKRLIYA |
| | | | VLQSSGLYSLSSVVTVPSSSLGTQ | | SAISGSG | | PCSFGQGTKLEI | | ASRLHR |
| | | | TYICNVNHKPSNTKVDKKVEPKS | | GTTFYA | | KRTVAAPSVFIF | | GVPSRFS |
| | | | CDKTHTCPPCPAPELLGGPSVFLF | | DSVKGR | | PPSDEQLKSGTA | | GSGSGT |
| | | | PPKPKDTLMISRTPEVTCVVVDV | | FTISRDN | | SVVCLLNNFYPR | | EFTLTIS |
| | | | SHEDPEVKFNWYVDGVEVHNA | | SRTTLY | | EAKVQWKVDN | | SLQPEDF |
| | | | KTKPREEQYNSTYRVVSVLTVL | | LQMNSL | | ALQSGNSQESVT | | ATYYCL |
| | | | HQDWLNGKEYKCKVSNKALPA | | RAEDTA | | EQDSKDSTYSLS | | QHNSYP |
| | | | PIEKTISKAKGQPREPQVYTLPPS | | VYYCAK | | STLTLSKADYEK | | CSFGQG |
| | | | RDELTKNQVSLWCLVKGFYPSDI | | DLGWSD | | HKVYACEVTHQ | | TKLEIK |
| | | | AVEWESNGQPENNYKTTPPVLD | | SYYYYY | | GLSSPVTKSFNR | | |
| | | | SDGSFFLYSKLTVDKSRWQQGN | | GMDVW | | GEC | | |
| | | | VFSCSVMHEALHNHYTQKSLSLS | | GQGTTV | | | | |
| | | | PGK | | TVSS | | | | |
| EPI1624 | IGF1R | 273 | EVQLVQSGAEVKKPGSSVKVSC | 274 | EVQLVQ | 275 | SSELTQDPAVSV | 276 | SSELTQ |
| EPI1744 | | | KASGGTFSSYAISWVRQAPGQGL | | SGAEVK | | ALGQTVRITCQG | | DPAVSV |
| | | | EWMGGIIPIFGTANYAQKFQGRV | | KPGSSV | | DSLRSYYATWY | | ALGQTV |
| | | | TITADKSTSTAYMELSSLRSEDT | | KVSCKA | | QQKPGQAPILVI | | RITCQG |
| | | | AVYYCARAPLRFLEWSTQDHYY | | SGGTFSS | | YGENKRPSGIPD | | DSLRSY |
| | | | YYYMDVWGKGTTVTVSSASTK | | YAISWV | | RFSGSSSGNTAS | | YATWY |
| | | | GPSVFPLAPSSKSTSGGTAALGC | | RQAPGQ | | LTITGAQAEDEA | | QQKPGQ |
| | | | LVKDYFPEPVTVSWNSGALTSG | | GLEWM | | DYYCKSRDGSG | | APILVIY |
| | | | VHTFPAVLQSSGLYSLSSVVTVP | | GGIIPIFG | | QHLVFGGGTKL | | GENKRP |
| | | | SSSLGTQTYICNVNHKPSNTKVD | | TANYAQ | | TVLRTVAAPSVF | | SGIPDRF |
| | | | KKVEPKSCDKTHTCPPCPAPELL | | KFQGRV | | IFPPSDEQLKSGT | | SGSSSG |
| | | | GGPSVFLFPPKPKDTLMISRTPEV | | TITADKS | | ASVVCLLNNFY | | NTASLTI |
| | | | TCVVVDVSHEDPEVKFNWYVD | | TSTAYM | | PREAKVQWKVD | | TGAQAE |
| | | | GVEVHNAKTKPREEQYNSTYRV | | ELSSLRS | | NALQSGNSQES | | DEADYY |
| | | | VSVLTVLHQDWLNGKEYKCKVS | | EDTAVY | | VTEQDSKDSTYS | | CKSRDG |
| | | | NKALPAPIEKTISKAKGQPREPQ | | YCARAP | | LSSTLTLSKADY | | SGQHLV |
| | | | VYTLPPSRDELTKNQVSLWCLV | | LRFLEW | | EKHKVYACEVT | | FGGGTK |
| | | | KGFYPSDIAVEWESNGQPENNY | | STQDHY | | HQGLSSPVTKSF | | LTVL |
| | | | KTTPPVLDSDGSFFLYSKLTVDK | | YYYYM | | NRGEC | | |
| | | | SRWQQGNVFSCSVMHEALHNH | | DVWGK | | | | |
| | | | YTQKSLSLSPGK | | GTTVTV | | | | |
| | | | | | SS | | | | |

The sequences listed in Table 1 (SEQ ID NOs: 1-276) are amino acid molecules. The sequences listed in Table 1 (SEQ ID NOs: 1-276) are amino acid molecules that are synthetic constructs. The sequences listed in Table 1 (SEQ ID NOs: 1-276) for HC sequences (heavy chain), VH sequence (variable heavy chain sequence), LC sequences (light chain), VL sequence (variable light chain sequence) are amino acid molecules that are synthetic constructs.

TABLE 2
Exemplary CDR sequences for antibodies targeting the
internalizing receptor protein.

| | | SEQ | | SEQ | | SEQ | |
|---|---|---|---|---|---|---|---|
| Antibody | Arm 1 | ID | | ID | | ID | |
| IDs | Target | NO | CDR H1 | NO | CDR H2 | NO | CDR H3 |
| EPI1092 | CD71 | 472 | SYWMH | 473 | AIYPGNSETGYAQKFQ | 474 | ENWDPGFAF |
| EPI1091 | | | | | G | | |
| EPI1090 | | | | | | | |
| EPI1093 | | | | | | | |
| EPI1177 | | | | | | | |
| EPI1237 | MUC1 | 478 | SHFMH | 479 | WIDPVTGGTKYAQNFQ | 480 | EARADRGQF |
| EPI1118 | | | | | G | | DK |
| EPI1238 | MUC1 | 484 | NYWMN | 485 | EIRLKSNQYTTHYAESV | 486 | HYYFDY |
| EPI1119 | | | | | KG | | |
| EPI1240 | MUC1 | 490 | AYWIE | 491 | EILPGSGNSRYNEKFKG | 492 | SYDFAWFAY |
| EPI221 | | | | | | | |
| EPI1121 | | | | | | | |
| EPI1241 | MUC1 | 496 | SYNMH | 497 | YIYPGNGATNYNQKFQ | 498 | GDSVPFAY |
| EPI1122 | | | | | G | | |
| EPI1242 | MUC1 | 502 | SYPMS | 503 | YINNGGGNPYYPDTVK | 504 | QYYGFDY |
| EPI2212 | | | | | G | | |
| EPI1123 | | | | | | | |
| EPI1243 | CD276 | 508 | SFGMH | 509 | YISSDSSAIYYADTVKG | 510 | GRENIYYGS |
| EPI1135 | | | | | | | RLDY |
| EPI1244 | CD276 | 514 | NYDIN | 515 | WIFPGDGSTQYNEKFK | 516 | QTTATWFAY |
| EPI1136 | | | | | G | | |
| EPI1245 | CD276 | 520 | SGYSWH | 521 | YIHSSGSTNYNPSLKS | 522 | YDDYFEY |
| EPI2213 | | | | | | | |
| EPI1137 | | | | | | | |
| EPI1246 | CD276 | 526 | NYVMH | 527 | YINPYNDDVKYNEKFK | 528 | WGYYGSPLY |
| EPI2214 | | | | | G | | YFDY |
| EPI1138 | | | | | | | |
| EPI1247 | TPBG | 532 | GYYMH | 533 | RINPNNGVTLYNQKFK | 534 | STMITNYVMDY |
| | | | | | D | | |
| EPI1248 | TPBG | 538 | NFGMN | 539 | WINTNTGEPRYAEEFK | 540 | DWDGAYFFDY |
| EPI1522 | | | | | G | | |
| EPI1249 | TPBG | 544 | TYAMN | 545 | RIRSKSNNYATYYADS | 546 | QWDYDVRAMNY |
| EPI1523 | | | | | VKD | | |
| EPI1250 | TPBG | 550 | HYVVG | 551 | IIYGSGRTYYANWAKG | 552 | DASVSVYYWGY |
| EPI1524 | | | | | | | FDL |
| EPI1251 | TPBG | 556 | SYGMG | 557 | IISRNSVTYYATWAKG | 558 | RATYSGALGYF |
| EPI1525 | | | | | | | DI |
| EPI1252 | TPBG | 562 | GYYWT | 563 | EIDHSESTNYNPSLKS | 564 | WFGELYHYYYG |
| EPI1526 | | | | | | | MDV |
| EPI1253 | MST1R | 568 | SYLMT | 569 | NIKQDGSEKYYVDSVK | 570 | DGYSSGRHYGM |
| EPI1139 | | | | | G | | DV |
| EPI1254 | MST1R | 574 | RHWMS | 575 | EINPDSRTINYAPSVKG | 576 | RVRIHYYGAMDS |
| EPI1140 | | | | | | | |
| EPI1255 | MST1R | 580 | DTYIH | 581 | RIDPADGNRKSDPKFQ | 582 | GYGNLNAMDS |
| EPI1141 | | | | | V | | |
| EPI1256 | MST1R | 586 | DYHMD | 587 | DINPNNGGAIYNQKFK | 588 | SHYDYAGGAWF |
| EPI1142 | | | | | G | | AY |
| EPI1257 | HER3 | 592 | GYYWS | 593 | EINHSGSTNYNPSLKS | 594 | DKWTWYFDL |
| EPI1258 | HER3 | 598 | SYAMS | 599 | AINSQGKSTYYADSVK | 600 | WGDEGFDI |
| EPI1168 | | | | | G | | |
| EPI1259 | HER3 | 604 | GDWIH | 605 | EISAAGGYTDYADSVK | 606 | ESRVSFEAAMDY |
| EPI1167 | | | | | G | | |
| EPI1260 | HER3 | 610 | SSYIS | 611 | WIYAGTGSPSYNQKLQ | 612 | HRDYYSNSLTY |
| | | | | | G | | |
| EPI1261 | HER3 | 616 | DYDMS | 617 | TIDLDSGSIYYADSVQG | 618 | DLHMGPEGPFDY |
| EPI1262 | ADAM9 | 622 | SYWMH | 623 | EIIPIFGHTNYNEKFKS | 624 | GGYYYYPRQGFL |
| | | | | | | | DY |
| EPI1263 | CDH3 | 628 | SYAMS | 629 | AISGSGGSTYYADSVK | 630 | TNSAKFDP |
| EPI2215 | | | | | G | | |
| EPI1606 | | | | | | | |
| EPI1264 | CDH3 | 634 | SYGVH | 635 | VIWSGGSTDYADSVKG | 636 | NSNNGFAY |
| EPI2216 | | | | | | | |
| EPI1607 | | | | | | | |
| EPI1265 | CDH3 | 640 | AYNMH | 641 | FIDPYSGIITYNQTFKG | 642 | RGYYDGGFDY |
| EPI2217 | | | | | | | |
| EPI1734 | | | | | | | |
| EPI1266 | CDH3 | 646 | RYWIN | 647 | NIYPGSNITNYNEKFKN | 648 | EGIYDGYFPLF |
| EPI1735 | | | | | | | PY |
| EPI1267 | CDH3 | 652 | SQSAAWN | 653 | RIYYRSKWYNDYALSV | 654 | GEGYGREGFAI |
| EPI1736 | | | | | KS | | |
| EPI1268 | EpCAM | 658 | NYGMN | 659 | WINTYTGEPTYGEDFK | 660 | FGNYVDY |
| EPI1147 | | | | | G | | |
| EPI1269 | EpCAM | 664 | KYGMN | 665 | WINTYTEEPTYGDDFK | 666 | FGSAVDY |
| EPI1148 | | | | | G | | |
| EPI1270 | EpCAM | 670 | NYGMN | 671 | WINTYTGESTYADSFK | 672 | FAIKGDY |
| EPI1149 | | | | | G | | |
| EPI1271 | EpCAM | 676 | SYAIS | 677 | GIVPIFGTANYAQKFQG | 678 | DPFLHY |
| EPI1272 | EpCAM | 682 | SYAIS | 683 | GIIPIFGTANYAQKFQG | 684 | GLLWNY |
| EPI1150 | | | | | | | |
| EPI1273 | TNFRSF10B | 688 | SYVMS | 689 | TISSGGSYTYYPDSVKG | 690 | RGDSMITTDY |
| EPI1151 | | | | | | | |
| EPI1274 | TNFRSF10B | 694 | DYAMS | 695 | GINWQGGSTGYADSVK | 696 | ILGAGRGWYFDY |
| EPI1152 | | | | | G | | |
| EPI1275 | TNFRSF10B | 700 | DTFIH | 701 | RIDPANTNTKYDPKFQ | 702 | GLYTYYFDY |
| EPI1153 | | | | | G | | |
| EPI1276 | TNFRSF10B | 706 | SGDYFWS | 707 | HIHNSGTTYYNPSLKS | 708 | DRGGDYYYGMDV |
| EPI1154 | | | | | | | |
| EPI1277 | TNFRSF10B | 712 | DYGMS | 713 | GINWNGGSTGYADSVK | 714 | ILGAGRGWYFDL |
| EPI1155 | | | | | G | | |
| EPI1278 | ITGB6 | 718 | RYVMS | 719 | SISSGGRMYYPDTVKG | 720 | GSIYDGYYVFPY |
| EPI1164 | | | | | | | |
| EPI1279 | ITGB6 | 724 | DYNVN | 725 | VINPKYGTTRYNQKFK | 726 | GLNAWDY |
| EPI1407 | | | | | G | | |
| EPI1280 | ITGB6 | 730 | GYFMN | 731 | LINPYNGDSFYNQKFK | 732 | GLRRDFDY |
| EPI1165 | | | | | G | | |
| EPI1281 | ITGB6 | 736 | SGGYYWS | 737 | YIYYSGRTYNNPSLKS | 738 | VATGRADYHFYA |
| EPI1608 | | | | | | | MDV |
| EPI1282 | PD-L1 | 742 | DSWIH | 743 | WISPYGGSTYYADSVK | 744 | RHWPGGFDY |
| | | | | | G | | |
| EPI1283 | PD-L1 | 748 | RYWMS | 749 | NIKQDGSEKYYVDSVK | 750 | EGGWFGELAFDY |
| | | | | | G | | |
| EPI1284 | PD-L1 | 754 | SYIMM | 755 | SIYPSGGITFYADTVKG | 756 | IKLGTVTTVDY |
| EPI1730 | | | | | | | |
| EPI1285 | PD-L1 | 760 | SYAMS | 761 | GISGSGGFTYYADSVK | 762 | PPRGYNYGPFDY |
| EPI1731 | | | | | G | | |
| EPI1296 | TROP2 | 766 | NYGMN | 767 | WINTYTGEPTYTDDFK | 768 | GGFGSSYWYFDV |
| EPI2218 | | | | | G | | |
| EPI1166 | | | | | | | |
| EPI1297 | TROP2 | 772 | TAGMQ | 773 | WINTHSGVPKYAEDFK | 774 | SGFGSSYWYFDV |
| EPI2219 | | | | | G | | |
| EPI1737 | | | | | | | |
| EPI1298 | TROP2 | 778 | SYGVH | 779 | VIWTSGVTDYNSALMG | 780 | DGDYDRYTMDY |
| EPI1738 | | | | | | | |
| EPI1299 | TROP2 | 784 | SYWIN | 785 | NIFPSDSYSNYNKKFKD | 786 | GSGFDY |
| EPI1739 | | | | | | | |
| EPI1300 | TROP2 | 790 | IYWLG | 791 | NIFPGSAYINYNEKFKG | 792 | EGSNSGY |
| EPI1740 | | | | | | | |
| EPI1301 | EphA2 | 796 | HYMMA | 797 | RIGPSGGPTHYADSVK | 798 | YDSGYDYVAVAG |
| EPI1732 | | | | | G | | PAEYFQH |
| EPI1302 | EphA2 | 802 | HYSMH | 803 | WINTYTGEPTYADDFK | 804 | YYRYERDFDY |
| EPI1733 | | | | | G | | |
| EPI1303 | EphA2 | 808 | GYYIH | 809 | WIYPGNFNTKYNERFK | 810 | EDGSPYYAMDY |
| EPI1609 | | | | | G | | |
| EPI1304 | EphA2 | 814 | YFYMN | 815 | QINPNNGGTAYAQKFQ | 816 | WVGTHYFDY |
| EPI1552 | | | | | G | | |
| EPI1323 | CD71 | 820 | SYWMH | 821 | EINPTNGRTNYIEKFKS | 822 | GTRAYHY |
| EPI1324 | CD71 | 826 | SYWMH | 827 | EIAPTNGRTNYIEKFKS | 828 | GTRAYHY |
| EPI1325 | CD71 | 832 | SYWMH | 833 | EINPANGRTNYIEKFKS | 834 | GTRAYHY |
| EPI1326 | CD71 | 838 | SYWMH | 839 | EINPANGRTNYIEKFKS | 840 | GTRAYHY |
| EPI1180 | | | | | | | |
| EPI1181 | | | | | | | |
| EPI1327 | CD71 | 844 | SYWMH | 845 | EINPTNGRTNYIEKFKS | 846 | GTRAYHY |
| EPI1178 | | | | | | | |
| EPI1182 | | | | | | | |
| EPI1328 | CD71 | 850 | SYWMH | 851 | EIAPTNGRTNYIEKFKS | 852 | GTRAYHY |
| EPI1179 | | | | | | | |
| EPI1183 | | | | | | | |
| EPI1329 | CD71 | 856 | GHYVH | 857 | WIFPGKVNTKYNEKFK | 858 | VGYDYPYYFDY |
| EPI1184 | | | | | G | | |
| EPI1621 | IGF1R | 862 | SSNWWS | 863 | EIYHSGSTNYNPSLKS | 864 | WTGRTDAFDI |
| EPI1741 | | | | | | | |
| EPI1622 | IGF1R | 868 | SYGMH | 869 | IIWFDGSSTYYADSVRG | 870 | ELGRRYFDL |
| EPI1742 | | | | | | | |
| EPI1623 | IGF1R | 874 | SYAMN | 875 | AISGSGGTTFYADSVKG | 876 | DLGWSDSYYYYY |
| EPI1743 | | | | | | | GMDV |
| EPI1624 | IGF1R | 880 | SYAIS | 881 | GIIPIFGTANYAQKFQG | 882 | APLRFLEWSTQD |
| EPI1744 | | | | | | | HYYYYYMDV |

TABLE 3
Exemplary CDR sequences for antibodies targeting the
internalizing receptor protein.

| | | SEQ | | SEQ | | SEQ | |
|---|---|---|---|---|---|---|---|
| Antibody | Arm 1 | ID | | ID | | ID | |
| IDs | Target | NO | CDR L1 | NO | CDR L2 | NO | CDR L3 |
| EPI1092 | CD71 | 475 | SASSSVYY | 476 | STSNLAS | 477 | QQRRNYPYT |
| EPI1091 | | | MY | | | | |
| EPI1090 | | | | | | | |
| EPI1093 | | | | | | | |
| EPI1177 | | | | | | | |
| EPI1237 | MUC1 | 481 | GGNNIGSKS | 482 | YGSNRPS | 483 | QVWDSSSDWV |
| EPI1118 | | | VH | | | | |
| EPI1238 | MUC1 | 487 | RSSKSLLHS | 488 | QMSNLAS | 489 | AQNLELPPT |
| EPI1119 | | | NGITYFF | | | | |
| EPI1240 | MUC1 | 493 | KSSQSLLYS | 494 | WASTRES | 495 | QQYYRYPRT |
| EPI221 | | | SNQKIYLA | | | | |
| EPI1121 | | | | | | | |
| EPI1241 | MUC1 | 499 | SAHSSVSFM | 500 | STSSLAS | 501 | QQRSSFPLT |
| EPI1122 | | | H | | | | |
| EPI1242 | MUC1 | 505 | RSSQTIVHS | 506 | RVSKRFS | 507 | FQGSHVPWT |
| EPI2212 | | | NGKIYLE | | | | |
| EPI1123 | | | | | | | |
| EPI1243 | D276 | 511 | KASQNVDT | 512 | SASYRYS | 513 | QQYNNYPFT |
| EPI1135 | | | NVA | | | | |
| EPI1244 | CD276 | 517 | RASQSISDY | 518 | YASQSIS | 519 | QNGHSFPLT |
| EPI1136 | | | LH | | | | |
| EPI1245 | CD276 | 523 | KASQNVGF | 524 | SASYRYS | 525 | QQYNWYPFT |
| EPI2213 | | | NVA | | | | |
| EPI1137 | | | | | | | |
| EPI1246 | CD276 | 529 | RASSRLIYM | 530 | ATSNLAS | 531 | QQWNSNPPT |
| EPI2214 | | | H | | | | |
| EPI1138 | | | | | | | |
| EPI1247 | TPBG | 535 | KASQSVSN | 536 | YTSSRYA | 537 | QQDYNSPPT |
| | | | DVA | | | | |
| EPI1248 | TPBG | 541 | KASQSVSN | 542 | FATNRYT | 543 | QQDYSSPWT |
| EPI1522 | | | DVA | | | | |
| EPI1249 | TPBG | 547 | KASQDVDT | 548 | WASTRLT | 549 | QQYSSYPYT |
| EPI1523 | | | AVA | | | | |
| EPI1250 | TPBG | 553 | QASQSIGSE | 554 | RASTLES | 555 | QQGYTYSEIDNA |
| EPI1524 | | | LA | | | | |
| EPI1251 | TPBG | 559 | QASENIYST | 560 | DAFDLAS | 561 | QQGYSGTNVDNA |
| EPI1525 | | | LA | | | | |
| EPI1252 | TPBG | 565 | RASQSVSSY | 566 | DASNRAT | 567 | QQRSNWPLT |
| EPI1526 | | | LA | | | | |
| EPI1253 | MST1R | 571 | RASQSVSRY | 572 | DASNRAT | 573 | QQRSNWPRT |
| EPI1139 | | | LA | | | | |
| EPI1254 | MST1R | 577 | RASQNVGSS | 578 | SASFLYS | 579 | QQYNNYPLT |
| EPI1140 | | | LV | | | | |
| EPI1255 | MST1R | 583 | HASQNINV | 584 | KASNLHT | 585 | QQGQSYPLT |
| EPI1141 | | | WLN | | | | |
| EPI1256 | MST1R | 589 | KSSQSLLFS | 590 | WASTRAS | 591 | QQYYSFPRT |
| EPI1142 | | | GNQKNYLA | | | | |
| EPI1257 | HER3 | 595 | RSSQSVLYS | 596 | WASTRES | 597 | QQYYSTPRT |
| | | | SSNRNYLA | | | | |
| EPI1258 | HER3 | 601 | RASQGISNW | 602 | GASSLQS | 603 | QQYSSFPTT |
| EPI1168 | | | LA | | | | |
| EPI1259 | HER3 | 607 | RASQNIATD | 608 | SASFLYS | 609 | QQSEPEPYT |
| EPI1167 | | | VA | | | | |
| EPI1260 | HER3 | 613 | KSSQSVLNS | 614 | WASTRES | 615 | QSDYSYPYT |
| | | | GNQKNYLT | | | | |
| EPI1261 | HER3 | 619 | SGSSSNIGS | 620 | SDNHRPS | 621 | QGWDTSLSGHV |
| | | | NSVS | | | | |
| EPI1262 | ADAM9 | 625 | KASQSVDY | 626 | AASDLES | 627 | QQSHEDPFT |
| | | | SGDSYMN | | | | |
| EPI1263 | CDH3 | 631 | TGTSNDVG | 632 | EVNKRPS | 633 | SSYTMGSTFML |
| EPI2215 | | | AYNYVS | | | | |
| EPI1606 | | | | | | | |
| EPI1264 | CDH3 | 637 | RASQNIYSN | 638 | AAKNLAS | 639 | QHFYDTPWT |
| EPI2216 | | | LA | | | | |
| EPI1607 | | | | | | | |
| EPI1265 | CDH3 | 643 | RASQDITNY | 644 | YTSRLHS | 645 | QQDSKHPRT |
| EPI2217 | | | LN | | | | |
| EPI1734 | | | | | | | |
| EPI1266 | CDH3 | 649 | SASSSVSSG | 650 | RTSNLAS | 651 | QQWSGYPWT |
| EPI1735 | | | NFH | | | | |
| EPI1267 | CDH3 | 655 | RASQTISNT | 656 | AASNLQS | 657 | QQYLSWFT |
| EPI1736 | | | LA | | | | |
| EPI1268 | EpCAM | 661 | RSSKNLLHS | 662 | QMSNLAS | 663 | AQNLEIPRT |
| EPI1147 | | | NGITYLY | | | | |
| EPI1269 | EpCAM | 667 | RSSKSLLHS | 668 | QMSNLAS | 669 | AQNLELPRT |
| EPI1148 | | | NGITYLY | | | | |
| EPI1270 | EpCAM | 673 | RSTKSLLHS | 674 | QMSNLAS | 675 | AQNLEIPRT |
| EPI1149 | | | NGITYLY | | | | |
| EPI1271 | EpCAM | 679 | RASQSVSSS | 680 | GASSRAT | 681 | AQGELYPRQ |
| | | | YLA | | | | |
| EPI1272 | EpCAM | 685 | RASQSVSSN | 686 | GASTTAS | 687 | QQYNNWPPAYT |
| EPI1150 | | | LA | | | | |
| EPI1273 | TNFRSF10B | 691 | KASQDVGT | 692 | WASTRHT | 693 | QQYSSYRT |
| EPI1151 | | | AVA | | | | |
| EPI1274 | TNFRSF10B | 697 | SGDSLRSYY | 698 | GANNRPS | 699 | NSADSSGNHVV |
| EPI1152 | | | AS | | | | |
| EPI1275 | TNFRSF10B | 703 | RASQSISNN | 704 | FASQSIT | 705 | QQGNSWPYT |
| EPI1153 | | | LH | | | | |
| EPI1276 | TNFRSF10B | 709 | RASQGISRS | 710 | GASSRAT | 711 | QQFGSSPWT |
| EPI1154 | | | YLA | | | | |
| EPI1277 | TNFRSF10B | 715 | QGDSLRSY | 716 | GKNNRPS | 717 | NSRDSSGNHVV |
| EPI1155 | | | YAS | | | | |
| EPI1278 | ITGB6 | 721 | SASSSVSSS | 722 | STSNLAS | 723 | HQWSTYPPT |
| EPI1164 | | | YLY | | | | |
| EPI1279 | ITGB6 | 727 | GASENIYGA | 728 | GATNLED | 729 | QNVLTTPYT |
| EPI1407 | | | LN | | | | |
| EPI1280 | ITGB6 | 733 | KSSQSLLDS | 734 | LVSELDS | 735 | WQGTHFPRT |
| EPI1165 | | | DGKTYLN | | | | |
| EPI1281 | ITGB6 | 739 | SGDVLAKK | 740 | KDSERPS | 741 | YSAADNNLV |
| EPI1608 | | | SAR | | | | |
| EPI1282 | PD-L1 | 745 | RASQDVST | 746 | SASFLYS | 747 | QQYLYHPAT |
| | | | AVA | | | | |
| EPI1283 | PD-L1 | 751 | RASQRVSSS | 752 | DASSRAT | 753 | QQYGSLPWT |
| | | | YLA | | | | |
| EPI1284 | PD-L1 | 757 | TGTSSDVGG | 758 | DVSNRPS | 759 | SSYTSSSTRV |
| EPI1730 | | | YNYVS | | | | |
| EPI1285 | PD-L1 | 763 | GGNNIGSKS | 764 | DDSDRPS | 765 | QVWDSSSDHVV |
| EPI1731 | | | VH | | | | |
| EPI1296 | TROP2 | 769 | KASQDVSIA | 770 | SASYRYT | 771 | QQHYITPLT |
| EPI2218 | | | VA | | | | |
| EPI1166 | | | | | | | |
| EPI1297 | TROP2 | 775 | KASQDVST | 776 | SASYRYT | 777 | QQHYITPLT |
| EPI2219 | | | AVA | | | | |
| EPI1737 | | | | | | | |
| EPI1298 | TROP2 | 781 | RASKSVSTS | 782 | LASNLES | 783 | QHSRELPYT |
| EPI1738 | | | GYSYMH | | | | |
| EPI1299 | TROP2 | 787 | RASQTIGTSI | 788 | YASESIS | 789 | SQSFSWPFT |
| EPI1739 | | | H | | | | |
| EPI1300 | TROP2 | 793 | KSSQSLLNS | 794 | GASTRES | 795 | QSDHIYPYT |
| EPI1740 | | | GNQQNYLA | | | | |
| EPI1301 | EphA2 | 799 | RASQSISTW | 800 | KASNLHT | 801 | QQYNSYSRT |
| EPI1732 | | | LA | | | | |
| EPI1302 | EphA2 | 805 | RSSQSIVHS | 806 | KVSNRFS | 807 | FQGSHVPYT |
| EPI1733 | | | NGNTYLE | | | | |
| EPI1303 | EphA2 | 811 | RSSQSLVHS | 812 | RVSNRFS | 813 | FQATHVPWT |
| EPI1609 | | | NGNTFLY | | | | |
| EPI1304 | EphA2 | 817 | RASQSVSSS | 818 | FASNLES | 819 | QHS WEIPPT |
| EPI1552 | | | SYTYIH | | | | |
| EPI1323 | CD71 | 823 | RASDNLYS | 824 | DATNLAD | 825 | QHFWGTPLT |
| | | | NLA | | | | |
| EPI1324 | CD71 | 829 | RASDNLYS | 830 | DATNLAD | 831 | QHFWGTPLT |
| | | | NLA | | | | |
| EPI1325 | CD71 | 835 | RASDNLYS | 836 | DATNLAD | 837 | QHFWGTPLT |
| | | | NLA | | | | |
| EPI1326 | CD71 | 841 | RASDNLYS | 842 | DATNLAD | 843 | QHFAGTPLT |
| EPI1180 | | | NLA | | | | |
| EPI1181 | | | | | | | |
| EPI1327 | CD71 | 847 | RASDNLYS | 848 | DATNLAD | 849 | QHFAGTPLT |
| EPI1178 | | | NLA | | | | |
| EPI1182 | | | | | | | |
| EPI1328 | CD71 | 853 | RASDNLYS | 854 | DATNLAD | 855 | QHFAGTPLT |
| EPI1179 | | | NLA | | | | |
| EPI1183 | | | | | | | |
| EPI1329 | CD71 | 859 | RASQSIGTSI | 860 | YASESIS | 861 | QQSSSWPFT |
| EPI1184 | | | H | | | | |
| EPI1621 | IGF1R | 865 | RSSQSLLHS | 866 | LGSNRAS | 867 | MQGTHWPLT |
| EPI1741 | | | NGYNYLD | | | | |
| EPI1622 | IGF1R | 871 | RASQSVSSY | 872 | DASKRAT | 873 | QQRSKWPPWT |
| EPI1742 | | | LA | | | | |
| EPI1623 | IGF1R | 877 | RASQGIRND | 878 | AASRLHR | 879 | LQHNSYPCS |
| EPI1743 | | | LG | | | | |
| EPI1624 | IGF1R | 883 | QGDSLRSY | 884 | GENKRPS | 885 | KSRDGSGQHLV |
| EPI1744 | | | YAT | | | | |

The sequences listed in Table 2 and 3 (SEQ ID NOs: 475-885) are amino acid molecules. The sequences listed in Table 2 or 3 (SEQ ID NOs: 475-885) are amino acid molecules that are synthetic constructs. The sequences listed in Table 2 or 3 (SEQ ID NOS: 475-885) for CDR (complementarity-determining regions) sequences are amino acid molecules that are synthetic constructs.

In some embodiments, the first binding domain comprises at least one complementarity-determining region (CDR) sequence. The first binding domain comprising at least one complementarity-determining region (CDR) sequence may comprise one or more sequences listed in Table 2 or 3. In some cases, the first binding domain comprising at least one complementarity-determining region (CDR) sequence comprises at least 90% sequence identity to one or more sequences listed in Table 2 or 3. In some cases, the first binding domain comprising an antibody comprising an HC sequence, a VH sequence, an LC sequence, and a VL sequence comprises at least 91% sequence identity to one or more sequences listed in Table 2 or 3. In some cases, the first binding domain comprising at least one complementarity-determining region (CDR) sequence comprises at least 92% sequence identity to one or more sequences listed in Table 2 or 3. In some cases, the first binding domain comprising at least one complementarity-determining region (CDR) sequence comprises at least 93% sequence identity to one or more sequences listed in Table 2 or 3. In some cases, the first binding domain comprising at least one complementarity-determining region (CDR) sequence comprises at least 94% sequence identity to one or more sequences listed in Table 2 or 3. In some cases, the first binding domain comprising at least one complementarity-determining region (CDR) sequence comprises at least 95% sequence identity to one or more sequences listed in Table 2 or 3. In some cases, the first binding domain comprising at least one complementarity-determining region (CDR) sequence comprises at least 96% sequence identity to one or more sequences listed in Table 2 or 3. In some cases, the first binding domain comprising at least one complementarity-determining region (CDR) sequence comprises at least 97% sequence identity to one or more sequences listed in Table 2 or 3. In some cases, the first binding domain comprising at least one complementarity-determining region (CDR) sequence comprises at least 98% sequence identity to one or more sequences listed in Table 2 or 3. In some cases, the first binding domain comprising at least one complementarity-determining region (CDR) sequence comprises at least 99% sequence identity to one or more sequences listed in Table 2 or 3. In some cases, the first binding domain comprising at least one complementarity-determining region (CDR) sequence comprises at least 99.5% sequence identity to one or more sequences listed in Table 2 or 3. In some cases, the first binding domain comprising at least one complementarity-determining region (CDR) sequence comprises at least 99.99% sequence identity to one or more sequences listed in Table 2 or 3.

In some embodiments, the first binding domain comprises at least one sequence listed Table 2 or 3. In some embodiments, the first binding domain comprises at least 90%, at least 91%, at least 92%, at least 93%, at least 94%, at least 95%, at least 96%, at least 97%, at least 98%, at least 99%, at least 99.5%, or at least 99.9% sequence identity to the sequences listed Table 2 or 3.

In some embodiments, the first binding domain comprises a sequence listed Table 4. In some embodiments, the first binding domain comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 4.

In some cases, the first binding domain may bind the same epitope as any one of the antibodies listed in Table 4. The first binding domain may bind to an epitope that comprises about 70% sequence identity to the epitope to which any one of the antibodies listed in Table 4 binds. The first binding domain may bind to an epitope that comprises about 75% sequence identity to the epitope to which any one of the antibodies listed in Table 4 binds. The first binding domain may bind to an epitope that comprises about 80% sequence identity to the epitope to which any one of the antibodies listed in Table 4 binds. The first binding domain may bind to an epitope that comprises about 85% sequence identity to the epitope to which any one of the antibodies listed in Table 4 binds. The first binding domain may bind to an epitope that comprises about 90% sequence identity to the epitope to which any one of the antibodies listed in Table 4 binds. The first binding domain may bind to an epitope that comprises about 95% sequence identity to the epitope to which any one of the antibodies listed in Table 4 binds. The first binding domain may bind to an epitope that comprises about 99% sequence identity to the epitope to which any one of the antibodies listed in Table 4 binds.

In some embodiments, the first binding domain may bind the same epitope as any one of the antibodies listed in Table 4 binds with a similar affinity as any one of the antibodies listed in Table 4. The first binding domain may bind to an epitope that comprises about 70% sequence identity to the epitope to which any one of the antibodies listed in Table 4 binds with a similar affinity as any one of the antibodies listed in Table 4. The first binding domain may bind to an epitope that comprises about 75% sequence identity to the epitope to which any one of the antibodies listed in Table 4 binds with a similar affinity as any one of the antibodies listed in Table 4. The first binding domain may bind to an epitope that comprises about 80% sequence identity to the epitope to which any one of the antibodies listed in Table 4 binds with a similar affinity as any one of the antibodies listed in Table 4. The first binding domain may bind to an epitope that comprises about 85% sequence identity to the epitope to which any one of the antibodies listed in Table 4 binds with a similar affinity as any one of the antibodies listed in Table 4. The first binding domain may bind to an epitope that comprises about 90% sequence identity to the epitope to which any one of the antibodies listed in Table 4 binds with a similar affinity as any one of the antibodies listed in Table 4. The first binding domain may bind to an epitope that comprises about 95% sequence identity to the epitope to which any one of the antibodies listed in Table 4 binds with a similar affinity as any one of the antibodies listed in Table 4. The first binding domain may bind to an epitope that comprises about 99% sequence identity to the epitope to which any one of the antibodies listed in Table 4 binds with a similar affinity as any one of the antibodies listed in Table 4.

In some embodiments, the first binding domain may bind the same epitope as any one of the antibodies listed in Table 4 binds with a different affinity as compared to any one of the antibodies listed in Table 4. The first binding domain may bind to an epitope that comprises about 70% sequence identity to the epitope to which any one of the antibodies listed in Table 4 binds with a different affinity as compared to any one of the antibodies listed in Table 4. The first binding domain may bind to an epitope that comprises about 75% sequence identity to the epitope to which any one of the antibodies listed in Table 4 binds with a different affinity as compared to any one of the antibodies listed in Table 4. The first binding domain may bind to an epitope that comprises about 80% sequence identity to the epitope to which any one of the antibodies listed in Table 4 binds with a different affinity as compared to any one of the antibodies listed in Table 4. The first binding domain may bind to an epitope that comprises about 85% sequence identity to the epitope to which any one of the antibodies listed in Table 4 binds with a different affinity as compared to any one of the antibodies listed in Table 4. The first binding domain may bind to an epitope that comprises about 90% sequence identity to the epitope to which any one of the antibodies listed in Table 4 binds with a different affinity as compared to any one of the antibodies listed in Table 4. The first binding domain may bind to an epitope that comprises about 95% sequence identity to the epitope to which any one of the antibodies listed in Table 4 binds with a different affinity as compared to any one of the antibodies listed in Table 4. The first binding domain may bind to an epitope that comprises about 99% sequence identity to the epitope to which any one of the antibodies listed in Table 4 binds with a different affinity as compared to any one of the antibodies listed in Table 4.

The first binding domain may bind to an epitope that comprises a different epitope than the epitope to which any one of the antibodies listed in Table 4 binds. The first binding domain may bind to an epitope that comprises a different epitope than the epitope to which any one of the antibodies listed in Table 4 binds, wherein the epitopes do not bind to any of the same amino acids on the internalizing receptor protein. The first binding domain may bind to an epitope that comprises a different epitope than the epitope to which any one of the antibodies listed in Table 4 binds, wherein the epitopes bind to any one or more of the same amino acids on the internalizing receptor protein. The first binding domain may bind to an epitope that comprises a different epitope than the epitope to which any one of the antibodies listed in Table 4 binds, wherein the epitopes bind to any two or more of the same amino acids on the internalizing receptor protein. The first binding domain may bind to an epitope that comprises a different epitope than the epitope to which any one of the antibodies listed in Table 4 binds, wherein the epitopes bind to any three or more of the same amino acids on the internalizing receptor protein. The first binding domain may bind to an epitope that comprises a different epitope than the epitope to which any one of the antibodies listed in Table 4 binds, wherein the epitopes bind to any four or more of the same amino acids on the internalizing receptor protein. The first binding domain may bind to an epitope that comprises a different epitope than the epitope to which any one of the antibodies listed in Table 4 binds, wherein the epitopes bind to any five or more of the same amino acids on the internalizing receptor protein. The first binding domain may bind to an epitope that comprises a different epitope than the epitope to which any one of the antibodies listed in Table 4 binds, wherein the epitopes bind to any six or more of the same amino acids on the internalizing receptor protein. The first binding domain may bind to an epitope that comprises a different epitope than the epitope to which any one of the antibodies listed in Table 4 binds, wherein the epitopes bind to any seven or more of the same amino acids on the internalizing receptor protein. The first binding domain may bind to an epitope that comprises a different epitope than the epitope to which any one of the antibodies listed in Table 4 binds, wherein the epitopes bind to any eight or more of the same amino acids on the internalizing receptor protein. The first binding domain may bind to an epitope that comprises a different epitope than the epitope to which any one of the antibodies listed in Table 4 binds, wherein the epitopes bind to any nine or more of the same amino acids on the internalizing receptor protein. The first binding domain may bind to an epitope that comprises a different epitope than the epitope to which any one of the antibodies listed in Table 4 binds, wherein the epitopes bind to any ten or more of the same amino acids on the internalizing receptor protein.

In some embodiments, the antibodies targeting the degrader protein comprises a sequence listed Table 4. In some embodiments, the antibodies targeting the degrader protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 4.

In some cases, the antibodies targeting the degrader protein may bind the same epitope as any one of the antibodies listed in Table 4. The antibodies targeting the degrader protein may bind to an epitope that comprises about 70% sequence identity to the epitope to which any one of the antibodies listed in Table 4 binds. The antibodies targeting the degrader protein may bind to an epitope that comprises about 75% sequence identity to the epitope to which any one of the antibodies listed in Table 4 binds. The antibodies targeting the degrader protein may bind to an epitope that comprises about 80% sequence identity to the epitope to which any one of the antibodies listed in Table 4 binds. The antibodies targeting the degrader protein may bind to an epitope that comprises about 85% sequence identity to the epitope to which any one of the antibodies listed in Table 4 binds. The antibodies targeting the degrader protein may bind to an epitope that comprises about 90% sequence identity to the epitope to which any one of the antibodies listed in Table 4 binds. The antibodies targeting the degrader protein may bind to an epitope that comprises about 95% sequence identity to the epitope to which any one of the antibodies listed in Table 4 binds. The antibodies targeting the degrader protein may bind to an epitope that comprises about 99% sequence identity to the epitope to which any one of the antibodies listed in Table 4 binds.

The antibodies targeting the degrader protein may bind to an epitope that comprises a different epitope than the epitope to which any one of the antibodies listed in Table 4 binds. The antibodies targeting the degrader protein may bind to an epitope that comprises a different epitope than the epitope to which any one of the antibodies listed in Table 4 binds, wherein the epitopes do not bind to any of the same amino acids on the degrader protein. The antibodies targeting the degrader protein may bind to an epitope that comprises a different epitope than the epitope to which any one of the antibodies listed in Table 4 binds, wherein the epitopes bind to any one or more of the same amino acids on the degrader protein. The antibodies targeting the degrader protein may bind to an epitope that comprises a different epitope than the epitope to which any one of the antibodies listed in Table 4 binds, wherein the epitopes bind to any two or more of the same amino acids on the degrader protein. The antibodies targeting the degrader protein may bind to an epitope that comprises a different epitope than the epitope to which any one of the antibodies listed in Table 4 binds, wherein the epitopes bind to any three or more of the same amino acids on the degrader protein. The antibodies targeting the degrader protein may bind to an epitope that comprises a different epitope than the epitope to which any one of the antibodies listed in Table 4 binds, wherein the epitopes bind to any four or more of the same amino acids on the degrader protein. The antibodies targeting the degrader protein may bind to an epitope that comprises a different epitope than the epitope to which any one of the antibodies listed in Table 4 binds, wherein the epitopes bind to any five or more of the same amino acids on the degrader protein. The antibodies targeting the degrader protein may bind to an epitope that comprises a different epitope than the epitope to which any one of the antibodies listed in Table 4 binds, wherein the epitopes bind to any six or more of the same amino acids on the degrader protein. The antibodies targeting the degrader protein may bind to an epitope that comprises a different epitope than the epitope to which any one of the antibodies listed in Table 4 binds, wherein the epitopes bind to any seven or more of the same amino acids on the degrader protein. The antibodies targeting the degrader protein may bind to an epitope that comprises a different epitope than the epitope to which any one of the antibodies listed in Table 4 binds, wherein the epitopes bind to any eight or more of the same amino acids on the degrader protein. The antibodies targeting the degrader protein may bind to an epitope that comprises a different epitope than the epitope to which any one of the antibodies listed in Table 4 binds, wherein the epitopes bind to any nine or more of the same amino acids on the degrader protein. The antibodies targeting the degrader protein may bind to an epitope that comprises a different epitope than the epitope to which any one of the antibodies listed in Table 4 binds, wherein the epitopes bind to any ten or more of the same amino acids on the degrader protein.

TABLE 4
Additional exemplary antibody sequences targeting the degrader protein.

| | | SEQ | | SEQ | | SEQ | | SEQ | |
|---|---|---|---|---|---|---|---|---|---|
| Antibody | Arm 1 | ID | | ID | VH | ID | | ID | VL |
| IDs | Target | NO | HC sequence | NO | sequence | NO | LC sequence | NO | sequence |
| EPI1286 | RNF43 | 277 | EVQLVQSGAEVKKPGASV | 278 | EVQLVQSG | 279 | EIVMTQSPATLS | 280 | EIVMTQ |
| EPI1156 | | | KVSCKASGYTFTTYTIHW | | AEVKKPGAS | | VSPGERATLSCK | | SPATLSV |
| | | | VRQAPGQGLEWMGYINPR | | VKVSCKAS | | ASQNVGINVAW | | SPGERA |
| | | | SGYTEYNQKFQDRVTMTR | | GYTFTTYTI | | YQQKPGQAPRA | | TLSCKA |
| | | | DTSTSTVYMELSSLRSEDT | | HWVRQAPG | | LIYSASYRYSGIP | | SQNVGI |
| | | | AVYYCARSYEFWGQGTT | | QGLEWMGY | | ARFSGSGSGTEF | | NVAWY |
| | | | VTVSSASTKGPSVFPLAPS | | INPRSGYTE | | TLTISSLQSEDFA | | QQKPGQ |
| | | | SKSTSGGTAALGCLVKDY | | YNQKFQDR | | VYYCHQYKTYP | | APRALIY |
| | | | FPEPVTVSWNSGALTSGV | | VTMTRDTST | | YTFGGGTKLEIK | | SASYRY |
| | | | HTFPAVLQSSGLYSLSSVV | | STVYMELSS | | RTVAAPSVFIFPP | | SGIPARF |
| | | | TVPSSSLGTQTYICNVNHK | | LRSEDTAVY | | SDEQLKSGTASV | | SGSGSG |
| | | | PSNTKVDKKVEPKSCDKT | | YCARSYEF | | VCLLNNFYPRE | | TEFTLTI |
| | | | HTCPPCPAPELLGGPSVFL | | WGQGTTVT | | AKVQWKVDNA | | SSLQSED |
| | | | FPPKPKDTLMISRTPEVTC | | VSS | | LQSGNSQESVTE | | FAVYYC |
| | | | VVVDVSHEDPEVKFNWY | | | | QDSKDSTYSLSS | | HQYKTY |
| | | | VDGVEVHNAKTKPREEQY | | | | TLTLSKADYEK | | PYTFGG |
| | | | NSTYRVVSVLTVLHQDWL | | | | HKVYACEVTHQ | | GTKLEIK |
| | | | NGKEYKCKVSNKALPAPI | | | | GLSSPVTKSFNR | | |
| | | | EKTISKAKGQPREPQVYTL | | | | GEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGK | | | | | | |
| EPI1287 | RNF43 | 281 | AVQLVESGGGSVQPGRSM | 282 | AVQLVESG | 283 | DVVLTQTPVSLS | 284 | DVVLTQ |
| EPI1157 | | | RLSCAASGFTFSNYDMTW | | GGSVQPGRS | | VTVGDQASISCR | | TPVSLSV |
| | | | VRQAPTKGLEWVASITSD | | MRLSCAAS | | SSQSLEYSDGYS | | TVGDQA |
| | | | GGSTYSRDSVKGRFTISRD | | GFTFSNYDM | | YLEWYLQKPGQ | | SISCRSS |
| | | | NAKSTLYLQMDSLRSEDT | | TWVRQAPT | | SPQLLIYEVSSRF | | QSLEYS |
| | | | ATYYCTTDRGRYLPYYFD | | KGLEWVASI | | SGVPDRFIGSGS | | DGYSYL |
| | | | YWGQGVMVTVSSASTKG | | TSDGGSTYS | | GTDFTLKISRVE | | EWYLQK |
| | | | PSVFPLAPSSKSTSGGTAA | | RDSVKGRFT | | PEDLGVYYCFQ | | PGQSPQ |
| | | | LGCLVKDYFPEPVTVSWN | | ISRDNAKST | | AIHDPTFGAGTK | | LLIYEVS |
| | | | SGALTSGVHTFPAVLQSSG | | LYLQMDSL | | LELKRTVAAPSV | | SRFSGVP |
| | | | LYSLSSVVTVPSSSLGTQT | | RSEDTATYY | | FIFPPSDEQLKSG | | DRFIGSG |
| | | | YICNVNHKPSNTKVDKKV | | CTTDRGRYL | | TASVVCLLNNF | | SGTDFT |
| | | | EPKSCDKTHTCPPCPAPEL | | PYYFDYWG | | YPREAKVQWKV | | LKISRVE |
| | | | LGGPSVFLFPPKPKDTLMI | | QGVMVTVS | | DNALQSGNSQE | | PEDLGV |
| | | | SRTPEVTCVVVDVSHEDP | | S | | SVTEQDSKDSTY | | YYCFQA |
| | | | EVKFNWYVDGVEVHNAK | | | | SLSSTLTLSKAD | | IHDPTFG |
| | | | TKPREEQYNSTYRVVSVL | | | | YEKHKVYACEV | | AGTKLE |
| | | | TVLHQDWLNGKEYKCKV | | | | THQGLSSPVTKS | | LK |
| | | | SNKALPAPIEKTISKAKGQ | | | | FNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1288 | RNF43 | 285 | QVQLKESGPGLVQPSQTLS | 286 | QVQLKESGP | 287 | DTVLTQSPALA | 288 | DTVLTQ |
| EPI1158 | | | LTCTVSGFSLTTYSVHWV | | GLVQPSQTL | | VSPGERVTISCR | | SPALAV |
| | | | RQHSGKNLEWMGRMWT | | SLTCTVSGF | | ASESVSKLMHW | | SPGERV |
| | | | AGDTSYNSAFTSRLNIFRD | | SLTTYSVHW | | YQQRPGQQPQL | | TISCRAS |
| | | | TSKSQVFLKMNSLQTEDT | | VRQHSGKN | | LIYLTSHLASGV | | ESVSKL |
| | | | GTYYCARSSYTSGYPFDS | | LEWMGRM | | PARFSGSGSGTD | | MHWYQ |
| | | | WGQGVMVTVSSASTKGPS | | WTAGDTSY | | FTLTIDPVEADD | | QRPGQQ |
| | | | VFPLAPSSKSTSGGTAALG | | NSAFTSRLNI | | TATYYCQQSRN | | PQLLIYL |
| | | | CLVKDYFPEPVTVSWNSG | | FRDTSKSQV | | DPTFGAGTKLEL | | TSHLAS |
| | | | ALTSGVHTFPAVLQSSGLY | | FLKMNSLQT | | KRTVAAPSVFIF | | GVPARF |
| | | | SLSSVVTVPSSSLGTQTYIC | | EDTGTYYC | | PPSDEQLKSGTA | | SGSGSG |
| | | | NVNHKPSNTKVDKKVEPK | | ARSSYTSGY | | SVVCLLNNFYPR | | TDFTLTI |
| | | | SCDKTHTCPPCPAPELLGG | | PFDSWGQG | | EAKVQWKVDN | | DPVEAD |
| | | | PSVFLFPPKPKDTLMISRTP | | VMVTVSS | | ALQSGNSQESVT | | DTATYY |
| | | | EVTCVVVDVSHEDPEVKF | | | | EQDSKDSTYSLS | | CQQSRN |
| | | | NWYVDGVEVHNAKTKPR | | | | STLTLSKADYEK | | DPTFGA |
| | | | EEQYNSTYRVVSVLTVLH | | | | HKVYACEVTHQ | | GTKLEL |
| | | | QDWLNGKEYKCKVSNKA | | | | GLSSPVTKSFNR | | K |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1289 | RNF43 | 289 | EVQLVESGGGLVQPGGSL | 290 | EVQLVESGG | 291 | DIQMTQSPSSLS | 292 | DIQMTQ |
| EPI1159 | | | RLSCVVSGFTFSYYDMHW | | GLVQPGGSL | | ASVGDRVTITCR | | SPSSLSA |
| | | | VRQVTGKGLEWVSAIGTA | | RLSCVVSGF | | ASQSISSYLNWY | | SVGDRV |
| | | | GATYYPGSVKGRFTISREN | | TFSYYDMH | | QQKPGKAPKLLI | | TITCRAS |
| | | | AKNSLYLQMNSLRAGDTA | | WVRQVTGK | | YAASSLQSGVPS | | QSISSYL |
| | | | VYYCARDRGYSGYDAYY | | GLEWVSAIG | | RFSGSGSGTDFT | | NWYQQ |
| | | | FDFWGQGTLVTVSSASTK | | TAGATYYP | | LTISSLQPEDFAT | | KPGKAP |
| | | | GPSVFPLAPSSKSTSGGTA | | GSVKGRFTI | | YYCQQSYSTPPT | | KLLIYA |
| | | | ALGCLVKDYFPEPVTVSW | | SRENAKNSL | | FGQGTKVEIKRT | | ASSLQS |
| | | | NSGALTSGVHTFPAVLQSS | | YLQMNSLR | | VAAPSVFIFPPSD | | GVPSRFS |
| | | | GLYSLSSVVTVPSSSLGTQ | | AGDTAVYY | | EQLKSGTASVV | | GSGSGT |
| | | | TYICNVNHKPSNTKVDKK | | CARDRGYS | | CLLNNFYPREA | | DFTLTIS |
| | | | VEPKSCDKTHTCPPCPAPE | | GYDAYYFD | | KVQWKVDNAL | | SLQPEDF |
| | | | LLGGPSVFLFPPKPKDTLM | | FWGQGTLV | | QSGNSQESVTEQ | | ATYYCQ |
| | | | ISRTPEVTCVVVDVSHEDP | | TVSS | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI1290 | RNF43 | 293 | QVQLQESGPGLVKPSETLS | 294 | QVQLQESGP | 295 | DIQMTQSPSSLS | 296 | DIQMTQ |
| EPI1160 | | | LTCTVSGGSISSSNYYWG | | GLVKPSETL | | ASVGDRVTITCR | | SPSSLSA |
| | | | WIRQPPGKGLEWIGNIYYR | | SLTCTVSGG | | ASQSISSYLNWY | | SVGDRV |
| | | | GYTYYNPSLKSRVTISVDT | | SISSSNYYW | | QQKPGKAPKLLI | | TITCRAS |
| | | | SKKQFSLTLSSVTAADTA | | GWIRQPPGK | | YAASSLQSGVPS | | QSISSYL |
| | | | MYYCAREGSDYGDYVGA | | GLEWIGNIY | | RFSGSGSGTDFT | | NWYQQ |
| | | | FDIWDQGTMVTVSSASTK | | YRGYTYYN | | LTISSLQPEDFAT | | KPGKAP |
| | | | GPSVFPLAPSSKSTSGGTA | | PSLKSRVTIS | | YYCQQSYSTPPT | | KLLIYA |
| | | | ALGCLVKDYFPEPVTVSW | | VDTSKKQFS | | FGQGTKVEIKRT | | ASSLQS |
| | | | NSGALTSGVHTFPAVLQSS | | LTLSSVTAA | | VAAPSVFIFPPSD | | GVPSRFS |
| | | | GLYSLSSVVTVPSSSLGTQ | | DTAMYYCA | | EQLKSGTASVV | | GSGSGT |
| | | | TYICNVNHKPSNTKVDKK | | REGSDYGD | | CLLNNFYPREA | | DFTLTIS |
| | | | VEPKSCDKTHTCPPCPAPE | | YVGAFDIW | | KVQWKVDNAL | | SLQPEDF |
| | | | LLGGPSVFLFPPKPKDTLM | | DQGTMVTV | | QSGNSQESVTEQ | | ATYYCQ |
| | | | ISRTPEVTCVVVDVSHEDP | | SS | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1291 | RNF43 | 297 | QVQLVQSGAEVKKPGASV | 298 | QVQLVQSG | 299 | DIVMTQSPDSLA | 300 | DIVMTQ |
| EPI1161 | | | KVSCKASGFNIKDTYIHW | | AEVKKPGAS | | VSLGERATINCR | | SPDSLA |
| | | | VRQAPGQGLEWMGRIDPA | | VKVSCKAS | | ASESVDSYGNSF | | VSLGER |
| | | | NGKANYDPKFQGRVTMT | | GFNIKDTYI | | MHWYQQKPGQ | | ATINCR |
| | | | RDTSTSTVYMELSSLRSED | | HWVRQAPG | | PPKLLIYLASNL | | ASESVD |
| | | | TAVYYCALGGGYYGMDY | | QGLEWMGR | | ESGVPDRFSGSG | | SYGNSF |
| | | | WGQGTLVTVSSASTKGPS | | IDPANGKAN | | SGTDFTLTISSLQ | | MHWYQ |
| | | | VFPLAPSSKSTSGGTAALG | | YDPKFQGR | | AEDVAVYYCQQ | | QKPGQP |
| | | | CLVKDYFPEPVTVSWNSG | | VTMTRDTST | | NNEDPLTFGQG | | PKLLIYL |
| | | | ALTSGVHTFPAVLQSSGLY | | STVYMELSS | | TKVEIKRTVAAP | | ASNLES |
| | | | SLSSVVTVPSSSLGTQTYIC | | LRSEDTAVY | | SVFIFPPSDEQLK | | GVPDRF |
| | | | NVNHKPSNTKVDKKVEPK | | YCALGGGY | | SGTASVVCLLN | | SGSGSG |
| | | | SCDKTHTCPPCPAPELLGG | | YGMDYWG | | NFYPREAKVQW | | TDFTLTI |
| | | | PSVFLFPPKPKDTLMISRTP | | QGTLVTVSS | | KVDNALQSGNS | | SSLQAE |
| | | | EVTCVVVDVSHEDPEVKF | | | | QESVTEQDSKDS | | DVAVYY |
| | | | NWYVDGVEVHNAKTKPR | | | | TYSLSSTLTLSK | | CQQNNE |
| | | | EEQYNSTYRVVSVLTVLH | | | | ADYEKHKVYAC | | DPLTFG |
| | | | QDWLNGKEYKCKVSNKA | | | | EVTHQGLSSPVT | | QGTKVE |
| | | | LPAPIEKTISKAKGQPREPQ | | | | KSFNRGEC | | IK |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1292 | RNF43 | 301 | QVQLVQSGAEVKKPGASV | 302 | QVQLVQSG | 303 | DIQMTQSPSSLS | 304 | DIQMTQ |
| EPI1489 | | | KVSCKASGYTFTRYWIEW | | AEVKKPGAS | | ASVGDRVTITCK | | SPSSLSA |
| | | | VRQAPGQRLEWMGEILPG | | VKVSCKAS | | ASEDIYNRLAW | | SVGDRV |
| | | | SGSTNYNEKFKGRVTITAD | | GYTFTRYWI | | YQQKPGKAPKL | | TITCKAS |
| | | | TSASTAYMELSSLRSEDTA | | EWVRQAPG | | LISGATSLETGV | | EDIYNR |
| | | | VYYCERRGAYWGQGTLV | | QRLEWMGE | | PSRFSGSGSGTD | | LAWYQ |
| | | | TVSSASTKGPSVFPLAPSS | | ILPGSGSTN | | YTLTISSLQPEDF | | QKPGKA |
| | | | KSTSGGTAALGCLVKDYF | | YNEKFKGR | | ATYYCQQQWST | | PKLLISG |
| | | | PEPVTVSWNSGALTSGVH | | VTITADTSA | | PPTFGGGTKVEI | | ATSLET |
| | | | TFPAVLQSSGLYSLSSVVT | | STAYMELSS | | KRTVAAPSVFIF | | GVPSRFS |
| | | | VPSSSLGTQTYICNVNHKP | | LRSEDTAVY | | PPSDEQLKSGTA | | GSGSGT |
| | | | SNTKVDKKVEPKSCDKTH | | YCERRGAY | | SVVCLLNNFYPR | | DYTLTIS |
| | | | TCPPCPAPELLGGPSVFLFP | | WGQGTLVT | | EAKVQWKVDN | | SLQPEDF |
| | | | PKPKDTLMISRTPEVTCVV | | VSS | | ALQSGNSQESVT | | ATYYCQ |
| | | | VDVSHEDPEVKFNWYVD | | | | EQDSKDSTYSLS | | QQWSTP |
| | | | GVEVHNAKTKPREEQYNS | | | | STLTLSKADYEK | | PTFGGG |
| | | | TYRVVSVLTVLHQDWLN | | | | HKVYACEVTHQ | | TKVEIK |
| | | | GKEYKCKVSNKALPAPIE | | | | GLSSPVTKSFNR | | |
| | | | KTISKAKGQPREPQVYTLP | | | | GEC | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGK | | | | | | |
| EPI1293 | RNF43 | 305 | QVQLQESGGGLVQAGGSL | 306 | QVQLQESG | | | | |
| EPI1615 | | | RLSCAASGSIFWKPVMGW | | GGLVQAGG | | | | |
| | | | YRQAPGKEREFVAAITSGT | | SLRLSCAAS | | | | |
| | | | NTYYADSVKGRFTISRDN | | GSIFWKPVM | | | | |
| | | | AKNTVYLQMNSLKPEDTA | | GWYRQAPG | | | | |
| | | | VYYCAVDDYDVVEYPYW | | KEREFVAAI | | | | |
| | | | GQGTQVTVSSGGGGSDKT | | TSGTNTYYA | | | | |
| | | | HTCPPCPAPELLGGPSVFL | | DSVKGRFTI | | | | |
| | | | FPPKPKDTLMISRTPEVTC | | SRDNAKNT | | | | |
| | | | VVVDVSHEDPEVKFNWY | | VYLQMNSL | | | | |
| | | | VDGVEVHNAKTKPREEQY | | KPEDTAVY | | | | |
| | | | NSTYRVVSVLTVLHQDWL | | YCAVDDYD | | | | |
| | | | NGKEYKCKVSNKALPAPI | | VVEYPYWG | | | | |
| | | | EKTISKAKGQPREPQVYTL | | QGTQVTVSS | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI1294 | RNF43 | 305 | QQQLEEYGGDLVQPEGSL | 306 | QQQLEEYG | 307 | AEIVMTQTPSSK | 308 | AEIVMT |
| EPI1162 | | | TLTCKASGLDFSSSYWMC | | GDLVQPEGS | | SAAVGDTVTIKC | | QTPSSKS |
| | | | WVRQAPGKGLEWIACIYT | | LTLTCKASG | | QASQSITSYLSW | | AAVGDT |
| | | | GSSGSTSYASWAKGRFTIS | | LDFSSSYW | | YQQKPGQPPKL | | VTIKCQ |
| | | | KTSSTTVTLQMTSLTAAD | | MCWVRQAP | | LIYRASTLASGV | | ASQSITS |
| | | | TATYFCARDYDYTAYAY | | GKGLEWIAC | | PSRFKGSGSGTQ | | YLSWYQ |
| | | | GIMSLWGPGTLVTVSSAST | | IYTGSSGSTS | | FTLTISDLECAD | | QKPGQP |
| | | | KGPSVFPLAPSSKSTSGGT | | YASWAKGR | | AATYYCQSNYG | | PKLLIYR |
| | | | AALGCLVKDYFPEPVTVS | | FTISKTSSTT | | SYSTNYGVTFG | | ASTLAS |
| | | | WNSGALTSGVHTFPAVLQ | | VTLQMTSLT | | GGTKVEIKRTV | | GVPSRF |
| | | | SSGLYSLSSVVTVPSSSLG | | AADTATYFC | | AAPSVFIFPPSDE | | KGSGSG |
| | | | TQTYICNVNHKPSNTKVD | | ARDYDYTA | | QLKSGTASVVC | | TQFTLTI |
| | | | KKVEPKSCDKTHTCPPCP | | YAYGIMSL | | LLNNFYPREAK | | SDLECA |
| | | | APELLGGPSVFLFPPKPKD | | WGPGTLVT | | VQWKVDNALQ | | DAATYY |
| | | | TLMISRTPEVTCVVVDVSH | | VSS | | SGNSQESVTEQD | | CQSNYG |
| | | | EDPEVKFNWYVDGVEVH | | | | SKDSTYSLSSTL | | SYSTNY |
| | | | NAKTKPREEQYNSTYRVV | | | | TLSKADYEKHK | | GVTFGG |
| | | | SVLTVLHQDWLNGKEYK | | | | VYACEVTHQGL | | GTKVEI |
| | | | CKVSNKALPAPIEKTISKA | | | | SSPVTKSFNRGE | | K |
| | | | KGQPREPQVYTLPPSRDEL | | | | C | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI1295 | RNF43 | 307 | QEQLVESGGGLVQPEGSL | 308 | QEQLVESGG | 309 | DVVMTQTPASV | 310 | DVVMT |
| EPI1163 | | | TLTCTASGFSFSSRYYMC | | GLVQPEGSL | | SEPVGGTVTIKC | | QTPASV |
| | | | WVRQAPGKGLEWIGCIYT | | TLTCTASGF | | QASQSIYSGLA | | SEPVGG |
| | | | GSGSTYYASWAKGRVTIS | | SFSSRYYMC | | WYQQKPGQPPK | | TVTIKC |
| | | | KTSSTTVTLQMTSLTAAD | | WVRQAPGK | | LLIYSASKLASG | | QASQSIY |
| | | | TATYFCAREAGSFNLWGP | | GLEWIGCIY | | VPSRFKGSGSGT | | SGLAWY |
| | | | GTLVTVSSASTKGPSVFPL | | TGSGSTYYA | | EYTLTISDLECA | | QQKPGQ |
| | | | APSSKSTSGGTAALGCLV | | SWAKGRVTI | | DAATYYCQNYY | | PPKLLIY |
| | | | KDYFPEPVTVSWNSGALT | | SKTSSTTVT | | YGISNGWTFGG | | SASKLA |
| | | | SGVHTFPAVLQSSGLYSLS | | LQMTSLTAA | | GTKVEIKRTVA | | SGVPSRF |
| | | | SVVTVPSSSLGTQTYICNV | | DTATYFCAR | | APSVFIFPPSDEQ | | KGSGSG |
| | | | NHKPSNTKVDKKVEPKSC | | EAGSFNLW | | LKSGTASVVCLL | | TEYTLTI |
| | | | DKTHTCPPCPAPELLGGPS | | GPGTLVTVS | | NNFYPREAKVQ | | SDLECA |
| | | | VFLFPPKPKDTLMISRTPE | | S | | WKVDNALQSG | | DAATYY |
| | | | VTCVVVDVSHEDPEVKFN | | | | NSQESVTEQDSK | | CQNYYY |
| | | | WYVDGVEVHNAKTKPRE | | | | DSTYSLSSTLTL | | GISNGW |
| | | | EQYNSTYRVVSVLTVLHQ | | | | SKADYEKHKVY | | TFGGGT |
| | | | DWLNGKEYKCKVSNKAL | | | | ACEVTHQGLSSP | | KVEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1305 | ZNRF3 | 311 | QVQLVQSGAEVKKPGASV | 312 | QVQLVQSG | 313 | DIELTQPPSVSVS | 314 | DIELTQP |
| EPI1610 | | | KVSCKASGYTFTSYHMH | | AEVKKPGAS | | PGQTASITCSGD | | PSVSVSP |
| | | | WVRQAPGQGLEWMGWIN | | VKVSCKAS | | SIPSKYAHWYQ | | GQTASIT |
| | | | PYTGDTNYAQKFQGRVT | | GYTFTSYH | | QKPGQAPVLVIY | | CSGDSIP |
| | | | MTRDTSISTAYMELSRLRS | | MHWVRQAP | | GKSHRPSGIPER | | SKYAH |
| | | | EDTAVYYCAREKVYMDI | | GQGLEWMG | | FSGSNSGNTATL | | WYQQK |
| | | | WGQGTLVTVSSASTKGPS | | WINPYTGDT | | TISGTQAEDEAD | | PGQAPV |
| | | | VFPLAPSSKSTSGGTAALG | | NYAQKFQG | | YYCAAWDLLG | | LVIYGK |
| | | | CLVKDYFPEPVTVSWNSG | | RVTMTRDTS | | DGWVFGGGTKL | | SHRPSGI |
| | | | ALTSGVHTFPAVLQSSGLY | | ISTAYMELS | | TVLRTVAAPSVF | | PERFSGS |
| | | | SLSSVVTVPSSSLGTQTYIC | | RLRSEDTAV | | IFPPSDEQLKSGT | | NSGNTA |
| | | | NVNHKPSNTKVDKKVEPK | | YYCAREKV | | ASVVCLLNNFY | | TLTISGT |
| | | | SCDKTHTCPPCPAPELLGG | | YMDIWGQG | | PREAKVQWKVD | | QAEDEA |
| | | | PSVFLFPPKPKDTLMISRTP | | TLVTVSS | | NALQSGNSQES | | DYYCAA |
| | | | EVTCVVVDVSHEDPEVKF | | | | VTEQDSKDSTYS | | WDLLGD |
| | | | NWYVDGVEVHNAKTKPR | | | | LSSTLTLSKADY | | GWVFG |
| | | | EEQYNSTYRVVSVLTVLH | | | | EKHKVYACEVT | | GGTKLT |
| | | | QDWLNGKEYKCKVSNKA | | | | HQGLSSPVTKSF | | VVL |
| | | | LPAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1306 | ZNRF3 | 315 | EVQLVESGGGLVKPGGSL | 316 | EVQLVESGG | 317 | DIELTQPPSVSVS | 318 | DIELTQP |
| EPI1611 | | | RLSCAASGFTFSDYGIHW | | GLVKPGGSL | | PGQTASITCSGD | | PSVSVSP |
| | | | VRQAPGKGLEWVGRIKSK | | RLSCAASGF | | SLGSYYVHWYQ | | GQTASIT |
| | | | TDGGITEYAAPVKGRFTIS | | TFSDYGIHW | | QKPGQAPVLVIY | | CSGDSL |
| | | | RDDSKNTLYLQMNSLKTE | | VRQAPGKG | | RNKQRPSGIPER | | GSYYVH |
| | | | DTAVYYCARAIYYLEAFD | | LEWVGRIKS | | FSGSNSGNTATL | | WYQQK |
| | | | VWGQGTLVTVSSASTKGP | | KTDGGITEY | | TISGTQAEDEAD | | PGQAPV |
| | | | SVFPLAPSSKSTSGGTAAL | | AAPVKGRFT | | YYCQTYDWMY | | LVIYRN |
| | | | GCLVKDYFPEPVTVSWNS | | ISRDDSKNT | | SSRVFGGGTKLT | | KQRPSGI |
| | | | GALTSGVHTFPAVLQSSGL | | LYLQMNSL | | VLRTVAAPSVFI | | PERFSGS |
| | | | YSLSSVVTVPSSSLGTQTYI | | KTEDTAVY | | FPPSDEQLKSGT | | NSGNTA |
| | | | CNVNHKPSNTKVDKKVEP | | YCARAIYYL | | ASVVCLLNNFY | | TLTISGT |
| | | | KSCDKTHTCPPCPAPELLG | | EAFDVWGQ | | PREAKVQWKVD | | QAEDEA |
| | | | GPSVFLFPPKPKDTLMISR | | GTLVTVSS | | NALQSGNSQES | | DYYCQT |
| | | | TPEVTCVVVDVSHEDPEV | | | | VTEQDSKDSTYS | | YDWMY |
| | | | KFNWYVDGVEVHNAKTK | | | | LSSTLTLSKADY | | SSRVFG |
| | | | PREEQYNSTYRVVSVLTV | | | | EKHKVYACEVT | | GGTKLT |
| | | | LHQDWLNGKEYKCKVSN | | | | HQGLSSPVTKSF | | VL |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI1307 | ZNRF3 | 319 | EVQLVESGGGLVPPGKSL | 320 | EVQLVESGG | 321 | DIVMTQSPFSLA | 322 | DIVMTQ |
| | | | KLSCSASGFPFSNYGMHW | | GLVPPGKSL | | VSEGDMVTIMC | | SPFSLAV |
| | | | IRQAPGKGLDWVGYISSNS | | KLSCSASGF | | RSSQSLLSSGNQ | | SEGDMV |
| | | | GTIYADAVKGRFTISRDNA | | PFSNYGMH | | KNYLAWYQQK | | TIMCRSS |
| | | | KNTLYLLINSLKSEDTAM | | WIRQAPGK | | PGQSPKLLIYHA | | QSLLSSG |
| | | | YYCARGYFDGYYRFWGQ | | GLDWVGYIS | | STRQSGVPDRFI | | NQKNYL |
| | | | GVMVTVSSASTKGPSVFP | | SNSGTIYAD | | GSGSGTDFTLTI | | AWYQQ |
| | | | LAPSSKSTSGGTAALGCLV | | AVKGRFTIS | | SDVQAEDLADY | | KPGQSP |
| | | | KDYFPEPVTVSWNSGALT | | RDNAKNTL | | YCLQHYSSPTFG | | KLLIYH |
| | | | SGVHTFPAVLQSSGLYSLS | | YLLINSLKSE | | SGTKLEIKRTVA | | ASTRQS |
| | | | SVVTVPSSSLGTQTYICNV | | DTAMYYCA | | APSVFIFPPSDEQ | | GVPDRFI |
| | | | NHKPSNTKVDKKVEPKSC | | RGYFDGYY | | LKSGTASVVCLL | | GSGSGT |
| | | | DKTHTCPPCPAPELLGGPS | | RFWGQGVM | | NNFYPREAKVQ | | DFTLTIS |
| | | | VFLFPPKPKDTLMISRTPE | | VTVSS | | WKVDNALQSG | | DVQAED |
| | | | VTCVVVDVSHEDPEVKFN | | | | NSQESVTEQDSK | | LADYYC |
| | | | WYVDGVEVHNAKTKPRE | | | | DSTYSLSSTLTL | | LQHYSS |
| | | | EQYNSTYRVVSVLTVLHQ | | | | SKADYEKHKVY | | PTFGSGT |
| | | | DWLNGKEYKCKVSNKAL | | | | ACEVTHQGLSSP | | KLEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1308 | ZNRF3 | 323 | EVHLVESGGGLVQPGGSL | 324 | EVHLVESGG | 325 | DTVLTQSPALTV | 326 | DTVLTQ |
| | | | KLSCAASGFTFSNYDMAW | | GLVQPGGSL | | SPGDKITISCRAS | | SPALTVS |
| | | | VRQAPTRGLEWVASISPG | | KLSCAASGF | | EGVNTRIHWYQ | | PGDKITI |
| | | | GGKTYYRDSVKGRLTISR | | TFSNYDMA | | QKSGQQPKLLIY | | SCRASE |
| | | | NNAENTQYLQIDSLRSEDT | | WVRQAPTR | | GASNLDSGVPD | | GVNTRI |
| | | | ATYYCSRLGPAYSGEWFA | | GLEWVASIS | | RFSGSGFGTDFT | | HWYQQ |
| | | | YWGQGTLVTVSSASTKGP | | PGGGKTYY | | LTIDPVEASDTA | | KSGQQP |
| | | | SVFPLAPSSKSTSGGTAAL | | RDSVKGRLT | | TYFCQQSWNVP | | KLLIYG |
| | | | GCLVKDYFPEPVTVSWNS | | ISRNNAENT | | HTFGGGTKLEL | | ASNLDS |
| | | | GALTSGVHTFPAVLQSSGL | | QYLQIDSLR | | KRTVAAPSVFIF | | GVPDRF |
| | | | YSLSSVVTVPSSSLGTQTYI | | SEDTATYYC | | PPSDEQLKSGTA | | SGSGFG |
| | | | CNVNHKPSNTKVDKKVEP | | SRLGPAYSG | | SVVCLLNNFYPR | | TDFTLTI |
| | | | KSCDKTHTCPPCPAPELLG | | EWFAYWGQ | | EAKVQWKVDN | | DPVEAS |
| | | | GPSVFLFPPKPKDTLMISR | | GTLVTVSS | | ALQSGNSQESVT | | DTATYF |
| | | | TPEVTCVVVDVSHEDPEV | | | | EQDSKDSTYSLS | | CQQSWN |
| | | | KFNWYVDGVEVHNAKTK | | | | STLTLSKADYEK | | VPHTFG |
| | | | PREEQYNSTYRVVSVLTV | | | | HKVYACEVTHQ | | GGTKLE |
| | | | LHQDWLNGKEYKCKVSN | | | | GLSSPVTKSFNR | | LK |
| | | | KALPAPIEKTISKAKGQPR | | | | GEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI1309 | ZNRF3 | 327 | QVQLVQSGSELKKPGASV | 328 | QVQLVQSGS | 329 | DIQMTQSPSSLS | 330 | DIQMTQ |
| EPI1612 | | | KVSCKASGYTFTRYPMN | | ELKKPGASV | | ASVGDRVTITCR | | SPSSLSA |
| | | | WVRQAPGQGLEWMGWIN | | KVSCKASG | | ASQSISSYLNWY | | SVGDRV |
| | | | TNTGNPTYAQGFTGRFVF | | YTFTRYPMN | | QQKPGKAPKLLI | | TITCRAS |
| | | | SLDTSVSTAFLQISSLKAE | | WVRQAPGQ | | YAASSLQSGVPS | | QSISSYL |
| | | | DTAVYYCARERTNFYDAF | | GLEWMGWI | | RFSGSGSGTDFT | | NWYQQ |
| | | | DIWGQGTMVTVSSASTKG | | NTNTGNPTY | | LTISSLQPEDFAT | | KPGKAP |
| | | | PSVFPLAPSSKSTSGGTAA | | AQGFTGRFV | | YYCQQSYSTPPT | | KLLIYA |
| | | | LGCLVKDYFPEPVTVSWN | | FSLDTSVST | | FGQGTKVEIKRT | | ASSLQS |
| | | | SGALTSGVHTFPAVLQSSG | | AFLQISSLK | | VAAPSVFIFPPSD | | GVPSRFS |
| | | | LYSLSSVVTVPSSSLGTQT | | AEDTAVYY | | EQLKSGTASVV | | GSGSGT |
| | | | YICNVNHKPSNTKVDKKV | | CARERTNFY | | CLLNNFYPREA | | DFTL TIS |
| | | | EPKSCDKTHTCPPCPAPEL | | DAFDIWGQ | | KVQWKVDNAL | | SLQPEDF |
| | | | LGGPSVFLFPPKPKDTLMI | | GTMVTVSS | | QSGNSQESVTEQ | | ATYYCQ |
| | | | SRTPEVTCVVVDVSHEDP | | | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1310 | ZNRF3 | 331 | QVQLVQSGSELKKPGASV | 332 | QVQLVQSGS | 333 | DIQMTQSPSSLS | 334 | DIQMTQ |
| EPI1613 | | | KVSCKASGYTFNSYAMD | | ELKKPGASV | | ASVGDRVTITCR | | SPSSLSA |
| | | | WVRQAPGQGLEWMGWIN | | KVSCKASG | | ASQSISSYLNWY | | SVGDRV |
| | | | TNTGNPTYAQAFTGRFVF | | YTFNSYAM | | QQKPGKAPKLLI | | TITCRAS |
| | | | SLDTSVSTAYLEISSLKAE | | DWVRQAPG | | YAASSLQSGVPS | | QSISSYL |
| | | | DTAVYYCARERHGYFEAF | | QGLEWMG | | RFSGSGSGTDFT | | NWYQQ |
| | | | DIWGQGTTVTVSSASTKG | | WINTNTGNP | | LTISSLQPEDFAT | | KPGKAP |
| | | | PSVFPLAPSSKSTSGGTAA | | TYAQAFTGR | | YYCQQSYSTPPT | | KLLIYA |
| | | | LGCLVKDYFPEPVTVSWN | | FVFSLDTSV | | FGQGTKVEIKRT | | ASSLQS |
| | | | SGALTSGVHTFPAVLQSSG | | STAYLEISSL | | VAAPSVFIFPPSD | | GVPSRFS |
| | | | LYSLSSVVTVPSSSLGTQT | | KAEDTAVY | | EQLKSGTASVV | | GSGSGT |
| | | | YICNVNHKPSNTKVDKKV | | YCARERHG | | CLLNNFYPREA | | DFTLTIS |
| | | | EPKSCDKTHTCPPCPAPEL | | YFEAFDIWG | | KVQWKVDNAL | | SLQPEDF |
| | | | LGGPSVFLFPPKPKDTLMI | | QGTTVTVSS | | QSGNSQESVTEQ | | ATYYCQ |
| | | | SRTPEVTCVVVDVSHEDP | | | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1311 | ZNRF3 | 335 | EVQLVQSGSELKKPGASV | 336 | EVQLVQSGS | 337 | DIQMTQSPSSLS | 338 | DIQMTQ |
| EPI1614 | | | KVSCKASGYTFTKYAMN | | ELKKPGASV | | ASVGDRVTITCR | | SPSSLSA |
| | | | WVRQVPGQGLEWMGWIN | | KVSCKASG | | ASQSISSYLNWY | | SVGDRV |
| | | | TNTGNPTYAQGFTGRFVF | | YTFTKYAM | | QQKPGKAPKLLI | | TITCRAS |
| | | | SLDTSVRTAYLQISSLKAE | | NWVRQVPG | | YAASSLQSGVPS | | QSISSYL |
| | | | DTAVYYCARKGGSYYDW | | QGLEWMG | | RFSGSGSGTDFT | | NWYQQ |
| | | | FDPWGQGTLVTVSSASTK | | WINTNTGNP | | LTISSLQPEDFAT | | KPGKAP |
| | | | GPSVFPLAPSSKSTSGGTA | | TYAQGFTGR | | YYCQQSYSTPPT | | KLLIYA |
| | | | ALGCLVKDYFPEPVTVSW | | FVFSLDTSV | | FGQGTKVEIKRT | | ASSLQS |
| | | | NSGALTSGVHTFPAVLQSS | | RTAYLQISS | | VAAPSVFIFPPSD | | GVPSRFS |
| | | | GLYSLSSVVTVPSSSLGTQ | | LKAEDTAV | | EQLKSGTASVV | | GSGSGT |
| | | | TYICNVNHKPSNTKVDKK | | YYCARKGG | | CLLNNFYPREA | | DFTLTIS |
| | | | VEPKSCDKTHTCPPCPAPE | | SYYDWFDP | | KVQWKVDNAL | | SLQPEDF |
| | | | LLGGPSVFLFPPKPKDTLM | | WGQGTLVT | | QSGNSQESVTEQ | | ATYYCQ |
| | | | ISRTPEVTCVVVDVSHEDP | | VSS | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1312 | ZNRF3 | 339 | QVQLQESGGGLVQAGGSL | 340 | QVQLQESG | | | | |
| EPI1406 | | | RLSCAASGTISYAHIMGW | | GGLVQAGG | | | | |
| | | | YRQAPGKEREL VAGISQG | | SLRLSCAAS | | | | |
| | | | SITNYADSVKGRFTISRDN | | GTISY AHIM | | | | |
| | | | AKNTVYLQMNSLKPEDTA | | GWYRQAPG | | | | |
| | | | VYYCAVISYDYIKSVPFRY | | KERELVAGI | | | | |
| | | | WGQGTQVTVSSGGGGSD | | SQGSITNYA | | | | |
| | | | KTHTCPPCPAPELLGGPSV | | DSVKGRFTI | | | | |
| | | | FLFPPKPKDTLMISRTPEVT | | SRDNAKNT | | | | |
| | | | CVVVDVSHEDPEVKFNW | | VYLQMNSL | | | | |
| | | | YVDGVEVHNAKTKPREEQ | | KPEDTAVY | | | | |
| | | | YNSTYRVVSVLTVLHQD | | YCAVISYDY | | | | |
| | | | WLNGKEYKCKVSNKALP | | IKSVPFRYW | | | | |
| | | | APIEKTISKAKGQPREPQV | | GQGTQVTV | | | | |
| | | | YTLPPSRDELTKNQVSLW | | SS | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGKGGSHHHHHH | | | | | | |
| EPI1318 | RNF167 | 341 | QVQLQESGGGLVQAGGSL | 342 | QVQLQESG | | | | |
| | | | RLSCAASGSIFRLWYMGW | | GGLVQAGG | | | | |
| | | | YRQAPGKEREFVASIGIGA | | SLRLSCAAS | | | | |
| | | | TTNYADSVKGRFTISRDN | | GSIFRL WYM | | | | |
| | | | AKNTVYLQMNSLKPEDTA | | GWYRQAPG | | | | |
| | | | VYYCAVFGWAYSGYHDD | | KEREFVASI | | | | |
| | | | FLYWGQGTQVTVSSGGG | | GIGATTNYA | | | | |
| | | | GSDKTHTCPPCPAPELLGG | | DSVKGRFTI | | | | |
| | | | PSVFLFPPKPKDTLMISRTP | | SRDNAKNT | | | | |
| | | | EVTCVVVDVSHEDPEVKF | | VYLQMNSL | | | | |
| | | | NWYVDGVEVHNAKTKPR | | KPEDTAVY | | | | |
| | | | EEQYNSTYRVVSVLTVLH | | YCAVFGWA | | | | |
| | | | QDWLNGKEYKCKVSNKA | | YSGYHDDFL | | | | |
| | | | LPAPIEKTISKAKGQPREPQ | | YWGQGTQV | | | | |
| | | | VYTLPPSRDELTKNQVSL | | TVSS | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1319 | RNF128 | 343 | QVQLQESGGGLVQAGGSL | 344 | QVQLQESG | | | | |
| EPI1173 | | | RLSCAASGNISVQLDMGW | | GGLVQAGG | | | | |
| | | | YRQAPGKEREFVAAINQG | | SLRLSCAAS | | | | |
| | | | TTTYYADSVKGRFTISRDN | | GNISVQLDM | | | | |
| | | | AKNTVYLQMNSLKPEDTA | | GWYRQAPG | | | | |
| | | | VYYCAVYLYDIWNHPYW | | KEREFVAAI | | | | |
| | | | GQGTQVTVSSGGGGSDKT | | NQGTTTYY | | | | |
| | | | HTCPPCPAPELLGGPSVFL | | ADSVKGRFT | | | | |
| | | | FPPKPKDTLMISRTPEVTC | | ISRDNAKNT | | | | |
| | | | VVVDVSHEDPEVKFNWY | | VYLQMNSL | | | | |
| | | | VDGVEVHNAKTKPREEQY | | KPEDTAVY | | | | |
| | | | NSTYRVVSVLTVLHQDWL | | YCAVYLYDI | | | | |
| | | | NGKEYKCKVSNKALPAPI | | WNHPYWGQ | | | | |
| | | | EKTISKAKGQPREPQVYTL | | GTQVTVSS | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI1320 | RNF128 | 345 | QVQLQESGGGLVQAGGSL | 346 | QVQLQESG | | | | |
| EPI1174 | | | RLSCAASGSISGGKGMGW | | GGLVQAGG | | | | |
| | | | YRQAPGKEREFVAAIGSG | | SLRLSCAAS | | | | |
| | | | AITYYADSVKGRFTISRDN | | GSISGGKGM | | | | |
| | | | AKNTVYLQMNSLKPEDTA | | GWYRQAPG | | | | |
| | | | VYYCAVYTTALDEYPYW | | KEREFVAAI | | | | |
| | | | GQGTQVTVSSGGGGSDKT | | GSGAITYYA | | | | |
| | | | HTCPPCPAPELLGGPSVFL | | DSVKGRFTI | | | | |
| | | | FPPKPKDTLMISRTPEVTC | | SRDNAKNT | | | | |
| | | | VVVDVSHEDPEVKFNWY | | VYLQMNSL | | | | |
| | | | VDGVEVHNAKTKPREEQY | | KPEDTAVY | | | | |
| | | | NSTYRVVSVLTVLHQDWL | | YCAVYTTA | | | | |
| | | | NGKEYKCKVSNKALPAPI | | LDEYPYWG | | | | |
| | | | EKTISKAKGQPREPQVYTL | | QGTQVTVSS | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI1321 | RNF130 | 347 | QVQLQESGGGLVQAGGSL | 348 | QVQLQESG | | | | |
| EPI1175 | | | RLSCAASGYISGYYVMGW | | GGLVQAGG | | | | |
| | | | YRQAPGKEREFVASISYGA | | SLRLSCAAS | | | | |
| | | | STYYADSVKGRFTISRDNA | | GYISGYYV | | | | |
| | | | KNTVYLQMNSLKPEDTAV | | MGWYRQAP | | | | |
| | | | YYCAVDFDSNYAHTYWG | | GKEREFVAS | | | | |
| | | | QGTQVTVSSGGGGSDKTH | | ISYGASTYY | | | | |
| | | | TCPPCPAPELLGGPSVFLFP | | ADSVKGRFT | | | | |
| | | | PKPKDTLMISRTPEVTCVV | | ISRDNAKNT | | | | |
| | | | VDVSHEDPEVKFNWYVD | | VYLQMNSL | | | | |
| | | | GVEVHNAKTKPREEQYNS | | KPEDTAVY | | | | |
| | | | TYRVVSVLTVLHQDWLN | | YCAVDFDS | | | | |
| | | | GKEYKCKVSNKALPAPIE | | NYAHTYWG | | | | |
| | | | KTISKAKGQPREPQVYTLP | | QGTQVTVSS | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |
| EPI1322 | RNF130 | 349 | QVQLQESGGGLVQAGGSL | 350 | QVQLQESG | | | | |
| EPI1176 | | | RLSCAASGTISFIGYMGWY | | GGLVQAGG | | | | |
| | | | RQAPGKERELVASIASGTS | | SLRLSCAAS | | | | |
| | | | TYYADSVKGRFTISRDNA | | GTISFIGYM | | | | |
| | | | KNTVYLQMNSLKPEDTAV | | GWYRQAPG | | | | |
| | | | YYCAATQYIQDVHRYWG | | KERELVASI | | | | |
| | | | QGTQVTVSSGGGGSDKTH | | ASGTSTYYA | | | | |
| | | | TCPPCPAPELLGGPSVFLFP | | DSVKGRFTI | | | | |
| | | | PKPKDTLMISRTPEVTCVV | | SRDNAKNT | | | | |
| | | | VDVSHEDPEVKFNWYVD | | VYLQMNSL | | | | |
| | | | GVEVHNAKTKPREEQYNS | | KPEDTAVY | | | | |
| | | | TYRVVSVLTVLHQDWLN | | YCAATQYIQ | | | | |
| | | | GKEYKCKVSNKALPAPIE | | DVHRYWGQ | | | | |
| | | | KTISKAKGQPREPQVYTLP | | GTQVTVSS | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |

The sequences listed in Table 4 (SEQ ID NOs: 277-350) are amino acid molecules. The sequences listed in Table 4 (SEQ ID NOs: 277-350) are amino acid molecules that are synthetic constructs. The sequences listed in Table 4 (SEQ ID NOs: 277-350) for HC sequences (heavy chain), VH sequence (variable heavy chain sequence), LC sequences (light chain), VL sequence (variable light chain sequence) are amino acid molecules that are synthetic constructs.

TABLE 5
Exemplary CDR sequences for antibodies targeting the
degrading receptor protein.

| | | SEQ | | SEQ | | SEQ | |
|---|---|---|---|---|---|---|---|
| Antibody | Arm 1 | ID | | ID | | ID | |
| IDs | Target | NO | CDR H1 | NO | CDR H2 | NO | CDR H3 |
| EPI1286 | RNF43 | 886 | TYTIH | 887 | YINPRSGYTEYNQKFQD | 888 | SYEF |
| EPI1156 | | | | | | | |
| EPI1287 | RNF43 | 892 | NYDMT | 893 | SITSDGGSTYSRDSVKG | 894 | DRGRYLPYYFDY |
| EPI1157 | | | | | | | |
| EPI1288 | RNF43 | 898 | TYSVH | 899 | RMWTAGDTSYNSAFTS | 900 | SSYTSGYPFDS |
| EPI1158 | | | | | | | |
| EPI1289 | RNF43 | 904 | YYDMH | 905 | AIGTAGATYYPGSVKG | 906 | DRGYSGYDAYYFDF |
| EPI1159 | | | | | | | |
| EPI1290 | RNF43 | 910 | SSNYYWG | 911 | NIYYRGYTYYNPSLKS | 912 | EGSDYGDYVGAFDI |
| EPI1160 | | | | | | | |
| EPI1291 | RNF43 | 916 | DTYIH | 917 | RIDPANGKANYDPKFQG | 918 | GGGYYGMDY |
| EPI1161 | | | | | | | |
| EPI1292 | RNF43 | 922 | RYWIE | 923 | EILPGSGSTNYNEKFKG | 924 | RGAY |
| EPI1489 | | | | | | | |
| EPI1293 | RNF43 | 928 | KPVMG | 929 | AITSGTNTYYADSVKG | 930 | DDYDVVEYPY |
| EPI1615 | | | | | | | |
| EPI1294 | RNF43 | 931 | SSYWMC | 932 | CIYTGSSGSTSYASWAKG | 933 | DYDYTAYAYGIMSL |
| EPI1162 | | | | | | | |
| EPI1295 | RNF43 | 937 | SRYYMC | 938 | CIYTGSGSTYYASWAKG | 939 | EAGSFNL |
| EPI1163 | | | | | | | |
| EPI1305 | ZNRF3 | 943 | SYHMH | 944 | WINPYTGDTNYAQKFQG | 945 | EKVYMDI |
| EPI1610 | | | | | | | |
| EPI1306 | ZNRF3 | 949 | DYGIH | 950 | RIKSKTDGGITEYAAPVKG | 951 | AIYYLEAFDV |
| EPI1611 | | | | | | | |
| EPI1307 | ZNRF3 | 955 | NYGMH | 956 | YISSNSGTIYADAVKG | 957 | GYFDGYYRF |
| EPI1308 | ZNRF3 | 961 | NYDMA | 962 | SISPGGGKTYYRDSVKG | 963 | LGPAYSGEWFAY |
| EPI1309 | ZNRF3 | 967 | RYPMN | 968 | WINTNTGNPTYAQGFTG | 969 | ERTNFYDAFDI |
| EPI1612 | | | | | | | |
| EPI1310 | ZNRF3 | 973 | SYAMD | 974 | WINTNTGNPTYAQAFTG | 975 | ERHGYFEAFDI |
| EPI1613 | | | | | | | |
| EPI1311 | ZNRF3 | 979 | KYAMN | 980 | WINTNTGNPTYAQGFTG | 981 | KGGSYYDWFDP |
| EPI1614 | | | | | | | |
| EPI1312 | ZNRF3 | 985 | AHIMG | 986 | GISQGSITNYADSVKG | 987 | ISYDYIKSVPFRY |
| EPI1406 | | | | | | | |
| EPI1318 | RNF167 | 988 | LWYMG | 989 | SIGIGATTNYADSVKG | 990 | FGWAYSGYHDDFLY |
| EPI1319 | RNF128 | 991 | QLDMG | 992 | AINQGTTTYYADSVKG | 993 | YLYDIWNHPY |
| EPI1173 | | | | | | | |
| EPI1320 | RNF128 | 994 | GKGMG | 995 | AIGSGAITYYADSVKG | 996 | YTTALDEYPY |
| EPI1174 | | | | | | | |
| EPI1321 | RNF130 | 997 | YYVMG | 998 | SISYGASTYYADSVKG | 999 | DFDSNYAHTY |
| EPI1175 | | | | | | | |
| EPI1322 | RNF130 | 1000 | IGYMG | 1001 | SIASGTSTYYADSVKG | 1002 | TQYIQDVHRY |
| EPI1176 | | | | | | | |

TABLE 6
Exemplary CDR sequences for antibodies targeting the degrading
receptor protein.

| | | SEQ | | SEQ | | SEQ | |
|---|---|---|---|---|---|---|---|
| Antibody | Arm 1 | ID | | ID | | ID | |
| IDs | Target | NO | CDR L1 | NO | CDR L2 | NO | CDR L3 |
| EPI1286 | RNF43 | 889 | KASQNVGINVA | 890 | SASYRYS | 891 | HQYKTYPYT |
| EPI1156 | | | | | | | |
| EPI1287 | RNF43 | 895 | RSSQSLEYSDGYSY | 896 | EVSSRFS | 897 | FQAIHDPT |
| EPI1157 | | | LE | | | | |
| EPI1288 | RNF43 | 901 | RASESVSKLMH | 902 | LTSHLAS | 903 | QQSRNDPT |
| EPI1158 | | | | | | | |
| EPI1289 | RNF43 | 907 | RASQSISSYLN | 908 | AASSLQS | 909 | QQSYSTPPT |
| EPI1159 | | | | | | | |
| EPI1290 | RNF43 | 913 | RASQSISSYLN | 914 | AASSLQS | 915 | QQSYSTPPT |
| EPI1160 | | | | | | | |
| EPI1291 | RNF43 | 919 | RASESVDSYGNSFMH | 920 | LASNLES | 921 | QQNNEDPLT |
| EPI1161 | | | | | | | |
| EPI1292 | RNF43 | 925 | KASEDIYNRLA | 926 | GATSLET | 927 | QQQWSTPPT |
| EPI1489 | | | | | | | |
| EPI1294 | RNF43 | 934 | QASQSITSYLS | 935 | RASTLAS | 936 | QSNYGSYSTNYGVT |
| EPI1162 | | | | | | | |
| EPI1295 | RNF43 | 940 | QASQSIYSGLA | 941 | SASKLAS | 942 | QNYYYGISNGWT |
| EPI1163 | | | | | | | |
| EPI1305 | ZNRF3 | 946 | SGDSIPSKYAH | 947 | GKSHRPS | 948 | AAWDLLGDGWV |
| EPI1610 | | | | | | | |
| EPI1306 | ZNRF3 | 952 | SGDSLGSYYVH | 953 | RNKQRPS | 954 | QTYDWMYSSRV |
| EPI1611 | | | | | | | |
| EPI1307 | ZNRF3 | 958 | RSSQSLLSSGNQKNY | 959 | HASTRQS | 960 | LQHYSSPT |
| | | | LA | | | | |
| EPI1308 | ZNRF3 | 964 | RASEGVNTRIH | 965 | GASNLDS | 966 | QQSWNVPHT |
| EPI1309 | ZNRF3 | 970 | RASQSISSYLN | 971 | AASSLQS | 972 | QQSYSTPPT |
| EPI1612 | | | | | | | |
| EPI1310 | ZNRF3 | 976 | RASQSISSYLN | 977 | AASSLQS | 978 | QQSYSTPPT |
| EPI1613 | | | | | | | |
| EPI1311 | ZNRF3 | 982 | RASQSISSYLN | 983 | AASSLQS | 984 | QQSYSTPPT |
| EPI1614 | | | | | | | |

The sequences listed in Table 5 or 6 (SEQ ID NOs: 886-984) are amino acid molecules. The sequences listed in Table 5 or 6 (SEQ ID NOs: 886-984) are amino acid molecules that are synthetic constructs. The sequences listed in Table 5 or 6 (SEQ ID NOs: 886-984) for CDR (complementarity-determining regions) sequences are amino acid molecules that are synthetic constructs.

In some embodiments, the first binding domain comprises at least one complementarity-determining region (CDR) sequence. The first binding domain comprising at least one complementarity-determining region (CDR) sequence may comprise one or more sequences listed in Table 5 or 6. In some cases, the first binding domain comprising at least one complementarity-determining region (CDR) sequence comprises at least 90% sequence identity to one or more sequences listed in Table 5 or 6. In some cases, the first binding domain comprising an antibody comprising an HC sequence, a VH sequence, an LC sequence, and a VL sequence comprises at least 91% sequence identity to one or more sequences listed in Table 5 or 6. In some cases, the first binding domain comprising at least one complementarity-determining region (CDR) sequence comprises at least 92% sequence identity to one or more sequences listed in Table 5 or 6. In some cases, the first binding domain comprising at least one complementarity-determining region (CDR) sequence comprises at least 93% sequence identity to one or more sequences listed in Table 5 or 6. In some cases, the first binding domain comprising at least one complementarity-determining region (CDR) sequence comprises at least 94% sequence identity to one or more sequences listed in Table 5 or 6. In some cases, the first binding domain comprising at least one complementarity-determining region (CDR) sequence comprises at least 95% sequence identity to one or more sequences listed in Table 5 or 6. In some cases, the first binding domain comprising at least one complementarity-determining region (CDR) sequence comprises at least 96% sequence identity to one or more sequences listed in Table 5 or 6. In some cases, the first binding domain comprising at least one complementarity-determining region (CDR) sequence comprises at least 97% sequence identity to one or more sequences listed in Table 5 or 6. In some cases, the first binding domain comprising at least one complementarity-determining region (CDR) sequence comprises at least 98% sequence identity to one or more sequences listed in Table 5 or 6. In some cases, the first binding domain comprising at least one complementarity-determining region (CDR) sequence comprises at least 99% sequence identity to one or more sequences listed in Table 5 or 6. In some cases, the first binding domain comprising at least one complementarity-determining region (CDR) sequence comprises at least 99.5% sequence identity to one or more sequences listed in Table 5 or 6. In some cases, the first binding domain comprising at least one complementarity-determining region (CDR) sequence comprises at least 99.99% sequence identity to one or more sequences listed in Table 5 or 6.

In some embodiments, the first binding domain comprises at least one sequence listed Table 5 or 6. In some embodiments, the first binding domain comprises at least 90%, at least 91%, at least 92%, at least 93%, at least 94%, at least 95%, at least 96%, at least 97%, at least 98%, at least 99%, at least 99.5%, or at least 99.9% sequence identity to the sequences listed Table 5 or 6.

### Second Binding Region

In some embodiments, the second binding domain (i.e., the cMET binding domain) comprises a cMET binding domain derived from an anti-cMET antibody (e.g., a CDR that specifically binds to cMET). Such antibodies are known to those skilled in the art and can be incorporated into methods and binding agents of the present disclosure. Antibodies targeting cMET are known in the art, and include, for example, the following anti-cMET antibodies: (i) onartuzumab, described in, for example, Lee, D., et al., “Development of antibody-based c-Met inhibitors for targeted cancer therapy.” ImmunoTargets and therapy 4 (2015): 35-44: (ii) amivantamab described in, for example, Neijssen, Joost, et al. “Discovery of amivantamab (JNJ-61186372), a bispecific antibody targeting EGFR and MET.” Journal of Biological Chemistry 296 (2021): (iii) telisotuzumab, described in, for example, Strickler, John H., et al. “Phase I dose-escalation and -expansion study of telisotuzumab (ABT-700), an anti-c-Met antibody, in patients with advanced solid tumors.” Molecular cancer therapeutics 19.5 (2020): 1210-1217: (iv) REGN5093s58, described in, for example, Oh, Seung Yeon, et al. “Preclinical Study of a Biparatopic METxMET Antibody-Drug Conjugate, REGN5093-M114, Overcomes MET-driven Acquired Resistance to EGFR TKIs in EGFR-mutant NSCLC.” Clinical Cancer Research 29.1 (2023): 221-232: (v) emibetuzumab, also known as LY2875358, described in, for example, Liu, L., et al., “LY2875358, a neutralizing and internalizing anti-MET bivalent antibody, inhibits HGF-dependent and HGF-independent MET activation and tumor growth.” Clinical Cancer Research 20.23 (2014): 6059-6070: (vi) 5D5, described in, for example, Jin, H., “MetMAb, the one-armed 5D5 anti-c-Met antibody, inhibits orthotopic pancreatic tumor growth and improves survival.” Cancer Research 68, 11 (2008): 4360-8; and (vii) F46, described in, for example, Young, M., “A new anti-c-Met antibody selected by a mechanism-based dual-screening method: therapeutic potential in cancer. Molecules and cells 34, 6 (2012): 523-9.

The antibodies described in the foregoing are merely exemplary and are not meant to limit in any way the scope of the present disclosure. Additional binding agents, including antibodies, suitable for incorporation into the methods and binding agents of the present disclosure will be evident to one of ordinary skill.

In some embodiments, the second binding domain binds to a mutant cMET protein. In some embodiments, the second binding domain selectively binds to a mutant cMET protein.

In some embodiments, the second binding domain comprises a heavy chain (HC) sequence, a variable heavy (VH) sequence, a light chain (LC) sequence, and a variable light (VL) sequence. In some embodiments, the second binding domain comprises an HC sequence and a VH sequence. The second binding domain comprising an HC sequence, a VH sequence, an LC sequence, and a VL sequence may comprises one or more sequences listed in Table 7. The second binding domain comprising an HC sequence, a VH sequence, an LC sequence, and a VL sequence may comprise at least 70% sequence identity to one or more sequences listed in Table 7. In some cases, the second binding domain comprising an HC sequence, a VH sequence, an LC sequence, and a VL sequence comprises at least 75% sequence identity to one or more sequences listed in Table 7. In some cases, the second binding domain comprising an HC sequence, a VH sequence, an LC sequence, and a VL sequence comprises at least 80% sequence identity to one or more sequences listed in Table 7. In some cases, the second binding domain comprising an HC sequence, a VH sequence, an LC sequence, and a VL sequence comprises at least 85% sequence identity to one or more sequences listed in Table 7. In some cases, the second binding domain comprising an HC sequence, a VH sequence, an LC sequence, and a VL sequence comprises at least 90% sequence identity to one or more sequences listed in Table 7. In some cases, the second binding domain comprising an HC sequence, a VH sequence, an LC sequence, and a VL sequence comprises at least 91% sequence identity to one or more sequences listed in Table 7. In some cases, the second binding domain comprising an HC sequence, a VH sequence, an LC sequence, and a VL sequence comprises at least 92% sequence identity to one or more sequences listed in Table 7. In some cases, the second binding domain comprising an HC sequence, a VH sequence, an LC sequence, and a VL sequence comprises at least 93% sequence identity to one or more sequences listed in Table 7. In some cases, the second binding domain comprising an HC sequence, a VH sequence, an LC sequence, and a VL sequence comprises at least 94% sequence identity to one or more sequences listed in Table 7. In some cases, the second binding domain comprising an HC sequence, a VH sequence, an LC sequence, and a VL sequence comprises at least 95% sequence identity to one or more sequences listed in Table 7. In some cases, the second binding domain comprising an HC sequence, a VH sequence, an LC sequence, and a VL sequence comprises at least 96% sequence identity to one or more sequences listed in Table 7. In some cases, the second binding domain comprising an HC sequence, a VH sequence, an LC sequence, and a VL sequence comprises at least 97% sequence identity to one or more sequences listed in Table 7. In some cases, the second binding domain comprising an HC sequence, a VH sequence, an LC sequence, and a VL sequence comprises at least 98% sequence identity to one or more sequences listed in Table 7. In some cases, the second binding domain comprising an HC sequence, a VH sequence, an LC sequence, and a VL sequence comprises at least 99% sequence identity to one or more sequences listed in Table 7. In some cases, the second binding domain comprising an HC sequence, a VH sequence, an LC sequence, and a VL sequence comprises at least 99.5% sequence identity to one or more sequences listed in Table 7. In some cases, the second binding domain comprising an HC sequence, a VH sequence, an LC sequence, and a VL sequence comprises at least 99.9% sequence identity to one or more sequences listed in Table 7.

In some embodiments, the second binding domain comprises a sequence listed Table 7. In some embodiments, the second binding domain comprises a sequence listed Table 7. In some embodiments, the second binding domain comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 7.

In some embodiments, the second binding domain comprises at least 70% sequence identity to Amivantamab. In some embodiments, the second binding domain comprises at least 75% sequence identity to Amivantamab. In some embodiments, the second binding domain comprises at least 80% sequence identity to Amivantamab. In some embodiments, the second binding domain comprises at least 85% sequence identity to Amivantamab. In some embodiments, the second binding domain comprises at least 90% sequence identity to Amivantamab. In some embodiments, the second binding domain comprises at least 91% sequence identity to Amivantamab. In some embodiments, the second binding domain comprises at least 92% sequence identity to Amivantamab. In some embodiments, the second binding domain comprises at least 93% sequence identity to Amivantamab. In some embodiments, the second binding domain comprises at least 94% sequence identity to Amivantamab. In some embodiments, the second binding domain comprises at least 95% sequence identity to Amivantamab. In some embodiments, the second binding domain comprises at least 96% sequence identity to Amivantamab. In some embodiments, the second binding domain comprises at least 97% sequence identity to Amivantamab. In some embodiments, the second binding domain comprises at least 98% sequence identity to Amivantamab. In some embodiments, the second binding domain comprises at least 99% sequence identity to Amivantamab. In some embodiments, the second binding domain comprises at least 99.5% sequence identity to Amivantamab. In some embodiments, the second binding domain comprises at least 99.9% sequence identity to Amivantamab.

In some embodiments, the second binding domain comprises at least 70% sequence identity to Telisotuzumab. In some embodiments, the second binding domain comprises at least 75% sequence identity to Telisotuzumab. In some embodiments, the second binding domain comprises at least 80% sequence identity to Telisotuzumab. In some embodiments, the second binding domain comprises at least 85% sequence identity to Telisotuzumab. In some embodiments, the second binding domain comprises at least 90% sequence identity to Telisotuzumab. In some embodiments, the second binding domain comprises at least 91% sequence identity to Telisotuzumab. In some embodiments, the second binding domain comprises at least 92% sequence identity to Telisotuzumab. In some embodiments, the second binding domain comprises at least 93% sequence identity to Telisotuzumab. In some embodiments, the second binding domain comprises at least 94% sequence identity to Telisotuzumab. In some embodiments, the second binding domain comprises at least 95% sequence identity to Telisotuzumab. In some embodiments, the second binding domain comprises at least 96% sequence identity to Telisotuzumab. In some embodiments, the second binding domain comprises at least 97% sequence identity to Telisotuzumab. In some embodiments, the second binding domain comprises at least 98% sequence identity to Telisotuzumab. In some embodiments, the second binding domain comprises at least 99% sequence identity to Telisotuzumab. In some embodiments, the second binding domain comprises at least 99.5% sequence identity to Telisotuzumab. In some embodiments, the second binding domain comprises at least 99.9% sequence identity to Telisotuzumab.

In some embodiments, the second binding domain comprises at least 70% sequence identity to Onartuzumab. In some embodiments, the second binding domain comprises at least 75% sequence identity to Onartuzumab. In some embodiments, the second binding domain comprises at least 80% sequence identity to Onartuzumab. In some embodiments, the second binding domain comprises at least 85% sequence identity to Onartuzumab. In some embodiments, the second binding domain comprises at least 90% sequence identity to Onartuzumab. In some embodiments, the second binding domain comprises at least 91% sequence identity to Onartuzumab. In some embodiments, the second binding domain comprises at least 92% sequence identity to Onartuzumab. In some embodiments, the second binding domain comprises at least 93% sequence identity to Onartuzumab. In some embodiments, the second binding domain comprises at least 94%-sequence identity to Onartuzumab. In some embodiments, the second binding domain comprises at least 95% sequence identity to Onartuzumab. In some embodiments, the second binding domain comprises at least 96% sequence identity to Onartuzumab. In some embodiments, the second binding domain comprises at least 97% sequence identity to Onartuzumab. In some embodiments, the second binding domain comprises at least 98% sequence identity to Onartuzumab. In some embodiments, the second binding domain comprises at least 99% sequence identity to Onartuzumab. In some embodiments, the second binding domain comprises at least 99.5% sequence identity to Onartuzumab. In some embodiments, the second binding domain comprises at least 99.9% sequence identity to Onartuzumab.

In some embodiments, the second binding domain comprises at least 70% sequence identity to REGN5093s58. In some embodiments, the second binding domain comprises at least 75% sequence identity to REGN5093s58. In some embodiments, the second binding domain comprises at least 80% sequence identity to REGN5093s58. In some embodiments, the second binding domain comprises at least 85% sequence identity to REGN5093s58. In some embodiments, the second binding domain comprises at least 90% sequence identity to REGN5093s58. In some embodiments, the second binding domain comprises at least 91% sequence identity to REGN5093s58. In some embodiments, the second binding domain comprises at least 92% sequence identity to REGN5093s58. In some embodiments, the second binding domain comprises at least 93% sequence identity to REGN5093s58. In some embodiments, the second binding domain comprises at least 94% sequence identity to REGN5093s58. In some embodiments, the second binding domain comprises at least 95% sequence identity to REGN5093s58. In some embodiments, the second binding domain comprises at least 96% sequence identity to REGN5093s58. In some embodiments, the second binding domain comprises at least 97% sequence identity to REGN5093s58. In some embodiments, the second binding domain comprises at least 98% sequence identity to REGN5093s58. In some embodiments, the second binding domain comprises at least 99% sequence identity to REGN5093s58. In some embodiments, the second binding domain comprises at least 99.5% sequence identity to REGN5093s58. In some embodiments, the second binding domain comprises at least 99.9% sequence identity to REGN5093s58.

In some embodiments, the second binding domain binds to an epitope of cMET on the target cell, wherein the epitope comprises at least 70% sequence identity to an epitope to which Amivantamab binds. In some embodiments, the second binding domain binds to an epitope of cMET on the target cell, wherein the epitope comprises at least 80% sequence identity to an epitope to which Amivantamab binds. In some cases, the second binding domain binds to an epitope of cMET on the target cell, wherein the epitope comprises at least 90% sequence identity to the epitope to which Amivantamab binds. In some embodiments, the second binding domain binds to an epitope of cMET on the target cell, wherein the epitope comprises at least 95% sequence identity to an epitope to which Amivantamab binds.

In some embodiments, the second binding domain binds to an epitope of cMET on the target cell that does not include any of the amino acids from the epitope to which Amivantamab binds. In some embodiments, the second binding domain binds to an epitope of cMET on the target cell that includes one, two, three, four, five, or six of the amino acids from the epitope to which Amivantamab binds. In some embodiments, the second binding domain binds to an epitope of cMET on the target cell that includes one or more of the amino acids from the epitope to which Amivantamab binds. In some embodiments, the second binding domain binds to an epitope of cMET on the target cell that includes two or more of the amino acids from the epitope to which Amivantamab binds. In some embodiments, the second binding domain binds to an epitope of cMET on the target cell that includes three or more of the amino acids from the epitope to which Amivantamab binds. In some embodiments, the second binding domain binds to an epitope of cMET on the target cell that includes four or more of the amino acids from the epitope to which Amivantamab binds.

In some embodiments, the second binding domain binds to an epitope of cMET on the target cell, wherein the epitope comprises at least 70% sequence identity to an epitope to which Telisotuzumab binds. In some embodiments, the second binding domain binds to an epitope of cMET on the target cell, wherein the epitope comprises at least 80% sequence identity to an epitope to which Telisotuzumab binds. In some cases, the second binding domain binds to an epitope of cMET on the target cell, wherein the epitope comprises at least 90% sequence identity to the epitope to which Telisotuzumab binds. In some embodiments, the second binding domain binds to an epitope of cMET on the target cell, wherein the epitope comprises at least 95% sequence identity to an epitope to which Telisotuzumab binds.

In some embodiments, the second binding domain binds to an epitope of cMET on the target cell that does not include any of the amino acids from the epitope to which Telisotuzumab binds. In some embodiments, the second binding domain binds to an epitope of cMET on the target cell that includes one, two, three, four, five, or six of the amino acids from the epitope to which Telisotuzumab binds. In some embodiments, the second binding domain binds to an epitope of cMET on the target cell that includes one or more of the amino acids from the epitope to which Telisotuzumab binds. In some embodiments, the second binding domain binds to an epitope of cMET on the target cell that includes two or more of the amino acids from the epitope to which Telisotuzumab binds. In some embodiments, the second binding domain binds to an epitope of cMET on the target cell that includes three or more of the amino acids from the epitope to which Telisotuzumab binds. In some embodiments, the second binding domain binds to an epitope of cMET on the target cell that includes four or more of the amino acids from the epitope to which Telisotuzumab binds.

In some embodiments, the second binding domain binds to an epitope of cMET on the target cell, wherein the epitope comprises at least 70% sequence identity to an epitope to which Onartuzumab binds. In some embodiments, the second binding domain binds to an epitope of cMET on the target cell, wherein the epitope comprises at least 80% sequence identity to an epitope to which Onartuzumab binds. In some cases, the second binding domain binds to an epitope of cMET on the target cell, wherein the epitope comprises at least 90% sequence identity to the epitope to which Onartuzumab binds. In some embodiments, the second binding domain binds to an epitope of cMET on the target cell, wherein the epitope comprises at least 95% sequence identity to an epitope to which Onartuzumab binds.

In some embodiments, the second binding domain binds to an epitope of cMET on the target cell that does not include any of the amino acids from the epitope to which Onartuzumab binds. In some embodiments, the second binding domain binds to an epitope of cMET on the target cell that includes one, two, three, four, five, or six of the amino acids from the epitope to which Onartuzumab binds. In some embodiments, the second binding domain binds to an epitope of cMET on the target cell that includes one or more of the amino acids from the epitope to which Onartuzumab binds. In some embodiments, the second binding domain binds to an epitope of cMET on the target cell that includes two or more of the amino acids from the epitope to which Onartuzumab binds. In some embodiments, the second binding domain binds to an epitope of cMET on the target cell that includes three or more of the amino acids from the epitope to which Onartuzumab binds. In some embodiments, the second binding domain binds to an epitope of cMET on the target cell that includes four or more of the amino acids from the epitope to which Onartuzumab binds.

In some embodiments, the second binding domain binds to an epitope of cMET on the target cell, wherein the epitope comprises at least 70% sequence identity to an epitope to which REGN5093s58 binds. In some embodiments, the second binding domain binds to an epitope of cMET on the target cell, wherein the epitope comprises at least 80% sequence identity to an epitope to which REGN5093s58 binds. In some cases, the second binding domain binds to an epitope of cMET on the target cell, wherein the epitope comprises at least 90% sequence identity to the epitope to which REGN5093s58 binds. In some embodiments, the second binding domain binds to an epitope of cMET on the target cell, wherein the epitope comprises at least 95% sequence identity to an epitope to which REGN5093s58 binds.

In some embodiments, the second binding domain binds to an epitope of cMET on the target cell that does not include any of the amino acids from the epitope to which REGN5093s58 binds. In some embodiments, the second binding domain binds to an epitope of cMET on the target cell that includes one, two, three, four, five, or six of the amino acids from the epitope to which REGN5093s58 binds. In some embodiments, the second binding domain binds to an epitope of cMET on the target cell that includes one or more of the amino acids from the epitope to which REGN5093s58 binds. In some embodiments, the second binding domain binds to an epitope of cMET on the target cell that includes two or more of the amino acids from the epitope to which REGN5093s58 binds. In some embodiments, the second binding domain binds to an epitope of cMET on the target cell that includes three or more of the amino acids from the epitope to which REGN5093s58 binds. In some embodiments, the second binding domain binds to an epitope of cMET on the target cell that includes four or more of the amino acids from the epitope to which REGN5093s58 binds.

In some embodiments, the epitope of cMET comprises the following amino acids of human cMET (UniProt ID: P08581): G326, A327, Q328, R331, Q332, 1333, G334, A335, S336, L337, N338, D339, K368, Y369, R426, 1446, G448, D449, and R469. The second binding domain may target the epitope comprising the amino acids G326, A327, Q328, R331, Q332, 1333, G334, A335, S336, L337, N338, D339, K368, Y369, R426, 1446, G448, D449, and R469 of human cMET. In some embodiments, the antibody targeting the amino acids G326, A327, Q328, R331, Q332, 1333, G334, A335, S336, L337, N338, D339, K368, Y369, R426, 1446, G448, D449, and R469 of human cMET comprises Onartuzumab. In some embodiments, the epitope of cMET comprises the following amino acids of human cMET: D94, F96, P97, C98, Q99, D100, S103, K104, A105, N106, H159, C160, F162, S163, P164, 1166, E167, T222, and D224. The second binding domain may target the epitope comprising the amino D94, F96, P97, C98, Q99, D100, S103, K104, A105, N106, H159, C160, F162, S163, P164, 1166, E167, T222, and D224 of human cMET. In some embodiments, the antibody targeting the amino acids D94, F96, P97, C98, Q99, D100, S103, K104, A105, N106, H159, C160, F162, S163, P164, 1166, E167, T222, and D224 of human cMET comprises Amivantamab.

In some cases, the second binding domain may bind the same epitope as Amivantamab. Telisotuzumab. Onartuzumab or REGN5093s58. The second binding domain may bind to an epitope that comprises about 70% sequence identity to the epitope to which Amivantamab. Telisotuzumab. Onartuzumab or REGN5093s58 binds. The second binding domain may bind to an epitope that comprises about 75% sequence identity to the epitope to which Amivantamab. Telisotuzumab. Onartuzumab or REGN5093s58 binds. The second binding domain may bind to an epitope that comprises about 80% sequence identity to the epitope to which Amivantamab. Telisotuzumab. Onartuzumab or REGN5093s58 binds. The second binding domain may bind to an epitope that comprises about 85% sequence identity to the epitope to which Amivantamab. Telisotuzumab. Onartuzumab or REGN5093s58 binds. The second binding domain may bind to an epitope that comprises about 90% sequence identity to the epitope to which Amivantamab. Telisotuzumab. Onartuzumab or REGN5093s58 binds. The second binding domain may bind to an epitope that comprises about 95% sequence identity to the epitope to which Amivantamab. Telisotuzumab. Onartuzumab or REGN5093s58 binds. The second binding domain may bind to an epitope that comprises about 99% sequence identity to the epitope to which Amivantamab. Telisotuzumab, Onartuzumab or REGN5093s58 binds.

The second binding domain may bind to an epitope that comprises a different epitope than the epitope to which Amivantamab. Telisotuzumab. Onartuzumab or REGN5093s58 binds. The second binding domain may bind to an epitope that comprises a different epitope than the epitope to which Amivantamab. Telisotuzumab. Onartuzumab or REGN5093s58 binds, wherein the epitopes do not bind to any of the same amino acids on cMET. The second binding domain may bind to an epitope that comprises a different epitope than the epitope to which Amivantamab. Telisotuzumab. Onartuzumab or REGN5093s58 binds, wherein the epitopes bind to any one or more of the same amino acids on cMET. The second binding domain may bind to an epitope that comprises a different epitope than the epitope to which Amivantamab. Telisotuzumab. Onartuzumab or REGN5093s58 binds, wherein the epitopes bind to any two or more of the same amino acids on cMET. The second binding domain may bind to an epitope that comprises a different epitope than the epitope to which Amivantamab. Telisotuzumab. Onartuzumab or REGN5093s58 binds, wherein the epitopes bind to any three or more of the same amino acids on cMET. The second binding domain may bind to an epitope that comprises a different epitope than the epitope to which Amivantamab. Telisotuzumab. Onartuzumab or REGN5093s58, wherein the epitopes bind to any four or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any five or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any six or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any seven or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any eight or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any nine or more of the same amino acids on cMET. The second binding domain may bind to an epitope that comprises a different epitope than the epitope to which Amivantamab, Telisotuzumab, Onartuzumab or REGN5093s58 binds, wherein the epitopes bind to any ten or more of the same amino acids on cMET.

In some cases, the second binding domain may bind the same epitope as any one of the antibodies listed in Table 7. The second binding domain may bind to an epitope that comprises about 70% sequence identity to the epitope to which any one of the antibodies listed in Table 7 binds. The second binding domain may bind to an epitope that comprises about 75% sequence identity to the epitope to which any one of the antibodies listed in Table 7 binds. The second binding domain may bind to an epitope that comprises about 80% sequence identity to the epitope to which any one of the antibodies listed in Table 7 binds. The second binding domain may bind to an epitope that comprises about 85% sequence identity to the epitope to which any one of the antibodies listed in Table 7 binds. The second binding domain may bind to an epitope that comprises about 90% sequence identity to the epitope to which any one of the antibodies listed in Table 7 binds. The second binding domain may bind to an epitope that comprises about 95% sequence identity to the epitope to which any one of the antibodies listed in Table 7 binds. The second binding domain may bind to an epitope that comprises about 99% sequence identity to the epitope to which any one of the antibodies listed in Table 7 binds.

In some embodiments, the first binding domain may bind the same epitope as any one of the antibodies listed in Table 7 binds with a similar affinity as any one of the antibodies listed in Table 7. The first binding domain may bind to an epitope that comprises about 70% sequence identity to the epitope to which any one of the antibodies listed in Table 7 binds with a similar affinity as any one of the antibodies listed in Table 7. The first binding domain may bind to an epitope that comprises about 75% sequence identity to the epitope to which any one of the antibodies listed in Table 7 binds with a similar affinity as any one of the antibodies listed in Table 7. The first binding domain may bind to an epitope that comprises about 80% sequence identity to the epitope to which any one of the antibodies listed in Table 7 binds with a similar affinity as any one of the antibodies listed in Table 7. The first binding domain may bind to an epitope that comprises about 85% sequence identity to the epitope to which any one of the antibodies listed in Table 7 binds with a similar affinity as any one of the antibodies listed in Table 7. The first binding domain may bind to an epitope that comprises about 90% sequence identity to the epitope to which any one of the antibodies listed in Table 7 binds with a similar affinity as any one of the antibodies listed in Table 7. The first binding domain may bind to an epitope that comprises about 95% sequence identity to the epitope to which any one of the antibodies listed in Table 7 binds with a similar affinity as any one of the antibodies listed in Table 7. The first binding domain may bind to an epitope that comprises about 99% sequence identity to the epitope to which any one of the antibodies listed in Table 7 binds with a similar affinity as any one of the antibodies listed in Table 7.

TABLE 7
Exemplary antibody sequences targeting cMET.

| | | SEQ | | SEQ | | SEQ | | SEQ | |
|---|---|---|---|---|---|---|---|---|---|
| Antibody | Arm 2 | ID | | ID | VH | ID | LC | ID | VL |
| IDs | Target | NO | HC sequence | NO | sequence | NO | sequence | NO | sequence |
| Amivanta- | cMET | 351 | QVQLVQSGAEVKKPG | 352 | QVQLVQS | 353 | DIQMTQSPSS | 354 | DIQMT |
| mab | | | ASVKVSCETSGYTFTS | | GAEVKKP | | VSASVGDRV | | QSPSSV |
| (EPI632 | | | YGISWVRQAPGHGLE | | GASVKVSC | | TITCRASQGIS | | SASVG |
| EPI635 | | | WMGWISAYNGYTNY | | ETSGYTFT | | NWLAWFQH | | DRVTIT |
| EPI1088 | | | AQKLQGRVTMTTDTS | | SYGISWVR | | KPGKAPKLLI | | CRASQ |
| EPI1092 | | | TSTAYMELRSLRSDDT | | QAPGHGLE | | YAASSLLSGV | | GISNW |
| EPI1237 | | | AVYYCARDLRGTNYF | | WMGWISA | | PSRFSGSGSG | | LAWFQ |
| EPI1238 | | | DYWGQGTLVTVSSAK | | YNGYTNY | | TDFTLTISSLQ | | HKPGK |
| EPI1240 | | | TTPPSVYPLAPGSAAQ | | AQKLQGR | | PEDFATYYC | | APKLLI |
| EPI1241 | | | TNSMVTLGCLVKGYF | | VTMTTDTS | | QQANSFPITF | | YAASS |
| EPI1242 | | | PEPVTVTWNSGSLSSG | | TSTAYMEL | | GQGTRLEIKR | | LLSGV |
| EPI1243 | | | VHTFPAVLQSDLYTLS | | RSLRSDDT | | ADAAPTVSIF | | PSRFSG |
| EPI1244 | | | SSVTVPSSPRPSETVTC | | AVYYCAR | | PPSSEQLTSG | | SGSGT |
| EPI1245 | | | NVAHPASSTKVDKKI | | DLRGTNYF | | GASVVCFLN | | DFTLTI |
| EPI1246 | | | VPRDCDKTHTCPPCPA | | DYWGQGT | | NFYPKDINVK | | SSLQPE |
| EPI1247 | | | PELLGGPSVFLFPPKP | | LVTVSS | | WKIDGSERQ | | DFATY |
| EPI1248 | | | KDTLMISRTPEVTCVV | | | | NGVLNSWTD | | YCQQA |
| EPI1249 | | | VDVSHEDPEVKFNWY | | | | QDSKDSTYS | | NSFPIT |
| EPI1250 | | | VDGVEVHNAKTKPRE | | | | MSSTLTLTKD | | FGQGT |
| EPI1251 | | | EQYNSTYRVVSVLTV | | | | EYERHNSYT | | RLEIK |
| EPI1252 | | | LHQDWLNGKEYKCK | | | | CEATHKTSTS | | |
| EPI1253 | | | VSNKALPAPIEKTISK | | | | PIVKSFNRNE | | |
| EPI1254 | | | AKGQPREPQVYTLPPS | | | | C | | |
| EPI1255 | | | RDELTKNQVSLSCAV | | | | | | |
| EPI1256 | | | KGFYPSDIAVEWESN | | | | | | |
| EPI1257 | | | GQPENNYKTTPPVLDS | | | | | | |
| EPI1258 | | | DGSFFLVSKLTVDKSR | | | | | | |
| EPI1259 | | | WQQGNVFSCSVMHE | | | | | | |
| EPI1260 | | | ALHNHYTQKSLSLSPG | | | | | | |
| EPI1261 | | | K | | | | | | |
| EPI1262 | | | | | | | | | |
| EPI1263 | | | | | | | | | |
| EPI1264 | | | | | | | | | |
| EPI1265 | | | | | | | | | |
| EPI1266 | | | | | | | | | |
| EPI1267 | | | | | | | | | |
| EPI1268 | | | | | | | | | |
| EPI1269 | | | | | | | | | |
| EPI1270 | | | | | | | | | |
| EPI1271 | | | | | | | | | |
| EPI1272 | | | | | | | | | |
| EPI1273 | | | | | | | | | |
| EPI1274 | | | | | | | | | |
| EPI1275 | | | | | | | | | |
| EPI1276 | | | | | | | | | |
| EPI1277 | | | | | | | | | |
| EPI1278 | | | | | | | | | |
| EPI1279 | | | | | | | | | |
| EPI1280 | | | | | | | | | |
| EPI1281 | | | | | | | | | |
| EPI1282 | | | | | | | | | |
| EPI1283 | | | | | | | | | |
| EPI1284 | | | | | | | | | |
| EPI1285 | | | | | | | | | |
| EPI1286 | | | | | | | | | |
| EPI1287 | | | | | | | | | |
| EPI1288 | | | | | | | | | |
| EPI1289 | | | | | | | | | |
| EPI1290 | | | | | | | | | |
| EPI1291 | | | | | | | | | |
| EPI1292 | | | | | | | | | |
| EPI1293 | | | | | | | | | |
| EPI1294 | | | | | | | | | |
| EPI1295 | | | | | | | | | |
| EPI1296 | | | | | | | | | |
| EPI1297 | | | | | | | | | |
| EPI1298 | | | | | | | | | |
| EPI1299 | | | | | | | | | |
| EPI1300 | | | | | | | | | |
| EPI1301 | | | | | | | | | |
| EPI1302 | | | | | | | | | |
| EPI1303 | | | | | | | | | |
| EPI1304 | | | | | | | | | |
| EPI1305 | | | | | | | | | |
| EPI1306 | | | | | | | | | |
| EPI1307 | | | | | | | | | |
| EPI1308 | | | | | | | | | |
| EPI1309 | | | | | | | | | |
| EPI1310 | | | | | | | | | |
| EPI1311 | | | | | | | | | |
| EPI1312 | | | | | | | | | |
| EPI1318 | | | | | | | | | |
| EPI1319 | | | | | | | | | |
| EPI1320 | | | | | | | | | |
| EPI1321 | | | | | | | | | |
| EPI1322 | | | | | | | | | |
| EPI1323 | | | | | | | | | |
| EPI1324 | | | | | | | | | |
| EPI1325 | | | | | | | | | |
| EPI1326 | | | | | | | | | |
| EPI1327 | | | | | | | | | |
| EPI1328 | | | | | | | | | |
| EPI1329 | | | | | | | | | |
| EPI1621 | | | | | | | | | |
| EPI1622 | | | | | | | | | |
| EPI1623 | | | | | | | | | |
| EPI1624 | | | | | | | | | |
| EPI2132 | | | | | | | | | |
| EPI2577) | | | | | | | | | |
| Onartuzu- | cMET | 355 | EVQLVESGGGLVQPGGS | 356 | EVQLVESGG | 357 | DIQMTQSPSSLS | 358 | DIQMTQ |
| mab | | | LRLSCAASGYTFTSYWL | | GLVQPGGSL | | ASVGDRVTITCK | | SPSSLSA |
| (EPI1087 | | | HWVRQAPGKGLEWVGM | | RLSCAASGY | | SSQSSLLYTSSQK | | SVGDRV |
| EPI2150 | | | IDPSNSDTRFNPNFKDRF | | TFTSYWLH | | NYLAWYQQKP | | TITCKSS |
| EPI1091 | | | TISADTSKNTAYLQMNSL | | WVRQAPGK | | GKAPKLLIYWA | | QSLLYT |
| EPI2211 | | | RAEDTAVYYCATYRSYV | | GLEWVGMI | | STRESGVPSRFS | | SSQKNY |
| EPI2212 | | | TPLDYWGQGTLVTVSSA | | DPSNSDTRF | | GSGSGTDFTLTI | | LAWYQ |
| EPI2213 | | | KTTPPSVYPLAPGSAAQT | | NPNFKDRFT | | SSLQPEDFATYY | | QKPGKA |
| EPI2214 | | | NSMVTLGCLVKGYFPEP | | ISADTSKNT | | CQQYYAYPWTF | | PKLLIY |
| EPI2215 | | | VTVTWNSGSLSSGVHTFP | | AYLQMNSL | | GQGTKVEIKRA | | WASTRE |
| EPI2216 | | | AVLQSDLYTLSSSVTVPS | | RAEDTAVY | | DAAPTVSIFPPSS | | SGVPSRF |
| EPI2217 | | | SPRPSETVTCNVAHPASS | | YCATYRSY | | EQLTSGGASVV | | SGSGSG |
| EPI2218 | | | TKVDKKIVPRDCDKTHT | | VTPLDYWG | | CFLNNFYPKDIN | | TDFTLTI |
| EPI2219 | | | CPPCPAPELLGGPSVFLFP | | QGTLVTVSS | | VKWKIDGSERQ | | SSLQPED |
| EPI2220) | | | PKPKDTLMISRTPEVTCV | | | | NGVLNSWTDQD | | FATYYC |
| | | | VVDVSHEDPEVKFNWYV | | | | SKDSTYSMSSTL | | QQYYAY |
| | | | DGVEVHNAKTKPREEQY | | | | TLTKDEYERHN | | PWTFGQ |
| | | | NSTYRVVSVLTVLHQDW | | | | SYTCEATHKTST | | GTKVEI |
| | | | LNGKEYKCKVSNKALPA | | | | SPIVKSFNRNEC | | K |
| | | | PIEKTISKAKGQPREPQV | | | | | | |
| | | | YTLPPSRDELTKNQVSLS | | | | | | |
| | | | CAVKGFYPSDIAVEWES | | | | | | |
| | | | NGQPENNYKTTPPVLDS | | | | | | |
| | | | DGSFFLVSKLTVDKSRW | | | | | | |
| | | | QQGNVFSCSVMHEALHN | | | | | | |
| | | | HYTQKSLSLSPGK | | | | | | |
| Teliso- | cMET | 359 | QVQLVQSGAEVKKPGAS | 360 | QVTLRESGP | 361 | DIVMTQSPDSLA | 362 | DIVMTQ |
| tuzumab | T | | VKVSCKASGYIFTAYTM | | ALVKPTQTL | | VSLGERATINCK | | SPDSLA |
| (EPI1086 | | | HWVRQAPGQGLEWMG | | TLTCTFSGFS | | SSESVDSYANSF | | VSLGER |
| EPI2153 | | | WIKPNNGLANYAQKFQG | | LSTSGMSVG | | LHWYQQKPGQP | | ATINCKS |
| EPI631 | | | RVTMTRDTSISTAYMELS | | WIRQPPGKA | | PKLLIYRASTRE | | SESVDS |
| EPI634 | | | RLRSDDTAVYYCARSEIT | | LEWLADIW | | SGVPDRFSGSGS | | YANSFL |
| EPI1090 | | | TEFDYWGQGTLVTVSSA | | WDDKKDYN | | GTDFTLTISSLQ | | HWYQQ |
| EPI1112) | | | KTTPPSVYPLAPGSAAQT | | PSLKSRLTIS | | AEDVAVYYCQQ | | KPGQPP |
| | | | NSMVTLGCLVKGYFPEP | | KDTSKNQV | | SKEDPLTFGGGT | | KLLIYR |
| | | | VTVTWNSGSLSSGVHTFP | | VLKVTNMD | | KVEIKRADAAPT | | ASTRES |
| | | | AVLQSDLYTLSSSVTVPS | | PADTATYYC | | VSIFPPSSEQLTS | | GVPDRF |
| | | | SPRPSETVTCNVAHPASS | | ARSMITNW | | GGASVVCFLNN | | SGSGSG |
| | | | TKVDKKIVPRDCDKTHT | | YFDVWGAG | | FYPKDINVKWKI | | TDFTLTI |
| | | | CPPCPAPELLGGPSVFLFP | | TTVTVSS | | DGSERQNGVLN | | SSLQAE |
| | | | PKPKDTLMISRTPEVTCV | | | | SWTDQDSKDST | | DVAVYY |
| | | | VVDVSHEDPEVKFNWYV | | | | YSMSSTLTLTKD | | CQQSKE |
| | | | DGVEVHNAKTKPREEQY | | | | EYERHNSYTCE | | DPLTFG |
| | | | NSTYRVVSVLTVLHQDW | | | | ATHKTSTSPIVK | | GGTKVE |
| | | | LNGKEYKCKVSNKALPA | | | | SFNRNEC | | IK |
| | | | PIEKTISKAKGQPREPQV | | | | | | |
| | | | YTLPPSRDELTKNQVSLS | | | | | | |
| | | | CAVKGFYPSDIAVEWES | | | | | | |
| | | | NGQPENNYKTTPPVLDS | | | | | | |
| | | | DGSFFLVSKLTVDKSRW | | | | | | |
| | | | QQGNVFSCSVMHEALHN | | | | | | |
| | | | HYTQKSLSLSPGK | | | | | | |
| REGN5093s58 | cMET | 363 | EVQLVESGGGLVQPGTS | 364 | EVQLVESGG | 365 | DIQMTQSPSSLS | 366 | DIQMT |
| (EPI1089 | | | LRLSCAASGFTFDDYAM | | GLVQPGTSL | | ASVGDRVTITCR | | QSPSSL |
| EPI1093) | | | HWVRQAPGKGLEWVSGI | | RLSCAASGF | | ASQSISSYLNWY | | SASVG |
| | | | TWNSYNIDYADSVKGRF | | TEDDYAMH | | QQKPGKAPKLLI | | DRVTIT |
| | | | TISRDNAKNSLYLQMNSL | | WVRQAPGK | | YAASSLQSGVPS | | CRASQ |
| | | | RAEDTALYYCAKDDDYS | | GLEWVSGIT | | RFSGSGSGTDFT | | SISSYL |
| | | | NYVYFDYWGQGTLVTV | | WNSYNIDY | | LTISSLQPEDFAT | | NWYQ |
| | | | SSAKTTPPSVYPLAPGSA | | ADSVKGRFT | | YYCQQSYSTPPI | | QKPGK |
| | | | AQTNSMVTLGCLVKGYF | | ISRDNAKNS | | TFGQGTRLEIKR | | APKLLI |
| | | | PEPVTVTWNSGSLSSGVH | | LYLQMNSL | | ADAAPTVSIFPP | | YAASS |
| | | | TFPAVLQSDLYTLSSSVT | | RAEDTALY | | SSEQLTSGGASV | | LQSGV |
| | | | VPSSPRPSETVTCNVAHP | | YCAKDDDY | | VCFLNNFYPKDI | | PSRFSG |
| | | | ASSTKVDKKIVPRDCDKT | | SNYVYFDY | | NVKWKIDGSER | | SGSGT |
| | | | HTCPPCPAPELLGGPSVF | | WGQGTLVT | | QNGVLNSWTDQ | | DFTLTI |
| | | | LFPPKPKDTLMISRTPEVT | | VSS | | DSKDSTYSMSST | | SSLQPE |
| | | | CVVVDVSHEDPEVKFNW | | | | LTLTKDEYERH | | DFATY |
| | | | YVDGVEVHNAKTKPREE | | | | NSYTCEATHKTS | | YCQQS |
| | | | QYNSTYRVVSVLTVLHQ | | | | TSPIVKSFNRNE | | YSTPPI |
| | | | DWLNGKEYKCKVSNKA | | | | C | | TFGQG |
| | | | LPAPIEKTISKAKGQPREP | | | | | | TRLEIK |
| | | | QVYTLPPSRDELTKNQVS | | | | | | |
| | | | LSCAVKGFYPSDIAVEWE | | | | | | |
| | | | SNGQPENNYKTTPPVLDS | | | | | | |
| | | | DGSFFLVSKLTVDKSRW | | | | | | |
| | | | QQGNVFSCSVMHEALHN | | | | | | |
| | | | HYTQKSLSLSPGK | | | | | | |
| Amivanta- | cMET | 367 | QVQLVQSGAEVKKPGAS | 368 | QVQLVQSG | 369 | DIQMTQSPSSVS | 370 | DIQMTQ |
| mab | | | VKVSCETSGYTFTSYGIS | | AEVKKPGAS | | ASVGDRVTITCR | | SPSSVSA |
| (EPI818 | | | WVRQAPGHGLEWMGWI | | VKVSCETSG | | ASQGISNWLAW | | SVGDRV |
| EPI2075) | | | SAYNGYTNYAQKLQGR | | YTFTSYGIS | | FQHKPGKAPKL | | TITCRAS |
| | | | VTMTTDTSTSTAYMELR | | WVRQAPGH | | LIYAASSLLSGV | | QGISNW |
| | | | SLRSDDTAVYYCARDLR | | GLEWMGWI | | PSRFSGSGSGTD | | LAWFQH |
| | | | GTNYFDYWGQGTLVTVS | | SAYNGYTN | | FTLTISSLQPEDF | | KPGKAP |
| | | | SASTKGPSVFPLAPSSKST | | YAQKLQGR | | ATYYCQQANSF | | KLLIYA |
| | | | SGGTAALGCLVKDYFPE | | VTMTTDTST | | PITFGQGTRLEIK | | ASSLLSG |
| | | | PVTVSWNSGALTSGVHT | | STAYMELRS | | RTVAAPSVFIFPP | | VPSRFSG |
| | | | FPAVLQSSGLYSLSSVVT | | LRSDDTAVY | | SDEQLKSGTASV | | SGSGTD |
| | | | VPSSSLGTQTYICNVNHK | | YCARDLRG | | VCLLNNFYPRE | | FTLTISS |
| | | | PSNTKVDKKVEPKSCDK | | TNYFDYWG | | AKVQWKVDNA | | LQPEDF |
| | | | THTCPPCPAPELLGGPSV | | QGTLVTVSS | | LQSGNSQESVTE | | ATYYCQ |
| | | | FLFPPKPKDTLMISRTPEV | | | | QDSKDSTYSLSS | | QANSFPI |
| | | | TCVVVDVSHEDPEVKFN | | | | TLTLSKADYEK | | TFGQGT |
| | | | WYVDGVEVHNAKTKPR | | | | HKVYACEVTHQ | | RLEIK |
| | | | EEQYNSTYRVVSVLTVL | | | | GLSSPVTKSFNR | | |
| | | | HQDWLNGKEYKCKVSN | | | | GEC | | |
| | | | KALPAPIEKTISKAKGQP | | | | | | |
| | | | REPQVYTLPPSRDELTKN | | | | | | |
| | | | QVSLWCLVKGFYPSDIA | | | | | | |
| | | | VEWESNGQPENNYKTTP | | | | | | |
| | | | PVLDSDGSFFLYSKLTVD | | | | | | |
| | | | KSRWQQGNVFSCSVMHE | | | | | | |
| | | | ALHNHYTQKSLSLSPGK | | | | | | |

The sequences listed in Table 7 (SEQ ID NOs: 351-370) are amino acid molecules. The sequences listed in Table 7 (SEQ ID NOs: 351-370) are amino acid molecules that are synthetic constructs. The sequences listed in Table 7 (SEQ ID NOs: 351-370) for HC sequences (heavy chain), VH sequence (variable heavy chain sequence), LC sequences (light chain), VL sequence (variable light chain sequence) are amino acid molecules that are synthetic constructs.

TABLE 8
Exemplary CDR sequences for antibodies targeting cMET.

| | | SEQ | | SEQ | | SEQ | |
|---|---|---|---|---|---|---|---|
| Antibody | Arm 2 | ID | | ID | | ID | |
| IDs | Target | NO | CDR H1 | NO | CDR H2 | NO | CDR H3 |
| Amivanta- | cMET | 1003 | SYGIS | 1004 | WISAYNGYT | 1005 | DLRGTNYFDY |
| mab | | | | | NYAQKLQG | | |
| (EPI632 | | | | | | | |
| EPI635 | | | | | | | |
| EPI1088 | | | | | | | |
| EPI1092 | | | | | | | |
| EPI1237 | | | | | | | |
| EPI1238 | | | | | | | |
| EPI1240 | | | | | | | |
| EPI1241 | | | | | | | |
| EPI1242 | | | | | | | |
| EPI1243 | | | | | | | |
| EPI1244 | | | | | | | |
| EPI1245 | | | | | | | |
| EPI1246 | | | | | | | |
| EPI1247 | | | | | | | |
| EPI1248 | | | | | | | |
| EPI1249 | | | | | | | |
| EPI1250 | | | | | | | |
| EPI1251 | | | | | | | |
| EPI1252 | | | | | | | |
| EPI1253 | | | | | | | |
| EPI1254 | | | | | | | |
| EPI1255 | | | | | | | |
| EPI1256 | | | | | | | |
| EPI1257 | | | | | | | |
| EPI1258 | | | | | | | |
| EPI1259 | | | | | | | |
| EPI1260 | | | | | | | |
| EPI1261 | | | | | | | |
| EPI1262 | | | | | | | |
| EPI1263 | | | | | | | |
| EPI1264 | | | | | | | |
| EPI1265 | | | | | | | |
| EPI1266 | | | | | | | |
| EPI1267 | | | | | | | |
| EPI1268 | | | | | | | |
| EPI1269 | | | | | | | |
| EPI1270 | | | | | | | |
| EPI1271 | | | | | | | |
| EPI1272 | | | | | | | |
| EPI1273 | | | | | | | |
| EPI1274 | | | | | | | |
| EPI1275 | | | | | | | |
| EPI1276 | | | | | | | |
| EPI1277 | | | | | | | |
| EPI1278 | | | | | | | |
| EPI1279 | | | | | | | |
| EPI1280 | | | | | | | |
| EPI1281 | | | | | | | |
| EPI1282 | | | | | | | |
| EPI1283 | | | | | | | |
| EPI1284 | | | | | | | |
| EPI1285 | | | | | | | |
| EPI1286 | | | | | | | |
| EPI1287 | | | | | | | |
| EPI1288 | | | | | | | |
| EPI1289 | | | | | | | |
| EPI1290 | | | | | | | |
| EPI1291 | | | | | | | |
| EPI1292 | | | | | | | |
| EPI1293 | | | | | | | |
| EPI1294 | | | | | | | |
| EPI1295 | | | | | | | |
| EPI1296 | | | | | | | |
| EPI1297 | | | | | | | |
| EPI1298 | | | | | | | |
| EPI1299 | | | | | | | |
| EPI1300 | | | | | | | |
| EPI1301 | | | | | | | |
| EPI1302 | | | | | | | |
| EPI1303 | | | | | | | |
| EPI1304 | | | | | | | |
| EPI1305 | | | | | | | |
| EPI1306 | | | | | | | |
| EPI1307 | | | | | | | |
| EPI1308 | | | | | | | |
| EPI1309 | | | | | | | |
| EPI1310 | | | | | | | |
| EPI1311 | | | | | | | |
| EPI1312 | | | | | | | |
| EPI1318 | | | | | | | |
| EPI1319 | | | | | | | |
| EPI1320 | | | | | | | |
| EPI1321 | | | | | | | |
| EPI1322 | | | | | | | |
| EPI1323 | | | | | | | |
| EPI1324 | | | | | | | |
| EPI1325 | | | | | | | |
| EPI1326 | | | | | | | |
| EPI1327 | | | | | | | |
| EPI1328 | | | | | | | |
| EPI1329 | | | | | | | |
| EPI1621 | | | | | | | |
| EPI1622 | | | | | | | |
| EPI1623 | | | | | | | |
| EPI1624 | | | | | | | |
| EPI2132 | | | | | | | |
| EPI2577) | | | | | | | |
| Onartuzu- | cMET | 1009 | SYWLH | 1010 | MIDPSNSD | 1011 | YRSYVTPLDY |
| mab | | | | | TRFNPNFKD | | |
| (EPI1087 | | | | | | | |
| EPI2150 | | | | | | | |
| EPI1091 | | | | | | | |
| EPI2211 | | | | | | | |
| EPI2212 | | | | | | | |
| EPI2213 | | | | | | | |
| EPI2214 | | | | | | | |
| EPI2215 | | | | | | | |
| EPI2216 | | | | | | | |
| EPI2217 | | | | | | | |
| EPI2218 | | | | | | | |
| EPI2219 | | | | | | | |
| EPI2220) | | | | | | | |
| Telisotu- | cMET | 1015 | AYTMH | 1016 | WIKPNNGL | 1017 | SEITTEFDY |
| zumab | T | | | | ANYAQKFQG | | |
| (EPI1086 | | | | | | | |
| EPI2153 | | | | | | | |
| EPI631 | | | | | | | |
| EPI634 | | | | | | | |
| EPI1090 | | | | | | | |
| EPI1112) | | | | | | | |
| REGN5093s58 | cMET | 1021 | DYAMH | 1022 | GITWNSYN | 1023 | DDDYSNYVY |
| (EPI1089 | | | | | IDYADSVKG | | FDY |
| EPI1093) | | | | | | | |

TABLE 9
Exemplary CDR sequences for antibodies targeting cMET.

| | | SEQ | | SEQ | | SEQ | |
|---|---|---|---|---|---|---|---|
| Antibody | Arm 2 | ID | | ID | | ID | |
| IDs | Target | NO | CDR L1 | NO | CDR L2 | NO | CDR L3 |
| Amivanta- | cMET | 1006 | RASQGISNWLA | 1007 | AASSLLS | 1008 | QQANSFPIT |
| mab | | | | | | | |
| (EPI632 | | | | | | | |
| EPI635 | | | | | | | |
| EPI1088 | | | | | | | |
| EPI1092 | | | | | | | |
| EPI1237 | | | | | | | |
| EPI1238 | | | | | | | |
| EPI1240 | | | | | | | |
| EPI1241 | | | | | | | |
| EPI1242 | | | | | | | |
| EPI1243 | | | | | | | |
| EPI1244 | | | | | | | |
| EPI1245 | | | | | | | |
| EPI1246 | | | | | | | |
| EPI1247 | | | | | | | |
| EPI1248 | | | | | | | |
| EPI1249 | | | | | | | |
| EPI1250 | | | | | | | |
| EPI1251 | | | | | | | |
| EPI1252 | | | | | | | |
| EPI1253 | | | | | | | |
| EPI1254 | | | | | | | |
| EPI1255 | | | | | | | |
| EPI1256 | | | | | | | |
| EPI1257 | | | | | | | |
| EPI1258 | | | | | | | |
| EPI1259 | | | | | | | |
| EPI1260 | | | | | | | |
| EPI1261 | | | | | | | |
| EPI1262 | | | | | | | |
| EPI1263 | | | | | | | |
| EPI1264 | | | | | | | |
| EPI1265 | | | | | | | |
| EPI1266 | | | | | | | |
| EPI1267 | | | | | | | |
| EPI1268 | | | | | | | |
| EPI1269 | | | | | | | |
| EPI1270 | | | | | | | |
| EPI1271 | | | | | | | |
| EPI1272 | | | | | | | |
| EPI1273 | | | | | | | |
| EPI1274 | | | | | | | |
| EPI1275 | | | | | | | |
| EPI1276 | | | | | | | |
| EPI1277 | | | | | | | |
| EPI1278 | | | | | | | |
| EPI1279 | | | | | | | |
| EPI1280 | | | | | | | |
| EPI1281 | | | | | | | |
| EPI1282 | | | | | | | |
| EPI1283 | | | | | | | |
| EPI1284 | | | | | | | |
| EPI1285 | | | | | | | |
| EPI1286 | | | | | | | |
| EPI1287 | | | | | | | |
| EPI1288 | | | | | | | |
| EPI1289 | | | | | | | |
| EPI1290 | | | | | | | |
| EPI1291 | | | | | | | |
| EPI1292 | | | | | | | |
| EPI1293 | | | | | | | |
| EPI1294 | | | | | | | |
| EPI1295 | | | | | | | |
| EPI1296 | | | | | | | |
| EPI1297 | | | | | | | |
| EPI1298 | | | | | | | |
| EPI1299 | | | | | | | |
| EPI1300 | | | | | | | |
| EPI1301 | | | | | | | |
| EPI1302 | | | | | | | |
| EPI1303 | | | | | | | |
| EPI1304 | | | | | | | |
| EPI1305 | | | | | | | |
| EPI1306 | | | | | | | |
| EPI1307 | | | | | | | |
| EPI1308 | | | | | | | |
| EPI1309 | | | | | | | |
| EPI1310 | | | | | | | |
| EPI1311 | | | | | | | |
| EPI1312 | | | | | | | |
| EPI1318 | | | | | | | |
| EPI1319 | | | | | | | |
| EPI1320 | | | | | | | |
| EPI1321 | | | | | | | |
| EPI1322 | | | | | | | |
| EPI1323 | | | | | | | |
| EPI1324 | | | | | | | |
| EPI1325 | | | | | | | |
| EPI1326 | | | | | | | |
| EPI1327 | | | | | | | |
| EPI1328 | | | | | | | |
| EPI1329 | | | | | | | |
| EPI1621 | | | | | | | |
| EPI1622 | | | | | | | |
| EPI1623 | | | | | | | |
| EPI1624 | | | | | | | |
| EPI2132 | | | | | | | |
| EPI2577) | | | | | | | |
| Onartuzu- | cMET | 1012 | KSSQSLLYTSSQKNY | 1013 | WASTRES | 1014 | QQYYAYPWT |
| mab | | | LA | | | | |
| (EPI1087 | | | | | | | |
| EPI2150 | | | | | | | |
| EPI1091 | | | | | | | |
| EPI2211 | | | | | | | |
| EPI2212 | | | | | | | |
| EPI2213 | | | | | | | |
| EPI2214 | | | | | | | |
| EPI2215 | | | | | | | |
| EPI2216 | | | | | | | |
| EPI2217 | | | | | | | |
| EPI2218 | | | | | | | |
| EPI2219 | | | | | | | |
| EPI2220) | | | | | | | |
| Telisotu- | cMET | 1018 | KSSESVDSYANSFLH | 1019 | RASTRES | 1020 | QQSKEDPLT |
| zumab | T | | | | | | |
| (EPI1086 | | | | | | | |
| EPI2153 | | | | | | | |
| EPI631 | | | | | | | |
| EPI634 | | | | | | | |
| EPI1090 | | | | | | | |
| EPI1112) | | | | | | | |
| REGN5093s58 | cMET | 1024 | RASQSISSYLN | 1025 | AASSLQS | 1026 | QQSYSTPPIT |
| (EPI1089 | | | | | | | |
| EPI1093) | | | | | | | |

The sequences listed in Table 8 or 9 (SEQ ID NOs: 1003-1026) are amino acid molecules. The sequences listed in Table 8 or 9 (SEQ ID NOs: 1003-1026) are amino acid molecules that are synthetic constructs. The sequences listed in Table 8 or 9 (SEQ ID NOs: 1003-1026) for CDR (complementarity-determining regions) sequences are amino acid molecules that are synthetic constructs.

In some embodiments, the first binding domain comprises at least one complementarity-determining region (CDR) sequence. The first binding domain comprising at least one complementarity-determining region (CDR) sequence may comprise one or more sequences listed in Table 8 or 9. In some cases, the first binding domain comprising at least one complementarity-determining region (CDR) sequence comprises at least 90% sequence identity to one or more sequences listed in Table 8 or 9. In some cases, the first binding domain comprising an antibody comprising an HC sequence, a VH sequence, an LC sequence, and a VL sequence comprises at least 91% sequence identity to one or more sequences listed in Table 8 or 9. In some cases, the first binding domain comprising at least one complementarity-determining region (CDR) sequence comprises at least 92% sequence identity to one or more sequences listed in Table 8 or 9. In some cases, the first binding domain comprising at least one complementarity-determining region (CDR) sequence comprises at least 93% sequence identity to one or more sequences listed in Table 8 or 9. In some cases, the first binding domain comprising at least one complementarity-determining region (CDR) sequence comprises at least 94% sequence identity to one or more sequences listed in Table 8 or 9. In some cases, the first binding domain comprising at least one complementarity-determining region (CDR) sequence comprises at least 95% sequence identity to one or more sequences listed in Table 8 or 9. In some cases, the first binding domain comprising at least one complementarity-determining region (CDR) sequence comprises at least 96% sequence identity to one or more sequences listed in Table 8 or 9. In some cases, the first binding domain comprising at least one complementarity-determining region (CDR) sequence comprises at least 97% sequence identity to one or more sequences listed in Table 8 or 9. In some cases, the first binding domain comprising at least one complementarity-determining region (CDR) sequence comprises at least 98% sequence identity to one or more sequences listed in Table 8 or 9. In some cases, the first binding domain comprising at least one complementarity-determining region (CDR) sequence comprises at least 99% sequence identity to one or more sequences listed in Table 8 or 9. In some cases, the first binding domain comprising at least one complementarity-determining region (CDR) sequence comprises at least 99.5% sequence identity to one or more sequences listed in Table 8 or 9. In some cases, the first binding domain comprising at least one complementarity-determining region (CDR) sequence comprises at least 99.99% sequence identity to one or more sequences listed in Table 8 or 9.

In some embodiments, the first binding domain comprises at least one sequence listed Table 8 or 9. In some embodiments, the first binding domain comprises at least 90%, at least 91%, at least 92%, at least 93%, at least 94%, at least 95%, at least 96%, at least 97%, at least 98%, at least 99%, at least 99.5%, or at least 99.9% sequence identity to the sequences listed Table 8 or 9.

### Synthesis

Binding agents are synthesized using the techniques of recombinant DNA and protein expression. For example, for the synthesis of DNA encoding a dual IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORER assays and/or KINEXAR assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Nucleic Acid Molecules

In one aspect, some embodiments disclosed herein relate to nucleic acid molecules comprising nucleotide sequences encoding the binding agents of the disclosure, including expression cassettes, and expression vectors containing these nucleic acid molecules operably linked to heterologous nucleic acid sequences such as, for example, regulatory sequences which direct in vivo expression of the protein in a host cell.

Also provided herein are vectors, plasmids, or viruses containing one or more of the nucleic acid molecules encoding any dual binding agent disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”): Ausubel, F. M. (1987). Current Protocols in Molecular Biology. New York, NY: Wiley (including supplements through 2014): Bollag, D. M. et al. (1996). Protein Methods. New York, NY: Wiley-Liss: Huang, L. et al. (2005). Nonviral Vectors for Gene Therapy. San Diego: Academic Press: Kaplitt, M. G. et al. (1995). Viral Vectors: Gene Therapy and Neuroscience Applications. San Diego, CA: Academic Press: Lefkovits, I. (1997). The Immunology Methods Manual: The Comprehensive Sourcebook of Techniques. San Diego, CA: Academic Press: Doyle, A. et al. (1998). Cell and Tissue Culture: Laboratory Procedures in Biotechnology. New York, NY: Wiley: Mullis, K. B., Ferré, F. & Gibbs, R. (1994). PCR: The Polymerase Chain Reaction. Boston: Birkhauser Publisher: Greenfield, E. A. (2014). Antibodies: A Laboratory Manual (2nd ed.). New York, NY: Cold Spring Harbor Laboratory Press: Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Methods of Binding On Target Cancer Cells

In some embodiments, the binding agent comprising a first binding domain which specifically binds to a membrane-associated internalizing or degrading protein and a second binding domain which specifically binds to cMET contacts a target cancer cell. In some embodiments, the binding agent comprising a first binding domain which specifically binds to a membrane-associated internalizing or degrading protein and a second binding domain which specifically binds to cMET contacts a gastric adenocarcinoma cancer cell. In some embodiments, the binding agent comprising a first binding domain which specifically binds to a membrane-associated internalizing or degrading protein and a second binding domain which specifically binds to cMET contacts a non-small cell lung cancer cell. In some embodiments, the binding agent comprising a first binding domain which specifically binds to a membrane-associated internalizing or degrading protein and a second binding domain which specifically binds to cMET contacts a target cell selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and head and neck cancer cell. In some embodiments, the cancer cell comprises a mutation in a gene selected from a cMET exon 14 skipping mutation or a cMET duplication mutation.

In some embodiments, the binding agent comprising a first binding domain which specifically binds to a membrane-associated internalizing or degrading protein and a second binding domain which specifically binds to cMET contacts a target cancer cell and decreases expression of cMET on the cancer cell by at least 40%, at least 50%, at least 55%, at least 60%, at least 65%, at least 70%, at least 75%, or at least 80%. In some embodiments, the binding agent comprising a first binding domain which specifically binds a membrane-associated internalizing or degrading protein and a second binding domain which specifically binds to cMET contacts a target cancer cell and decreases expression of cMET on the cancer cell by about 40%-80%, about 50%-80%, about 60%-80%, about 70%-80%, about 40%-70%, about 50%-70%, about 60%-70%, about 40%-60%, or about 50%-60%. In some embodiments, the binding agent comprising a first binding domain which specifically binds to a membrane-associated internalizing or degrading protein and a second binding domain which specifically binds to cMET contacts a target cancer cell and expression of cMET in the cancer cell following the contacting with the binding agent is at least 40%, at least 50%, at least 55%, at least 60%, at least 65%, at least 70%, at least 75%, or at least 80% less than the expression of cMET in a control cancer cell contacted with a monospecific cMET binding agent. In some embodiments, the binding agent comprising a first binding domain which specifically binds a membrane-associated internalizing or degrading protein and a second binding domain which specifically binds to cMET contacts a target cancer cell and expression of cMET in the cancer cell following the contacting with the binding agent is at least 40%-80%, about 50%-80%, about 60%-80%, about 70%-80%, about 40%-70%, about 50%-70%, about 60%-70%, about 40%-60%, or about 50%-60% less than the expression of cMET in a control cancer cell contacted with a monospecific cMET binding agent. In some embodiments, the binding agent comprising a first binding domain which specifically binds to a membrane-associated internalizing or degrading protein and a second binding domain which specifically binds to cMET contacts a target cancer cell and expression of cMET in the cancer cell following the contacting with the binding agent is at least 40%, at least 50%, at least 55%, at least 60%, at least 65%, at least 70%, at least 75%, or at least 80% less than the expression of cMET in a control cancer cell not contacted with a binding agent. In some embodiments, the binding agent comprising a first binding domain which specifically binds a membrane-associated internalizing or degrading protein and a second binding domain which specifically binds to cMET contacts a target cancer cell and expression of cMET in the cancer cell following the contacting with the binding agent is at least 40%-80%, about 50%-80%, about 60%-80%, about 70%-80%, about 40%-70%, about 50%-70%, about 60%-70%, about 40%-60%, or about 50%-60% less than the expression of cMET in a control cancer cell not contacted with a binding agent.

In some embodiments, the binding agent comprising a first binding domain which specifically binds to a membrane-associated internalizing or degrading protein and a second binding domain which specifically binds to cMET contacts a target cancer cell and increases surface removal of cMET on a target cancer cell by at least 20%, at least 30%, at least 40%, at least 50%, at least 60%, at least 65%, at least 70%, at least 75%, at least 80%, at least 85%, or at least 90%. In some embodiments, the binding agent comprising a first binding domain which specifically binds to a membrane-associated internalizing or degrading protein and a second binding domain which specifically binds to cMET on a target cancer cell and increases cell surface removal of cMET by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, the binding agent comprising a first binding domain which specifically binds to a membrane-associated internalizing or degrading protein and a second binding domain which specifically binds to cMET contacts a target cancer cell amount of cMET on the surface of the cancer cell following the contacting with the binding agent is at least 20%, at least 30%, at least 40%, at least 50%, at least 60%, at least 65%, at least 70%, at least 75%, at least 80%, at least 85%, or at least 90% less than amount of cMET on a surface of a control cancer cell not contacted with the binding agent. In some embodiments, the binding agent comprising a first binding domain which specifically binds to a membrane-associated internalizing or degrading protein and a second binding domain which specifically binds to cMET on a target cancer cell and amount of cMET on the surface of the cancer cell following the contacting with the binding agent is about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% less than amount of cMET on a surface of a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell amount of cMET on the surface of the cancer cell following the contacting with the binding agent is at least 20%, at least 30%, at least 40%, at least 50%, at least 60%, at least 65%, at least 70%, at least 75%, at least 80%, at least 85%, or at least 90% less than amount of cMET on a surface of a control cancer cell contacted with a monospecific cMET binding agent. In some embodiments, the binding agent comprising a first binding domain which specifically binds to a membrane-associated internalizing or degrading protein and a second binding domain which specifically binds to cMET on a target cancer cell and amount of cMET on the surface of the cancer cell following the contacting with the binding agent is about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% less than amount of cMET on a surface of a control cancer cell contacted with a monospecific cMET binding agent.

In some embodiments, the binding agent comprising a first binding domain which specifically binds to a membrane-associated internalizing or degrading protein and a second binding domain which specifically binds to cMET contacts a target cancer cell and increases internalization of cMET on a target cancer cell by at least 20%, at least 30%, at least 40%, at least 50%, at least 60%, at least 65%, at least 70%, at least 75%, at least 80%, at least 85%, or at least 90%. In some embodiments, the binding agent comprising a first binding domain which specifically binds to a membrane-associated internalizing or degrading protein and a second binding domain which specifically binds to cMET on a target cancer cell and increases internalization of cMET by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40)-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, internalization of cMET on a target cell is determined relative to internalization of cMET on a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell and amount of cMET internalized for the cancer cell following the contacting with the binding agent is at least 20%, at least 30%, at least 40%, at least 50%, at least 60%, at least 65%, at least 70%, at least 75%, at least 80%, at least 85%, or at least 90% more than amount of cMET internalized for a control cancer cell not contacted with the binding agent. In some embodiments, the binding agent comprising a first binding domain which specifically binds to a membrane-associated internalizing or degrading protein and a second binding domain which specifically binds to cMET on a target cancer cell and amount of cMET internalized for the cancer cell following the contacting with the binding agent is about 20-90%, about 30-90%, about 40)-90%, about 50-90%, about 60-90%, about 70-90%, about 80-90%, about 20-80%, about 30)-80%, about 40-80%, about 50-80%, about 60-80%, about 70-80%, about 20-70%, about 30)-70%, about 40-70%, about 50-70%, about 60-70%, about 20-60%, about 30-60%, about 40)-60%, about 50-60%, about 20-50%, about 30-50%, about 40-50%, about 20-40%, about 30)-40%, or about 20-30% more than amount of cMET internalized for a control cancer cell not contacted with the binding agent. In some embodiments, internalization of cMET on a target cell is determined relative to internalization of cMET on a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell and amount of cMET internalized for the cancer cell following the contacting with the binding agent is at least 20%, at least 30%, at least 40%, at least 50%, at least 60%, at least 65%, at least 70%, at least 75%, at least 80%, at least 85%, or at least 90% more than amount of cMET internalized for a control cancer cell contacted with a monospecific cMET binding agent. In some embodiments, the binding agent comprising a first binding domain which specifically binds to a membrane-associated internalizing or degrading protein and a second binding domain which specifically binds to cMET on a target cancer cell and amount of cMET internalized for the cancer cell following the contacting with the binding agent is about 20)-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% more than amount of cMET internalized for a control cancer cell contacted with a monospecific cMET binding agent.

In some embodiments, the binding agent comprising a first binding domain which specifically binds to a membrane-associated internalizing or degrading protein and a second binding domain which specifically binds to cMET contacts a target cancer cell and increases degradation of cMET on a target cancer cell by at least 20%, at least 30%, at least 40%, at least 50%, at least 60%, at least 65%, at least 70%, at least 75%, at least 80%, at least 85%, or at least 90%. In some embodiments, the binding agent comprising a first binding domain which specifically binds to a membrane-associated internalizing or degrading protein and a second binding domain which specifically binds to cMET on a target cancer cell and increases degradation of cMET by about 20-90%, about 30-90%, about 40-90%, about 50-90%, about 60-90%, about 70-90%, about 80-90%, about 20-80%, about 30-80%, about 40)-80%, about 50-80%, about 60-80%, about 70-80%, about 20-70%, about 30-70%, about 40)-70%, about 50-70%, about 60-70%, about 20-60%, about 30-60%, about 40-60%, about 50)-60%, about 20-50%, about 30-50%, about 40-50%, about 20-40%, about 30-40%, or about 20-30%. In some embodiments, degradation of cMET on a target cell is determined relative to degradation of cMET on a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell and amount of cMET degraded in the cancer cell following the contacting with the binding agent is at least 20%, at least 30%, at least 40%, at least 50%, at least 60%, at least 65%, at least 70%, at least 75%, at least 80%, at least 85%, or at least 90% more than amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET on a target cancer cell and amount of cMET degraded in the cancer cell following the contacting with the binding agent is about 20-90%, about 30-90%, about 40-90%, about 50-90%, about 60-90%, about 70-90%, about 80-90%, about 20-80%, about 30-80%, about 40)-80%, about 50-80%, about 60-80%, about 70-80%, about 20-70%, about 30-70%, about 40-70%, about 50-70%, about 60-70%, about 20-60%, about 30-60%, about 40-60%, about 50)-60%, about 20-50%, about 30-50%, about 40-50%, about 20-40%, about 30-40%, or about 20-30% more than amount of cMET degraded in a control cancer cell not contacted with the binding agent. In some embodiments, degradation of cMET on a target cell is determined relative to degradation of cMET on a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell and amount of cMET degraded in the cancer cell following the contacting with the binding agent is at least 20%, at least 30%, at least 40%, at least 50%, at least 60%, at least 65%, at least 70%, at least 75%, at least 80%, at least 85%, or at least 90% more than amount of cMET degraded in a control cancer cell contacted with a monospecific cMET binding agent. In some embodiments, the binding agent comprising a first binding domain which specifically binds to a membrane-associated internalizing or degrading protein and a second binding domain which specifically binds to cMET on a target cancer cell and amount of cMET degraded in the cancer cell following the contacting with the binding agent is about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% more than amount of cMET degraded in a control cancer cell contacted with a monospecific cMET binding agent.

In some embodiments, the binding agent comprising a first binding domain which specifically binds to a membrane-associated internalizing or degrading protein and a second binding domain which specifically binds to cMET contacts a target cancer cell and amount of cMET dimers on the cancer cell following the contacting with the binding agent is at least 20%, at least 30%, at least 40%, at least 50%, at least 60%, at least 65%, at least 70%, at least 75%, at least 80%, at least 85%, or at least 90% less than amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET on a target cancer cell and amount of cMET dimers on the cancer cell following the contacting with the binding agent is about 20-90%, about 30-90%, about 40-90%, about 50-90%, about 60-90%, about 70-90%, about 80-90%, about 20-80%, about 30-80%, about 40)-80%, about 50-80%, about 60-80%, about 70-80%, about 20-70%, about 30-70%, about 40)-70%, about 50-70%, about 60-70%, about 20-60%, about 30-60%, about 40-60%, about 50)-60%, about 20-50%, about 30-50%, about 40-50%, about 20-40%, about 30-40%, or about 20-30% less than amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell and amount of cMET dimers on the cancer cell following the contacting with the binding agent is at least 20%, at least 30%, at least 40%, at least 50%, at least 60%, at least 65%, at least 70%, at least 75%, at least 80%, at least 85%, or at least 90% less than amount of cMET degraded in a control cancer cell contacted with a monospecific cMET binding agent. In some embodiments, the binding agent comprising a first binding domain which specifically binds to a membrane-associated internalizing or degrading protein and a second binding domain which specifically binds to cMET on a target cancer cell and amount of cMET dimers on the cancer cell following the contacting with the binding agent is about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% less than amount of cMET degraded in a control cancer cell contacted with a monospecific cMET binding agent.

In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is less than an amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is more than an amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is within 20%, 30%, 40%, 50%, 60%, 65%, 70%, 75%, 80%, 85%, 90%, 100%, 150%, 200%, or 300% of than an amount of cMET degraded in a control cancer cell not contacted with the binding agent. In some embodiments, the binding agent comprising a first binding domain which specifically binds to a membrane-associated internalizing or degrading protein and a second binding domain which specifically binds to cMET on a target cancer cell and ana amount of cMET activation in the cancer cell following the contacting with the binding agent is within about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% of an amount of cMET activation in a control cancer cell not contacted with the binding agent.

In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is less than an amount of cMET degraded in a control cancer cell contacted with a monospecific binding agent. In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is more than an amount of cMET degraded in a control cancer cell contacted with a monospecific binding agent. In some embodiments, the binding agent comprising a first binding domain which specifically binds to a membrane-associated internalizing or degrading protein and a second binding domain which specifically binds to cMET contacts a target cancer cell an amount of cMET activation in the cancer cell following the contacting with the binding agent is within 20%, 30%, 40%, 50%, 60%, 65%, 70%, 75%, 80%, 85%, 90%, 100%, 150%, 200%, or 300% of than an amount of cMET degraded in a control cancer cell contacted with a monospecific binding agent. In some embodiments, the binding agent comprising a first binding domain which specifically binds to a membrane-associated internalizing or degrading protein and a second binding domain which specifically binds to cMET on a target cancer cell and ana amount of cMET activation in the cancer cell following the contacting with the binding agent is within about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30% of an amount of cMET activation in a control cancer cell contacted with a monospecific binding agent.

In some embodiments, the binding agent comprising a first binding domain which specifically binds to a membrane-associated internalizing or degrading protein and a second binding domain which specifically binds to cMET contacts a target cancer cell and increases susceptibility of the cancer cell to cancer therapeutic agents. In some embodiments, the binding agent comprising a first binding domain which specifically binds to a membrane-associated internalizing or degrading protein and a second binding domain which specifically binds to cMET contacts a target cancer cell and increases susceptibility of the cancer cell to cytotoxic agents. In some embodiments, the binding agent comprising a first binding domain which specifically binds to a membrane-associated internalizing or degrading protein and a second binding domain which specifically binds to cMET contacts a target cancer cell and reduces proliferation of the target cancer cell. In some embodiments, the binding agent comprising a first binding domain which specifically binds to a membrane-associated internalizing or degrading protein and a second binding domain which specifically binds to cMET contacts a target cancer cell and increases death of the cancer cell.

In some embodiments, the binding agent comprising a first binding domain which specifically binds to a membrane-associated internalizing or degrading protein and a second binding domain which specifically binds to cMET contacts a target cancer cell in vivo.

### Pharmaceutical Compositions

In some embodiments, the binding agents, nucleic acids, and recombinant cells of the disclosure can be incorporated into compositions, including pharmaceutical compositions. Such compositions typically include the binding agents, and a pharmaceutically acceptable excipient, e.g., a carrier. Binding agents of the disclosure can be administered using formulations used for administering antibodies and antibody-based therapeutics, or formulations based thereon.

Pharmaceutical compositions suitable for injectable use include sterile aqueous solutions (where water soluble) or dispersions and sterile powders for the extemporaneous preparation of sterile injectable solutions or dispersion.

### Administration of Binding Agents

Administration of any one or more of the therapeutic compositions described herein, e.g., binding agents and pharmaceutical compositions, can be used to treat individuals having a neoplastic disease, such as cancers.

Accordingly, in one aspect, provided herein are methods for inhibiting an activity of a target cell in an individual, the methods comprising the step of administering to the individual a first therapy including one or more of the binding agents and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Generally, the target cell of the disclosed methods can be any cancer cell.

In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises cMET.

In some embodiments, the binding agents as disclosed herein can be compared to other binding agents. In some cases, the other binding agents are monospecific binding agents. In some embodiments, the monospecific binding agent is Telisotuxumab. In some embodiments, the monospecific binding agent is Onartuzumab. In some embodiments, the monospecific binding agent is REGN5093s58. In some cases, the other binding agents may target membrane associated proteins that are not cMET. In some cases, the other binding agents may target degrading proteins that not cMET. In some cases, the other binding agents may bind to the RSV F Protein. In some cases, the other binding agents may not bind to a target. In some embodiments, a binding domain configured to bind to a control (e.g., RSV) comprises a sequence listed in Table 10. In some embodiments, a binding domain configured to bind to a control (e.g., RSV) comprises a sequence listed in Table 11. In some cases, the other binding agents are monospecific binding agents. In some embodiments, a monospecific binding agent comprises a sequence listed in Table 12.

TABLE 10
Exemplary binding agent control arms for Arm 1

| | | SEQ | | SEQ | | SEQ | | SEQ | |
|---|---|---|---|---|---|---|---|---|---|
| Antibody | Arm 1 | ID | | ID | VH | ID | | ID | VL |
| IDs | Target | NO | HC sequence | NO | sequence | NO | LC sequence | NO | sequence |
| EPI632 | None | 371 | EPKSCDKTHTCPPCPA | | | | | | |
| EPI631 | | | PELLGGPSVFLFPPKP | | | | | | |
| | | | KDTLMISRTPEVTCVV | | | | | | |
| | | | VDVSHEDPEVKFNWY | | | | | | |
| | | | VDGVEVHNAKTKPRE | | | | | | |
| | | | EQYNSTYRVVSVLTV | | | | | | |
| | | | LHQDWLNGKEYKCK | | | | | | |
| | | | VSNKALPAPIEKTISK | | | | | | |
| | | | AKGQPREPQVYTLPPS | | | | | | |
| | | | RDELTKNQVSLWCLV | | | | | | |
| | | | KGFYPSDIAVEWESN | | | | | | |
| | | | GQPENNYKTTPPVLDS | | | | | | |
| | | | DGSFFLYSKLTVDKSR | | | | | | |
| | | | WQQGNVFSCSVMHE | | | | | | |
| | | | ALHNHYTQKSLSLSPG | | | | | | |
| | | | KGGSHHHHHH | | | | | | |
| Farletu- | RSV | 372 | QVTLRESGPALVKPTQ | 373 | QVTLRESGP | 374 | DIQMTQSPSTLS | 375 | DIQMTQ |
| zumab | | | TLTLTCTFSGFSLSTSG | | ALVKPTQTL | | ASVGDRVTITCK | | SPSTLSA |
| (EPI1088 | | | MSVGWIRQPPGKALE | | TLTCTFSGFS | | CQLSVGYMHW | | SVGDRV |
| EPI1086) | | | WLADIWWDDKKDYN | | LSTSGMSVG | | YQQKPGKAPKL | | TITCKCQ |
| | | | PSLKSRLTISKDTSKN | | WIRQPPGKA | | LIYDTSKLASGV | | LSVGYM |
| | | | QVVLKVTNMDPADT | | LEWLADIW | | PSRFSGSGSGTE | | HWYQQ |
| | | | ATYYCARSMITNWYF | | WDDKKDYN | | FTLTISSLQPDDF | | KPGKAP |
| | | | DVWGAGTTVTVSSAS | | PSLKSRLTIS | | ATYYCFQGSGY | | KLLIYDT |
| | | | TKGPSVFPLAPSSKSTS | | KDTSKNQV | | PFTFGGGTKLEI | | SKLASG |
| | | | GGTAALGCLVKDYFP | | VLKVTNMD | | KRTVAAPSVFIF | | VPSRFSG |
| | | | EPVTVSWNSGALTSG | | PADTATYYC | | PPSDEQLKSGTA | | SGSGTEF |
| | | | VHTFPAVLQSSGLYSL | | ARSMITNW | | SVVCLLNNFYPR | | TLTISSL |
| | | | SSVVTVPSSSLGTQTYI | | YFDVWGAG | | EAKVQWKVDN | | QPDDFA |
| | | | CNVNHKPSNTKVDKK | | TTVTVSS | | ALQSGNSQESVT | | TYYCFQ |
| | | | VEPKSCDKTHTCPPCP | | | | EQDSKDSTYSLS | | GSGYPF |
| | | | APELLGGPSVFLFPPK | | | | STLTLSKADYEK | | TFGGGT |
| | | | PKDTLMISRTPEVTCV | | | | HKVYACEVTHQ | | KLEIK |
| | | | VVDVSHEDPEVKFNW | | | | GLSSPVTKSFNR | | |
| | | | YVDGVEVHNAKTKPR | | | | GEC | | |
| | | | EEQYNSTYRVVSVLT | | | | | | |
| | | | VLHQDWLNGKEYKC | | | | | | |
| | | | KVSNKALPAPIEKTIS | | | | | | |
| | | | KAKGQPREPQVYTLP | | | | | | |
| | | | PSRDELTKNQVSLWC | | | | | | |
| | | | LVKGFYPSDIAVEWES | | | | | | |
| | | | NGQPENNYKTTPPVL | | | | | | |
| | | | DSDGSFFLYSKLTVDK | | | | | | |
| | | | SRWQQGNVFSCSVMH | | | | | | |
| | | | EALHNHYTQKSLSLSP | | | | | | |
| | | | GKGGSHHHHHH | | | | | | |
| Palivizu- | RSV | 376 | QVTLRESGPALVKPTQ | 377 | QVTLRESGP | 378 | DIQMTQSPSTLS | 379 | DIQMTQ |
| mab | | | TLTLTCTFSGFSLSTSG | | ALVKPTQTL | | ASVGDRVTITCK | | SPSTLSA |
| (EPI2132 | | | MSVGWIRQPPGKALE | | TLTCTFSGFS | | CQLSVGYMHW | | SVGDRV |
| EPI2150 | | | WLADIWWDDKKDYN | | LSTSGMSVG | | YQQKPGKAPKL | | TITCKCQ |
| EPI2153 | | | PSLKSRLTISKDTSKN | | WIRQPPGKA | | LIYDTSKLASGV | | LSVGYM |
| EPI1089) | | | QVVLKVTNMDPADT | | LEWLADIW | | PSRFSGSGSGTE | | HWYQQ |
| | | | ATYYCARSMITNWYF | | WDDKKDYN | | FTLTISSLQPDDF | | KPGKAP |
| | | | DVWGAGTTVTVSSAS | | PSLKSRLTIS | | ATYYCFQGSGY | | KLLIYDT |
| | | | TKGPSVFPLAPSSKSTS | | KDTSKNQV | | PFTFGGGTKLEI | | SKLASG |
| | | | GGTAALGCLVKDYFP | | VLKVTNMD | | KRTVAAPSVFIF | | VPSRFSG |
| | | | EPVTVSWNSGALTSG | | PADTATYYC | | PPSDEQLKSGTA | | SGSGTEF |
| | | | VHTFPAVLQSSGLYSL | | ARSMITNW | | SVVCLLNNFYPR | | TLTISSL |
| | | | SSVVTVPSSSLGTQTYI | | YFDVWGAG | | EAKVQWKVDN | | QPDDFA |
| | | | CNVNHKPSNTKVDKK | | TTVTVSS | | ALQSGNSQESVT | | TYYCFQ |
| | | | VEPKSCDKTHTCPPCP | | | | EQDSKDSTYSLS | | GSGYPF |
| | | | APELLGGPSVFLFPPK | | | | STLTLSKADYEK | | TFGGGT |
| | | | PKDTLMISRTPEVTCV | | | | HKVYACEVTHQ | | KLEIK |
| | | | VVDVSHEDPEVKFNW | | | | GLSSPVTKSFNR | | |
| | | | YVDGVEVHNAKTKPR | | | | GEC | | |
| | | | EEQYNSTYRVVSVLT | | | | | | |
| | | | VLHQDWLNGKEYKC | | | | | | |
| | | | KVSNKALPAPIEKTIS | | | | | | |
| | | | KAKGQPREPQVYTLP | | | | | | |
| | | | PSRDELTKNQVSLWC | | | | | | |
| | | | LVKGFYPSDIAVEWES | | | | | | |
| | | | NGQPENNYKTTPPVL | | | | | | |
| | | | DSDGSFFLYSKLTVDK | | | | | | |
| | | | SRWQQGNVFSCSVMH | | | | | | |
| | | | EALHNHYTQKSLSLSP | | | | | | |
| | | | GK | | | | | | |
| SN82 | cMET | 380 | EVQLVESGGGLVQPG | 381 | EVQLVESGG | 382 | DIQMTQSPSSLS | 383 | DIQMTQ |
| (EPI2577 | | | GSLRLSCAASGFIVTT | | GLVQPGGSL | | ASVGDRVTITCR | | SPSSLSA |
| EPI1578) | | | NYMTWLRQAPGKGL | | RLSCAASGF | | ASQSISSYLNWY | | SVGDRV |
| | | | EWVSLIYSSGHTYYA | | IVTTNYMT | | QQKPGKAPKLLI | | TITCRAS |
| | | | DSVKGRFTISRHNSKN | | WLRQAPGK | | YAASSLQSGVPS | | QSISSYL |
| | | | TLYLQMDSLRAEDTA | | GLEWVSLIY | | RFSGSGSGTDFT | | NWYQQ |
| | | | VYYCASAFAADVFDI | | SSGHTYYAD | | LTISSLQPEDFAT | | KPGKAP |
| | | | WGQGTMVTVSSASTK | | SVKGRFTIS | | YYCQQSYSTPPI | | KLLIYA |
| | | | GPSVFPLAPSSKSTSG | | RHNSKNTLY | | TFGQGTRLEIKR | | ASSLQS |
| | | | GTAALGCLVKDYFPE | | LQMDSLRA | | TVAAPSVFIFPPS | | GVPSRFS |
| | | | PVTVSWNSGALTSGV | | EDTAVYYC | | DEQLKSGTASV | | GSGSGT |
| | | | HTFPAVLQSSGLYSLS | | ASAFAADVF | | VCLLNNFYPRE | | DFTLTIS |
| | | | SVVTVPSSSLGTQTYI | | DIWGQGTM | | AKVQWKVDNA | | SLQPEDF |
| | | | CNVNHKPSNTKVDKK | | VTVSS | | LQSGNSQESVTE | | ATYYCQ |
| | | | VEPKSCDKTHTCPPCP | | | | QDSKDSTYSLSS | | QSYSTPP |
| | | | APELLGGPSVFLFPPK | | | | TLTLSKADYEK | | ITFGQGT |
| | | | PKDTLMISRTPEVTCV | | | | HKVYACEVTHQ | | RLEIK |
| | | | VVDVSHEDPEVKFNW | | | | GLSSPVTKSFNR | | |
| | | | YVDGVEVHNAKTKPR | | | | GEC | | |
| | | | EEQYNSTYRVVSVLT | | | | | | |
| | | | VLHQDWLNGKEYKC | | | | | | |
| | | | KVSNKALPAPIEKTIS | | | | | | |
| | | | KAKGQPREPQVYTLP | | | | | | |
| | | | PSRDELTKNQVSLWC | | | | | | |
| | | | LVKGFYPSDIAVEWES | | | | | | |
| | | | NGQPENNYKTTPPVL | | | | | | |
| | | | DSDGSFFLYSKLTVDK | | | | | | |
| | | | SRWQQGNVFSCSVMH | | | | | | |
| | | | EALHNHYTQKSLSLSP | | | | | | |
| | | | GK | | | | | | |
| Zalutumu- | EGFR | 384 | QVQLVESGGGVVQPG | 385 | QVQLVESG | 386 | AIQLTQSPSS | 387 | AIQLT |
| mab | | | RSLRLSCAASGFTFST | | GGVVQPG | | LSASVGDRV | | QSPSSL |
| (EPI818) | | | YGMHWVRQAPGKGL | | RSLRLSCA | | TITCRASQDIS | | SASVG |
| | | | EWVAVIWDDGSYKY | | ASGFTFST | | SALVWYQQK | | DRVTIT |
| | | | YGDSVKGRFTISRDNS | | YGMHWVR | | PGKAPKLLIY | | CRASQ |
| | | | KNTLYLQMNSLRAED | | QAPGKGLE | | DASSLESGVP | | DISSAL |
| | | | TAVYYCARDGITMVR | | WVAVIWD | | SRFSGSESGT | | VWYQ |
| | | | GVMKDYFDYWGQGT | | DGSYKYY | | DFTLTISSLQP | | QKPGK |
| | | | LVTVSSAKTTPPSVYP | | GDSVKGRF | | EDFATYYCQ | | APKLLI |
| | | | LAPGSAAQTNSMVTL | | TISRDNSK | | QFNSYPLTFG | | YDASS |
| | | | GCLVKGYFPEPVTVT | | NTLYLQM | | GGTKVEIKR | | LESGV |
| | | | WNSGSLSSGVHTFPA | | NSLRAEDT | | ADAAPTVSIF | | PSRFSG |
| | | | VLQSDLYTLSSSVTVP | | AVYYCAR | | PPSSEQLTSG | | SESGT |
| | | | SSPRPSETVTCNVAHP | | DGITMVRG | | GASVVCFLN | | DFTLTI |
| | | | ASSTKVDKKIVPRDCD | | VMKDYFD | | NFYPKDINVK | | SSLQPE |
| | | | KTHTCPPCPAPELLGG | | YWGQGTL | | WKIDGSERQ | | DFATY |
| | | | PSVFLFPPKPKDTLMIS | | VTVSS | | NGVLNSWTD | | YCQQF |
| | | | RTPEVTCVVVDVSHE | | | | QDSKDSTYS | | NSYPL |
| | | | DPEVKFNWYVDGVE | | | | MSSTLTLTKD | | TFGGG |
| | | | VHNAKTKPREEQYNS | | | | EYERHNSYT | | TKVEI |
| | | | TYRVVSVLTVLHQDW | | | | CEATHKTSTS | | K |
| | | | LNGKEYKCKVSNKAL | | | | PIVKSFNRNE | | |
| | | | PAPIEKTISKAKGQPRE | | | | C | | |
| | | | PQVYTLPPSRDELTKN | | | | | | |
| | | | QVSLSCAVKGFYPSDI | | | | | | |
| | | | AVEWESNGQPENNYK | | | | | | |
| | | | TTPPVLDSDGSFFLVS | | | | | | |
| | | | KLTVDKSRWQQGNVF | | | | | | |
| | | | SCSVMHEALHNHYTQ | | | | | | |
| | | | KSLSLSPGK | | | | | | |
| Mouse | Con- | 388 | QVTLRESGPALVKPTQ | 389 | QVTLRESG | 390 | DIQMTQSPSTLS | 391 | DIQMTQ |
| IgG1 CH1 | tro1 | | TLTLTCTFSGFSLSTSG | | PALVKPTQ | | ASVGDRVTITCK | | SPSTLSA |
| (EPI1086/ | | | MSVGWIRQPPGKALE | | TLTLTCTFS | | CQLSVGYMHW | | SVGDRV |
| EPI1087) | | | WLADIWWDDKKDYN | | GFSLSTSG | | YQQKPGKAPKL | | TITCKCQ |
| | | | PSLKSRLTISKDTSKN | | MSVGWIR | | LIYDTSKLASGV | | LSVGYM |
| | | | QVVLKVTNMDPADT | | QPPGKALE | | PSRFSGSGSGTE | | HWYQQ |
| | | | ATYYCARSMITNWYF | | WLADIWW | | FTLTISSLQPDDF | | KPGKAP |
| | | | DVWGAGTTVTVSSAS | | DDKKDYN | | ATYYCFQGSGY | | KLLIYDT |
| | | | TKGPSVFPLAPSSKSTS | | PSLKSRLTI | | PFTFGGGTKLEI | | SKLASG |
| | | | GGTAALGCLVKDYFP | | SKDTSKNQ | | KRTVAAPSVFIF | | VPSRFSG |
| | | | EPVTVSWNSGALTSG | | VVLKVTN | | PPSDEQLKSGTA | | SGSGTEF |
| | | | VHTFPAVLQSSGLYSL | | MDPADTA | | SVVCLLNNFYPR | | TLTISSL |
| | | | SSVVTVPSSSLGTQTYI | | TYYCARS | | EAKVQWKVDN | | QPDDFA |
| | | | CNVNHKPSNTKVDKK | | MITNWYFD | | ALQSGNSQESVT | | TYYCFQ |
| | | | VEPKSCDKTHTCPPCP | | VWGAGTT | | EQDSKDSTYSLS | | GSGYPF |
| | | | APELLGGPSVFLFPPK | | VTVSS | | STLTLSKADYEK | | TFGGGT |
| | | | PKDTLMISRTPEVTCV | | | | HKVYACEVTHQ | | KLEIK |
| | | | VVDVSHEDPEVKFNW | | | | GLSSPVTKSFNR | | |
| | | | YVDGVEVHNAKTKPR | | | | GEC | | |
| | | | EEQYNSTYRVVSVLT | | | | | | |
| | | | VLHQDWLNGKEYKC | | | | | | |
| | | | KVSNKALPAPIEKTIS | | | | | | |
| | | | KAKGQPREPQVYTLP | | | | | | |
| | | | PSRDELTKNQVSLWC | | | | | | |
| | | | LVKGFYPSDIAVEWES | | | | | | |
| | | | NGQPENNYKTTPPVL | | | | | | |
| | | | DSDGSFFLYSKLTVDK | | | | | | |
| | | | SRWQQGNVFSCSVMH | | | | | | |
| | | | EALHNHYTQKSLSLSP | | | | | | |
| | | | GKGGSHHHHHH | | | | | | |
| Farletu- | FR- | 392 | QVTLRESGPALVKPTQ | 393 | EVQLVESG | 394 | DIQMTQSPST | 395 | DIQMT |
| zumab | alpha | | TLTLTCTFSGFSLSTSG | | GGVVQPG | | LSASVGDRV | | QSPSTL |
| EPI632/ | | | MSVGWIRQPPGKALE | | RSLRLSCS | | TITCKCQLSV | | SASVG |
| EPI634 | | | WLADIWWDDKKDYN | | ASGFTFSG | | GYMHWYQQ | | DRVTIT |
| | | | PSLKSRLTISKDTSKN | | YGLSWVR | | KPGKAPKLLI | | CKCQL |
| | | | QVVLKVTNMDPADT | | QAPGKGLE | | YDTSKLASG | | SVGYM |
| | | | ATYYCARSMITNWYF | | WVAMISSG | | VPSRFSGSGS | | HWYQ |
| | | | DVWGAGTTVTVSSAS | | GSYTYYAD | | GTEFTLTISSL | | QKPGK |
| | | | TKGPSVFPLAPSSKSTS | | SVKGRFAI | | QPDDFATYY | | APKLLI |
| | | | GGTAALGCLVKDYFP | | SRDNAKNT | | CFQGSGYPFT | | YDTSK |
| | | | EPVTVSWNSGALTSG | | LFLQMDSL | | FGGGTKLEIK | | LASGV |
| | | | VHTFPAVLQSSGLYSL | | RPEDTGVY | | RTVAAPSVFI | | PSRFSG |
| | | | SSVVTVPSSSLGTQTYI | | FCARHGDD | | FPPSDEQLKS | | SGSGT |
| | | | CNVNHKPSNTKVDKK | | PAWFAYW | | GTASVVCLL | | EFTLTI |
| | | | VEPKSCDKTHTCPPCP | | GQGTPVTV | | NNFYPREAK | | SSLQP |
| | | | APELLGGPSVFLFPPK | | SS | | VQWKVDNA | | DDFAT |
| | | | PKDTLMISRTPEVTCV | | | | LQSGNSQESV | | YYCFQ |
| | | | VVDVSHEDPEVKFNW | | | | TEQDSKDST | | GSGYP |
| | | | YVDGVEVHNAKTKPR | | | | YSLSSTLTLS | | FTFGG |
| | | | EEQYNSTYRVVSVLT | | | | KADYEKHKV | | GTKLEI |
| | | | VLHQDWLNGKEYKC | | | | YACEVTHQG | | K |
| | | | KVSNKALPAPIEKTIS | | | | LSSPVTKSFN | | |
| | | | KAKGQPREPQVYTLP | | | | RGEC | | |
| | | | PSRDELTKNQVSLWC | | | | | | |
| | | | LVKGFYPSDIAVEWES | | | | | | |
| | | | NGQPENNYKTTPPVL | | | | | | |
| | | | DSDGSFFLYSKLTVDK | | | | | | |
| | | | SRWQQGNVFSCSVMH | | | | | | |
| | | | EALHNHYTQKSLSLSP | | | | | | |
| | | | GKGGSHHHHHH | | | | | | |
| Tixagevi- | COVID | 396 | QMQLVQSGPEVKKPG | 397 | QMQLVQS | 398 | EIVLTQSPGT | 399 | EIVLTQ |
| mab | RBD | | TSVKVSCKASGFTFMS | | GPEVKKPG | | LSLSPGERAT | | SPGTLS |
| (EPI1112) | | | SAVQWVRQARGQRL | | TSVKVSCK | | LSCRASQSVS | | LSPGE |
| | | | EWIGWIVIGSGNTNYA | | ASGFTFMS | | SSYLAWYQQ | | RATLS |
| | | | QKFQERVTITRDMSTS | | SAVQWVR | | KPGQAPRLLI | | CRASQ |
| | | | TAYMELSSLRSEDTA | | QARGQRLE | | YGASSRATGI | | SVSSSY |
| | | | VYYCAAPYCSSISCND | | WIGWIVIG | | PDRFSGSGSG | | LAWY |
| | | | GFDIWGQGTMVTVSS | | SGNTNYAQ | | TDFTLTISRLE | | QQKPG |
| | | | ASTKGPSVFPLAPSSK | | KFQERVTI | | PEDFAVYYC | | QAPRL |
| | | | STSGGTAALGCLVKD | | TRDMSTST | | QHYGSSRGW | | LIYGAS |
| | | | YFPEPVTVSWNSGAL | | AYMELSSL | | TFGQGTKVEI | | SRATGI |
| | | | TSGVHTFPAVLQSSGL | | RSEDTAVY | | KRTVAAPSV | | PDRFS |
| | | | YSLSSVVTVPSSSLGT | | YCAAPYCS | | FIFPPSDEQLK | | GSGSG |
| | | | QTYICNVNHKPSNTK | | SISCNDGF | | SGTASVVCLL | | TDFTL |
| | | | VDKKVEPKSCDKTHT | | DIWGQGT | | NNFYPREAK | | TISRLE |
| | | | CPPCPAPELLGGPSVF | | MVTVSSGG | | VQWKVDNA | | PEDFA |
| | | | LFPPKPKDTLMISRTPE | | S | | LQSGNSQESV | | VYYCQ |
| | | | VTCVVVDVSHEDPEV | | | | TEQDSKDST | | HYGSS |
| | | | KFNWYVDGVEVHNA | | | | YSLSSTLTLS | | RGWTF |
| | | | KTKPREEQYNSTYRV | | | | KADYEKHKV | | GQGTK |
| | | | VSVLTVLHQDWLNGK | | | | YACEVTHQG | | VEIK |
| | | | EYKCKVSNKALPAPIE | | | | LSSPVTKSFN | | |
| | | | KTISKAKGQPREPQVY | | | | RGEC | | |
| | | | TLPPSRDELTKNQVSL | | | | | | |
| | | | WCLVKGFYPSDIAVE | | | | | | |
| | | | WESNGQPENNYKTTP | | | | | | |
| | | | PVLDSDGSFFLYSKLT | | | | | | |
| | | | VDKSRWQQGNVFSCS | | | | | | |
| | | | VMHEALHNHYTQKSL | | | | | | |
| | | | SLSPGKGGSHHHHHH | | | | | | |

TABLE 11
Exemplary binding agent control arms for Arm 2

| | Arm 2 | SEQ | | SEQ | | SEQ | | SEQ | |
|---|---|---|---|---|---|---|---|---|---|
| Antibody | Tar- | ID | | ID | VH | ID | LC | ID | VL |
| IDs | get | NO | HC sequence | NO | sequence | NO | sequence | NO | sequence |
| Palivi- | RSV | 400 | QVTLRESGPALVKPTQ | 401 | QVTLRESG | 402 | DIQMTQSPST | 403 | DIQMT |
| zumab | | | TLTLTCTFSGFSLSTSG | | PALVKPTQ | | LSASVGDRV | | QSPSTL |
| (EPI1118 | | | MSVGWIRQPPGKALEW | | TLTLTCTF | | TITCKCQLSV | | SASVG |
| EPI1119 | | | LADIWWDDKKDYNPS | | SGFSLSTS | | GYMHWYQQ | | DRVTIT |
| EPI1121 | | | LKSRLTISKDTSKNQVV | | GMSVGWI | | KPGKAPKLLI | | CKCQL |
| EPI1122 | | | LKVTNMDPADTATYY | | RQPPGKAL | | YDTSKLASG | | SVGYM |
| EPI1123 | | | CARSMITNWYFDVWG | | EWLADIW | | VPSRFSGSGS | | HWYQ |
| EPI1135 | | | AGTTVTVSSAKTTPPSV | | WDDKKDY | | GTEFTLTISSL | | QKPGK |
| EPI1136 | | | YPLAPGSAAQTNSMVT | | NPSLKSRL | | QPDDFATYY | | APKLLI |
| EPI1137 | | | LGCLVKGYFPEPVTVT | | TISKDTSK | | CFQGSGYPFT | | YDTSK |
| EPI1138 | | | WNSGSLSSGVHTFPAV | | NQVVLKV | | FGGGTKLEIK | | LASGV |
| EPI1139 | | | LQSDLYTLSSSVTVPSS | | TNMDPAD | | RADAAPTVSI | | PSRFSG |
| EPI1140 | | | PRPSETVTCNVAHPASS | | TATYYCA | | FPPSSEQLTS | | SGSGT |
| EPI1141 | | | TKVDKKIVPRDCDKTH | | RSMITNW | | GGASVVCFL | | EFTLTI |
| EPI1142 | | | TCPPCPAPELLGGPSVF | | YFDVWGA | | NNFYPKDINV | | SSLQP |
| EPI1147 | | | LFPPKPKDTLMISRTPE | | GTTVTVSS | | KWKIDGSER | | DDFAT |
| EPI1148 | | | VTCVVVDVSHEDPEVK | | | | QNGVLNSWT | | YYCFQ |
| EPI1149 | | | FNWYVDGVEVHNAKT | | | | DQDSKDSTY | | GSGYP |
| EPI1150 | | | KPREEQYNSTYRVVSV | | | | SMSSTLTLTK | | FTFGG |
| EPI1151 | | | LTVLHQDWLNGKEYK | | | | DEYERHNSY | | GTKLEIK |
| EPI1152 | | | CKVSNKALPAPIEKTIS | | | | TCEATHKTST | | |
| EPI1153 | | | KAKGQPREPQVYTLPP | | | | SPIVKSFNRN | | |
| EPI1154 | | | SRDELTKNQVSLSCAV | | | | EC | | |
| EPI1155 | | | KGFYPSDIAVEWESNG | | | | | | |
| EPI1156 | | | QPENNYKTTPPVLDSD | | | | | | |
| EPI1157 | | | GSFFLVSKLTVDKSRW | | | | | | |
| EPI1158 | | | QQGNVFSCSVMHEALH | | | | | | |
| EPI1159 | | | NHYTQKSLSLSPGK | | | | | | |
| EPI1160 | | | | | | | | | |
| EPI1161 | | | | | | | | | |
| EPI1162 | | | | | | | | | |
| EPI1163 | | | | | | | | | |
| EPI1164 | | | | | | | | | |
| EPI1165 | | | | | | | | | |
| EPI1166 | | | | | | | | | |
| EPI1167 | | | | | | | | | |
| EPI1168 | | | | | | | | | |
| EPI1169 | | | | | | | | | |
| EPI1173 | | | | | | | | | |
| EPI1174 | | | | | | | | | |
| EPI1175 | | | | | | | | | |
| EPI1176 | | | | | | | | | |
| EPI1177 | | | | | | | | | |
| EPI1178 | | | | | | | | | |
| EPI1179 | | | | | | | | | |
| EPI1180 | | | | | | | | | |
| EPI1181 | | | | | | | | | |
| EPI1182 | | | | | | | | | |
| EPI1183 | | | | | | | | | |
| EPI1184 | | | | | | | | | |
| EPI1406 | | | | | | | | | |
| EPI1407 | | | | | | | | | |
| EPI1489 | | | | | | | | | |
| EPI1522 | | | | | | | | | |
| EPI1523 | | | | | | | | | |
| EPI1524 | | | | | | | | | |
| EPI1525 | | | | | | | | | |
| EPI1526 | | | | | | | | | |
| EPI1552 | | | | | | | | | |
| EPI1606 | | | | | | | | | |
| EPI1607 | | | | | | | | | |
| EPI1608 | | | | | | | | | |
| EPI1609 | | | | | | | | | |
| EPI1610 | | | | | | | | | |
| EPI1611 | | | | | | | | | |
| EPI1612 | | | | | | | | | |
| EPI1613 | | | | | | | | | |
| EPI1614 | | | | | | | | | |
| EPI1615 | | | | | | | | | |
| EPI1627 | | | | | | | | | |
| EPI1628 | | | | | | | | | |
| EPI1730 | | | | | | | | | |
| EPI1731 | | | | | | | | | |
| EPI1732 | | | | | | | | | |
| EPI1733 | | | | | | | | | |
| EPI1734 | | | | | | | | | |
| EPI1735 | | | | | | | | | |
| EPI1736 | | | | | | | | | |
| EPI1737 | | | | | | | | | |
| EPI1738 | | | | | | | | | |
| EPI1739 | | | | | | | | | |
| EPI1740 | | | | | | | | | |
| EPI1741 | | | | | | | | | |
| EPI1742 | | | | | | | | | |
| EPI1743 | | | | | | | | | |
| EPI1744 | | | | | | | | | |
| EPI2075 | | | | | | | | | |
| EPI2086 | | | | | | | | | |
| EPI2322 | | | | | | | | | |
| EPI2499 | | | | | | | | | |
| EPI2500 | | | | | | | | | |
| EPI2501 | | | | | | | | | |
| EPI2502 | | | | | | | | | |
| EPI2578) | | | | | | | | | |

TABLE 12
Exemplary monospecific antibody controls

| Anti- | | SEQ | | SEQ | | SEQ | | SEQ | |
|---|---|---|---|---|---|---|---|---|---|
| body | Tar- | ID | | ID | VH | ID | | ID | VL |
| IDs | get | NO | HC sequence | NO | sequence | NO | LC sequence | NO | sequence |
| Palivi- | RSV | 404 | QVTLRESGPALVKPTQ | 405 | QVTLRESG | 506 | DIQMTQSPST | 407 | DIQMT |
| zumab | | | TLTLTCTFSGFSLSTSG | | PALVKPTQ | | LSASVGDRV | | QSPSTL |
| (EPI692) | | | MSVGWIRQPPGKALEW | | TLTLTCTF | | TITCKCQLSV | | SASVG |
| | | | LADIWWDDKKDYNPS | | SGFSLSTS | | GYMHWYQQ | | DRVTIT |
| | | | LKSRLTISKDTSKNQVV | | GMSVGWI | | KPGKAPKLLI | | CKCQL |
| | | | LKVTNMDPADTATYY | | RQPPGKAL | | YDTSKLASG | | SVGYM |
| | | | CARSMITNWYFDVWG | | EWLADIW | | VPSRFSGSGS | | HWYQ |
| | | | AGTTVTVSSASTKGPSV | | WDDKKDY | | GTEFTLTISSL | | QKPGK |
| | | | FPLAPSSKSTSGGTAAL | | NPSLKSRL | | QPDDFATYY | | APKLLI |
| | | | GCLVKDYFPEPVTVSW | | TISKDTSK | | CFQGSGYPFT | | YDTSK |
| | | | NSGALTSGVHTFPAVL | | NQVVLKV | | FGGGTKLEIK | | LASGV |
| | | | QSSGLYSLSSVVTVPSS | | TNMDPAD | | RTVAAPSVFI | | PSRFSG |
| | | | SLGTQTYICNVNHKPSN | | TATYYCA | | FPPSDEQLKS | | SGSGT |
| | | | TKVDKKVEPKSCDKTH | | RSMITNW | | GTASVVCLL | | EFTLTI |
| | | | TCPPCPAPELLGGPSVF | | YFDVWGA | | NNFYPREAK | | SSLQP |
| | | | LFPPKPKDTLMISRTPE | | GTTVTVSS | | VQWKVDNA | | DDFAT |
| | | | VTCVVVDVSHEDPEVK | | | | LQSGNSQESV | | YYCFQ |
| | | | FNWYVDGVEVHNAKT | | | | TEQDSKDST | | GSGYP |
| | | | KPREEQYNSTYRVVSV | | | | YSLSSTLTLS | | FTFGG |
| | | | LTVLHQDWLNGKEYK | | | | KADYEKHKV | | GTKLEI |
| | | | CKVSNKALPAPIEKTIS | | | | YACEVTHQG | | K |
| | | | KAKGQPREPQVYTLPP | | | | LSSPVTKSFN | | |
| | | | SRDELTKNQVSLTCLV | | | | RGEC | | |
| | | | KGFYPSDIAVEWESNG | | | | | | |
| | | | QPENNYKTTPPVLDSD | | | | | | |
| | | | GSFFLYSKLTVDKSRW | | | | | | |
| | | | QQGNVFSCSVMHEALH | | | | | | |
| | | | NHYTQKSLSLSPGK | | | | | | |
| Human | None | | | | | | | | |
| IgG | | | | | | | | | |
| isotype | | | | | | | | | |
| (EPI1102/ | | | | | | | | | |
| RG196) | | | | | | | | | |
| Telisotu- | cMET | 408 | QVQLVQSGAEVKKPGA | 409 | QVQLVQSG | 410 | DIVMTQSPDSLA | 411 | DIVMTQ |
| zumab | | | SVKVSCKASGYIFTAYT | | AEVKKPGAS | | VSLGERATINCK | | SPDSLA |
| (EPI443) | | | MHWVRQAPGQGLEW | | VKVSCKAS | | SSESVDSYANSF | | VSLGER |
| | | | MGWIKPNNGLANYAQ | | GYIFTAYTM | | LHWYQQKPGQP | | ATINCKS |
| | | | KFQGRVTMTRDTSISTA | | HWVRQAPG | | PKLLIYRASTRE | | SESVDS |
| | | | YMELSRLRSDDTAVYY | | QGLEWMG | | SGVPDRFSGSGS | | YANSFL |
| | | | CARSEITTEFDYWGQG | | WIKPNNGLA | | GTDFTLTISSLQ | | HWYQQ |
| | | | TLVTVSSASTKGPSVFP | | NYAQKFQG | | AEDVAVYYCQQ | | KPGQPP |
| | | | LAPSSKSTSGGTAALGC | | RVTMTRDTS | | SKEDPLTFGGGT | | KLLIYR |
| | | | LVKDYFPEPVTVSWNS | | ISTAYMELS | | KVEIKRTVAAPS | | ASTRES |
| | | | GALTSGVHTFPAVLQSS | | RLRSDDTAV | | VFIFPPSDEQLKS | | GVPDRF |
| | | | GLYSLSSVVTVPSSSLG | | YYCARSEIT | | GTASVVCLLNN | | SGSGSG |
| | | | TQTYICNVNHKPSNTK | | TEFDYWGQ | | FYPREAKVQWK | | TDFTLTI |
| | | | VDKRVEPKSCDKTHTC | | GTLVTVSS | | VDNALQSGNSQ | | SSLQAE |
| | | | PPCPAPELLGGPSVFLFP | | | | ESVTEQDSKDST | | DVAVYY |
| | | | PKPKDTLMISRTPEVTC | | | | YSLSSTLTLSKA | | CQQSKE |
| | | | VVVDVSHEDPEVKFN | | | | DYEKHKVYACE | | DPLTFG |
| | | | WYVDGVEVHNAKTKP | | | | VTHQGLSSPVTK | | GGTKVE |
| | | | REEQYNSTYRVVSVLT | | | | SFNRGEC | | IK |
| | | | VLHQDWLNGKEYKCK | | | | | | |
| | | | VSNKALPAPIEKTISKA | | | | | | |
| | | | KGQPREPQVYTLPPSRD | | | | | | |
| | | | ELTKNQVSLTCLVKGF | | | | | | |
| | | | YPSDIAVEWESNGQPE | | | | | | |
| | | | NNYKTTPPVLDSDGSFF | | | | | | |
| | | | LYSKLTVDKSRWQQG | | | | | | |
| | | | NVFSCSVMHEALHNHY | | | | | | |
| | | | TQKSLSLSPGK | | | | | | |
| Onartu- | cMET | 412 | EVQLVESGGGLVQPGG | 413 | EVQLVESGG | 414 | DIQMTQSPSSLS | 415 | DIQMTQ |
| zumab | | | SLRLSCAASGYTFTSY | | GLVQPGGSL | | ASVGDRVTITCK | | SPSSLSA |
| (EPI444) | | | WLHWVRQAPGKGLEW | | RLSCAASGY | | SSQSLLYTSSQK | | SVGDRV |
| | | | VGMIDPSNSDTRFNPNF | | TFTSYWLH | | NYLAWYQQKP | | TITCKSS |
| | | | KDRFTISADTSKNTAYL | | WVRQAPGK | | GKAPKLLIYWA | | QSLLYT |
| | | | QMNSLRAEDTAVYYC | | GLEWVGMI | | STRESGVPSRFS | | SSQKNY |
| | | | ATYRSYVTPLDYWGQ | | DPSNSDTRF | | GSGSGTDFTLTI | | LAWYQ |
| | | | GTLVTVSSASTKGPSVF | | NPNFKDRFT | | SSLQPEDFATYY | | QKPGKA |
| | | | PLAPSSKSTSGGTAALG | | ISADTSKNT | | CQQYYAYPWTF | | PKLLIY |
| | | | CLVKDYFPEPVTVSWN | | AYLQMNSL | | GQGTKVEIKRT | | WASTRE |
| | | | SGALTSGVHTFPAVLQS | | RAEDTAVY | | VAAPSVFIFPPSD | | SGVPSRF |
| | | | SGLYSLSSVVTVPSSSL | | YCATYRSY | | EQLKSGTASVV | | SGSGSG |
| | | | GTQTYICNVNHKPSNT | | VTPLDYWG | | CLLNNFYPREA | | TDFTLTI |
| | | | KVDKKVEPKSCDKTHT | | QGTLVTVSS | | KVQWKVDNAL | | SSLQPED |
| | | | CPPCPAPELLGGPSVFL | | | | QSGNSQESVTEQ | | FATYYC |
| | | | FPPKPKDTLMISRTPEV | | | | DSKDSTYSLSST | | QQYYAY |
| | | | TCVVVDVSHEDPEVKF | | | | LTLSKADYEKH | | PWTFGQ |
| | | | NWYVDGVEVHNAKTK | | | | KVYACEVTHQG | | GTKVEI |
| | | | PREEQYNSTYRVVSVL | | | | LSSPVTKSFNRG | | K |
| | | | TVLHQDWLNGKEYKC | | | | EC | | |
| | | | KVSNKALPAPIEKTISK | | | | | | |
| | | | AKGQPREPQVYTLPPSR | | | | | | |
| | | | DELTKNQVSLTCLVKG | | | | | | |
| | | | FYPSDIAVEWESNGQPE | | | | | | |
| | | | NNYKTTPPVLDSDGSFF | | | | | | |
| | | | LYSKLTVDKSRWQQG | | | | | | |
| | | | NVFSCSVMHEALHNHY | | | | | | |
| | | | TQKSLSLSPGK | | | | | | |
| Amivanta- | cMET | 416 | QVQLVQSGAEVKKPGA | 417 | QVQLVQSG | 418 | DIQMTQSPSSVS | 419 | DIQMTQ |
| mab | | | SVKVSCETSGYTFTSYG | | AEVKKPGAS | | ASVGDRVTITCR | | SPSSVSA |
| (EPI445) | | | ISWVRQAPGHGLEWM | | VKVSCETSG | | ASQGISNWLAW | | SVGDRV |
| | | | GWISAYNGYTNYAQKL | | YTFTSYGIS | | FQHKPGKAPKL | | TITCRAS |
| | | | QGRVTMTTDTSTSTAY | | WVRQAPGH | | LIYAASSLLSGV | | QGISNW |
| | | | MELRSLRSDDTAVYYC | | GLEWMGWI | | PSRFSGSGSGTD | | LAWFQH |
| | | | ARDLRGTNYFDYWGQ | | SAYNGYTN | | FTLTISSLQPEDF | | KPGKAP |
| | | | GTLVTVSSASTKGPSVF | | YAQKLQGR | | ATYYCQQANSF | | KLLIYA |
| | | | PLAPSSKSTSGGTAALG | | VTMTTDTST | | PITFGQGTRLEIK | | ASSLLSG |
| | | | CLVKDYFPEPVTVSWN | | STAYMELRS | | RTVAAPSVFIFPP | | VPSRFSG |
| | | | SGALTSGVHTFPAVLQS | | LRSDDTAVY | | SDEQLKSGTASV | | SGSGTD |
| | | | SGLYSLSSVVTVPSSSL | | YCARDLRG | | VCLLNNFYPRE | | FTLTISS |
| | | | GTQTYICNVNHKPSNT | | TNYFDYWG | | AKVQWKVDNA | | LQPEDF |
| | | | KVDKRVEPKSCDKTHT | | QGTLVTVSS | | LQSGNSQESVTE | | ATYYCQ |
| | | | CPPCPAPELLGGPSVFL | | | | QDSKDSTYSLSS | | QANSFPI |
| | | | FPPKPKDTLMISRTPEV | | | | TLTLSKADYEK | | TFGQGT |
| | | | TCVVVDVSHEDPEVKF | | | | HKVYACEVTHQ | | RLEIK |
| | | | NWYVDGVEVHNAKTK | | | | GLSSPVTKSFNR | | |
| | | | PREEQYNSTYRVVSVL | | | | GEC | | |
| | | | TVLHQDWLNGKEYKC | | | | | | |
| | | | KVSNKALPAPIEKTISK | | | | | | |
| | | | AKGQPREPQVYTLPPSR | | | | | | |
| | | | DELTKNQVSLTCLVKG | | | | | | |
| | | | FYPSDIAVEWESNGQPE | | | | | | |
| | | | NNYKTTPPVLDSDGSFF | | | | | | |
| | | | LYSKLTVDKSRWQQG | | | | | | |
| | | | NVFSCSVMHEALHNHY | | | | | | |
| | | | TQKSLSLSPGK | | | | | | |
| SN58 | cMET | 420 | EVQLVESGGGLVQPGT | 421 | EVQLVESGG | 422 | DIQMTQSPSSLS | 423 | DIQMTQ |
| (EPI446) | | | SLRLSCAASGFTFDDYA | | GLVQPGTSL | | ASVGDRVTITCR | | SPSSLSA |
| | | | MHWVRQAPGKGLEWV | | RLSCAASGF | | ASQSISSYLNWY | | SVGDRV |
| | | | SGITWNSYNIDYADSV | | TEDDYAMH | | QQKPGKAPKLLI | | TITCRAS |
| | | | KGRFTISRDNAKNSLYL | | WVRQAPGK | | YAASSLQSGVPS | | QSISSYL |
| | | | QMNSLRAEDTALYYCA | | GLEWVSGIT | | RFSGSGSGTDFT | | NWYQQ |
| | | | KDDDYSNYVYFDYWG | | WNSYNIDY | | LTISSLQPEDFAT | | KPGKAP |
| | | | QGTLVTVSSASTKGPSV | | ADSVKGRFT | | YYCQQSYSTPPI | | KLLIYA |
| | | | LPLAPSSKSTSGGTAAL | | ISRDNAKNS | | TFGQGTRLEIKR | | ASSLQS |
| | | | GCLVKDYFPEPVTVSW | | LYLQMNSL | | TVAAPSVFIFPPS | | GVPSRFS |
| | | | NSGALTSGVHTFPAVL | | RAEDTALY | | DEQLKSGTASV | | GSGSGT |
| | | | QSSGLYSLSSVVTVPSS | | YCAKDDDY | | VCLLNNFYPRE | | DFTLTIS |
| | | | SLGTQTYICNVNHKPSN | | SNYVYFDY | | AKVQWKVDNA | | SLQPEDF |
| | | | TKVDKKVEPKSCDKTH | | WGQGTLVT | | LQSGNSQESVTE | | ATYYCQ |
| | | | TCPPCPAPELLGGPSVF | | VSS | | QDSKDSTYSLSS | | QSYSTPP |
| | | | LFPPKPKDTLMISRTPE | | | | TLTLSKADYEK | | ITFGQGT |
| | | | VTCVVVDVSHEDPEVK | | | | HKVYACEVTHQ | | RLEIK |
| | | | FNWYVDGVEVHNAKT | | | | GLSSPVTKSFNR | | |
| | | | KPREEQYNSTYRVVSV | | | | GEC | | |
| | | | LTVLHQDWLNGKEYK | | | | | | |
| | | | CKVSNKALPAPIEKTIS | | | | | | |
| | | | KAKGQPREPQVYTLPP | | | | | | |
| | | | SRDELTKNQVSLTCLV | | | | | | |
| | | | KGFYPSDIAVEWESNG | | | | | | |
| | | | QPENNYKTTPPVLDSD | | | | | | |
| | | | GSFFLYSKLTVDKSRW | | | | | | |
| | | | QQGNVFSCSVMHEALH | | | | | | |
| | | | NHYTQKSLSLSPGK | | | | | | |
| SN82 | cMET | 424 | EVQLVESGGGLVQPGG | 425 | EVQLVESGG | 426 | DIQMTQSPSSLS | 427 | DIQMTQ |
| (EPI447) | | | SLRLSCAASGFIVTTNY | | GLVQPGGSL | | ASVGDRVTITCR | | SPSSLSA |
| | | | MTWLRQAPGKGLEWV | | RLSCAASGF | | ASQSISSYLNWY | | SVGDRV |
| | | | SLIYSSGHTYYADSVKG | | IVTTNYMT | | QQKPGKAPKLLI | | TITCRAS |
| | | | RFTISRHNSKNTLYLQM | | WLRQAPGK | | YAASSLQSGVPS | | QSISSYL |
| | | | DSLRAEDTAVYYCASA | | GLEWVSLIY | | RFSGSGSGTDFT | | NWYQQ |
| | | | FAADVFDIWGQGTMVT | | SSGHTYYAD | | LTISSLQPEDFAT | | KPGKAP |
| | | | VSSASTKGPSVLPLAPS | | SVKGRFTIS | | YYCQQSYSTPPI | | KLLIYA |
| | | | SKSTSGGTAALGCLVK | | RHNSKNTLY | | TFGQGTRLEIKR | | ASSLQS |
| | | | DYFPEPVTVSWNSGAL | | LQMDSLRA | | TVAAPSVFIFPPS | | GVPSRFS |
| | | | TSGVHTFPAVLQSSGLY | | EDTAVYYC | | DEQLKSGTASV | | GSGSGT |
| | | | SLSSVVTVPSSSLGTQT | | ASAFAADVF | | VCLLNNFYPRE | | DFTLTIS |
| | | | YICNVNHKPSNTKVDK | | DIWGQGTM | | AKVQWKVDNA | | SLQPEDF |
| | | | KVEPKSCDKTHTCPPCP | | VTVSS | | LQSGNSQESVTE | | ATYYCQ |
| | | | APELLGGPSVFLFPPKP | | | | QDSKDSTYSLSS | | QSYSTPP |
| | | | KDTLMISRTPEVTCVV | | | | TLTLSKADYEK | | ITFGQGT |
| | | | VDVSHEDPEVKFNWY | | | | HKVYACEVTHQ | | RLEIK |
| | | | VDGVEVHNAKTKPREE | | | | GLSSPVTKSFNR | | |
| | | | QYNSTYRVVSVLTVLH | | | | GEC | | |
| | | | QDWLNGKEYKCKVSN | | | | | | |
| | | | KALPAPIEKTISKAKGQ | | | | | | |
| | | | PREPQVYTLPPSRDELT | | | | | | |
| | | | KNQVSLTCLVKGFYPS | | | | | | |
| | | | DIAVEWESNGQPENNY | | | | | | |
| | | | KTTPPVLDSDGSFFLYS | | | | | | |
| | | | KLTVDKSRWQQGNVFS | | | | | | |
| | | | CSVMHEALHNHYTQKS | | | | | | |
| | | | LSLSPGK | | | | | | |
| Emibetu- | cMET | 428 | QVQLVQSGAEVKKPGA | 429 | QVQLVQSG | 430 | DIQMTQSPSSLS | 431 | DIQMTQ |
| zumab | | | SVKVSCKASGYTFTDY | | AEVKKPGAS | | ASVGDRVTITCS | | SPSSLSA |
| (EPI1444) | | | YMHWVRQAPGQGLEW | | VKVSCKAS | | VSSSVSSIYLHW | | SVGDRV |
| | | | MGRVNPNRRGTTYNQ | | GYTFTDYY | | YQQKPGKAPKL | | TITCSVS |
| | | | KFEGRVTMTTDTSTST | | MHWVRQAP | | LIYSTSNLASGV | | SSVSSIY |
| | | | AYMELRSLRSDDTAVY | | GQGLEWMG | | PSRFSGSGSGTD | | LHWYQ |
| | | | YCARANWLDYWGQGT | | RVNPNRRGT | | FTLTISSLQPEDF | | QKPGKA |
| | | | TVTVSSASTKGPSVFPL | | TYNQKFEGR | | ATYYCQVYSGY | | PKLLIYS |
| | | | APCSRSTSESTAALGCL | | VTMTTDTST | | PLTFGGGTKVEI | | TSNLAS |
| | | | VKDYFPEPVTVSWNSG | | STAYMELRS | | KRTVAAPSVFIF | | GVPSRFS |
| | | | ALTSGVHTFPAVLQSSG | | LRSDDTAVY | | PPSDEQLKSGTA | | GSGSGT |
| | | | LYSLSSVVTVPSSNFGT | | YCARANWL | | SVVCLLNNFYPR | | DFTLTIS |
| | | | QTYTCNVDHKPSNTKV | | DYWGQGTT | | EAKVQWKVDN | | SLQPEDF |
| | | | DKTVERKCCVECPPCP | | VTVSS | | ALQSGNSQESVT | | ATYYCQ |
| | | | APPVAGPSVFLFPPKPK | | | | EQDSKDSTYSLS | | VYSGYP |
| | | | DTLMISRTPEVTCVVV | | | | STLTLSKADYEK | | LTFGGG |
| | | | DVSHEDPEVQFNWYV | | | | HKVYACEVTHQ | | TKVEIK |
| | | | DGMEVHNAKTKPREE | | | | GLSSPVTKSFNR | | |
| | | | QFNSTFRVVSVLTVVH | | | | GEC | | |
| | | | QDWLNGKEYKCKVSN | | | | | | |
| | | | KGLPAPIEKTISKTKGQ | | | | | | |
| | | | PREPQVYTLPPSREEMT | | | | | | |
| | | | KNQVSLTCLVKGFYPS | | | | | | |
| | | | DIAVEWESNGQPENNY | | | | | | |
| | | | KTTPPMLDSDGSFFLYS | | | | | | |
| | | | KLTVDKSRWQQGNVFS | | | | | | |
| | | | CSVMHEALHNHYTQKS | | | | | | |
| | | | LSLSPG | | | | | | |
| SC37.3 | RNF43 | 432 | QIQLVQSGPELKKPGET | 433 | QIQLVQSGP | 434 | QAVVTQESALT | 435 | QAVVTQ |
| (EPI1784) | | | VKISCKASGYTFTNYG | | ELKKPGETV | | TSPGETVTFTCR | | ESALTTS |
| | | | MNWVRQAPGKGLKW | | KISCKASGY | | SSTGAVTTSNYA | | PGETVT |
| | | | VGWINTNTGEPTYADD | | TFTNYGMN | | NWVQEKPDHLF | | FTCRSST |
| | | | FKGRFAFSLETSASTAY | | WVRQAPGK | | TGLIGGTNNRAP | | GAVTTS |
| | | | LQISNLKNEDMSTYFC | | GLKWVGWI | | GVPARFSGSLIG | | NYANW |
| | | | AGSHDYSFAYWGQGT | | NTNTGEPTY | | DKAALTITGAQT | | VQEKPD |
| | | | LVTVSAASTKGPSVFPL | | ADDFKGRF | | EDEAIYFCALW | | HLFTGLI |
| | | | APSSKSTSGGTAALGCL | | AFSLETSAS | | YSNHWVFGGGT | | GGTNNR |
| | | | VKDYFPEPVTVSWNSG | | TAYLQISNL | | KLTVLRTVAAPS | | APGVPA |
| | | | ALTSGVHTFPAVLQSSG | | KNEDMSTY | | VFIFPPSDEQLKS | | RFSGSLI |
| | | | LYSLSSVVTVPSSSLGT | | FCAGSHDYS | | GTASVVCLLNN | | GDKAAL |
| | | | QTYICNVNHKPSNTKV | | FAYWGQGT | | FYPREAKVQWK | | TITGAQT |
| | | | DKKVEPKSCDKTHTCP | | LVTVSA | | VDNALQSGNSQ | | EDEAIYF |
| | | | PCPAPELLGGPSVFLFPP | | | | ESVTEQDSKDST | | CALWYS |
| | | | KPKDTLMISRTPEVTCV | | | | YSLSSTLTLSKA | | NHWVF |
| | | | VVDVSHEDPEVKFNW | | | | DYEKHKVYACE | | GGGTKL |
| | | | YVDGVEVHNAKTKPRE | | | | VTHQGLSSPVTK | | TVL |
| | | | EQYNSTYRVVSVLTVL | | | | SFNRGEC | | |
| | | | HQDWLNGKEYKCKVS | | | | | | |
| | | | NKALPAPIEKTISKAKG | | | | | | |
| | | | QPREPQVYTLPPSRDEL | | | | | | |
| | | | TKNQVSLTCLVKGFYP | | | | | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| SC37.23 | RNF43 | 436 | QVQLQQSGTELMKPGA | 437 | QVQLQQSG | 438 | DIQMTQSSSSFS | 439 | DIQMTQ |
| (EPI1785) | | | SVKISCKATGYTFSSYW | | TELMKPGAS | | VSLGDRVTITCK | | SSSSFSV |
| | | | IEWVKQRPGHGLEWIG | | VKISCKATG | | ASEDIYNRLAW | | SLGDRV |
| | | | EILPGSGSTNYNEKFKG | | YTFSSYWIE | | FQQKPGNAPRL | | TITCKAS |
| | | | KATFTVDTSSNTAYMQ | | WVKQRPGH | | LISGATSLETGV | | EDIYNR |
| | | | LSSLTSEDSAVYYCARII | | GLEWIGEILP | | PSRFSGSRSGED | | LAWFQQ |
| | | | RDFWGQGTTLTVSSAS | | GSGSTNYNE | | YTLIITSLQTEDV | | KPGNAP |
| | | | TKGPSVFPLAPSSKSTS | | KFKGKATFT | | ATYYCQQFWTT | | RLLISGA |
| | | | GGTAALGCLVKDYFPE | | VDTSSNTAY | | PPTFGGGTKLEI | | TSLETG |
| | | | PVTVSWNSGALTSGVH | | MQLSSLTSE | | KRTVAAPSVFIF | | VPSRFSG |
| | | | TFPAVLQSSGLYSLSSV | | DSAVYYCA | | PPSDEQLKSGTA | | SRSGED |
| | | | VTVPSSSLGTQTYICNV | | RIIRDFWGQ | | SVVCLLNNFYPR | | YTLIITS |
| | | | NHKPSNTKVDKKVEPK | | GTTLTVSS | | EAKVQWKVDN | | LQTEDV |
| | | | SCDKTHTCPPCPAPELL | | | | ALQSGNSQESVT | | ATYYCQ |
| | | | GGPSVFLFPPKPKDTLM | | | | EQDSKDSTYSLS | | QFWTTP |
| | | | ISRTPEVTCVVVDVSHE | | | | STLTLSKADYEK | | PTFGGG |
| | | | DPEVKFNWYVDGVEV | | | | HKVYACEVTHQ | | TKLEIK |
| | | | HNAKTKPREEQYNSTY | | | | GLSSPVTKSFNR | | |
| | | | RVVSVLTVLHQDWLN | | | | GEC | | |
| | | | GKEYKCKVSNKALPAP | | | | | | |
| | | | IEKTISKAKGQPREPQV | | | | | | |
| | | | YTLPPSRDELTKNQVSL | | | | | | |
| | | | TCLVKGFYPSDIAVEW | | | | | | |
| | | | ESNGQPENNYKTTPPV | | | | | | |
| | | | LDSDGSFFLYSKLTVDK | | | | | | |
| | | | SRWQQGNVFSCSVMH | | | | | | |
| | | | EALHNHYTQKSLSLSP | | | | | | |
| | | | GK | | | | | | |
| SC37.45 | RNF43 | 440 | QVQLQQSGAELMKPG | 441 | QVQLQQSG | 442 | DIQMTQSSSSFS | 443 | DIQMTQ |
| (EPI1789) | | | ASVKISCKATGYTFSRY | | AELMKPGA | | VSLGDRVTITCK | | SSSSFSV |
| | | | WIEWVKQRPGHGLEWI | | SVKISCKAT | | ASEDIYNRLAW | | SLGDRV |
| | | | GEILPGSGSTNYNEKFK | | GYTFSRYWI | | YQQKPGNAPRL | | TITCKAS |
| | | | GKATFTADTSSNTAYM | | EWVKQRPG | | LISGATSLETGV | | EDIYNR |
| | | | QLTSLTSEDSAVYFCER | | HGLEWIGEI | | PSRFSGSGSGKD | | LAWYQ |
| | | | RGAYWGQGTLVTVSA | | LPGSGSTNY | | YTLSITSLQTED | | QKPGNA |
| | | | ASTKGPSVFPLAPSSKS | | NEKFKGKA | | VATYHCQQNWS | | PRLLISG |
| | | | TSGGTAALGCLVKDYF | | TFTADTSSN | | TPPTFGGGTKLE | | ATSLET |
| | | | PEPVTVSWNSGALTSG | | TAYMQLTSL | | IKRTVAAPSVFIF | | GVPSRFS |
| | | | VHTFPAVLQSSGLYSLS | | TSEDSAVYF | | PPSDEQLKSGTA | | GSGSGK |
| | | | SVVTVPSSSLGTQTYIC | | CERRGAYW | | SVVCLLNNFYPR | | DYTLSIT |
| | | | NVNHKPSNTKVDKKVE | | GQGTLVTVS | | EAKVQWKVDN | | SLQTED |
| | | | PKSCDKTHTCPPCPAPE | | A | | ALQSGNSQESVT | | VATYHC |
| | | | LLGGPSVFLFPPKPKDT | | | | EQDSKDSTYSLS | | QQNWST |
| | | | LMISRTPEVTCVVVDVS | | | | STLTLSKADYEK | | PPTFGG |
| | | | HEDPEVKFNWYVDGV | | | | HKVYACEVTHQ | | GTKLEIK |
| | | | EVHNAKTKPREEQYNS | | | | GLSSPVTKSFNR | | |
| | | | TYRVVSVLTVLHQDWL | | | | GEC | | |
| | | | NGKEYKCKVSNKALPA | | | | | | |
| | | | PIEKTISKAKGQPREPQ | | | | | | |
| | | | VYTLPPSRDELTKNQVS | | | | | | |
| | | | LTCLVKGFYPSDIAVE | | | | | | |
| | | | WESNGQPENNYKTTPP | | | | | | |
| | | | VLDSDGSFFLYSKLTVD | | | | | | |
| | | | KSRWQQGNVFSCSVM | | | | | | |
| | | | HEALHNHYTQKSLSLS | | | | | | |
| | | | PGK | | | | | | |
| SC37.141 | RNF43 | 444 | QVQLQQPGAELVRPGA | 445 | QVQLQQPG | 446 | DIQMTQSTSSLS | 447 | DIQMTQ |
| (EPI1791) | | | SVKLSCKASGYTFTSY | | AELVRPGAS | | ASLGDRVTISCR | | STSSLSA |
| | | | WMNWVKQRPGQGLE | | VKLSCKASG | | ASQDISNYLNW | | SLGDRV |
| | | | WIGMIDPSDSETHYSQ | | YTFTSYWM | | YQQKPDGTVKP | | TISCRAS |
| | | | MFKDKATLTVDQSSNT | | NWVKQRPG | | LIYYTSRLHSGV | | QDISNY |
| | | | AYMHLSSLTSEDSAVY | | QGLEWIGMI | | PSRFSGSGSGTD | | LNWYQ |
| | | | YCAREGYYFDGTRGIA | | DPSDSETHY | | YSLTISNLDQEDI | | QKPDGT |
| | | | YWGQGTLVTVSVASTK | | SQMFKDKA | | ATYFCQQGNTL | | VKPLIY |
| | | | GPSVFPLAPSSKSTSGG | | TLTVDQSSN | | PFTFGSGTKLAI | | YTSRLH |
| | | | TAALGCLVKDYFPEPV | | TAYMHLSSL | | ERTVAAPSVFIF | | SGVPSRF |
| | | | TVSWNSGALTSGVHTF | | TSEDSAVYY | | PPSDEQLKSGTA | | SGSGSG |
| | | | PAVLQSSGLYSLSSVVT | | CAREGYYF | | SVVCLLNNFYPR | | TDYSLTI |
| | | | VPSSSLGTQTYICNVNH | | DGTRGIAY | | EAKVQWKVDN | | SNLDQE |
| | | | KPSNTKVDKKVEPKSC | | WGQGTLVT | | ALQSGNSQESVT | | DIATYFC |
| | | | DKTHTCPPCPAPELLGG | | VSV | | EQDSKDSTYSLS | | QQGNTL |
| | | | PSVFLFPPKPKDTLMIS | | | | STLTLSKADYEK | | PFTFGSG |
| | | | RTPEVTCVVVDVSHED | | | | HKVYACEVTHQ | | TKLAIE |
| | | | PEVKFNWYVDGVEVH | | | | GLSSPVTKSFNR | | |
| | | | NAKTKPREEQYNSTYR | | | | GEC | | |
| | | | VVSVLTVLHQDWLNG | | | | | | |
| | | | KEYKCKVSNKALPAPIE | | | | | | |
| | | | KTISKAKGQPREPQVYT | | | | | | |
| | | | LPPSRDELTKNQVSLTC | | | | | | |
| | | | LVKGFYPSDIAVEWES | | | | | | |
| | | | NGQPENNYKTTPPVLD | | | | | | |
| | | | SDGSFFLYSKLTVDKSR | | | | | | |
| | | | WQQGNVFSCSVMHEA | | | | | | |
| | | | LHNHYTQKSLSLSPGK | | | | | | |
| SC37.169 | RNF43 | 448 | QVQLQQSGAEVMKPG | 449 | QVQLQQSG | 450 | DIVMTQSQKFM | 451 | DIVMTQ |
| (EPI1792) | | | ASVKLSCKATGYTFTG | | AEVMKPGA | | STSVGDRVSVTC | | SQKFMS |
| | | | YWIEWVKERPGHGLE | | SVKLSCKAT | | KASQNVGTNVA | | TSVGDR |
| | | | WIGEILPGSGSTNYNEK | | GYTFTGYWI | | WFQQKPGQSPK | | VSVTCK |
| | | | FKGKATFTADPSSNTA | | EWVKERPG | | LLIYSASYRYSG | | ASQNVG |
| | | | YMQLSSLTTEDSAIYYC | | HGLEWIGEI | | VPDRFTGSGSGT | | TNVAWF |
| | | | ARDYGSFGYWGQGTL | | LPGSGSTNY | | DFTLTISNVQSE | | QQKPGQ |
| | | | VTVSAASTKGPSVFPLA | | NEKFKGKA | | DLAEFFCQQYN | | SPKLLIY |
| | | | PSSKSTSGGTAALGCLV | | TFTADPSSN | | TYPLTFGAGTKL | | SASYRY |
| | | | KDYFPEPVTVSWNSGA | | TAYMQLSSL | | ELKRTVAAPSVF | | SGVPDR |
| | | | LTSGVHTFPAVLQSSGL | | TTEDSAIYY | | IFPPSDEQLKSGT | | FTGSGS |
| | | | YSLSSVVTVPSSSLGTQ | | CARDYGSFG | | ASVVCLLNNFY | | GTDFTL |
| | | | TYICNVNHKPSNTKVD | | YWGQGTLV | | PREAKVQWKVD | | TISNVQS |
| | | | KKVEPKSCDKTHTCPP | | TVSA | | NALQSGNSQES | | EDLAEF |
| | | | CPAPELLGGPSVFLFPP | | | | VTEQDSKDSTYS | | FCQQYN |
| | | | KPKDTLMISRTPEVTCV | | | | LSSTLTLSKADY | | TYPLTF |
| | | | VVDVSHEDPEVKFNW | | | | EKHKVYACEVT | | GAGTKL |
| | | | YVDGVEVHNAKTKPRE | | | | HQGLSSPVTKSF | | ELK |
| | | | EQYNSTYRVVSVLTVL | | | | NRGEC | | |
| | | | HQDWLNGKEYKCKVS | | | | | | |
| | | | NKALPAPIEKTISKAKG | | | | | | |
| | | | QPREPQVYTLPPSRDEL | | | | | | |
| | | | TKNQVSLTCLVKGFYP | | | | | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| SC37.193 | RNF43 | 452 | EVQLQQSGAELVRPGA | 453 | EVQLQQSG | 454 | DIVMTQDAPSIP | 455 | DIVMTQ |
| (EPI1796) | | | SVKLSCTASGFNIKDDY | | AELVRPGAS | | VTPGESVSISCRS | | DAPSIPV |
| | | | MHWMKQRPEQGLEWL | | VKLSCTASG | | SKSLLHSNGNTY | | TPGESVS |
| | | | GWIDPEIGATEYASKFQ | | FNIKDDYM | | LYWFLQRPGQS | | ISCRSSK |
| | | | GKATMTADTSSNTAYL | | HWMKQRPE | | PQLLIYRMSNLA | | SLLHSN |
| | | | QLSSLTSEDTAVYYCV | | QGLEWLGW | | SGVPDRFSGSGS | | GNTYLY |
| | | | DDRRGMDYWGQGTSV | | IDPEIGATEY | | GTAFTLRISRVE | | WFLQRP |
| | | | TVSSASTKGPSVFPLAP | | ASKFQGKAT | | AEDVGVYYCM | | GOSPQL |
| | | | SSKSTSGGTAALGCLV | | MTADTSSNT | | QHLEYPFTFGSG | | LIYRMS |
| | | | KDYFPEPVTVSWNSGA | | AYLQLSSLT | | TKLEIKRTVAAP | | NLASGV |
| | | | LTSGVHTFPAVLQSSGL | | SEDTAVYYC | | SVFIFPPSDEQLK | | PDRFSGS |
| | | | YSLSSVVTVPSSSLGTQ | | VDDRRGMD | | SGTASVVCLLN | | GSGTAF |
| | | | TYICNVNHKPSNTKVD | | YWGQGTSV | | NFYPREAKVQW | | TLRISRV |
| | | | KKVEPKSCDKTHTCPP | | TVSS | | KVDNALQSGNS | | EAEDVG |
| | | | CPAPELLGGPSVFLFPP | | | | QESVTEQDSKDS | | VYYCM |
| | | | KPKDTLMISRTPEVTCV | | | | TYSLSSTLTLSK | | QHLEYP |
| | | | VVDVSHEDPEVKFNW | | | | ADYEKHKVYAC | | FTFGSGT |
| | | | YVDGVEVHNAKTKPRE | | | | EVTHQGLSSPVT | | KLEIK |
| | | | EQYNSTYRVVSVLTVL | | | | KSFNRGEC | | |
| | | | HQDWLNGKEYKCKVS | | | | | | |
| | | | NKALPAPIEKTISKAKG | | | | | | |
| | | | QPREPQVYTLPPSRDEL | | | | | | |
| | | | TKNQVSLTCLVKGFYP | | | | | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| SC37.202 | RNF43 | 456 | QVSLKESGPGILQPSQT | 457 | QVSLKESGP | 458 | DIVMSQSPSSLA | 459 | DIVMSQ |
| (EPI1797) | | | LSLTCSFSGFSLSTSGM | | GILQPSQTLS | | VSVGEEVTMSC | | SPSSLAV |
| | | | AVGWIRQPSGRGLEWL | | LTCSFSGFSL | | KSSQSLLYSTNQ | | SVGEEV |
| | | | ANIWWDDSQHYNAAL | | STSGMAVG | | KNYLAWYQQK | | TMSCKS |
| | | | KSRLTISKDTSKNQVFL | | WIRQPSGRG | | PGQSPKLLIYWA | | SQSLLYS |
| | | | KIASVDTADTATYYCA | | LEWLANIW | | STRESGVPDRFT | | TNQKNY |
| | | | RSNWGRYFDYWGQGT | | WDDSQHYN | | GSGSGTDFTLTI | | LAWYQ |
| | | | TLTVSSASTKGPSVFPL | | AALKSRLTI | | SSVKAEDLAVY | | QKPGQS |
| | | | APSSKSTSGGTAALGCL | | SKDTSKNQV | | YCQQYYDYYTF | | PKLLIY |
| | | | VKDYFPEPVTVSWNSG | | FLKIASVDT | | GGGTKLEIKRTV | | WASTRE |
| | | | ALTSGVHTFPAVLQSSG | | ADTATYYC | | AAPSVFIFPPSDE | | SGVPDR |
| | | | LYSLSSVVTVPSSSLGT | | ARSNWGRY | | QLKSGTASVVC | | FTGSGS |
| | | | QTYICNVNHKPSNTKV | | FDYWGQGT | | LLNNFYPREAK | | GTDFTL |
| | | | DKKVEPKSCDKTHTCP | | TLTVSS | | VQWKVDNALQ | | TISSVKA |
| | | | PCPAPELLGGPSVFLFPP | | | | SGNSQESVTEQD | | EDLAVY |
| | | | KPKDTLMISRTPEVTCV | | | | SKDSTYSLSSTL | | YCQQYY |
| | | | VVDVSHEDPEVKFNW | | | | TLSKADYEKHK | | DYYTFG |
| | | | YVDGVEVHNAKTKPRE | | | | VYACEVTHQGL | | GGTKLEI |
| | | | EQYNSTYRVVSVLTVL | | | | SSPVTKSFNRGE | | K |
| | | | HQDWLNGKEYKCKVS | | | | C | | |
| | | | NKALPAPIEKTISKAKG | | | | | | |
| | | | QPREPQVYTLPPSRDEL | | | | | | |
| | | | TKNQVSLTCLVKGFYP | | | | | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| SC37.223 | RNF43 | 460 | QVQLQQPGAELVKPGA | 461 | QVQLQQPG | 462 | DIQMTQTTSSLS | 463 | DIQMTQ |
| (EPI1798) | | | SVKLSCKASGYTFTTY | | AELVKPGAS | | ASLGDRVTISCS | | TTSSLSA |
| | | | YIYWVKQRPGQGLEWI | | VKLSCKASG | | ASQGIGNYLNW | | SLGDRV |
| | | | GGINPRNGGTNFNEKF | | YTFTTYYIY | | YQQKPDGTVKL | | TISCSAS |
| | | | KTRATLTVDKSSSTAY | | WVKQRPGQ | | LIYYTSSLNSGV | | QGIGNY |
| | | | MQLSSLTSEDSAVYYC | | GLEWIGGIN | | PSRFSGSGSGTD | | LNWYQ |
| | | | TRTFYWGQGTTLTVSS | | PRNGGTNFN | | YSLTISNLEPEDI | | QKPDGT |
| | | | ASTKGPSVFPLAPSSKS | | EKFKTRATL | | ATYFCQQYSKL | | VKLLIY |
| | | | TSGGTAALGCLVKDYF | | TVDKSSSTA | | PYTFGGGTKLEI | | YTSSLNS |
| | | | PEPVTVSWNSGALTSG | | YMQLSSLTS | | KRTVAAPSVFIF | | GVPSRFS |
| | | | VHTFPAVLQSSGLYSLS | | EDSAVYYCT | | PPSDEQLKSGTA | | GSGSGT |
| | | | SVVTVPSSSLGTQTYIC | | RTFYWGQG | | SVVCLLNNFYPR | | DYSLTIS |
| | | | NVNHKPSNTKVDKKVE | | TTLTVSS | | EAKVQWKVDN | | NLEPEDI |
| | | | PKSCDKTHTCPPCPAPE | | | | ALQSGNSQESVT | | ATYFCQ |
| | | | LLGGPSVFLFPPKPKDT | | | | EQDSKDSTYSLS | | QYSKLP |
| | | | LMISRTPEVTCVVVDVS | | | | STLTLSKADYEK | | YTFGGG |
| | | | HEDPEVKFNWYVDGV | | | | HKVYACEVTHQ | | TKLEIK |
| | | | EVHNAKTKPREEQYNS | | | | GLSSPVTKSFNR | | |
| | | | TYRVVSVLTVLHQDWL | | | | GEC | | |
| | | | NGKEYKCKVSNKALPA | | | | | | |
| | | | PIEKTISKAKGQPREPQ | | | | | | |
| | | | VYTLPPSRDELTKNQVS | | | | | | |
| | | | LTCLVKGFYPSDIAVE | | | | | | |
| | | | WESNGQPENNYKTTPP | | | | | | |
| | | | VLDSDGSFFLYSKLTVD | | | | | | |
| | | | KSRWQQGNVFSCSVM | | | | | | |
| | | | HEALHNHYTQKSLSLS | | | | | | |
| | | | PGK | | | | | | |
| SC37.226 | RNF43 | 464 | EVQLQQSGAELVRPGA | 465 | EVQLQQSG | 466 | DIVMTQAAPSVP | 467 | DIVMTQ |
| (EPI1799) | | | SVKLSCTASGFNIIVDY | | AELVRPGAS | | VTPGESVSISCRS | | AAPSVP |
| | | | LHWVRQRPEQGLEWIG | | VKLSCTASG | | SKSLLHSNGNTY | | VTPGES |
| | | | WIDPEIGSTEYASKFQG | | FNIIVDYLH | | LYWFLQRPGQS | | VSISCRS |
| | | | KATMTADTSSNTAYLQ | | WVRQRPEQ | | PQVLIYRMSNLA | | SKSLLHS |
| | | | LSSLTSEDTAVYYCIID | | GLEWIGWID | | SGVPDRFSGSGS | | NGNTYL |
| | | | GTMDYWGQGTSVTVS | | PEIGSTEYAS | | GTAFTLRISRVE | | YWFLQR |
| | | | SASTKGPSVFPLAPSSK | | KFQGKATM | | AEDVGVYYCM | | PGQSPQ |
| | | | STSGGTAALGCLVKDY | | TADTSSNTA | | QHLEYPFTFGSG | | VLIYRM |
| | | | FPEPVTVSWNSGALTS | | YLQLSSLTS | | TKLEIKRTVAAP | | SNLASG |
| | | | GVHTFPAVLQSSGLYSL | | EDTAVYYCI | | SVFIFPPSDEQLK | | VPDRFS |
| | | | SSVVTVPSSSLGTQTYI | | IDGTMDYW | | SGTASVVCLLN | | GSGSGT |
| | | | CNVNHKPSNTKVDKK | | GQGTSVTVS | | NFYPREAKVQW | | AFTLRIS |
| | | | VEPKSCDKTHTCPPCPA | | S | | KVDNALQSGNS | | RVEAED |
| | | | PELLGGPSVFLFPPKPK | | | | QESVTEQDSKDS | | VGVYYC |
| | | | DTLMISRTPEVTCVVV | | | | TYSLSSTLTLSK | | MQHLEY |
| | | | DVSHEDPEVKFNWYV | | | | ADYEKHKVYAC | | PFTFGSG |
| | | | DGVEVHNAKTKPREEQ | | | | EVTHQGLSSPVT | | TKLEIK |
| | | | YNSTYRVVSVLTVLHQ | | | | KSFNRGEC | | |
| | | | DWLNGKEYKCKVSNK | | | | | | |
| | | | ALPAPIEKTISKAKGQP | | | | | | |
| | | | REPQVYTLPPSRDELTK | | | | | | |
| | | | NQVSLTCLVKGFYPSDI | | | | | | |
| | | | AVEWESNGQPENNYKT | | | | | | |
| | | | TPPVLDSDGSFFLYSKL | | | | | | |
| | | | TVDKSRWQQGNVFSCS | | | | | | |
| | | | VMHEALHNHYTQKSLS | | | | | | |
| | | | LSPGK | | | | | | |
| SC37.231 | RNF43 | 468 | EVQLQQSGAELVRPGA | 469 | EVQLQQSG | 470 | DIVMTQSQKFM | 471 | DIVMTQ |
| (EPI1800) | | | SVKLSCTASGFNIKDDY | | AELVRPGAS | | STSVGDRVSITC | | SQKFMS |
| | | | IHWVKQRPEQGLEWIG | | VKLSCTASG | | KASQSVRPAVA | | TSVGDR |
| | | | WIDPENGDTKYASKFP | | FNIKDDYIH | | WYQQKPGQSPK | | VSITCKA |
| | | | GKATMTADTSSNTAYL | | WVKQRPEQ | | ALIYLASNRHTG | | SQSVRP |
| | | | QLSSLTSEDTAVYYCT | | GLEWIGWID | | VPDRFTGSGSGT | | AVAWY |
| | | | ASRTTALDYWGPGTTL | | PENGDTKY | | DFTLTISNVQSE | | QQKPGQ |
| | | | TVSSASTKGPSVFPLAP | | ASKFPGKAT | | DLADYFCLQHW | | SPKALIY |
| | | | SSKSTSGGTAALGCLV | | MTADTSSNT | | NYPYTFGGGTK | | LASNRH |
| | | | KDYFPEPVTVSWNSGA | | AYLQLSSLT | | LEIKRTVAAPSV | | TGVPDR |
| | | | LTSGVHTFPAVLQSSGL | | SEDTAVYYC | | FIFPPSDEQLKSG | | FTGSGS |
| | | | YSLSSVVTVPSSSLGTQ | | TASRTTALD | | TASVVCLLNNF | | GTDFTL |
| | | | TYICNVNHKPSNTKVD | | YWGPGTTL | | YPREAKVQWKV | | TISNVQS |
| | | | KKVEPKSCDKTHTCPP | | TVSS | | DNALQSGNSQE | | EDLADY |
| | | | CPAPELLGGPSVFLFPP | | | | SVTEQDSKDSTY | | FCLQHW |
| | | | KPKDTLMISRTPEVTCV | | | | SLSSTLTLSKAD | | NYPYTF |
| | | | VVDVSHEDPEVKFNW | | | | YEKHKVYACEV | | GGGTKL |
| | | | YVDGVEVHNAKTKPRE | | | | THQGLSSPVTKS | | EIK |
| | | | EQYNSTYRVVSVLTVL | | | | FNRGEC | | |
| | | | HQDWLNGKEYKCKVS | | | | | | |
| | | | NKALPAPIEKTISKAKG | | | | | | |
| | | | QPREPQVYTLPPSRDEL | | | | | | |
| | | | TKNQVSLTCLVKGFYP | | | | | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |

The sequences listed in Table 10, Table 11, and Table 12 (SEQ ID NOs: 371-471) are amino acid molecules. The sequences listed in Table 10, Table 11, and Table 12 (SEQ ID NOs: 371-471) are amino acid molecules that are synthetic constructs. The sequences listed in Table 10, Table 11, and Table 12 (SEQ ID NOs: 371-471) for HC sequences (heavy chain), VH sequence (variable heavy chain sequence), LC sequences (light chain), VL sequence (variable light chain sequence) are amino acid molecules that are synthetic constructs.

### EXAMPLES

The following examples are illustrative and non-limiting to the scope of the compositions, devices, and methods disclosed herein.

### Cell Lines:

Cells are grown in complete growth medium and maintained at 37° C. and 5% CO₂

### Example 1—Bispecific Antibody Expression

Bispecifics are expressed and purified from mammalian cells (exemplary: Expi293F, ExpiCHO-S) using transient transfection following the manufacturer's protocol. At designated time point (exemplary: 4-14 days), media is harvested by centrifugation at 4,000×g for 20 min. Tagged bispecifics and knob half IgGs are purified by Ni-NTA or Protein A affinity chromatography and buffer exchanged into PBS containing 20% glycerol, concentrated, and flash frozen for storage at −80° C. IgGs and hole half IgGs are purified by Protein A affinity chromatography and buffer exchanged into PBS containing 20% glycerol. Knob and hole half IgGs are recombined under reducing conditions (exemplary: 10 mM Tris pH 7.5, 100 mM NaCl, 20% 800 mM L-Arg pH 10 plus 200 fold excess reduced glutathione), and then purified by Ni-NTA affinity chromatography, buffer exchanged into PBS containing 20% glycerol, concentrated, and flash frozen for storage at −80° C. Purity and integrity of all proteins are assessed by SDS-PAGE and SEC

### Example 2—Stable Cell Line Generation

N-terminally epitope tagged (exemplary: alfa, HA, Myc, etc.) receptors (e.g., cMET) are cloned into a pLVX lentiviral vector. Lentivirus is produced by transfecting HEK293T cells with standard packaging vectors. Stable cell lines expressing epitope tagged receptors are selected with puromycin and validated for expression by flow cytometry using anti-epitope tag primary antibody.

### Example 3—Degradation Experiments

Cells (exemplary examples: human cancer cell lines, primary human immune cells, or stable cell lines generated herein) are plated (exemplary examples: in 6, 12, 24, 48, 96, or 348-well plates) and grown to ˜70% confluency before treatment. Media is aspirated and cells are treated with (concentration range: 0.001 to 1000 nM; time range: 0-7 days) bispecifics (including, for example, any antibody disclosed herein) or control antibodies in complete growth medium. After incubation at 37° C., cells are washed with phosphate-buffered saline (PBS). Samples are then tested following western blotting, in-cell western blotting, or flow cytometry protocols to quantify target protein levels.

### Example 4—cMET Level Quantification by Western Blotting

Cells are lifted with versene and harvested by centrifugation at 300×g for 5 min at 4° C. Cell pellets are lysed with Ix RIPA buffer containing complete mini protease inhibitor cocktail (Sigma-Aldrich) at 4° C. for 30 min. Lysates are centrifuged at 2,000 (for 96-well plate) or 16,000×g for 10 min at 4° C. 4×NuPAGE LDS sample buffer (Invitrogen) and 2-mercaptoethanol (BME) is added to the lysates and boiled for 10 min. Equal amounts of lysates is loaded onto a 4-12% Bis-Tris gel and ran at 200V for 37 min. The gel is incubated in 20% ethanol for 10 min and transferred onto a polyvinylidene difluoride (PVDF) membrane. The membrane is blocked in PBS with 0.1% Tween-20+5% bovine serum albumin (BSA) for 30 min at room temperature with gentle shaking. Membranes are incubated for 1 hr with primary antibodies at respective dilutions at room temp with gentle shaking in PBS+0.2% Tween-20+5% BSA. Membranes are washed four times with tris-buffered saline (TBS)+0.1% Tween-20 and then co-incubated with secondary antibodies in PBS+0).2% Tween-20+5% BSA for 1 hr at room temperature. Membranes are washed four times with TBS+0.1% Tween-20, then washed with PBS. Membranes are imaged using an Odyssey CLx Imager (LI-COR). Band intensities are quantified using Image Studio software (LI-COR).

### Example 5—cMET Level Quantification by in-Cell Western Blotting

Fixation solution (exemplary: 4% paraformaldehyde in PBS) is added to cells and incubated for 20 min at room temperature without agitation. The fixation solution is then removed, and cells washed with PBS. Permeabilization solution (exemplary: 0.1% Triton-X100 in PBS) is added to cells and incubated for 20 min with shaking. Permeabilization solution is removed and cells are incubated in blocking buffer for 1 hr at room temperature with shaking. Blocking buffer is removed and cells are incubated with primary antibodies for 2 hr with shaking. Cells are washed four times with TBS+0.1% Tween-20. Cells are then incubated with secondary antibodies for 1 hr with shaking. Cells are then washed four times with TBS+0.1% Tween-20. Wash solution is removed and plates are imaged using an Odyssey CLx Imager (LI-COR). Well intensities are quantified using Empiria Studo software (LI-COR).

### Example 6—cMET Level Quantification by Flow Cytometry

Cells are lifted with versene and harvested by centrifugation at 300×g for 5 min at 4° C. Cell pellets are washed with cold PBS and centrifuged at 300×g for 5 min. Cells are blocked with cold PBS+3% BSA and centrifuged (300×g for 5 min). Cells are incubated with primary antibodies diluted in PBS+3% BSA for 30 min at 4° C. Cells are washed three times with cold PBS+3% BSA and secondary antibodies (if applicable) diluted in PBS+3% BSA added and incubated for 30 min at 4° C. Cells are washed three times with cold PBS+3% BSA and resuspended in cold PBS. Flow cytometry is performed on a CytoFLEX cytometer (Beckman Coulter) and gating is performed on single cells and live cells before acquisition of 10,000 cells. Analysis is performed using the FlowJo software package.

### Example 7—Cell Surface Removal of cMET Using Bispecifics that Bind to cMET and a Degrader Protein

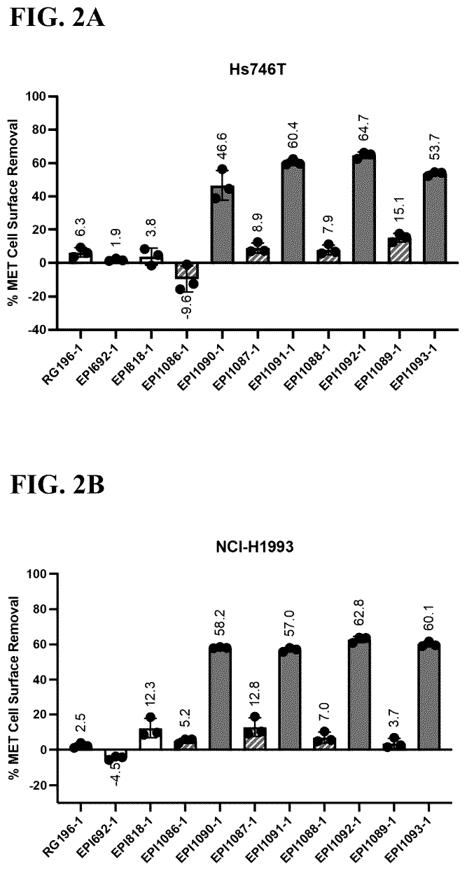

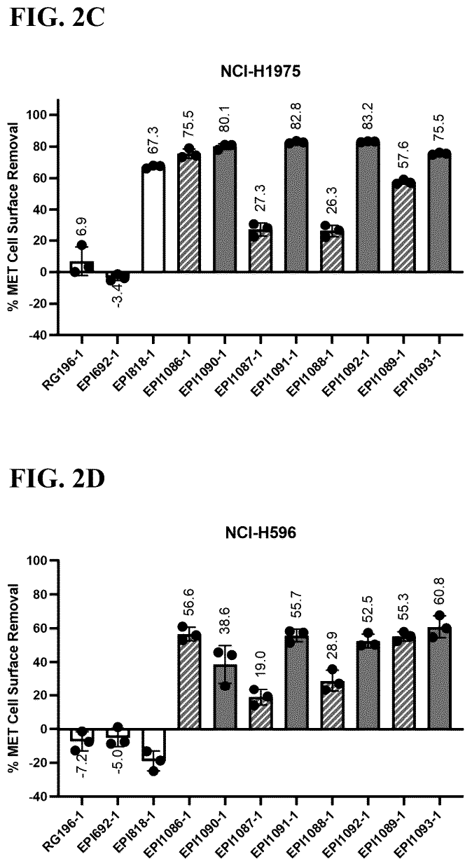

To determine cMET cell surface removal of bispecifics (bispecific antibodies) that bind to cMET and a degrader protein, cell surface removal assays were conducted using cMETxCD71 bispecific antibodies (antibodies that bind to cMET and CD71: FIGS. 1A-2D; Table 13). cMET-targeting bispecifics had Telisotuzmab, Onartuzumab, Amivantamab or REGN5093s58, which are four published c-MET binders that bind different epitopes on the extracellular portion of cMET, as the cMET binding domain and ABBV2029 as the CD71 binding arm.

Additionally, an IgG1 isotype control (RG196-1) and Palivizumab IgG against RSV (EPI692-1) were used as non-targeted controls. Palivizumab/Telisotuzmab (EPI1086-1), Palivizumab/Onartuzumab (EPI1087-1), Palivizumab/Amivantamab (EPI1088-1), and Palivizumab/Regeneron Seq58 (EPI1098-1) (RSV×cMET bispecifics) were tested as a single-arm cMET binding controls with a second arm that did not bind to the target cell. Amivantamab/Zalutumumab (EPI818-1; cMET×EGFR) was used as an additional control.

TABLE 13
Antibodies tested in cell surface removal assay

| | | Arm 1 | Arm 2 | |
|---|---|---|---|---|
| Molecule ID | Name | Binder | Binder | Purpose |
| RG196-1 | Human IgG1 Isotype Control | | | Negative control |
| | (BioXcell) | | | |
| EPI692-1 | Palivizumab IgG | RSV | RSV | Negative control |
| EPI818-1 | Amivantamab (c-Met | cMET | EGFR | SoC control |
| | binder)/Zalutumumab | | | |
| EPI1086-1 | Palivizumab/Telisotuzumab | cMET | RSV F | Single-arm control |
| EPI1090-1 | ABBV2029/Telisotuzumab | cMET | CD71 | Test antibody |
| EPI1087-1 | Palivizumab/Onartuzumab | cMET | RSV F | Single-arm control |
| EPI1091-1 | ABBV2029/Onartuzumab | cMET | CD71 | Test antibody |
| EPI1088-1 | Palivizumab/Amivantamab (c-Met | cMET | RSV F | Single-arm control |
| | binder) | | | |
| EPI1092-1 | ABBV2029/Amivantamab (c-Met | cMET | CD71 | Test antibody |
| | binder) | | | |
| EPI1089-1 | Palivizumab/Regeneron Seq58 | cMET | RSV F | Single-arm control |
| EPI1093-1 | ABBV2029/Regeneron Seq58 | cMET | CD71 | Test antibody |

The various constructs were tested in the gastric adenocarcinoma cell line Hs746T (FIG. 2A) and non-small cell lung cancer cell lines with multiple different cMET isotypes, NCI-H1993 (FIG. 2B), NCI-H1975 (FIG. 2C), and NCI-H596 (FIG. 2D), at 50 nM concentrations (Table 14). In these assays, HS746T, NCI-H1993, HCl-1975, or NCI-H586 cells were seeded in 96-well plates and incubated overnight at 37 C and 5% CO₂. The next morning, cells were treated with either 50 or 500 nM of test antibody. After 24 hours of treatment, cells were harvested using a dissociation reagent and stained using a fluorescently labeled anti-cMET antibodies. Fluorescent intensity of the cells was measured on a Cytek Northern Lights flow cytometer. Percent cMET cell surface removal was calculated as the relative difference between the amount of cMET on the cell surface using the test antibody and the amount of cMET on the cell surface using an untreated control sample after accounting for background with an isotype control.

TABLE 14
Cancer cell lines tested in cell surface removal assay

| | | | | | CD71 per |
|---|---|---|---|---|---|
| Cell Line | Cancer type | MET status | MET/cell | CD71/cell | MET ratio |
| Hs746T | Gastric | METΔex14 | 223,239* | 177,341* | 0.794 |
| | adenocarcinoma | | | | |
| NCI-H1993 | NSCLC | MET amplification | 284,545* | 194,728* | 0.684 |
| NCI-H1975 | NSCLC | MET normal | 78,916* | 75,041* | 0.951 |
| NCI-H596 | NSCLC | METΔex14 | 15,745 | 391,938* | 24.89 |

All four cMET×CD71 bispecific antibodies induced removal of cMET from the cell surface in Hs746T cells (FIG. 2A), NCI-H1993 cells (FIG. 2B), NCI-H1975 cells (FIG. 2C), and NCI-H596 cells (FIG. 2D). The cMET×CD71 bispecifics had higher levels of cMET cell surface removal than Amivantamab, a standard of care molecule, and Palivizumab across multiple cell lines (Hs746T, NCI-H1993, NCI-H1975, NCI-H596). The cMET×CD71 bispecifics also had higher levels of cMET cell surface removal than single arm controls for almost all respective pairs. This effect is durable across multiple epitopes as the four cMET binders bind different epitopes on the extracellular portion of cMET. It is also durable across a spectrum of cMET mutational statuses including MET amplification, MET normal, and METAex14. cMET genomics are variable in lung cancer. There are several genomic alterations that are now well defined in clinical samples. Met exon 14 skipping (METAex 14) mutations cause “skipping” of the 14th exon, via disruption to splicing sites. There are also cMET “amplifications,” or multiple copies of the MET gene in the genome. From these results, Amivantamab was selected as the cMET binding arm for bispecifics in additional testing because it has superior activity across all the cell lines tested and low baseline activity in the single arm control.

This data indicates the effectiveness of bispecifics that bind to both cMET and a degrader protein to remove cMET from the surface of a target cell in multiple contexts.

### Example 8—cMET Cell Surface Removal Screen to Identify Effective Degrader Protein Binding Domains for cMET-Targeting Bispecific Antibodies

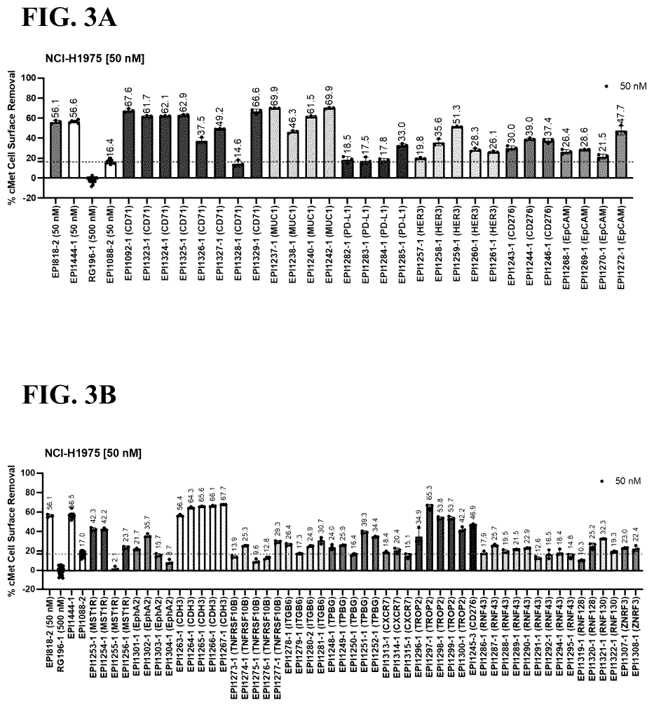

To identify degrader protein binding domains on cMET-targeting bispecifics that resulted in high cMET cell surface removal, a screen was preformed using 78 bispecifics (FIGS. 3A-3B). The 78 bispecifics bound to 18 unique degrader proteins. For most degrader proteins, multiple binding domains that bind to different epitopes were tested. Additionally, Amivantamab (EPI818) Emibetuzumab (EPI1444), an IgG1 isotype control (RG196-1), and cMET×RSV (EPI1088) were tested as an SoC control, a cMET only control, a negative control, and a single-arm control, respectively, with cMET×RSV used as a baseline for comparison. The first binding arm for bispecifics in this example are listed in Table 1 or Table 4. The second binding arm for the bispecifics in this example are listed in Table 7. The control arms and antibodies are also listed in Table 10, Table 11, and Table 12.

The screen was performed on NCI-H1975 cell line (non-small cell lung cancer) using the methods previously described in Example 7. Briefly, cells were seeded in 96-well plates and incubated overnight at 37° C. and 5% CO₂. The next morning, cells were treated with 50 nM of the bispecific or control antibody. After 24 hours of treatment, cells were harvested using a dissociation reagent and stained using a fluorescently labeled anti-cMET antibodies. Fluorescent intensity of the cells was measured on a Cytek Northern Lights flow cytometer. Percent cMET cell surface removal was calculated as the relative difference between the amount of cMET on the cell surface using the test antibody and the amount of cMET on the cell surface using an untreated control sample after accounting for background with an isotype control.

Results of screen identified degrader protein groups and specific molecular epitopes that, when paired with cMET in a bispecific antibody format, demonstrated improved capacity to induce cMET cell surface removal as compared to Palivizumab×cMET, a bispecific pairing cMET with a non-targeting control arm (FIGS. 3A and 3B).

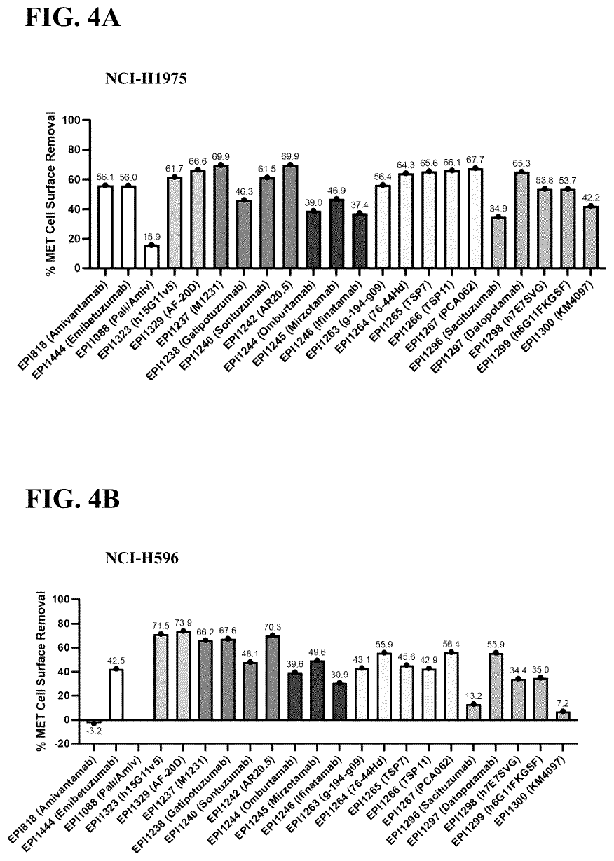

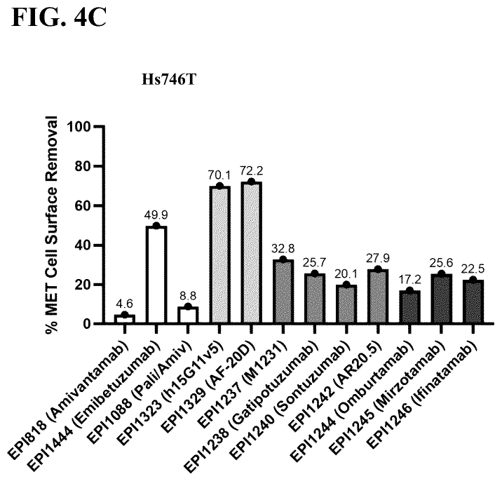

To extend the applicability of findings from the initial screening effort to more functionally relevant cMET models, the cMET cell surface removal assay was repeated for prioritized molecules in cell lines with MET exon 14 deletion and/or MET copy number amplification commonly observed in patients (FIGS. 4A-C). Top hits showed robust cMET cell surface removal activity across all cell lines tested. Additionally, these experiments demonstrate the cell-specificity of cMET degradation using various bispecific antibody pairs.

### Example 9—cMET Internalization Screen to Identify Effective Degrader Protein Binding Domains for cMET-Targeting Bispecific Antibodies

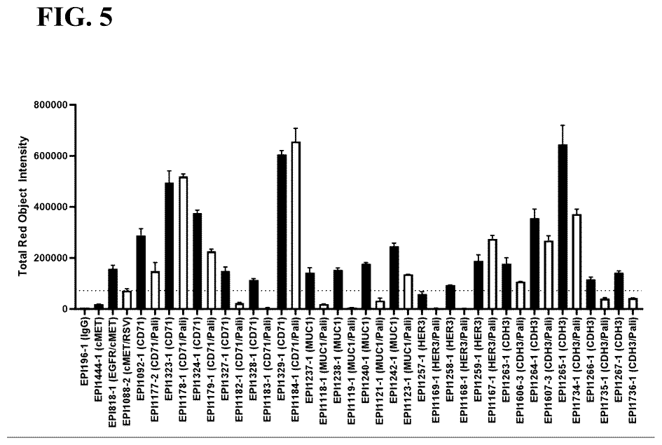

To further screen and validate effective degrader protein binding domains identified in the cell surface removal screen, an cMET internalization assay was performed using 20 bispecifics (FIG. 5). The 19 bispecific antibodies bound to cMET as the target protein and 5 unique degrader proteins. For previously identified degrader proteins, multiple binding domains that bind to different epitopes were tested. Amivantamab (EPI818), Emibetuzumab (EPI1444), an IgG1 isotype control (RG196), and cMET×RSV (EPI1088) were tested as an SoC control, a cMET only control, a negative control, and a single-arm control, respectively, with cMET×RSV used as a baseline for comparison. Additionally for each bispecific antibody, a single arm control which contained the same degrader protein binding arm as the bispecific antibody and an RSV binding arm was tested. The first binding arm for bispecifics in this example are listed in Table 1 or Table 4. The second binding arm for the bispecifics in this example are listed in Table 7. The control arms and antibodies are also listed in Table 10, Table 11, and Table 12.

For internalization assays, NCI-H1975 cells were plated into 96-well, clear culture plates at a density of 7×10³ cells per well. After approximately 16 hours of culture, test antibodies were mixed with rehydrated pH Antibody Labeling Reagent at a 1:3 molar ratio of test antibody to antibody labeling reagent for 15 min at 37° C. Labeled antibodies were dispensed onto cells at a concentration of 50 nM. Plates were placed into the Incucyte R Live-Cell Analysis System where images were acquired. Sampling of internalization images were taken at 0) minutes and at 45 minutes intervals over 72 hours. Image analysis was performed by using Incucyte's Base Analysis software. The “Top-Hat” background subtraction method was used to subtract background to give percent “Red Object Intensity”.

Results of screen identified degrader protein groups and specific molecular epitopes that, when paired with cMET in a bispecific antibody format, demonstrated improved cMET internalization as compared to cMET×RSV, a bispecific pairing cMET with a non-targeting control arm. Many of the bispecific antibodies demonstrated high internalization. Multiple of these hits (MUC1, CDH3, and ITGB6) showed synergistic internalization activity, two of which were also identified in the cell surface removal assay. This demonstrates consistency amongst assays and that cMET bispecific antibodies can cause synergistic internalization through identifiable degrader protein binding domains.

### Example 10—Whole Cell Degradation Screen to Identify Effective Degrader Protein Binding Domains for cMET-Targeting Bispecific Antibodies

To measure degradation of the target protein, whole cell degradation of cMET was tested using an AlphaLISA assay and western blots.

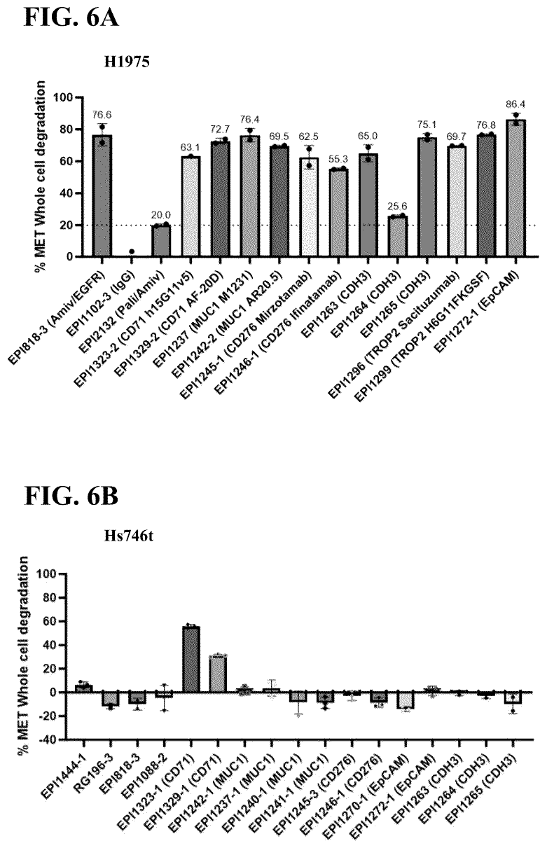

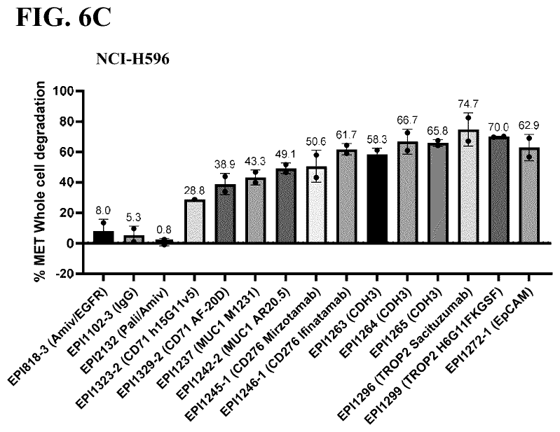

For the AlphaLISA, 12 bispecifics which bound to 6 unique degrader proteins were screened using this assay (FIG. 6A-C). For previously identified degrader proteins, multiple binding domains that bind to different epitopes were tested. Amivantamab (EPI818-2), Emibetuzumab (EPI1444-1), an IgG1 isotype control (RG196-1), and cMET×RSV (EPI1088-2; EPI2132) were tested as an SoC control, a cMET only control, a negative control, and a single-arm control, respectively, with cMET×RSV used as a baseline for comparison. The first binding arm for bispecifics in this example are listed in Table 1 or Table 4. The second binding arm for the bispecifics in this example are listed in Table 7. The control arms and antibodies are also listed in Table 10, Table 11, and Table 12.

In the AlphaLISA assay, NCI-H1975 cells, NCI-H596 cells, or Hs746T cells were seeded in 384-well plate in reduced serum media. After approximately 16 hours of culture, a single concentration of antibodies was added to cells in reduced serum media and treated for 48 hours. Media was removed and cells were lysed. AlphaLISA acceptor beads and biotinylated antibodies were added to the lysate and incubated for 1 hour at room temperature. AlphaLISA donor beads were added to the lysate and incubated for 2 hours at room temperature. The plate was read on Perkin Elmer Envision to determine total cMET levels.

The results of the AlphaLISA screen identified degrader protein groups and specific molecular epitopes that, when paired with cMET in a bispecific antibody format, demonstrated improved whole cell degradation of cMET as compared to Palivizumab×cMET, a bispecific pairing cMET with a non-targeting control arm. The degrader proteins identified as efficacious in inducing cMET degradation include CD71. MUC1. CD276. CDH3. TROP2 and EpCAM. The results also shows that cMET degradation was more robust in NCI-H1975 cells than in Ns746T cells.

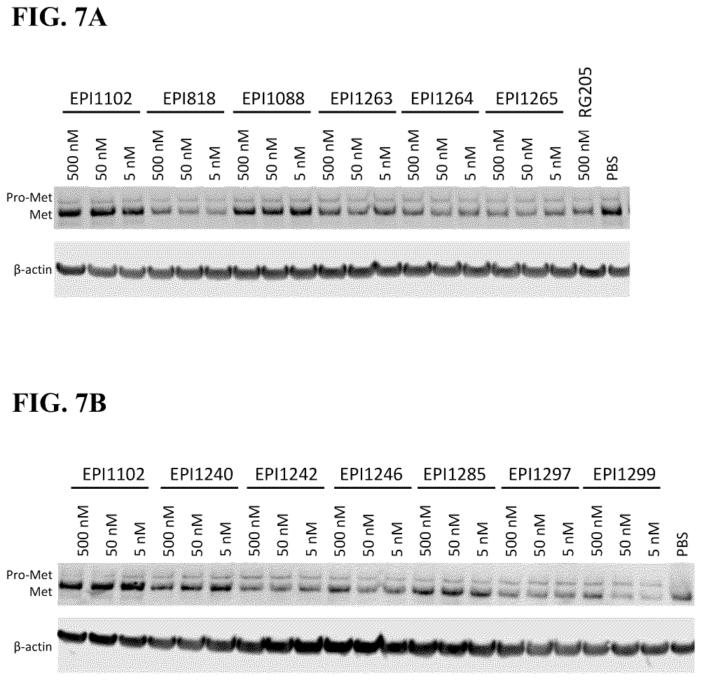

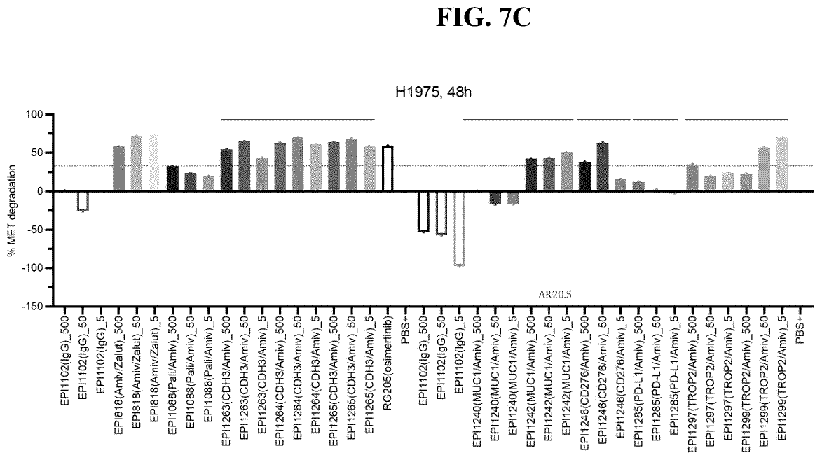

Additionally. 9 bispecific antibodies which bound to 5 different degrader proteins were assessed for whole cell degradation using western blot (FIGS. 7A-C). Amivantamab (EPI818-2). Emibetuzumab (EPI1444-1), an IgG1 isotype control (RG196-1), and cMET×RSV (EPI1088-2; EPI2132) were tested as an SoC control, a cMET only control, a negative control, and a single-arm control, respectively, with cMET×RSV used as a baseline for comparison. The first binding arm for bispecifics in this example are listed in Table 1 or Table 4. The second binding arm for the bispecifics in this example are listed in Table 7. The control arms and antibodies are also listed in Table 10. Table 11, and Table 12.

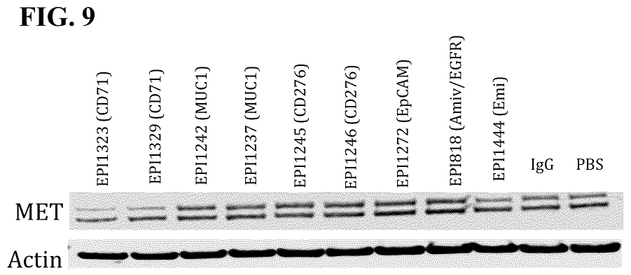

For the western blot. NCI-H1975 cells were seeded at a density of 4e5 cells in a 6 well tissue culture plate. After approximately 16 hours of culture, antibodies were added to cells at concentrations of 5, 50 or 500 nM in serum-starved media and treated for 48 hours. Media was removed and cells were lysed. Prepared samples were loaded onto a 4-12% BisTris gel and transferred to a PVDF membrane. The membrane was probed for cMET or the housekeeping gene β-actin (FIG. 7A-B). Data was quantified using Empiria studio and the percent degradation was normalized to β-actin and compared to PBS control (FIG. 7C). By western blot, many of bispecific antibodies demonstrated improved whole cell degradation of cMET as compared to cMET×RSV, a bispecific pairing cMET with a non-targeting control arm. Specifically, the degrader proteins identified as efficacious in inducing cMET degradation include MUC1, CD276, CDH3, and TROP2, validating the results of the AlphaLISA. Further validating the results of the AlphaLISA, this experiment was repeated using Hs746t cells, and resulted in no significant cMET degradation (FIG. 9).

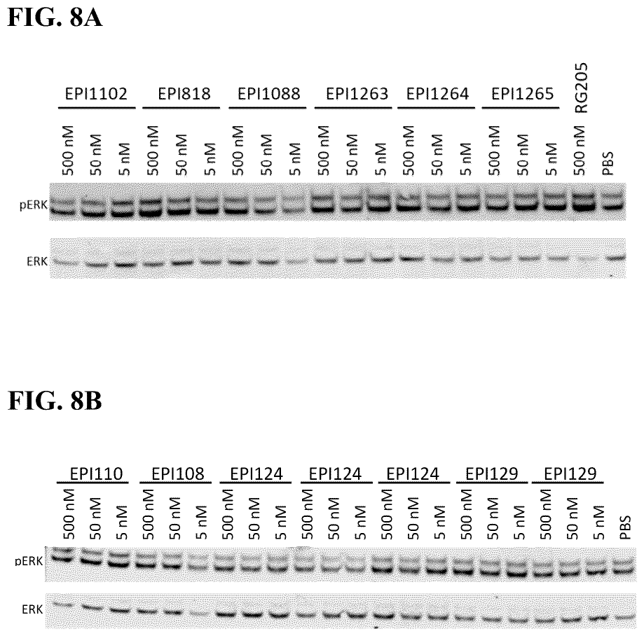

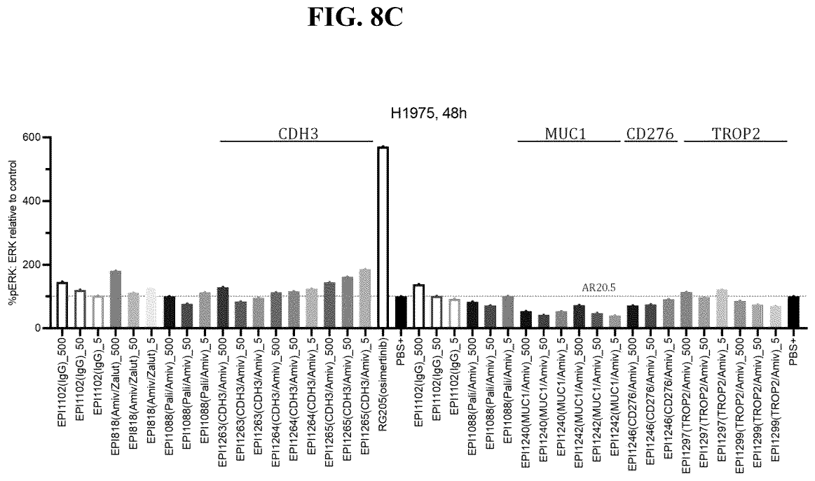

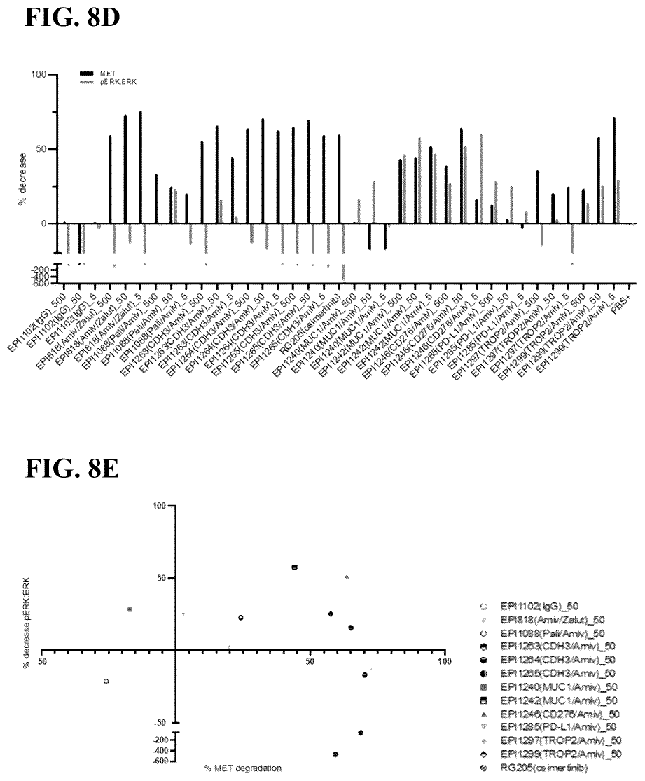

Subsequently, efficacious bispecific antibodies were tested for their ability to inhibit signaling events in NCI-H1975 cells. This was done by measuring the ratio of pERK and ERK using a western blot as described above (FIG. 8A-C). The results showed that many of the efficacious bispecific antibodies caused reduced levels of pERK, indicating the bispecific binder-induced degradation can inhibit signaling events downstream of the degradation target.

### Example 11—Reduced Dimerization for cMET-Targeting Bispecific Antibodies

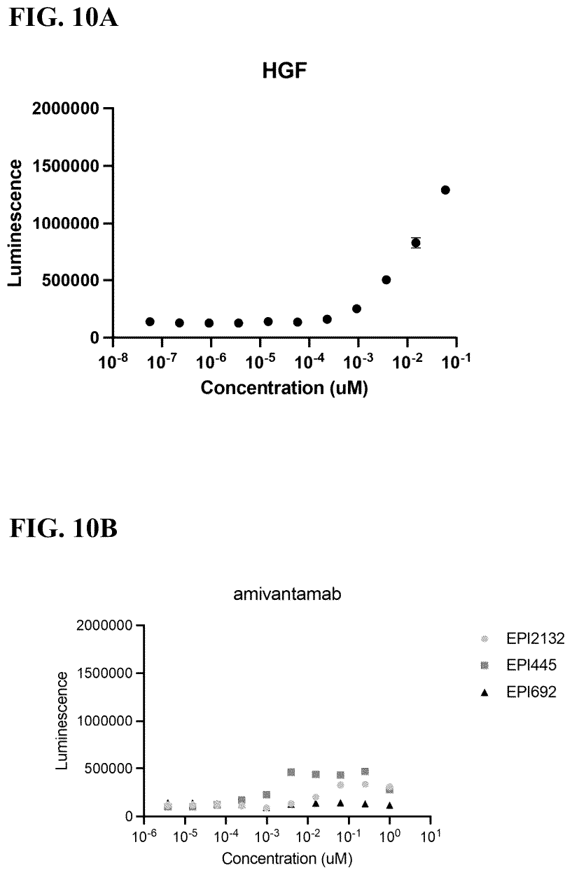

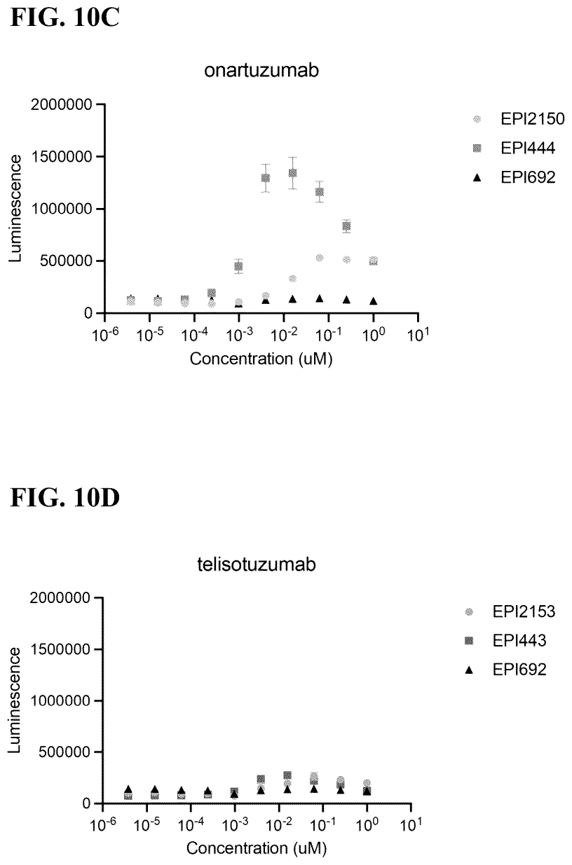

Current clinical cMET antibodies dimerize cMET and activate downstream signaling involved in oncogenicity. To determine the amount of cMET dimerization that occurs on the cell surface as a result of the bispecific antibodies, a dimerization assay was performed (FIG. 10A-D). Amivantamab (cMET×EGFR; EPI445) or monospecific antibodies specific to cMET (onartuzumab, EPI444; telisotuzumab, EPI443) were compared to bispecific cMET×RSV antibodies (EPI2150; EPI2153; EPI2132). Palivizumab hlgG1 (RSV) was used as an additional control. The first binding arm for bispecifics in this example are listed in Table 1 or Table 4. The second binding arm for the bispecifics in this example are listed in Table 7. The control arms and antibodies are also listed in Table 10, Table 11, and Table 12.

In the dimerization assay, the PathHunterR U2OS c-MET/c-MET dimerization cell line was used. This cell line uses enzyme fragment complementation (EFC) technology, where β-galactosidase (β-gal) is split into two fragments (termed ProLink and Enzyme Acceptor), to detect ligand-induced dimerization of c-MET receptors. These cells were engineered to overexpress c-MET fused to ProLink and c-MET fused to Enzyme Acceptor. Upon dimerization of c-MET, forced complementation of these fragments produces functional β-gal that can be used to produce a chemiluminescent signal after addition of substrate. Cells were cultured in 96 well plates. Antibodies or hepatocyte growth factor (HGF is c-MET's native ligand, positive control) were added. After 48 hours, media was removed, and flash detection reagent, containing a substrate for β-gal, was added to each well. Hydrolyzation of the substrate by β-gal results in the generation of a chemiluminescent signal. Following 1 hour incubation, luminescence was detected on a standard luminescence plate reader (Perkin Elmer).

Consistent with the literature, cMET antibodies that are currently in the clinic (e.g., onartuzumab) induce robust cMET dimerization at low concentrations. Comparatively, the bispecific antibodies, albeit cMET dimerizing, require higher concentrations and activate to a lesser extent. This data demonstrates that bispecific antibodies may have decreased signaling that results from dimerization than currently available monospecific antibodies.

### ADDITIONAL EMBODIMENTS

Embodiment 1: A method of degrading a cMET protein on a target cell, the method comprising: contacting the cMET protein and a membrane-associated internalizing protein on the target cell with a bispecific binding agent, wherein the contacting of the cMET protein and the membrane-associated internalizing protein with the bispecific binding agent leads to internalization and degradation of the cMET protein; and wherein the bispecific binding agent comprises: (a) a first binding domain that specifically binds to an extracellular epitope the membrane associated internalizing protein; and (b) a second binding domain that specifically binds to an extracellular epitope on the cMET protein; wherein the membrane associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228A, MUC5A, CD44, ITGB1, STn, KAAG1, DLK1, 5T4, SEZ6, ADAM9, I-Ag7, ENPP3, CD37, CD46, CD56, CD74, IGF1R, ROR1, CDH6, ROR2, GPR20, TM4SF1, B7-H4, ALPP, LY6E, CLDN18, LY6G6D, GPR56, and CD71.
Embodiment 2: The method of embodiment 1, wherein the membrane associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71.
Embodiment 3: The method of embodiment 1 or embodiment 2, wherein the membrane associated internalizing protein is CEACAM5.
Embodiment 4: The method of embodiment 1 or embodiment 2, wherein the membrane associated internalizing protein is CEACAM6.
Embodiment 5: The method of embodiment 1 or embodiment 2, wherein the membrane associated internalizing protein is HER3.
Embodiment 6: The method of embodiment 1 or embodiment 2, wherein the membrane associated internalizing protein is MUC1.
Embodiment 7: The method of embodiment 1 or embodiment 2, wherein the membrane associated internalizing protein is CD205.
Embodiment 8: The method of embodiment 1 or embodiment 2, wherein the membrane associated internalizing protein is CD166.
Embodiment 9: The method of embodiment 1 or embodiment 2, wherein the membrane associated internalizing protein is PRLR.
Embodiment 10: The method of embodiment 1 or embodiment 2, wherein the membrane associated internalizing protein is SLC34A2.
Embodiment 11: The method of embodiment 1 or embodiment 2, wherein the membrane associated internalizing protein is ITGB6.
Embodiment 12: The method of embodiment 1 or embodiment 2, wherein the membrane associated internalizing protein is LRRC15.
Embodiment 13: The method of embodiment 1 or embodiment 2, wherein the membrane associated internalizing protein is MUC16.
Embodiment 14: The method of any one of embodiments 1 to 13, wherein the bispecific binding agent comprises an antibody or portion thereof.
Embodiment 15: The method of any one of embodiments 1 to 13, wherein the bispecific binding agent comprises a bispecific antibody or portion thereof.
Embodiment 16: The method of any one of embodiments 1 to 13, wherein the bispecific binding agent comprises a knob and hole bispecific IgG.
Embodiment 17: The method of any one of embodiments 1 to 13, wherein the bispecific binding agent does not comprise an antibody-drug conjugate.
Embodiment 18: A bispecific binding agent comprising a bispecific antibody or antibody derivative, the bispecific binding agent comprising: a) a first binding domain that specifically binds to an extracellular epitope of a cMET protein of a target cell; and b) a second binding domain that specifically binds to an extracellular epitope of a membrane-associated internalizing protein on a target cell; wherein the membrane associated internalizing protein is selected from CD205, CD166, SLC34A2, ITGB6, LRRC15, and MUC16 SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, and CD71.
Embodiment 19: The bispecific binding agent of embodiment 18, wherein the membrane associated internalizing protein is CD205.
Embodiment 20: The bispecific binding agent of embodiment 18, wherein the membrane associated internalizing protein is CD166.
Embodiment 21: The bispecific binding agent of embodiment 18, wherein the membrane associated internalizing protein is SLC34A2.
Embodiment 22: The bispecific binding agent of embodiment 18, wherein membrane associated internalizing protein is ITGB6.
Embodiment 23: The bispecific binding agent of embodiment 18, wherein membrane associated internalizing protein is LRRC15.
Embodiment 24: The bispecific binding agent of embodiment 18, wherein the membrane associated internalizing protein is MUC16.
Embodiment 25: The bispecific binding agent of any one of embodiments 18 to 24, wherein the bispecific binding agent comprises a knob and hole bispecific IgG.
Embodiment 26: The bispecific binding agent of any one of embodiments 18 to 25, wherein the bispecific binding agent does not comprise an antibody-drug conjugate.
Embodiment 27: A pharmaceutical composition comprising a bispecific binding agent of agent of any one of embodiments 18 to 26 and a pharmaceutically acceptable excipient.
Embodiment 28: A method of treating cancer in a subject in need thereof, the method comprising administering to the subject a bispecific binding agent of any one of embodiments 18 to 26 or a pharmaceutical composition of embodiment 27.
Embodiment 29: A method of arresting growth of a target cell, the method comprising contacting the cell with a bispecific binding agent of any one of embodiments 18 to 26 or a pharmaceutical composition of embodiment 27.
Embodiment 30: The method of embodiment 29, wherein the cell is a cancer cell.

1. A method of degrading a target protein on a surface of a target cell, the method comprising:
contacting an endogenous internalizing receptor and the target protein on the surface of the target cell with an antibody, wherein the antibody comprises:
i. a first binding domain that specifically binds to the endogenous internalizing receptor, wherein the endogenous internalizing receptor is MUC1; and
ii. a second binding domain that specifically binds to the target protein, wherein the target protein is cMET.

2-255. (canceled)

256. The method of claim 1, wherein the endogenous internalizing receptor is recycled to the target cell surface following the internalization of the antibody.

257. The method of claim 1, wherein the endogenous internalizing receptor is degraded.

258. The method of claim 1, wherein the target cell is a cancer cell.

259. The method of claim 1, wherein the target cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and a head and neck cancer cell.

260. The method of claim 1, wherein expression of cMET on the target cell decreases following contact with the antibody, as compared to a control target cell that is not contacted with the antibody.

261. The method of claim 1, wherein expression of cMET on the target cell decreases by 50% or more following contact with the antibody relative to expression of cMET on a control target cell not contacted with the antibody.

262. The method of claim 1, wherein expression of cMET on the target cell decreases by 50% or more following contact with the antibody relative to expression of cMET on a control target cell contacted with a monospecific cMET antibody.

263. The method of claim 1, wherein cell surface removal of cMET on the target cell is at least 20% or more following contact with the antibody relative to cMET on a control target cell not contacted with the antibody.

264. The method of claim 1, wherein cell surface removal of cMET on the target cell is at least 20% or more following contact with the antibody relative to cMET on a control target cell contacted with a monospecific cMET antibody.

265. The method of claim 1, wherein internalization of cMET in the target cell is at least 20% or more following contact with the antibody relative to internalizing of cMET in a control target cell not contacted with the antibody or contacted with a monospecific cMET antibody.

266. The method of claim 1, wherein degradation of cMET in the target cell is at least 20% or more following contact with the antibody relative to degradation of cMET in a control target cell not contacted with the antibody or contacted with a monospecific cMET antibody.

267. The method of claim 1, wherein the antibody is a bispecific antibody.

268. An antibody comprising:
a) a first binding domain that specifically binds to an endogenous internalizing receptor, wherein the endogenous internalizing receptor MUC1; and
b) a second binding domain that specifically binds to a target protein, wherein the target protein is cMET.

269. The antibody of claim 268, wherein the antibody is a bispecific antibody.

270. A pharmaceutical composition comprising the antibody of claim 268.

271. A method comprising:
selecting a subject with tumor expressing cMET and an endogenous internalizing receptor, wherein the endogenous internalizing receptor is MUC1; and
administering to said subject the antibody of claim 268.

272. The method of claim 271, wherein the volume of the tumor decreases by 20% or more after administration of said antibody relative to the volume of a tumor not contacted with the antibody.

273. The method of claim 271, wherein the volume of the tumor is 80% or less after administration of said antibody relative to the volume of a tumor not contacted with the antibody.

274. A kit comprising an antibody comprising:
a) a first binding domain that specifically binds to endogenous internalizing receptor, wherein the endogenous internalizing receptor is MUC1; and
b) a second binding domain that specifically binds to a target protein, wherein the target protein is cMET.
